SEQUENCE LISTING (38 sequences)

SEQ ID NO: 1 - protein, 1810 aa, Homo sapiens
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGVFFLTLSLLGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVQQGYRGSEENSLEEDKGLRPMTPRSLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 2 - DNA, 5433 bp, Homo sapiens
  source 1..5433: mol_type = unassigned DNA
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggagttttcttccttactctttccctcctaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtacagcagggctacagagggtcagaggaaaacagtttggaggaagacaaagggttaagacccatgactcctcgcagcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatga

SEQ ID NO: 3 - DNA, 5433 bp, synthetic construct
  source 1..5433: mol_type = other DNA
  misc_feature 1..5433: note = Codon optimized human Stereocilin cDNA
atggctctgtctctgtggcctctgctgctgctcctgttgctgcttctgctgctcagcttcgccgtgacactggctccaacaggaccccactctctggatcctggcctgagctttctgaagtccctgctgagcaccctggatcaggctcctcagggcagcctgagcagatccagattcttcaccttcctggccaacatcagcagcagcttcgagcctggcagaatgggagaaggacctgtgggagaacctcctccactgcaacctccagctctgcggctgcacgattttctggtcacactgagaggcagccccgactgggaacctatgctgggactgctgggagatatgctggccctgctcggacaagagcagacccctagagatttcctggtgcatcaggctggcgtgctcggaggactggttgaagttctgcttggagcactggtgcctggcggacctcctacacctacaagacctccatgcaccagagatggccccagcgattgtgtgctggctgctgattggctgcctagcctgctcctgcttctggaaggcacaagatggcaggccctggtgcaggttcagccttctgtggatcctaccaatgccaccggcctggatggaagagaagccgctccacactttctgcagggactgcttggactgctcacacctactggcgagctgggctctaaagaagccctttggggaggcctgctgagaacagttggagcccctctgtacgccgcctttcaagagggactcctgagagtgacacacagcctgcaggacgaggtgttcagcatcctgggacagccagagcctgacaccaatggacagtgtcagggcgtgttctttctgaccctgagcctgctgggcaacctgcagcaactgcttctgtggggcgtcagacacaacctgagctgggatgtgcaggcactgggctttctgtctggaagccctccacctccaccagcactgctgcattgtctgtctactggcgtgccactgcctagagcctctcagcctagcgctcacatcagccccagacagagaagggccatcaccgtggaagctctgtgcgagaatcacctgggacctgctcctccatacagcatcagcaacttctccatccatctgctgtgtcagcacaccaagcctgccacacctcagcctcatcctagcaccacagccatctgtcagaccgccgtttggtacgccgtttcttgggctcctggtgctcaaggatggctgcaggcctgtcacgatcagttccccgacgagttcctggacgccatctgcagcaatctgagcttctctgccctgtccggcagcaatcggagactggtcaaaagactgtgcgccggactgctgcctcctccaacatcttgtcctgagggactgcctcctgtgcctctgactcccgatatcttttggggctgcttcctggaaaacgagacactgtgggccgagagactgtgtggcgaagcctctctgcaagccgtgcctccatctaatcaggcctgggtgcagcacgtttgtcagggccctacacctgacgtgacagcctctcctccttgtcacatcggaccttgcggcgagagatgtcctgatggcggcagctttctcgtgatggtctgcgccaacgacactatgtacgaggtgctggtgcccttctggccttggctggctggccagtgtagaatctccagaggcggcaacgatacctgtttcctggaaggactcctgggaccactgcttccatctctgcctccgcttggaccctctcctctgtgtcttacccctggacctttcctgctggggatgctgtctcagctgcctagatgccagtctagcgtgccagctctggcccatccaaccagactgcactatctgttgcggctgctgaccttcctccttggacctggcgctggcggagctgaagctcaaggcatgcttggcagagccctgctgctgtcatccctgcctgacaactgcagcttctgggacgccttcagacctgaaggacgcagaagcgtgctgaggaccatcggcgagtacctggaacaggatgaggaacagcctacaccaagcggcttcgaacccaccgtgaatcctagcagcggcatctccaagatggaactgctggcctgcttcagccccgtgctgtgggatctgctgcagagggaaaaaagcgtgtgggccctgcagatcctggtccaggcctatctgcacatgcctccagagaacctgcaacagctggtgctgtctgccgagagagaagctgcccagggattcctgactctgatgctgcagggaaagctgcagggcaaactccaggtgccacctagcgaagaacaggcccttggcagactgacagcactcctgctccagagataccccagactgacctctcagctgtttatcgatctgagccctctgatcccattcctggccgtgtccgacctgatgagattccctcctagtctgctggccaacgactccgtgcagcagggatacagaggcagcgaggaaaacagcctggaagaggacaaaggcctgcggcctatgacacctagatctctggccgccatccgggattacagccctggaatgaggcccgagcagaaagaggccctggctaagagactgctggctcccgagctgtttggcgaagttccagcctggcctcaagaactgctgtgggctgttctgcccctgctgccacatctgcctctggaaaacttcctgcagctgagcccacaccagattcaggccctcgaggattcttggcctgccgctggactcggacctggacatgctagacacgtgctgagaagcctggtcaaccagagcgttcaggacggcgaggaacaagtgcggagacttggacccctggcctgttttctgagccccgaggaactgcagagtctggtgcctctgtctgaccctacaggccctgttgaaaggggcctgctggaatgtgccgccaatggaacactgagccctgagggcagagtggcctatgaacttctgggagtgctgagatctagcggcggagccgttctgtcccctagggaacttagagtgtgggcacccctgtttagccagctgggcctgagattcctgcaagagctgtctgagccacagctgagggctatgctgcctgtgctccagggcacatctgtgacaccagctcaagccgtcctgctgttgggcagactgctccctagacacgatctgtccctggaagaactgtgcagcctgcacttgctgctgcctggactgtctcctcagacactgcaggccattcctcggagagtgcttgtgggagcctgtagctgtctggcccctgagctgtctagactgagcgcctgtcaaacagccgctctcctgcagaccttcagagtgaaggatggcgtgaagaacatgggcaccacaggcgctggacctgccgtgtgtattcctggacagcagcccattcctaccacctggccagattgtctgctcccactgctgcccctgaaactgctgcagctggattctctggctctgctggctaaccggcggagatattgggaactgccttggagcgaacagcaggcacagttcctgtggaagaagatgcaggtccccaccaatctgacactgcggaatctgcaggctctcggcacacttgctggcggaatgagctgcgagttcctccagcagatcaacagcatggtggactttctggaagtggtgcacatgatctaccagctgccaaccagagtgcggggaagcctgagagcttgtatttgggctgaactgcagcggcggatggccatgcctgaacctgaatggacaacagtgggccccgagctgaacggcctggactctaaacttctgctggatctccccatccagctgatggacagactgagcaacgagagcatcatgctggtggtggaactggtgcagagagccccagaacagctgctggcactgacacctctgcatcaagctgctctggccgaacgggcccttcagaatctggctcccaaagaaacccctgtgtccggggaagtgctggaaacactgggacctcttgtgggcttcctgggcaccgagtctaccagacagattcctctccagatcctgctgtcccacctgagccagctccagggattttgtctgggcgagacattcgccaccgaactcggatggttgctgctccaagagagcgtgctgggaaagcccgaactgtggtcacaggacgaagtggaacaggccggcagactggtgtttaccctgtctaccgaggccatcagtctgatccccagagaagcactgggccctgaaacactcgagaggctgctggaaaagcagcagtcttgggaacagagcagagtgggccagctgtgtagagaacctcagctggccgctaaaaaggccgcactggttgctggcgttgtcagacctgctgccgaggatctgccagagccagtgcctaattgtgccgatgtgcggggcacatttcctgccgcttggagcgctacacagatcgccgaaatggaactgagcgacttcgaggactgtctgactctgtttgccggcgatcctggactgggaccagaagaactgagagccgccatgggcaaagccaagcaactttggggacctccaagaggcttcagacccgaacagattctgcagctcggccgcctgcttatcggactgggcgatagagagctgcaagaactgatcctggtggactggggcgtgctgtctactctgggacaaatcgacggctggtccaccacacagctgcggattgtggtgtccagcttcctgaggcagtctggcagacatgtgtctcacctggacttcgtgcacctgactgccctgggctacacactgtgtggactgcggcctgaggaacttcagcacatcagctcttgggagttcagccaggcagccctgtttctgggaaccctgcatctgcagtgctccgaagaacaactggaagtgctcgcccatctgctcgtgctgccaggcggatttggccccatctctaattggggacccgagatcttcaccgagatcggcaccattgccgctggcatccctgatctggccctgtctgcactgctgagaggacagatccagggcgtgacaccactggccattagcgtgatccctccaccaaagttcgccgtggtgtttagccccattcagctgtccagcctgacatctgcccaagccgtggctgtgacccctgaacagatggcatttctgtctcccgagcagcggagagctgttgcttgggctcaacacgagggcaaagagagtcctgagcaacagggaagaagcaccgcttggggactgcaggattggtccagaccttcttggtcactggtgctgaccatcagctttctgggccacctcctgtga

SEQ ID NO: 4 - DNA, 19238 bp, Homo sapiens
  source 1..19238: mol_type = unassigned DNA
gccctgccctcacctggctatcccacacaggtgagaataaccagaactcacctccggtaccagtgttcacttggaaacatggctctcagcctctggcccctgctgctgctgctgctgctgctgctgctgctgtcctttgcaggtaagaagaacagtgagcagaactggggatgaggaggagggtggctggaaaaagactttaagaatatggaggtgaacctgttagatagaaggacaaaggagagaggcagagacttgtgcaaaagggaaaaatgagggttaagaaaagcaggccaagacttactgtaggccagtgaaaggggttcagctcaccatcccctcacctcatctttagatccaggtagggaactgtgctcaggggcagggttgagtttgggctctgtgttcctctccttcagtgacctctggtttctctccttacagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtgagtgtggccagggctgggactgggatgtggcagggcaaggaaagtgaaattggggtagttttcttccttactctttccctcctaggtaaccttcaacagctgctcttatggtaagtaacaggagaccagttctgagggattgggcctggaaaatctggaggtgaagagctgaagacctcagcctctagagaggaaaactgatgggaggagtgtagtttagtggttttggggtgtgactgtctgggttggtgtcccagctccacctcttcctagccatatgaccttgagcaggttacatagtctttctatacctcagtttccccatttataaaatgagaatgataatattagttaccacagagttgttgcacccggttaaatgagttgatactgtgtatgcaaacgacttaaaaccgtgctggcacatagcgcttaataatgttagctagtaaagatgggatttggaaaataaggacacagctggattcctctacccccttactacttcagtacaacaatgccagacagtagttagacatattgagttgctgagcagatttcctaacatgaggcccgctgagggttgtgtttaagctatctaaaagcatacgaagaaaggagacagaagggggccaggtggacagaaagaattccaactggggcttctcctaggtgattttggaccttggcagggcagctttctcttttttgccccgttgcagcatttcaaccagtaacgcctaaactctcagggacctcgcttgtagaaaagcctatgcttgccatgccccttgagggctctgagtcagggtcagaatcttcagctggaggaaatgtgaactgaccagatcctgcctgctcctccctctgcacccaggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtgtgagtgctctgccagagggaaagctcctagaacagtgagaaggccctccaggggaattcctcgaatactcagaggcagtagtgtggggtagtagttgaagcacacagctctagagtcagacaggcttggattcatatcttggttctgtgaccagccttgaatgagttatttaacttctctgagcaatatttttctcgtctcatttataaactagggatgataatggtatatgagataatacatgctgtgggcttagcacagtgcatgatacacaaacatgcaataaatattaccttgttattcttttgggctctttgactctctcactttctgcaccagaaagaaaaaggatcaagttagaggactctaaatttttcccctagagagtgagaattggaggctggcagaatacaggaagataaggtaggaatgagaaagattcagggacactaccaatcagaagactttggttctaggttcaactgtgccacaaattagtgtgatcttaggcaagcaatttcatttagtttttctgggcttcagtttttagtctgtagaatggaggggtgagaatatgttaaacaccataattaattcactgagtgcctattatatgcaaggcactttgctaggttctgtaggatatataaagatttcttactccatgttggggccacctttttcaaaccctgggcccagtaaaatggaattagatagtctcatagtatttggttcaggtctacaagtattaattgagccaactatggacctggcatgggagagggtacaagagaaattagagatatgatcccggacctaaaagagcttaatatctgaagaatcacacttgagatgatggacaagcatcccagcaagtggagctggaatgcctgggggagctgcaggagagacagagaagacagctctgttggcatattgtctttcttcccaccagcctgtgctgtgggatctgctccagagggaaaagagtgtttgggccctgcagattctagtgcaggtaacaggtggagggcacatgggtgggctgggtgacagccatggctggaggtccctgccccgtgaggtgaggccatacccaccatgacctcctattcgcaggcgtacctgcatatgcccccagaaaacctccagcagctggtgctttcagcagagagggaggctgcacagggcttcctgacactcatgctgcaggggaagctgcaggggaagctgcaggtgagcactgagaaaggggagcaagggcacctggagcctagtgttcagagggcttgctttagtgggaggaggaactccagagaggaaatggcagggatactgagcatctccagaggcagaatccattcctgtgcccctacaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtgtaaggttcttgcactactcctcctgctcctgtcacggtcaggccaaccgcatccacctggagcagccccttccggagctcctctctgtttttttctttcatgccagataggcaatgtgccaacatcgtagcaaggtttgagagaggcacatctcacgcctgagtgtgaaaacccaatcattatgctaatgaactacaaaaggatcagagagctcctctctattaaaaccagggagaggatgggcgtggtggctcatgcctgtaatcccagcacgttgggagcccgaggcaggtggatcactaggtccgcctagtgagttcgagaccagcctggccaatatggtgaaaccccgtctctattaaactacaaaaattagccaggcatggtggtgggcgcttgtagtcccagttactctggaggctgaggcaggaggatagcttgaacctgggaggcagaggttgcagtgaaccaagatcgtgccactgcactccagcctgggtgacagagcgagactccgtcttaaaaaaaacaaaaaacaaaacaaaacaaaaaaacagggagagtctccttcctatctagacagcagggctacagagggtcagaggaaaacagtttggaggaagacaaagggttaagacccatgactcctcgcagcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccaggtatgagaatcatcttctttacttgactggcccatcttctgctagtggggacaaagagtcaatggcatgtctctcagtggcccctccctgcaagaaccctatagtgaccccagtgcgagctaaccttccccatctcagatccaggccctggaggatagctggccagcagcaggtctggggccagggcatgcccgccatgtgctgcgcagcctggtaaaccagagtgtccaggatggtgaggagcaggtacgcaggtgagttgttgtgggatcagtaaccaaggcaagagtggaagaggtagagagaggaaggcacagctgtcacgctgggtcggtgttctaggaagaaaggggcaagagagtaggcagtggcctcaggcagcatagagttccaggagagaggtctatagatggtgcccctgtgtagtggtgtagtgtcagagtgcccagtgtatgtacccataccatctgctgccaggcctgccttagtgctagtcttggggaccacacaaaggtcagcttcatgccctcctcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtgagcccctcagcacaagcctacaagactttaggcttcccctgggtctgtgtggatggctttcccattgtgtcaacttgagcacagtggtgccagcccccatcccacttttgcaacctccattccttactccatggccattcttacctgttaccacctcttcctggcccttctctatctggtctgtagcaccccaaacataccctttgccattttgaacctaatctactccagtccaatccctagttccaaaccctagcccaggccctgggaaattcagatgtgggattagagaggaagttcaaggttcatctgtcttttctctccagtcctaaaccttctttggttacaggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggtttgcctgtctcactccctggcatgtaccctccatccccgcttgagccccagtcaagagaatcccattcagggataaaagcagcccctcctttccctgggtgaacagtagaggtaaactctgtctgcaggaggacgccttcattccctttcctcagatcaagaagggacctgagtcactgaggatggttactagggatggttaagaggcagcgggaagttttggagggtttgccttaggaacccacttaggacctggctgctgggtcctgagagctgttgttttcggtcccatcccaacacaggctgtcctgctgcttggacggctccttcctaggcacgatgtgagtagcagcaacttctcagcctcccgccagaggtctctatcctcttttaacctggctcctgcatctgcccctcctctctctccgctcccctcatacttactgccttgctgcattgtgattgttgtcttccccaacacccttcccttcttcttcaggcctcttgtctctcttgctctttagctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggtatgagagtggcaaggaggatgagataatcagggataccggctctttctggttgggaggaaggcatcttccctgaggccagggaaggcctttcatacctccccacttacacacacacacacacacacacacacacacacacacacaaccaattctcatgcaggttaaagatggtgttaaaaatatgggtacaacaggtgctggtccagctgtgtgtatccctggtcaggtaagtgtgagatctcccaactgagctcctctccccattctggggcagtttcatatggctggtgctacctcccacactaccctgcagtggccctgagagttctggttagctctgtgcccattagcagccctccccagtgccagatgcaggacagcatgatccactcacattgtcctagactaatgtcaaagctggaagggcctgagaaatcttccaggccacccaccctgctttcagatgaaaagaccaaggctgggagaagctaagggactttgtttgcctggtgcctaactagcagcaacacttgaccacagcagcctgcagtgtgaggctcttaggcgtttattgctacagtggcaaatgccattccacttctgtcctagctttggtccctttccacccccatggttccttttctctgagtgctaagtacagactctctcacctatcactacactgctatacccatcaccgccagcagcctattcccaccacctggccagactgcctgcttcccctgctcccattaaagctgctacaactggattccttggctcttctggcaaatcgaagacgctactgggagctgccctggtctgagcagcaggtaattctccccacttaatttcagaacttcctccctcaatgtagtctaccttctttacctatcccttagccctatttggccagcttatccctactatcctttatttgattgtttgagatacagtctcactctgttgcccaggctgcagtgcagtggcatgatcagagttcgctgtaacctcaaactcctgagctcaggcaatctttctgcctcagcctcctgaatagctaggacgacaggtggttaccaccatgcctggctaatttttaaattttttttttgttttttgagatgaagtcttgctctgtcacccaggcttgagtacagtggcacaagcttggctcactgcaacctctgtctcccgggttcaagcgattctcctgcctcagcctcccgagtagctgggactacaggcactccccacaatgcctggctaattttttttttgttttagtagagacagggtttcaccatattggccaggctggtctcgaactgctgaccttgtgatctgcctgcctctgcctctcaaagtgctgggattacaggtgtgagccaccatgcccggccaatttttaaattttttgtagagacagacaatacaaaaatgtggacactatgtggagacactatgttgaggtactatgctgtccagattggtcttgaactcctggcctcaagcaatcctcctgccttggcctcccaaagtgctgggattacagacctgagccactgcacccagccccctagtatctcttataatgtgacttgcttttctttttctttctccttcccttttctttcatttctttctcactctcgagagaagagtgggcatctgggagagtgggaggctggtgggtcccacagagtgaggaggcaggactgggtccaaggcagtcctgcctctccactctagggggtatccttggacagtgtctcttctgggaaggggctcgtctttctttctcttgtaggcacagtttctctggaagaagatgcaagtacccaccaaccttaccctcaggaatctgcagtgagtaacttgtgttgagcagtgcgctgaattcgaccaacatttttttgagtgcttactatgtgccaggcaccatgtgatatggaatgggggatatagggatgaatgatgcatagtccctgcctcgtggacgttctcctagcacctccctttgccctcctttccttccacagtgccatgcctatcctgactagagccaaaggactcagaaaacctggattcaggttccagtcctgtcacctacttgtcctcttgggcaagtcatttaacgtccctgtgtcagttttcccttctttaaatgagaattacaatggcaccagcctcataggtagttactgtgaagattaaatgaggtaggtcatgtaagatatttaacacagtgtttggtccattgtaaagtcccagtagtcatttgctactgttagtttacttcaggatgacttcagaggcactggccaagcaagaataaataggaataagaaggtatcactttacttacacccacattagaagaacaatgggcttcagaatctttttttttttttttttttcgagacagtcttgctctgttgcccaggctggagtgcagtggcgcgatttcggctcactgcaacctctgcctcccaggttcaagcgattctcctgtctcagcctctggagtagctgggattacaggaatgtgccaccatacccagctaatttttgtatttttagtagagatggggtttcaccattttggccaggctggtctcaaactcctgacctcaggtgatccacccgcctcagcctcccaagggcttcagaatctaagacatggctctagtttcagtttaccacatttctagcagaatgatgttgggaatgtcacctgacttccataaatccttattttctcctctgataaacagcagtgatgttatggggagctgatgagatatctatgtaaaaacatttctcaaaccataaattacggtggatgaacatctgtacttgtgttgagagtactgatatcaaggagcaaacaggctgttgtatgtgttgaatgagcctctccccactcacacacccacagggctctgggcaccctggcaggaggcatgtcctgtgagtttctgcagcagatcaactccatggtagacttccttgaagtggtgcacatgatctatcagctgcccactagagttcgagggagcctggtgagagggggtgcctggactttagtgggagcagggaggctgggaccctaggtatagaacccagctcctatgttctgctctggcctcacactgcttccctacagagggcctgtatctgggcagagctacagcggaggatggcaatgccagaaccagaatggacaactgtagggccagaactgaacgggctggatagcaagctactcctggacttaccgtaagtactgcagctagagatattggcccctcagaaagctcaatctggggtgaagatctgcccttagggaatgccctggaggaggtagtttttctgtctggtagttccctgacataatttatagcccaaagcagaggattttattcaaagttgctctatgtattgactggttcccagaatatgctccagcacagggcagctgagggtggcaacactgtattgaagcctgccaagtaatcttacaataacctagtccacattaattgagattgagacagagcatctgaagtgagggaggcaatgctccaaatctgccccagaggattgtagtttgctcagggcactgtgttcttagtgcattcagaggagtagatcgagagaaaaatatatgaaaaatgtgataaataccttcaaatacctgaggggctatcaagtagaaattagattgtcatatttatgagtggccccattgggcaagactaagagtagttaacggagatcagatttttacatagtataagaaaaactaaggtagtgagttcctggtccttggagctgttcgagcctaagccagatggccccatggcaggaatgttgtagagcacgttcatatacaggttgtgggaagaaaaggctataggaacccaaggctcctccctacccatggagaaatttattagtatgttactcatatgctgcttttctcattttacccctaccaccaccccgttgccatccgcactgtaagtcaggataggaaaatgctggtgttacagtcttcctggggaatatggagctgaagtggagtaaaagcagttgacttcattcctacttttttcttttttttctttttttttttttttgagacagagttttgctgtgtcaccaaggctggagtgcagtgacgtgatctcggctcactgcaacctccatcttccaggttcaagcaattctcctgcctcagtctcccgagtagctgggactgtaggtgtgcaccaccatgccaggctaatttttgtatttgttgtagggacgagctttcaccatgttggccaggctggtcttgaactcctggcttcaagtgatctgcccacctcggcttcccaacattcttatatttttataggcctttccacagatttcagctcttgtatgacttagcccagttccagaactggtaatcctaggtagggtacaggttatcacctctgatttcgggtaaaagggatttatttatttatttgtttatttatttatatttttgagacagagtctcgctctgtcacccaggctggagtgcaatggtgccatctcggctcactgcaacctctccctctggggttcaagcaattctcctgcctcagcctgctgagtagctgggattacaggcgcgtgccaccacacccggctaatttttgcatttttagtagagacggggtttcaccatgttgctcagggtggtctcgaatttctgaccctgtgatctgcctgcctcggcctcccaaagtgctgggattacaggcatgagccactgcgtccggcctgtttttacttttttttaatgccattcagatctgtttaaatatgtgggttctgtgagataatttagaatcccaaggttacagatgaggtgaaagatcctagaccatgcatcaaaaaacttgagtttctcatttgtgaaagaaggataagagaaacacctattttgtctgggtgcagtggctcatgcctataatcccagcatttggggaggccaaggtgggtggatcacggaggtcaggtgttcaagaccagactggccaacatggcaaaacaccatctctactaaaaatacaaaagttagctgggcgtggtggcacgtgcgtgtaattccagctattcgggaggctgaggcacgagaattgcttgaacctgggaggtgcgggttgcagtgaactgagatcgcagcaccactgtgctccagcctgagtgatggagtgaggccaggtcttgttgtaggatcaaatgagataacacctgaaagaactttgtaaattgtatagcacgtacaaacaagaagggacctcttcacaagcagaggaagggtggtcctgtggaaaaaaacgggaattgggagtgagagacctcaacatttgatctctgtgaacctcagttttttaatctataaaatggggaaatgttaatggtacttaatatttggagcttttgagtccattagatcaggtaggattgttcgttattttttttttttaggaagactagaaatatgttgctccctttttctcccccactcaagcttgatggtgggaattggccctggagctgtttactatcagttcctgtccagcttcactaaatttggtctggggtcacatcttagctgcggactgtggggttttgtggtcccttctcgacttggcccagctccacctgaatcctgttgttgtcaaattgctgtaataggatccagttgatggacagactatccaatgaatccattatgttggtggtggagctggtgcaaagagctccagagcagctgctggcactgacccccctccaccaggcagccctggcagagagggcactacaaaacctggtaagagtccaccctaccagactcagatttgctgccctgggcaattcttgctcctcagacaatgctctctgactgtcccccaaccctctacttcttgctttcttgctgccaaacagattcctgtctacaaggcctggcccctgttttgcctctgggttctgttccttgataatatgcttcacgttacttgtccatacctcttggagtccgagaaatctcttggagtccacctctcagtctttctgcctgctcctatctgggctcattgcttaaggaagtgaacaaaggtagtgagcatcatagggtgctgagctgggagcaggagggagggaaggttagggggcttggtgtcttgatcaaggtgtctggtattctgagtcagaagtgcattgtccaagttctgatgctcttctccaggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggtatggaccttcgagaacttcagattctaactcattctatacccagtccctcagccaccatcatcagtggcagcctgttccatattcttaaggtcccctggagccctgtgtccgaaatcctagcatgtcctcttttccccttccttttcctcacagttccctcagctccccagcccccgattttcttcctgtccccaggaaaccagagttgtggagccaggatgaagtagagcaagctggacgcctagtattcactctgtctactgaggcaatttccttgatccccagggtgagatgaaggaagaagggaagggagtaaatgcatagaggggactggtgagctggttatggggacccgtggccaaagagggcaaaggatatgaagcctagatctggggggagactgcaaaacagagacaggactttggacttagagctatagcagcaggtcctgatctgtccagatctccccactctccttctaccttctcatgcaggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccaggtgaaactacccaaatacttatatgtccagcaggatgtacagggagtatcaaacggtctgggttctacatgtgctcttccctgggactgggttttctaatttataaagcaaagagtttagagggatgatcttcaagcctcttgtagttctagaattctgtagttctgggagtttgtaaactattaagttttcttttagcccagaacttccattttcctgctctctcgtgtctgctctagactcagctctagctcggctaagtgtggagctctctgctggggagatccctagaagctttgaaggagacattgtgaggctggagaactgggttcaaattcagtgctaccattaaatctctgaataacatcctcagtcttccatctataaaagtcttggcatctccaatcacttcttgttctattatctcctaagccctatacatattactctgtaatactcctttgatccctatttctcacagtgctctatcctccaaaggttggaagactcactctatctacagatatctctctgggcatattttattactgcgctgacctcctggccctgccttcccccttcagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacaggttagggatggagagccaactggggttggccatgaggaagctatttgggtgtgatgtaggacacaaagagaatggagagttggatgagaggtgggggaagcaagagatagaagagttagaagatttgggtcacaagtaggaggtgaagggagataaatattgaggaaagagagctagtataatgaatagagggacgaaagcagtggttaccaaattttaatgcatatcacgatcatcaagggaacagatttttttctttatttttttttctttcttaaaaaaataatggcatgcttcggctgggtgcagcggctcacgcctataatctcagaactttgggaggccaaggcgggcagatcacgaggtcaggagatcaagaccatcctgtctaacacggcgaaacacggtctctactaaaaatacaaaaaagttagccgggcatggtggtgcacacttgttgtcccagctacttgggaggctgaggcaggagaatggcgtgaacctgggagggggagcttgcagtgagccgaagtcaagccaatgcactccatcctgggtgacagagcaagactccatctcaaaaaaaaaaaaaaaaaaaaaaggcatgcttcatgaatttgcgtgttatccttgcacaggcgccatgcaaatctctgtatcattccaattttttggggtatgtgctgctgaactgagcatgggaacagtgccagtgccagattaccatgcttcactgacttaataaaaacctttggggaggctgggcgcagtgactcatgcctgtaatcacagcactttgggaggcggaggcaggtggattgcttgagcccaggagttagagaccagactgggcaacatggtgaaaccctgtctctactaaaaatagaaaaaacattagctgggtgtggcggcacatgcctgtaatcccagctactcaggaggctggggtaggagaatcccatgagtgcaggaggtggagggtgcaatgtgccaagatcgcaccactgccctccagcctgggtgtcagagcaagaccctgtctcataaattaaaaaataagcctctgggggaaagagtctagacatctgcatctcctttttttttttttttttttttttttgagacagagtctcactctgtcacccagcatccaggctggagtgcagtggtgtgatcttggctcactgtaacctctacatcctgggttcaaacgatcctcctgcctcagcctctcaagtagctgggactacaggtgcaccacacctggctaatttttgtatctttggtagagatggggtttcactatgttgcccaggatggtctcgaacttctgggctcaagcaatcctcccacctcagcctcccaaagtgctgggattacagctgttagccactgtgctgggccctaggcatctgttttaataagcgtctctgtgtctgatgcacataaaagtgtggaactcatggactagagttagtttgctcttcttttccactgattgtaatgtctttcaaaacaccttagaggaactgtaaggcaacggtctcattttatagtggaggaaactaaagaaaaggcaaatgatttacctagagttatacagctaagggcagaggcaagacttaaaacccagcagtatgactcccaatccactgcttttccactcacattgttcctgtctttctcctagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcaggtaacacttttcctcctccctacggcttcccaaacacccatcccacagacccagccctatagatcatctaaagcccaaggaatttttttcctgtgaccctacctggtccttctttctatcttttgttgataccccatactagtgaccttcaggactctgatttattcactctgaggccctggacacataatactgtctcctacctcttttcctggaggcttcctctttttctttccttttcttttctgagtcctcagccttccccatgactccttaggtcttaatagtaacagaatataacccagtaacacctatcacttccctgtccattaattctccataactttcctccttcccctcttctcccaccccccaccccagctccgcattgtggtctccagtttcctacggcagagtggtcggcatgtgagccacctggacttcgttcatctgacagcgctgggttatactctctgtggactgcggccagaggagctccagcacatcagcagttgggagttcaggtcatttgtgaaggggctgagggtggtggtgctgaggtaaaggtggacttactggggaaagaaggatcatgaaggtctggtcccatggaggaagggaactcatttgaagccatctcttcctttgtctcatgaccacagcccctttcactgaagccgaattcttcttccttccttcctactgttctacagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcaggtggggagctgggccactgctggtgcaagttggtttggtttctataccatgggtggactggatggaagactgccctgcaattcttaaggtgggggcctgagggtgtttaaataaggggctagagacatattggggaaggtctatgatagggcactttgggagtagttagagaaggtctataggtttgaagagagggaaggtcagtctaagacaatgtttggatgccacttgcttcaacagctgggatcccagacctggctctttcagcactgctgcggggacagatccagggcgttactcctcttgccatttctgtcatccctcctcctaaatttgctgtaagtattaatggactggggtgaccacaggagagccagggcccaatggggactacatgcatgcactgattcctacccctgccctcaggtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtgagttcccagctgcacagcttgatcctccatctcctgacccagaatcaaacccctaatttggtgctgtctggctcttagagtgcacccagggagatccctggagtgaaggagtctacaggcagagcgctaatttccaagtatcaatgctcctggagagctgagttgtgatattactcccattccctgtctattataggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaata

SEQ ID NO: 5 - DNA, 18884 bp, Homo sapiens
  source 1..18884: mol_type = unassigned DNA
gccctgccctcacctggctatcccacacaggtgagaataaccagaactcacctccggtaccagtgttcacttggaaacatggctctcagcctctggcccctgctgctgctgctgctgctgctgctgctgctgtcctttgcaggtaagaagaacagtgagcagaactggggatgaggaggagggtggctggaaaaagactttaagaatatggaggtgaacctgttagatagaaggacaaaggagagaggcagagacttgtgcaaaagggaaaaatgagggttaagaaaagcaggccaagacttactgtaggccagtgaaaggggttcagctcaccatcccctcacctcatctttagatccaggtagggaactgtgctcaggggcagggttgagtttgggctctgtgttcctctccttcagtgacctctggtttctctccttacagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcagcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtgagtgtggccagggctgggactgggatgtggcagggcaaggaaagtgaaattggggtagttttcttccttactctttccctcctaggtaaccttcaacagctgctcttatggtaagtaacaggagaccagttctgagggattgggcctggaaaatctggaggtgaagagctgaagacctcagcctctagagaggaaaactgatgggaggagtgtagtttagtggttttggggtgtgactgtctgggttggtgtcccagctccacctcttcctagccatatgaccttgagcaggttacatagtctttctatacctcagtttccccatttataaaatgagaatgataatattagttaccacagagttgttgcacccggttaaatgagttgatactgtgtatgcaaacgacttaaaaccgtgctggcacatagcgcttaataatgttagctagtaaagatgggatttggaaaataaggacacagctggattcctctacccccttactacttcagtacaacaatgccagacagtagttagacatattgagttgctgagcagatttcctaacatgaggcccgctgagggttgtgtttaagctatctaaaagcatatgaagaaaggagacagaagggggccaggtggacagaaagaattccaactggggcttctcctaggtgattttggaccttggcagggcagctttctcttttttgccccgttgcagcatttcaaccagtaacgcctaaactctcagggacctcgcttgtagaaaagcctatgcttgccatgccccttgagggctctgagtcagggtcagaatcttcagctggaggaaatgtgaactgaccagatcctgcctgctcctccctctgcacccaggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtgtgagtgctctgccagagggaaagctcctagaacagtgagaaggccctccaggggaattcctcgaatactcagaggcagtagtgtggggtagtagttgaagcacacagctctagagtcagacaggcttggattcatatcttggttctgtgaccagccttgaatgagttatttaacttctctgagcaatatttttctcgtctcatttataaactagggatgataatggtatatgagataatacatgctgtgggcttagcacagtgcatgatacacaaacatgcaataaatattaccttgttattcttttgggctctttgactctctcactttctgcaccagaaagaaaaaggatcaagttagaggactctaaatttttcccctagagagtgagaattggaggctggcagaatacaggaagataaggtaggaatgagaaagattcagggacactaccaatcagaagactttggttctaggttcaactgtgccacaaattagtgtgatcttaggcaagcaatttcatttagtttttctgggcttcagtttttagtctgtagaatggaggggtgagaatatgttaaacaccataattaattcactgagtgcctattatatgcaaggcactttgctaggttctgtaggatatataaagatttcttactccatgttggggccacctttttcaaaccctgggcccagtaaaatggaattagatagtctcatagtatttggttcaggtctacaagtattaattgagccaactatggacctggcatgggagagggtacaagagaaattagagatatgatcccggacctaaaagagcttaatatctgaagaatcacacttgagatgatggacaagcatcccagcaagtggagttggaatgcctgggggagctgcaggagagacagagaagacagctctgttggcatattgtctttcttcccaccagcctgtgctgtgggatctgctccagagggaaaagagtgtttgggccctgcagattctagtgcaggtaacaggtggagggcacatgggtgggctgggtgacagccatggctggaggtccctgccccgtgaggtgaggccatacccaccatgacctcctattcgcaggcgtacctgcatatgcccccagaaaacctccagcagctggtgctttcagcagagagggaggctgcacagggcttcctgacactcatgctgcaggggaagctgcaggggaagctgcaggtgagcactgagaaaggggagcaagggcacctggagcctagtgttcagagggcttgctttagtgggaggaggaactccagagaggaaatggcagggatactgagcatctccagaggcagaatccattcctgtgcccctacaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtgtaaggttcttgcactactcctcctgctcctgtcacggtcaggccaaccgcatccacctggagcagccccttccggagctcctctctgtttttttctttcatgccagataggcaatgtgccaacatcgtagcaaggtttgagagaggcacatctcacgcctgagtgtgaaaacccaatcattatgctaatgaactacaaaaggatcagagagctcctctctattaaaaccagggagaggatgggcgtggtggctcatgcctgtaatcccagcacgttgggagcccgaggcaggtggatcactaggtccgcctagtgagttcgagaccagcctggccaatatggtgaaaccctgtctctattaaactacaaaaattagccaggcatggtggtgggcgcttgtagtcccagttactctggaggctgaggcaggaggatagcttgaacctgggaggcagaggttgcagtgagccaagatcgtgccactgcactccagcctgggtgacagagcgagactccgtcttaaaaaaaacaaaaaacaaaacaaaacaaaaaaacagggagagtctccttcctatctagacagcagggctacagagggtcagaggaaaacagtttggaggaagacaaagggttaagacccatgactcctcgcagcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccaggtatgagaatcatcttctttacttgactggcccatcttctgctagtggggacaaagagtcaatggcatgtctctcagtggcccctccctgcaagaaccctatagtgaccccagtgcgagctaaccttccccatctcagatccaggccctggaggatagctggccagcagcaggtctggggccagggcatgcccgccatgtgctgcgcagcctggtaaaccagagtgtccaggatggtgaggagcaggtacgcaggtgagttgttgtgggatcagtaaccaaggcaagagtggaagaggtagagagaggaaggcacagctgtcacgctgggtcggtgttctaggaagaaaggggcaagagagtaggcagtggcctcaggcagcatagagttccaggagagaggtctatagatggtgcccctgtgtagtggtgtagtgtcagagtgcccagtgtatgtacccataccatctgctgccaggcctgccttagtgctagtcttggggaccacacaaaggtcagcttcatgccctcctcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtgagcccctcagcacaagcctacaagactttaggcttcccctgggtctgtgtggatggctttcccattgtgtcaacttgagcacagtggtgccagcccccatcccacttttgcaacctccattccttactccatggccattcttacctgttaccacctcttcctggcccttctctatctggtctgtagcaccccaaacataccctttgccattttgaacctaatctactccagtccaatccctagttccaaaccctagcccaggccctgggaaattcagatgtgggattagagaggaagttcaaggttcatctgtcttttctctccagtcctaaaccttctttggttacaggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggtttgcctgtctcactccctggcatgtaccctccatccccgcttgagccccagtcaagagaatcccattcagggataaaagcagcccctcctttccctgggtgaacagtagaggtaaactctgtctgcaggaggacgccttcattccctttcctcagatcaagaagggacctgagtcactgaggatggttactggggatggttaagaggcagcgggaagttttggagggtttgccttaggaacccacttaggacctggctgctgggtcctgagagctgttgttttcggtcccatcccaacacaggctgtcctgctgcttggacggctccttcctaggcacgatgtgagtagcagcaacttctcagcctcccgccagaggtctctatcctcttttaacctggctcctgcatctgcccctcctctctctccgctcccctcatacttactgccttgctgcattgtgattgttgtcttccccaacacccttcccttcttcttcaggcctcttgtctctcttgctctttagctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggtatgagagtggcaaggaggatgagataatcagggataccggctctttctggttgggaggaaggcatcttccctgaggccagggaaggcctttcatacctccccacttacacacacacacacacacacacacacacaaccaattctcatgcaggttaaagatggtgttaaaaatatgggtacaacaggtgctggtccagctgtgtgcatccctggtcaggtaagtgtgagatctcccaactgagctcctctccccattctggggcagtttcatatggctggtgctacctcccacactaccctgcagtggccctgagagttctggttagctctgtgcccattagcagccctccccagtgccagatgcaggacagcatgatccactcacattgtcctagactaatgtcaaagctggaagggcctgagaaatcttccaggccacccaccctgctttcagatgaaaagaccaaggctgggagaagctaagggactttgtttgcctggtgcctaactagcagcaacacttgaccacagcagcctgcagtgtgaggctcttaggcgtttattgctacagtggcaaatgccattccacttctgtcctagctttggtccctttccacccccatggttccttttctctgagtgctaagtacagactctctcacctatcactacactgctatacccatcaccgccagcagcctattcccaccacctggccagactgcctgcttcccctgctcccattaaagctgctacaactggattccttggctcttctggcaaatcgaagacgctactgggagctgccctggtctgagcagcaggtaattctccccacttaatttcagaacttcctccctcaatgtagtctaccttctttacctatcccttagccctatttggccagcttatccctactatcctttatttgattgtttgagatacagtctcactctgttgcccaggctgcagtgcagtggcatgatcagagttcgctgtaacctcaaactcatgagctcaggcaatctttctgcctcagcctcctgaatagctaggatgacaggtggttaccaccatgcctggctaatttttaaattttttttttgttttttgagatgaagtcttgctctgtcacccaggcttgagtacagtggcacaagcttggctcactgcaacctctgtctcccgggttcaagcgattctcctgcctcagcctcccgagtagctgggactacaggcactccccacaatgcccggctaattttttttttgttttagtagagacagggtttcaccatattggccaggctggtctcgaactgctgaccttgtgatctgcctgcctctgcctctcaaagtgctgggattacaggtgtgagccaccatgcccggccaatttttaaattttttgtagagacagacaatacaaaaatgtggacactatgtggagacactatgttgaggtactatgctgtccagattggtcttgaactcctggcctcaagcaatcctcctgccttggcctcccaaagtgctgggattacagacctgagccactgcacccagccccctagtatcccttataatgtgacttgcttttctttttctttctccttcccttttctttcatttctttctcactctcgagagaagagtgggcatctgggagagtgggaggctggtgggtcccacagagtgaggaggcaggactgggtccaaggcagtcctgcctctccactctagggggtatccttggacagtgtctcttctgggaaggggctcgtctttctttctcttgtaggcacagtttctctggaagaagatgcaagtacccaccaacctgaccctcaggaatctacagtgagtaacttgtgttgagcagtgcgctgaattcgaccaacatttttttgagtgcttactatgtgccaggcaccatatgatatggaatgggggatatagggatgaatgatgcatagtccctgcctcgtggacgttctcctagcacctccctttgccctcctttccttccacagtgccatgcctatcctgactagagccaaaggactcagaaaacctggattcaggttccagtcctgtcacctacttgtcctcttgggcaagtcatttaacgtccctgtgtcagttttcccttctttaaatgagaattacaatggcaccagcctcataggtagttactgtgaagattaaatgaggtaggtcatgtaagatatttaacacagtgtttggtccattgtaaagttccagtagtcatttgctactgttagtttacttcaggatgacttcagaggcactggccaagcaagaataaataggaataagaaggtatcactttacttatacccacattagaagaacaatgggcttcagaatctttttttttttttttttcgagacagtcttgctctgttgcccaggctggagtgcagtggcgcgatttcggctcactgcaacctctgcctcccaggttcaagcgattctcctgtctcagcctctggagtagctgggattacaggaatgtgccaccatacccagctaatttttgtatttttagtagagatggggtttcaccattttggccaggctggtctcaaactcctgacctcaggtgatccacccgcctcagcctcccaagggcttcagaatctaagacatgtctctagtttcagtttaccacatttctagcagaatgatgttgggaatgtcacctgacttccataaatccttattttctcctctgataaacagcagtgatgttatggggagctgatgagatatctatgtaaaaacatttctcaaaccataaattacggtggatgaacatctgtacttgtgttgagagtactgatatcaaggagcaaacaggctgttgtatgtgttgaatgagcctctccccactcacacacccacagggctctgggcaccctggcaggaggcatgtcctgtgagtttctgcagcagatcaactccatggtagacttccttgaagtggtgcacatgatctatcagctgcccactagagttcgagggagcctggtgagaggggatgcctggactttagtgggagcagggaggctgggaccctaggtatagaacccagctcctatgttctgctctggcctcactctgcttccctacagagggcctgtatctgggcagagctacagcggaggatggcaatgccagaaccagaatggacaactgtagggccagaactgaacgggctggatagcaagctactcctggacttaccgtaagtactgcagctagagatattggcccctcagaaagctcaatctggggtgaagatctgcccttagggaatgccctggaggaggtagtttttctgtctggtagttccctgacataatttatagcccaaagcagaggattttattcaaagttgctctatgtattgactggttcccagaatatgctccagcacagggcagctgagggtggcaacactgtattgaagcctgccaagtaatcttacaataacctagtccacattaattgagattgagacagagcatctgaagtgagggaggcaatgctccaaatctgccccagaggattgtagtttgctcagggcactgtgttcttagtgcattcagaggagtggatcgagagaaaaatatatgaaaaatgtgataaataccttcaaatacctgaggggctatcaagtagaaattagattgtcatatttatgagtggccccattgggcaagactaagagtagttaacggagatcagatttttacatagtataagaaaaactaaggtagtgagttcctggtccttggagctgttcgagcctaagccagatggccccatggcaggaatgttgtagagcacgttcatatacaggttgtgggaagaaaaggctataggaacccaaggctcctccctacccatggagaaatttattagtatgttactcatatgctgcttttctcattttacccctaccaccaccccgttgccatccgcactgtaagtcaggataggaaaatgctggtgttacagtcttcctggggaatatggagctgaagtggagtaaaagcagttgacttcattcttacttttttctttttctttttttttttttttttgagacagagttttgctgtgtcaccaaggctggagtgcagtgacgtgatctcggctcactgcaacctccatcttccaggttcaagcaattctcctgcctcagtctcccgagtagctgggactgtaggtgtgcaccaccatgccaggctaatttttgtatttgttgtagggacgagctttcaccatgttggccaggctggtcttgaactcctggcttcaagtgatctgcccacctcggcttcccaacattcttatatttttataggcctttccacagatttcagctcttgtatgacttagcccagttccagaactggtaatcctaggtagggtacaggttatcacctctgatttcgggtaaaagggatttatttatttatttgtttatttatttatatttttgagacagagtctcgctctgtcacccaggctggagtgcaatggtgccatctcggctcactgcaacctctccctctggggttcaagcacttctgcctcagcctcctgagtagctgggattacaggcgcgtgccaccacacccggctaatttttgcatttttagtagagacggggtttcaccatgttgctcagggtggtctcgaatttctgaccctgtgatctgcctgcctcggcctcccaaagtgctgggattacaggcatgagccactgcgtccggcctgtttttacttttttttaatgccattcagatctgtttaaatatgtgggttctgtgagataatttagaatcccaaggttacagatgaggtgaaagatcctagaccatgcatcaaaaaacttgagtttctcatttgtgaaagaaggataagagaaacacctattttgtctgggtgcagtggctcatgcctataatcccagcatttggggaggccaaggtgggtggatcacggaggtcaggtgttcaagaccagactggccaacatggcaaaacaccatctctactaaaaatacaaaagttagctgggcgtggtggcacgtgcgtgtaattccagctattcgggaggctgaggcacgagaattgcttgaacctgggaggtgcgggttgcagtgaactgagatcgcagcaccactgtgctccagcctgagtgatggagtgaggccaggtcttgttgtaggatcaaatgagataacacctgaaagaactttgtaaattgtatagcacgtacaaacaagaagggacctcttcacaagcagaggaagggtggtcctgtggaaaaaaacgggaattgggagtgagagacctcaacatttgatctctgtgaacctcagttttttaatctataaaatggggaaatgttaatggtacttaatatttggagcttttgagtccattagatcaggtaggattgttcgttattttttttttttaggaagactagaaatatgttgctccctttttctcccccactcaagcttgatggtgggaattggccctggagctgtttactgtcagttcctgtccagcttcactaaatttggtctggggtcacatcttagctggggactgtggggttctgtggtcccttctcgacttggcccagctccacttgaatgatattgttgtcaaattgctataatagaatccagttgatggacagactatccaatgaatccattatgttggtggtggagctggtgcaaagagctccagagcagctgctggcactgacccccctccaccaggcagccctggcagagagggcactacaaaacctggtaagagtccaccctaccagactcagatttgctgccctgggcaattcttgctcctcagacaatgctctctgactgtcctccaaccctctacttcttgctttcttgctgccaaacaggttcctgtctacaaggcctggcccgttttgcctctgggttctgttccttgataatatgcttcacgttacttgtccatacctcttggagtccgagaaatctcttggagtccacctctcagtctttctgcctgctcctatctggtctcattgcttaagaaagtgaacaaaggtagtgagcatcatagggtgctgagttgggagcaggagggagggaaggtcagggggcttggtgtcttgatcaaggtgtctggtattctgagtcagaagtgcattgtccaagttctgatgctcttctccaggctccaaaggagactccagtctcaggggaagtgctggagaccttaggccctttggttggattcctggggacagagagcacacgacagatccccctacagatcctgctgtcccatctcagtcagctgtaaggcttctgcctaggagagacatttgccacagagctgggatggctgctattgcaggagtctgttcttgggtatggatcttcgagaacttcagattctaactcattctatacccagtccctcagccaccatcatcagtggcagcctgttccatattcctaagggcccttggagccctgtgtccgaaatcctagcatgtcctcttttccccttccttttcctcacagttccctcagctccccagcccccgattttcttcctgtccccaggaaaccagagttgtggagccaggatgaagtagagcaagctggacgcctagtattcactctgtctactgaggcaatttccttgatccccagggtgagatgaaggaagaagggaagggagtaaatgcatagaggggactggtgagctggttatggggacccgtggccaacgagggcaaaggatatgaagcctagatctggggggatactgcgaaacagagacaggactttggacttatagctatagcagcaggtcctgatctgtccagatctccccactctccttctaccttctcatgcaggaggccttgggtccagagaccctggagcggcttctagaaaagcagcagagctgggagcagagcagagttggacagctgtgtagggggccacagcttgctgccaagaaagcagccctggtagcaggggtggtgcgaccagctgctgaggatcttccaggtgaaactacccaaatacttatatgtccagcaggatgtacagggagtatcaaacggtctgggttctacatgtgcttttctctgggactgggttttctaatttataaagcaaagagtttagagggatgatcttcaagtctcgtctagttctaaaattctatagctctgggagtgtgtaaactattaagttttcctttagcccagaacttccattttcctgctgtcttgtctctttaaacagctgactcagctctagctcggctaagtgtggagctctctgctggggagatccctcgaaggagacattgtgaggctagagaactggtttcaaattcagctctgccatgaaatccctgaccaacatcctcagtcttccatctataaaatgagggacttggcatctccaatcacttcttgttcaattcacttctaagccctatacatcttaacctgtaatactccctcgatccctacttcccacagtgctccatcctccaaaggttggaagagtcattccatctacagatacctcctggccctgccttcccccttcagaacctgtgccaaattgtgcagatgtacgagggacattcccagcagcctgctctgcaacccagattgctgagatggagctctcagactttaaggactgcctgacactatttgcaggagacccaggacttgggcctgaggaaccacgggcagccatgggcaaagcaaaatgggtcagggacggagagccaactgaggttggccatgaggaagctgtgtgggtgtgatgtaggacacgaagagaatagagagttggatgagaggtgggggaagcagaagataaaagagttagaagatttgggtcacaagtagaaggtgaggagagataaatattgaggaaagagagctactataatgaatagagggactaaagcagtgattaccaaattttaatgcatatcacaatcatcaagggaacagatttttttcttttttctttttttttctttcttaaaaaataatggcatacttcatgaatttgtgtgttatccttgcgcaggcgccatgctaatctctgtatcattccaatttttttttgtatatgtgctgctgaaccgagcacgggaacagtgccaatgctggattaccatgctagactgacttaataaaaacctttgcggaggctggacggagtggctcgtgcctgtaatcacagcactttgggaggctgaggcaggtggattgcttgagcccaggagtttgagaccagactgggcaacatggtgaagccctgtctctactaaaaatagaaaaaaaattagctgggtgtggcggcacatgcctgtaatcccagctactcaggaggctggggtaggagaatcccctgagcgcaggaggtggagggtgcaatgagccaagatcacaccactgcactccagcctgggtgtcagagcaagaccctgtctcataaattaaaaaataagcctctgggagaaagtctagacatctgcatctgcttttttttttttttttttttttgagacacagtctcactctgtcacccagcatccaggctggagtgcggtggtgtgatcttggctcactgtaacctctacatcctgggctcaaacgatcctcctgcctcagcctctcgagaagctgggactacaggtgcacaccactacacctggctaatttttgtatttttggtagagatgaggtttcgctgtgttgcccaggatggtcttgaactcctgggctcaagcattcctcccacctcagcctcccaaagtgctgggattacagctgtgagccactgtgctgggccccaggcatctgcattttaataagcgtctctgtgtctgatgcacataaaagtgtggaactcatggactagagttagtttgctcttcttttccactgattgtaatgtctttcaaaacaccttagaggaactgtaaggcaacggtctcattttatagtggaggaaaccaaagaaaaggcaagtgacttgcctagagttatacagctaaggtcagaggcaagacttaaaacccagcattctgactcccaatctactggttttccactcacattgttcctctctttctcctagttgtggggtcccccccggggatttggtcctgagcagatcctgcagctcggtagactcttaataggtctgggagatcaggaactacaggagctgatcctagtggactggggagtgctgagcaccctggggcagatagatggctggagctccactcaggtaacacttttactcctccctaccgcttcccaaacacccatcccacagacccagccctatagatcatctaaagcccaaggaattttttcctgtgaccctacctggtcattctttctgtcttttgttaatatcccatactagtgaccttcaggactcttgatttattcactctgaggccctggacacataatactgtctcctacctcttttcctggaggcttcctctttttctttccttttcttttctgagtcctcagccttccccatgactccttaggtcttaatagtaacagaatataacccagtaacacctatcacttccctgtccattaattctccataactttcctccttcccctcttctcccaccccccaccccagctccgcattgtggtctccagtttcctacggcagagtggtcggcatgtgagccacctggacttcgttcatctgacagcgctgggttatactctctgtggactgcggccagaggagctccagcacatcagcagttgggagtttaggtcatttgtgaaggggctgagggtggtcgtgttcaggtaaaggtggacttgctggggaaaggaggatcatgaagttatagtcccatggaggaagggaactcatttgaagccatctcttcctttgtctcatgaccacagtccctttcactgaagccgaattcttcttccttccttcccactgttctacagccaagcagctctcttcctgggcaccctgcatctgcagtgctctgaggaacaactggagtttctggcccacctctttgtactgcctggtgggtttggcccaatcagtaactgggggcctgagatcttcactgaaattggcaccatagcaggtggggagctgggccactgctggtgcaagttggtttggtttctataccatgggtggactggatggaagactgccctgcaattcttcaggtgtgggcctgagagggtgtttaaataaggggctagagacatattggggaaggtctatgatagggcactttgggagtagttagagaaggtctataggtttgaagagagggaaggtcagtctaagacaatgtttggatgccacttgcttcaacagctgggatcccagacctggctctttcagcactgctgcggggacagatccagggcgttactcctcttgccatttctgtcatccctcctcctaaatttgctgtaagtattaatggactggggtgaccacaggagagccagggcccaatggggactacatgcatgcactgattcctacccctgccctcaggtggtgtttagtcccatccaactatctagtctcgccagtgctcaggctgtggctgtcactcctgagcaaatggcctttctgagtcctgagcagcgacgagcagttgcatgggcccaacatgagggaaaggagagcccagaacagcaaggtgagttcccagctgcacagcttgatcctccatctcctgacccagaatcaaacccctaatttggtgctgtctggctcttagagtgcacccagggagatccctggagtgaaggagtctacaggcagagcgctaatttccaagtatcaatgctcctggagagctgagttgtgatattactcccattccctgtctattataggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaata

SEQ ID NO: 6 - DNA, 82 bp, synthetic construct
  misc_feature 1..82: note = Splicing donor signal
  source 1..82: mol_type = other DNA
gtaagtatcaaggttacaagacaggtttaaggagaccaatagaaactgggcttgtcgagacagagaagactcttgcgtttct

SEQ ID NO: 7 - DNA, 51 bp, synthetic construct
  source 1..51: mol_type = other DNA
  misc_feature 1..51: note = Splicing accepting signal
gataggcacctattggtcttactgacatccactttgcctttctctccacag

SEQ ID NO: 8 - protein, 1809 aa, Mus musculus
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPNRRLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLLPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVAGLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTLCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 9 - protein, 1767 aa, Canis lupus
  source 1..1767: note = Canis lupus familiaris
MALSLWPVLLLLTCAATLVSSGIQSLDPDLSLLKSLLSTMDQAPQGSLSRSQFSAFLANISSSFESGRMGEGPVGEPPPLQSPALRLHDFLVTLRGSPDWEPMLGLLGDVLALLGQEQTPRDFLGHQAGVLGGLAEVLLGALVPIGPPTPTRPPCIRDGPSDCVLVADWLPSLLLLLEGTRWQALVQVQPSVDPTNATGLDGREPAPHFLQGLLGLLTPVGELGSEEALWGGLLRTVGAPLYAAFQEGLLRVTDSLRNEVFSILGQPEPDANGQCQGGNLRQLLLWGIRHNLSWDVQALGFLSGSPPPPPALLHCLSTGVPLPRASQPSAHINPRQRRAISVEALCENHSGPAPPYSISNFSIHLLCQHAQPATPQPPPSTAAVCQTAMWYAVSWAPGAQGWLQACHDQFPDQFLEAICSNLSFSALSGPNRRLVKQLCAGLLPPPTNCPEGLPPAPLTPEVFWGCFLENETLWAERLCGEAGLQAVPPSNQAWVQHVCQGPTPDVTAFPPCHVGPCGERCPDGGSFLMMVCANDTMYEALVPFWPWLAGQCRISRGGNDTCFLEGLLGPLLPSLPPLGPSPLCLAPAPFLLGMLSQLPRCQSSVPALAHSTRLHYLLRLLTFLLGPGAGGTEAQGMLGQALMLSSLPDNCSFWDAFRPEGRRSVLRTVGEYLEREEQLTPPGFEPTASPSSGITKMELLACFSPVLWDLLQREKSVWALQILVQAYLHMPPENLQQLVLSAEREAAQGFLTLMHRSWAQLQVPPSEEQALGRLTALLLQRYPRLTSQLFIDLSPLIPFLAVSDLMRFPPSLLANDSVLAAIRDYSPGMRPEQKEALAKRLLAPELFGEVPAWSQELLWAVLPLLPHLPLENFLQLSPHQIQALEDSWPAAGLGPGHARHVLRSLVNQSVQDGEEQVRRLGPLACFLSPEELQSLVPLSDPMGPVERGLLECAANGTLSPQGRVAYELLGVLRSSGGAVLSPLELRVWAPLFPQLGLRFLQELSEPQLRAMLPALQGTSVTPAQAVLLLGRLLPRHDLSLEELCSLHPLLPGLSSQTLQAIPRRVLIGACSCLAPELSRLSACQTAALLQTFRVKDGVKNIGTTGASAAVCIPGQQPIPTTWPDCLLPLLPLKLLQLDSAALLANRRRYRDLPWSEQQAQFLWKKMQVPTNLTLRNLQALGTLAGGMSCEFLQQINLMADFLEVVHMIYQLPTGVRGSLRACIWAELQRKMTMPEPELATLGSELSGLDTKLLLDSPIYLMDRLSNESIMLMVELVRRAPEQLLALTPLHRVALAERALQNLAPKETTVSREVLETLGPLVGFLGIESTRRIPLPILLAHLNQLQGFCLGEPFATELGWLLSQEPILGKPELWSEGEVEQAGRLVLTLSTEAISLIPREALGPETLERLLEKQQSWEQSRVGQLCGTPQLAPKKAALVAGVVRPTAEDLSEPVPNCADVRGTFPAAWSATQIAEMELSDFEDCLALFAGDPGLGPEELRAAMGKAKQLWGPPRGFRPEQILQLSRLLIGLGERELQELILVDWGVLSTLGQIDGWSSIQLRVVVSSFLRQSGRHVSHLDFLHLTALGYTLCGLRPEELQHISSWEFSQAALFLGNLHLQCSEEQLEVLAQLLVLPGGFGPVSNWGPEIFTEIGTIAAGIPDLALSALLQEQIQGLTPLAISVIPAPKFAVVFSPTQLSSLTSVQAMAVTPEQMAFLSPEQRRAVVWAQHEGKESPEQQGRSTAWGLQDWSQPSWAMALTICFLGNLI

SEQ ID NO: 10 - protein, 22 aa, Homo sapiens
MALSLWPLLLLLLLLLLLSFAV

SEQ ID NO: 11 - protein, 1775 aa, Homo sapiens
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 12 - DNA, 4324 bp, synthetic construct
  misc_feature 1..4324: note = pITR-201
  source 1..4324: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtagacttgcccaaaaactctggcaaagttgaattgcttccaaaagttcaactttatcagaaggacctattccctacggaaactagctgaaggtctcctggcactctggatctcgtggaagggagccttcttcagggaacagagggagcgattaagtggtgaaaagcaaacagacctggaaaagttccctttctgagagtagcaacagaaagctctgcaaagactccctccaagctattggatcctcttgcttgggataaccacttgagtactcagataccaaaagaagagtggaaatcccaagagaagtcaccagaaaaaacagcttttaagaaaaaggatacacttttgtccctgaacgcttgtgaaagcaatctgacaatagcagcaattgaaaagggacaaaataagcccgaaatagaagtcacctgggcaaagcaaggtaggactgaaaggctgtgctctcaaaacccaccagtcttgaaacgcactcaacgggtgaaagtagtaggaaagggtgaatttacaaaggacgtaggactcaaagagtgagtttttccaagcagcagaaacctatttcttactaacttggataatttactgaaaaataatacacacaatcaagaaaaaaaaattcaggaagaaatagaaaagaaggaaaacttaatccaagagtgaatagttttgcctcagataactacagtgactggcactaagaatttctgaaagaaccttttcttactgagcactaggctgaaaatagaaggttacttgaacggggacttgactccagtacttcaagattttaggtactttgaaaattcaacaaatagaacaaagaaacacacagctactttctcaaaaaaaggggaggaagaaaacttggaaggcttgggaaatcaaaccaagcaaattgtagagaaattgactgacaccacaaggatatctcctaatacaagccagcagaattttgtcacgcaacgtagtaagagagctttgaaactagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtggtgccctgccctcacctggctatcccacacaggtgagaataaccagaactcacctccggtaccagtgttcacttggccaccatggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccttgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggtaagtatcaaggttacaagacaggtttaaggagaccaatagaaactgggcttgtcgagacagagaagactcttgcgtttctgggattttgccgatttcggcctattggttaaaaaatgagctgatttaacaaaaatttaacgcgaattttaacaaaataagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - DNA, 4434 bp, synthetic construct
  source 1..4434: mol_type = other DNA
  misc_feature 1..4434: note = pITR-202
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggattttgccgatttcggcctattggttaaaaaatgagctgatttaacaaaaatttaacgcgaattttaacaaaatgataggcacctattggtcttactgacatccactttgcctttctctccacaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctccagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaataactagtgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaattcagactcccactagaagaaacagaacttgaaaaaaggataattgtggtgaacacctcaacccagtggtccaaaaactgaaaaactttgaccccgagcaccctcacacagatagactactgaaagaaggagaaaggggcacttactcagtctcccttatcagattgccttacgaggagtactagactccctcaagcaaatagatctcacttaccacttgcaaaggtatactactttcactctattagacctatatatctgaccagggtcctattccaagacaactcttctactcttccagcagactcttatagaaagaaagattctggggtccaagaaagcagtactttcttacaaggagccaaaaaaaataacctttctttagcacttctaaccttggagtgaactggtgatcaaagagaggttggctccctggggacaagtgccacaaattcagtcaactacaagaaagttgagaacactgttctcccgaaaccaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 14 - DNA, 4693 bp, synthetic construct
  misc_feature 1..4693: note = pITR-202GFP
  source 1..4693: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggattttgccgatttcggcctattggttaaaaaatgagctgatttaacaaaaatttaacgcgaattttaacaaaatgataggcacctattggtcttactgacatccactttgcctttctctccacaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctccagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctaggctccggagagggcagaggaagtctgctaacatgcggtgacgtcgaggagaatcctggcccaatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataagcctgtctctacagtagaaggagattgtggggagagaaatcttaagtcataatgaataaagtgcaaacagaagtgcatcctgattattttcagaagctgatgaggaataactagtgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 4619 bp, synthetic construct
  misc_feature 1..4619: note = pITR-203
  source 1..4619: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtagacttgcccaaaaactctggcaaagttgaattgcttccaaaagttcaactttatcagaaggacctattccctacggaaactagctgaaggtctcctggcactctggatctcgtggaagggagccttcttcagggaacagagggagcgattaagtggtgaaaagcaaacagacctggaaaagttccctttctgagagtagcaacagaaagctctgcaaagactccctccaagctattggatcctcttgcttgggataaccacttgagtactcagataccaaaagaagagtggaaatcccaagagaagtcaccagaaaaaacagcttttaagaaaaaggatacacttttgtccctgaacgcttgtgaaagcaatctgacaatagcagcaattgaaaagggacaaaataagcccgaaatagaagtcacctgggcaaagcaaggtaggactgaaaggctgtgctctcaaaacccaccagtcttgaaacgcactcaacgggtgactagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtggtgccctgccctcacctggctatcccacacaggtgagaataaccagaactcacctccggtaccagtgttcacttggccaccatggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccttgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggtaagtatcaaggttacaagacaggtttaaggagaccaatagaaactgggcttgtcgagacagagaagactcttgcgtttctctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcgaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 4729 bp, synthetic construct
  source 1..4729: mol_type = other DNA
  misc_feature 1..4729: note = pITR-204
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggataggcacctattggtcttactgacatccactttgcctttctctccacaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctccagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaataactagtgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 4460 bp, synthetic construct
  misc_feature 1..4460: note = pITR-205
  source 1..4460: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtggtgccctgccctcacctggctatcccacacaggtgagaataaccagaactcacctccggtaccagtgttcacttggccaccatggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccttgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggtaagtatcaaggttacaagacaggtttaaggagaccaatagaaactgggcttgtcgagacagagaagactcttgcgtttctgggattttgccgatttcggcctattggttaaaaaatgagctgatttaacaaaaatttaacgcgaattttaacaaaataagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 130 bp, synthetic construct
  misc_feature 1..130: note = 5' ITR
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 19 - DNA, 1000 bp, synthetic construct
  source 1..1000: mol_type = other DNA
  misc_feature 1..1000: note = 5' FVIII stuffer_1000
agacttgcccaaaaactctggcaaagttgaattgcttccaaaagttcaactttatcagaaggacctattccctacggaaactagctgaaggtctcctggcactctggatctcgtggaagggagccttcttcagggaacagagggagcgattaagtggtgaaaagcaaacagacctggaaaagttccctttctgagagtagcaacagaaagctctgcaaagactccctccaagctattggatcctcttgcttgggataaccacttgagtactcagataccaaaagaagagtggaaatcccaagagaagtcaccagaaaaaacagcttttaagaaaaaggatacacttttgtccctgaacgcttgtgaaagcaatctgacaatagcagcaattgaaaagggacaaaataagcccgaaatagaagtcacctgggcaaagcaaggtaggactgaaaggctgtgctctcaaaacccaccagtcttgaaacgcactcaacgggtgaaagtagtaggaaagggtgaatttacaaaggacgtaggactcaaagagtgagtttttccaagcagcagaaacctatttcttactaacttggataatttactgaaaaataatacacacaatcaagaaaaaaaaattcaggaagaaatagaaaagaaggaaaacttaatccaagagtgaatagttttgcctcagataactacagtgactggcactaagaatttctgaaagaaccttttcttactgagcactaggctgaaaatagaaggttacttgaacggggacttgactccagtacttcaagattttaggtactttgaaaattcaacaaatagaacaaagaaacacacagctactttctcaaaaaaaggggaggaagaaaacttggaaggcttgggaaatcaaaccaagcaaattgtagagaaattgactgacaccacaaggatatctcctaatacaagccagcagaattttgtcacgcaacgtagtaagagagctttgaaa

SEQ ID NO: 20 - DNA, 342 bp, synthetic construct
  misc_feature 1..342: note = CMV enhancer
  source 1..342: mol_type = other DNA
ctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttg

SEQ ID NO: 21 - DNA, 219 bp, synthetic construct
  misc_feature 1..219: note = CMV promoter
  source 1..219: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 22 - DNA, 276 bp, synthetic construct
  misc_feature 1..276: note = CBA promoter
  source 1..276: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 23 - DNA, 1013 bp, synthetic construct
  misc_feature 1..1013: note = Chimeric intron
  source 1..1013: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 24 - DNA, 73 bp, synthetic construct
  source 1..73: mol_type = other DNA
  misc_feature 1..73: note = 5' UTR
gccctgccctcacctggctatcccacacaggtgagaataaccagaactcacctccggtaccagtgttcacttg

SEQ ID NO: 25 - DNA, 2196 bp, synthetic construct
  misc_feature 1..2196: note = 5' STRC
  source 1..2196: mol_type = other DNA
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccttgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcag

SEQ ID NO: 26 - DNA, 77 bp, synthetic construct
  misc_feature 1..77: note = AK sequence
  source 1..77: mol_type = other DNA
gggattttgccgatttcggcctattggttaaaaaatgagctgatttaacaaaaatttaacgcgaattttaacaaaat

SEQ ID NO: 27 - DNA, 872 bp, synthetic construct
  misc_feature 1..872: note = AP sequence
  source 1..872: mol_type = other DNA
ctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcg

SEQ ID NO: 28 - DNA, 3129 bp, synthetic construct
  misc_feature 1..3129: note = 3' STRC
  source 1..3129: mol_type = other DNA
gcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctccagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgcta

SEQ ID NO: 29 - DNA, 54 bp, synthetic construct
  misc_feature 1..54: note = T2A sequence
  source 1..54: mol_type = other DNA
gagggcagaggaagtctgctaacatgcggtgacgtcgaggagaatcctggccca

SEQ ID NO: 30 - protein, 21 aa, synthetic construct
  REGION 1..21: note = T2A sequence
GSGEGRGSLLTCGDVEENPGP

SEQ ID NO: 31 - DNA, 696 bp, synthetic construct
  misc_feature 1..696: note = tGFP
  source 1..696: mol_type = other DNA
atggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaa

SEQ ID NO: 32 - protein, 232 aa, synthetic construct
  REGION 1..232: note = tGFP
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEE

SEQ ID NO: 33 - DNA, 109 bp, synthetic construct
  misc_feature 1..109: note = 3' UTR
  source 1..109: mol_type = other DNA
gcctgtctctacagtagaaggagattgtggggagagaaatcttaagtcataatgaataaagtgcaaacagaagtgcatcctgattattttcagaagctgatgaggaata

SEQ ID NO: 34 - DNA, 240 bp, synthetic construct
  misc_feature 1..240: note = bGHpA
  source 1..240: mol_type = other DNA
gctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 35 - DNA, 500 bp, synthetic construct
  misc_feature 1..500: note = 3' FVIII stuffer
  source 1..500: mol_type = other DNA
caattcagactcccactagaagaaacagaacttgaaaaaaggataattgtggtgaacacctcaacccagtggtccaaaaactgaaaaactttgaccccgagcaccctcacacagatagactactgaaagaaggagaaaggggcacttactcagtctcccttatcagattgccttacgaggagtactagactccctcaagcaaatagatctcacttaccacttgcaaaggtatactactttcactctattagacctatatatctgaccagggtcctattccaagacaactcttctactcttccagcagactcttatagaaagaaagattctggggtccaagaaagcagtactttcttacaaggagccaaaaaaaataacctttctttagcacttctaaccttggagtgaactggtgatcaaagagaggttggctccctggggacaagtgccacaaattcagtcaactacaagaaagttgagaacactgttctcccgaaacc

SEQ ID NO: 36 - DNA, 141 bp, synthetic construct
  source 1..141: mol_type = other DNA
  misc_feature 1..141: note = 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 500 bp, synthetic construct
  misc_feature 1..500: note = 5' FVIII stuffer_500
  source 1..500: mol_type = other DNA
agacttgcccaaaaactctggcaaagttgaattgcttccaaaagttcaactttatcagaaggacctattccctacggaaactagctgaaggtctcctggcactctggatctcgtggaagggagccttcttcagggaacagagggagcgattaagtggtgaaaagcaaacagacctggaaaagttccctttctgagagtagcaacagaaagctctgcaaagactccctccaagctattggatcctcttgcttgggataaccacttgagtactcagataccaaaagaagagtggaaatcccaagagaagtcaccagaaaaaacagcttttaagaaaaaggatacacttttgtccctgaacgcttgtgaaagcaatctgacaatagcagcaattgaaaagggacaaaataagcccgaaatagaagtcacctgggcaaagcaaggtaggactgaaaggctgtgctctcaaaacccaccagtcttgaaacgcactcaacgggtga

SEQ ID NO: 38 - DNA, 17 bp, Pan troglodytes
  source 1..17: mol_type = unassigned DNA
aaataaaatacgaaatg

(19) United States
(12) Patent Application Publication
(10) Pub. No.: US 2026/0000787 A1
(43) Pub. Date: Jan. 1, 2026

(54) METHODS OF TREATING NON-SYNDROMIC SENSORINEURAL HEARING LOSS

(71) Applicant: Akouos, Inc., Boston, MA (US)

(72) Inventors: Emmanuel John Simons, Brookline, MA (US); Robert Ng, Newton, MA (US)

(21) Appl. No.: 19/015,387

(22) Filed: Jan. 9, 2025

Related U.S. Application Data
(62) Division of application No. 17/259,661, filed on Jan. 12, 2021, now Pat. No. 12,226,493.
(60) Provisional application No. 62/697,652, filed on Jul. 13, 2018.

(51) Int. Cl. A61K 48/00; C07K 14/47; C12N 9/22; C12N 15/11; C12N 15/86; C12N 15/90

(52) U.S. Cl. CPC A61K 48/005; C07K 14/47; C12N 9/22; C12N 15/11; C12N 15/86; C12N 15/907; C12N 2310/20; C12N 2320/33; C12N 2750/14143; C12N 2830/50; C12N 2840/007

(57) ABSTRACT

Provided herein are compositions that include at least two different nucleic acid vectors, where each of the at least two different vectors includes a coding sequence that encodes a different portion of a stereocilin protein, and the use of these compositions to treat non-syndromic sensorineural hearing loss in a subject.

### CROSS-REFERENCE TO RELATED APPLICATIONS

This application is a divisional application of U.S. Ser. No. 17/259,661, filed Jan. 12, 2021, which is a 371 national stage application of International Patent Application No. PCT/US19/41625, filed on Jul. 12, 2019, which claims priority to U.S. Provisional Patent Application Ser. No. 62/697,652, filed Jul. 13, 2018; the entire contents of which are herein incorporated by reference.

### SEQUENCE LISTING

The instant application contains a Sequence Listing which has been submitted electronically in XML format and is hereby incorporated by reference in its entirety. The sequence listing, created on Sep. 22, 2025, is named 2013615-0816_SL.xml and is 140,288 bytes in size.

### TECHNICAL FIELD

The present disclosure relates generally to the use of nucleic acids to treat hearing loss in a human subject.

### BACKGROUND OF THE INVENTION

Non-syndromic deafness, in contrast to syndromic deafness, is hearing loss that is not associated with other signs and symptoms. Seventy to eighty percent of cases of genetic deafness are non-syndromic. While the causes of non-syndromic deafness are complex, researchers have identified more than 30 genes that, when altered, are associated with non-syndromic deafness (e.g., stereocilin (STRC)). Current treatments consist mainly of hearing amplification for mild to severe hearing loss and cochlear implants for severe to profound hearing loss; however, a long-felt need remains for agents and methods for preventing or reversing non-syndromic deafness.

Hearing loss can be conductive (arising from the ear canal or middle ear), sensorineural (arising from the inner ear or auditory nerve), or mixed. Most forms of non-syndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present invention is based on the discovery that a composition including at least two different nucleic acid vectors, where each of the at least two different vectors includes a coding sequence that encodes a different portion of a stereocilin protein, can be used to generate a sequence encoding an active stereocilin protein (e.g., a full-length stereocilin protein) in a mammalian cell, and thereby treat non-syndromic sensorineural hearing loss in a subject in need thereof.

Provided herein are compositions that include at least two different nucleic acid vectors, where: each of the at least two different vectors includes a coding sequence that encodes a different portion of a stereocilin protein, each of the encoded portions being at least 30 amino acid residues in length, wherein the amino acid sequence of each of the encoded portions may optionally partially overlap with the amino acid sequence of a different one of the encoded portions; no single vector of the at least two different vectors encodes a full-length stereocilin protein; at least one of the coding sequences includes a nucleotide sequence spanning two neighboring exons of stereocilin genomic DNA, and lacks an intronic sequence between the two neighboring exons; and when introduced into a mammalian cell the at least two different vectors undergo homologous recombination with each other, thereby forming a recombined nucleic acid that encodes a full-length stereocilin protein. In some embodiments of any of the compositions described herein, each of the at least two different vectors is a plasmid, a transposon, a cosmid, an artificial chromosome, or a viral vector. In some embodiments of any of the compositions described herein, each of the at least two different vectors is a human artificial chromosome (HAC), yeast artificial chromosome (YAC), bacterial artificial chromosome (BAC), or a P1-derived artificial chromosome (PAC). In some embodiments of any of the compositions described herein, each of the at least two different vectors is a viral vector selected from an adeno-associated virus (AAV) vector, an adenovirus vector, a lentivirus vector, or a retrovirus vector. In some embodiments of any of the compositions described herein, the viral vector is an AAV vector.

In some embodiments of any of the compositions described herein, the amino acid sequence of none of the encoded portions overlaps with the amino acid sequence of a different one of the encoded portions. In some embodiments of any of the compositions described herein, the amino acid sequence of each of the encoded portions partially overlaps with the amino acid sequence of a different one of the encoded portions. In some embodiments of any of the compositions described herein, the overlapping amino acid sequence is between about 30 amino acid residues to about 1000 amino acid residues in length.

In some embodiments of any of the compositions described herein, the vectors include two different vectors, each of which includes a different segment of an intron, wherein the intron includes the nucleotide sequence of an intron that is present in stereocilin genomic DNA, and wherein the two different segments overlap in sequence by at least 100 nucleotides. In some embodiments of any of the compositions described herein, the two different segments overlap in sequence by about 100 nucleotides to about 800 nucleotides. In some embodiments of any of the compositions described herein, the nucleotide sequence of each of the at least two different vectors is between about 500 nucleotides to about 10,000 nucleotides in length (e.g., between about 500 nucleotides to about 5,000 nucleotides in length).

In some embodiments of any of the compositions described herein, the number of different vectors in the composition is two. In some embodiments, one of the two vectors includes SEQ ID NO: 13 and the second of the two vectors includes SEQ ID NO: 17.

In some embodiments of any of the compositions described herein, one of the at least two different vectors includes a sequence encoding a stereocilin protein. In some embodiments, the sequence encoding a stereocilin protein is at least 90% (e.g., at least 92%, at least 94%, at least 95%, at least 96%, at least 97%, at least 98%, at least 99%, or 100%) identical to SEQ ID NO: 11.

In some embodiments of any of the compositions described herein, a first of the two different vectors includes a coding sequence that encodes an N-terminal portion of the stereocilin protein. In some embodiments of any of the compositions described herein, the N-terminal portion of the stereocilin protein is between 30 amino acids to 1600 amino acids in length (e.g., between about 200 amino acids to about 1500 amino acids in length). In some embodiments of any of the compositions described herein, the first vector further includes one or both of a promoter and a Kozak sequence. In some embodiments of any of the compositions described herein, the first vector includes a promoter that is an inducible promoter, a constitutive promoter, or a tissue-specific promoter.

In some embodiments of any of the compositions described herein, the second of the two different vectors includes a coding sequence that encodes a C-terminal portion of the stereocilin protein. In some embodiments of any of the compositions described herein, the C-terminal portion of the stereocilin protein is between 30 amino acids to 1600 amino acids in length (e.g., between 200 amino acids to 1500 amino acids in length). In some embodiments of any of the compositions described herein, the second vector further includes a poly(dA) sequence. Some embodiments of any of the compositions described herein further include a pharmaceutically acceptable excipient.

Also provided herein are kits that include any of the compositions described herein. Some embodiments of any of the kits described herein further include a pre-loaded syringe containing the composition.

Also provided herein are methods that include introducing into a cochlea of a mammal a therapeutically effective amount of any of the compositions described herein. In some embodiments of any of the methods described herein, the mammal is a human. In some embodiments of any of the methods described herein, the mammal has been previously identified as having a defective stereocilin gene.

Also provided herein are methods of increasing expression of a full-length stereocilin protein in a mammalian cell that include introducing any of the compositions described herein into the mammalian cell. In some embodiments of any of the methods described herein, the mammalian cell is a cochlear outer hair cell. In some embodiments of any of the methods described herein, the mammalian cell is a human cell. In some embodiments of any of the methods described herein, the mammalian cell has previously been determined to have a defective stereocilin gene.

Also provided herein are methods of increasing expression of a full-length stereocilin protein in an outer hair cell in a cochlea of a mammal that include introducing into the cochlea of the mammal a therapeutically effective amount of any of the compositions described herein. In some embodiments of any of the methods described herein, the mammal has been previously identified as having a defective stereocilin gene. In some embodiments of any of the methods described herein, the mammal is a human.

Also provided herein are methods of treating non-symptomatic sensorineural hearing loss in a subject identified as having a defective stereocilin gene that include administering a therapeutically effective amount of any of the compositions described herein into the cochlea of the subject. In some embodiments of any of the methods described herein, the subject is a human. Some embodiments of any of the methods described herein further include, prior to the administering step, determining that the subject has a defective stereocilin gene.

Also provided herein are compositions that include two different nucleic acid vectors, where: a first nucleic acid vector of the two different nucleic acid vectors includes a promoter, a first coding sequence that encodes an N-terminal portion of a stereocilin protein positioned 3′ of the promoter, and a splicing donor signal sequence positioned at the 3′ end of the first coding sequence; and a second nucleic acid vector of the two different nucleic acid vectors including a splicing acceptor signal sequence, a second coding sequence that encodes a C-terminal portion of a stereocilin protein positioned at the 3′ end of the splicing acceptor signal sequence, and a polyadenylation sequence at the 3′ end of the second coding sequence; where each of the encoded portions is at least 30 amino acid residues in length, where the amino acid sequence of each of the encoded portions do not overlap; where no single vector of the two different vectors encodes a full-length stereocilin protein; and when introduced into a mammalian cell, splicing occurs between the splicing donor signal sequence and the splicing acceptor signal sequence, thereby forming a recombined nucleic acid that encodes a full-length stereocilin protein. In some embodiments of any of the compositions described herein, at least one of the coding sequences includes a nucleotide sequence spanning two neighboring exons of stereocilin genomic DNA, and lacks an intronic sequence between the two neighboring exons.

Also provided are compositions that include two different nucleic acid vectors, where: a first nucleic acid vector of the two different nucleic acid vectors includes a promoter, a first coding sequence that encodes an N-terminal portion of a stereocilin protein positioned 3′ of the promoter, a splicing donor signal sequence positioned at the 3′ end of the first coding sequence, and a first detectable marker gene positioned 3′ of the splicing donor signal sequence; and a second nucleic acid vector of the two different nucleic acid vectors includes a second detectable marker gene, a splicing acceptor signal sequence positioned 3′ of the second detectable marker gene, a second coding sequence that encodes a C-terminal portion of a stereocilin protein positioned at the 3′ end of the splicing acceptor signal sequence, and a polyadenylation sequence positioned at the 3′ end of the second coding sequence; where each of the encoded portions is at least 30 amino acid residues in length, where the amino acid sequence of each of the encoded portions do not overlap; where no single vector of the two different vectors encodes a full-length stereocilin protein; and when introduced into a mammalian cell, splicing occurs between the splicing donor signal and the splicing acceptor signal, thereby forming a recombined nucleic acid that encodes a full-length stereocilin protein. In some embodiments of any of the compositions described herein, at least one of the coding sequences includes a nucleotide sequence spanning two neighboring exons of stereocilin genomic DNA, and lacks an intronic sequence between the two neighboring exons. In some embodiments of any of the compositions described herein, the first or second detectable marker gene is alkaline phosphatase. In some embodiments of any of the compositions described herein, the first and second detectable marker genes are the same.

Also provided herein are compositions that include two different nucleic acid vectors, where: a first nucleic acid vector of the two different nucleic acid vectors includes a promoter, a first coding sequence that encodes an N-terminal portion of a stereocilin protein positioned 3′ to the promoter, a splicing donor signal sequence positioned at the 3′ end of the first coding sequence, and a F1 phage recombinogenic region positioned 3′ to the splicing donor signal sequence; and a second nucleic acid vector of the two different nucleic acid vectors includes a F1 phage recombinogenic region, a splicing acceptor signal sequence positioned 3′ of the F1 phage recombinogenic region, a second coding sequence that encodes a C-terminal portion of a stereocilin protein positioned at the 3′ end of the splicing acceptor signal sequence, and a polyadenylation sequence positioned at the 3′ end of the second coding sequence; where each of the encoded portions is at least 30 amino acid residues in length, where the amino acid sequence of each of the encoded portions do not overlap; where no single vector of the two different vectors encodes a full-length stereocilin protein; and when introduced into a mammalian cell, splicing occurs between the splicing donor signal and the splicing acceptor signal, thereby forming a recombined nucleic acid that encodes a full-length stereocilin protein. In some embodiments of any of the compositions described herein, at least one of the coding sequences includes a nucleotide sequence spanning two neighboring exons of stereocilin genomic DNA, and lacks an intronic sequence between the two neighboring exons.

Also provided herein are compositions that include: a Cas9 nuclease; a guide RNA that includes at the 5′ end of the guide RNA a complementary region consisting of 20 nucleotides that are complementary to 20 consecutive nucleotides within positions 13955-14151 of SEQ ID NO:5, that can be used in CRISPR/Cas9 RNA-guided genome editing to remove a nonsense mutation at a predetermined site in the endogenous stereocilin pseudogene sequence of SEQ ID NO: 5; and when introduced into a mammalian cell, a nucleic acid encoding a full-length stereocilin protein is reconstituted at the locus of the stereocilin pseudogene.

Also provided are compositions that include at least one nucleic acid vector, wherein the at least one nucleic acid vector includes an adeno-associated virus (AAV) vector that includes an antisense oligonucleotide that is at least 80% complementary to a contiguous nucleotide sequence of SEQ ID NO: 5 at positions 13955-14151, where the antisense oligonucleotide is between 15-30 nucleotides in length; and when introduced into a mammalian cell, a nucleic acid encoding a full-length stereocilin protein is generated at the locus of the stereocilin pseudogene.

Also provided herein are kits that include any of the compositions described herein. Some embodiments of any of the kits described herein further include a pre-loaded syringe containing the composition.

Also provided are methods that include introducing into a cochlea of a mammal a therapeutically effective amount of any of the compositions described herein. In some embodiments of any of the methods described herein, the mammal is a human. In some embodiments of any of the methods described herein, the mammal has been previously identified as having a defective stereocilin gene.

Also provided herein are methods of increasing expression of a full-length stereocilin protein in a mammalian cell that include introducing any of the compositions described herein into the mammalian cell. In some embodiments of any of the methods described herein, the mammalian cell is a cochlear outer hair cell. In some embodiments of any of the methods described herein, the mammalian cell is a human cell. In some embodiments of any of the methods described herein, the mammalian cell has previously been determined to have a defective stereocilin gene.

Also provided herein are methods of increasing expression of a full-length stereocilin protein in an outer hair cell in a cochlea of a mammal that include introducing into the cochlea of the mammal a therapeutically effective amount of any of the compositions described herein. In some embodiments of any of the methods described herein, the mammal has been previously identified as having a defective stereocilin gene. In some embodiments of any of the methods described herein, the mammal is a human.

Also provided herein are methods of treating non-symptomatic sensorineural hearing loss in a subject identified as having a defective stereocilin gene that include administering a therapeutically effective amount of any of the compositions described herein into the cochlea of the subject. In some embodiments of any of the methods described herein, the subject is a human. Some embodiments of any of the methods described herein further include, prior to the administering step, determining that the subject has a defective stereocilin gene.

The term “a” and “an” refers to one or to more than one (i.e., at least one) of the grammatical object of the article. By way of example, “an element” encompasses one element and more than one element.

The term “mutation in a stereocilin gene” refers to a modification in a wildtype stereocilin gene that results in the production of a stereocilin protein having one or more of: a deletion in one or more amino acids, one or more amino acid substitutions, and one or more amino acid insertions as compared to the wildtype stereocilin protein, and/or results in a decrease in the expressed level of the encoded stereocilin protein in a mammalian cell as compared to the expressed level of the encoded stereocilin protein in a mammalian cell not having a mutation. In some embodiments, a mutation can result in the production of a stereocilin protein having a deletion in one or more amino acids (e.g., 2, 3, 4, 5, 6, 7, 8, 9, 10, 11, 12, 13, 14, 15 16, 17, 18, 19, or 20 amino acids). In some embodiments, the mutation can result in a frameshift in the stereocilin gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding wildtype protein) that may or may not be functional. In some embodiments, the mutation can result in the loss (or a decrease in the level) of expression of stereocilin mRNA or stereocilin protein or both the mRNA and protein. In some embodiments, the mutation can result in the production of an altered stereocilin protein having a loss or decrease in one or more biological activities (functions) as compared to a wildtype stereocilin protein.

In some embodiments, the mutation is an insertion of one or more nucleotides into a stereocilin gene. In some embodiments, the mutation is in a regulatory sequence of the stereocilin gene, i.e., a portion of the gene that is not coding sequence. In some embodiments, a mutation in a regulatory sequence may be in a promoter or enhancer region and prevent or reduce the proper transcription of the stereocilin gene. The term “conservative mutation” refers to a mutation that does not change the amino acid encoded at the site of the mutation (due to codon degeneracy).

Modifications can be introduced into a nucleotide sequence by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine, and isoleucine), and aromatic side chains (e.g., tyrosine, phenylalanine, tryptophan, and histidine).

Unless otherwise specified, a “nucleotide sequence encoding an amino acid sequence” includes all nucleotide sequences that are degenerate versions of each other and thus encode the same amino acid sequence.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” mammalian cell is one that has been transfected, transformed or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell (e.g., a mammalian cell) is lost upon multiple rounds of cell division.

The term “subject” is intended to include any mammal. In some embodiments, the subject is a rodent (e.g., a rat or mouse), a rabbit, a non-human primate, or a human. In some embodiments, the subject has or is at risk of developing non-syndromic deafness. In some embodiments, the subject has been previously identified as having a mutation in a stereocilin gene. In some embodiments, the subject has been identified as having a mutation in a stereocilin gene and has been diagnosed with non-symptomatic sensorineural hearing loss. In some embodiments, the subject has been identified as having non-symptomatic sensorineural hearing loss.

A treatment is “therapeutically effective” when it results in a reduction in one or more of the number, severity, and frequency of one or more symptoms of a disease state (e.g., non-symptomatic sensorineural hearing loss) in a subject (e.g., a human). In some embodiments, a therapeutically effective amount of a composition can result in an increase in the expression level of an active stereocilin protein (e.g., a wildtype, full-length stereocilin protein or of a variant of a stereocilin protein that has the desired activity) (e.g., as compared to the expression level prior to treatment with the composition). In some embodiments, a therapeutically effective amount of a composition can result in an increase in the expression level of an active stereocilin protein (e.g., a wildtype, full-length stereocilin protein or active variant) in a target cell (e.g., a cochlear outer hair cell). In some embodiments, a therapeutically effective amount of a composition can result in an increase in the expression level of an active stereocilin protein (e.g., a wildtype, full-length stereocilin protein or active variant), and/or an increase in one or more activities of a stereocilin protein in a target cell (e.g., as compared to a reference level, such as the level(s) in a subject prior to treatment, the level(s) in a subject having a mutation in a stereocilin gene, or the level(s) in a subject or a population of subjects having non-symptomatic sensorineural hearing loss).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “active stereocilin protein” means a protein encoded by DNA that, if substituted for both wildtype alleles encoding full-length stereocilin protein in auditory hair cells of what is otherwise a wildtype mammal, and if expressed in the auditory hair cells of that mammal, results in that mammal's having a level of hearing approximating the normal level of hearing of a similar mammal that is entirely wildtype. Non-limiting examples of active stereocilin proteins are full-length stereocilin proteins (e.g., any of the full-length stereocilin proteins described herein).

For example, an active stereocilin protein can include a sequence of a wildtype, full-length stereocilin protein (e.g., a wildtype, human, full-length stereocilin protein) including 1 amino acid substitution to about 160 amino acid substitutions, 1 amino acid substitution to about 155 amino acid substitutions, 1 amino acid substitution to about 150 amino acid substitutions, 1 amino acid substitution to about 145 amino acid substitutions, 1 amino acid substitution to about 140 amino acid substitutions, 1 amino acid substitution to about 135 amino acid substitutions, 1 amino acid substitution to about 130 amino acid substitutions, 1 amino acid substitution to about 125 amino acid substitutions, 1 amino acid substitution to about 120 amino acid substitutions, 1 amino acid substitution to about 115 amino acid substitutions, 1 amino acid substitution to about 110 amino acid substitutions, 1 amino acid substitution to about 105 amino acid substitutions, 1 amino acid substitution to about 100 amino acid substitutions, 1 amino acid substitution to about 95 amino acid substitutions, 1 amino acid substitution to about 90 amino acid substitutions, 1 amino acid substitution to about 85 amino acid substitutions, 1 amino acid substitution to about 80 amino acid substitutions, 1 amino acid substitution to about 75 amino acid substitutions, 1 amino acid substitution to about 70 amino acid substitutions, 1 amino acid substitution to about 65 amino acid substitutions, 1 amino acid substitution to about 60 amino acid substitutions, 1 amino acid substitution to about 55 amino acid substitutions, 1 amino acid substitution to about 50 amino acid substitutions, 1 amino acid substitution to about 45 amino acid substitutions, 1 amino acid substitution to about 40 amino acid substitutions, 1 amino acid substitution to about 35 amino acid substitutions, 1 amino acid substitution to about 30 amino acid substitutions, 1 amino acid substitution to about 25 amino acid substitutions, 1 amino acid substitution to about 20 amino acid substitutions, 1 amino acid substitution to about 15 amino acid substitutions, 1 amino acid substitution to about 10 amino acid substitutions, 1 amino acid substitution to about 9 amino acid substitutions, 1 amino acid substitution to about 8 amino acid substitutions, 1 amino acid substitution to about 7 amino acid substitutions, 1 amino acid substitution to about 6 amino acid substitutions, 1 amino acid substitution to about 5 amino acid substitutions, 1 amino acid substitution to about 4 amino acid substitutions, 1 amino acid substitution to about 3 amino acid substitutions, between about 2 amino acid substitutions to about 160 amino acid substitutions, about 2 amino acid substitutions to about 155 amino acid substitutions, about 2 amino acid substitutions to about 150 amino acid substitutions, about 2 amino acid substitutions to about 145 amino acid substitutions, about 2 amino acid substitutions to about 140 amino acid substitutions, about 2 amino acid substitutions to about 135 amino acid substitutions, about 2 amino acid substitutions to about 130 amino acid substitutions, about 2 amino acid substitutions to about 125 amino acid substitutions, about 2 amino acid substitutions to about 120 amino acid substitutions, about 2 amino acid substitutions to about 115 amino acid substitutions, about 2 amino acid substitutions to about 110 amino acid substitutions, about 2 amino acid substitutions to about 105 amino acid substitutions, about 2 amino acid substitutions to about 100 amino acid substitutions, about 2 amino acid substitutions to about 95 amino acid substitutions, about 2 amino acid substitutions to about 90 amino acid substitutions, about 2 amino acid substitutions to about 85 amino acid substitutions, about 2 amino acid substitutions to about 80 amino acid substitutions, about 2 amino acid substitutions to about 75 amino acid substitutions, about 2 amino acid substitutions to about 70 amino acid substitutions, about 2 amino acid substitutions to about 65 amino acid substitutions, about 2 amino acid substitutions to about 60 amino acid substitutions, about 2 amino acid substitutions to about 55 amino acid substitutions, about 2 amino acid substitutions to about 50 amino acid substitutions, about 2 amino acid substitutions to about 45 amino acid substitutions, about 2 amino acid substitutions to about 40 amino acid substitutions, about 2 amino acid substitutions to about 35 amino acid substitutions, about 2 amino acid substitutions to about 30 amino acid substitutions, about 2 amino acid substitutions to about 25 amino acid substitutions, about 2 amino acid substitutions to about 20 amino acid substitutions, about 2 amino acid substitutions to about 15 amino acid substitutions, about 2 amino acid substitutions to about 10 amino acid substitutions, about 2 amino acid substitutions to about 9 amino acid substitutions, about 2 amino acid substitutions to about 8 amino acid substitutions, about 2 amino acid substitutions to about 7 amino acid substitutions, about 2 amino acid substitutions to about 6 amino acid substitutions, about 2 amino acid substitutions to about 5 amino acid substitutions, about 2 amino acid substitutions to about 4 amino acid substitutions, between about 3 amino acid substitutions to about 160 amino acid substitutions, about 3 amino acid substitutions to about 155 amino acid substitutions, about 3 amino acid substitutions to about 150 amino acid substitutions, about 3 amino acid substitutions to about 145 amino acid substitutions, about 3 amino acid substitutions to about 140 amino acid substitutions, about 3 amino acid substitutions to about 135 amino acid substitutions, about 3 amino acid substitutions to about 130 amino acid substitutions, about 3 amino acid substitutions to about 125 amino acid substitutions, about 3 amino acid substitutions to about 120 amino acid substitutions, about 3 amino acid substitutions to about 115 amino acid substitutions, about 3 amino acid substitutions to about 110 amino acid substitutions, about 3 amino acid substitutions to about 105 amino acid substitutions, about 3 amino acid substitutions to about 100 amino acid substitutions, about 3 amino acid substitutions to about 95 amino acid substitutions, about 3 amino acid substitutions to about 90 amino acid substitutions, about 3 amino acid substitutions to about 85 amino acid substitutions, about 3 amino acid substitutions to about 80 amino acid substitutions, about 3 amino acid substitutions to about 75 amino acid substitutions, about 3 amino acid substitutions to about 70 amino acid substitutions, about 3 amino acid substitutions to about 65 amino acid substitutions, about 3 amino acid substitutions to about 60 amino acid substitutions, about 3 amino acid substitutions to about 55 amino acid substitutions, about 3 amino acid substitutions to about 50 amino acid substitutions, about 3 amino acid substitutions to about 45 amino acid substitutions, about 3 amino acid substitutions to about 40 amino acid substitutions, about 3 amino acid substitutions to about 35 amino acid substitutions, about 3 amino acid substitutions to about 30 amino acid substitutions, about 3 amino acid substitutions to about 25 amino acid substitutions, about 3 amino acid substitutions to about 20 amino acid substitutions, about 3 amino acid substitutions to about 15 amino acid substitutions, about 3 amino acid substitutions to about 10 amino acid substitutions, about 3 amino acid substitutions to about 9 amino acid substitutions, about 3 amino acid substitutions to about 8 amino acid substitutions, about 3 amino acid substitutions to about 7 amino acid substitutions, about 3 amino acid substitutions to about 6 amino acid substitutions, about 3 amino acid substitutions to about 5 amino acid substitutions, between about 4 amino acid substitutions to about 160 amino acid substitutions, about 4 amino acid substitutions to about 155 amino acid substitutions, about 4 amino acid substitutions to about 150 amino acid substitutions, about 4 amino acid substitutions to about 145 amino acid substitutions, about 4 amino acid substitutions to about 140 amino acid substitutions, about 4 amino acid substitutions to about 135 amino acid substitutions, about 4 amino acid substitutions to about 130 amino acid substitutions, about 4 amino acid substitutions to about 125 amino acid substitutions, about 4 amino acid substitutions to about 120 amino acid substitutions, about 4 amino acid substitutions to about 115 amino acid substitutions, about 4 amino acid substitutions to about 110 amino acid substitutions, about 4 amino acid substitutions to about 105 amino acid substitutions, about 4 amino acid substitutions to about 100 amino acid substitutions, about 4 amino acid substitutions to about 95 amino acid substitutions, about 4 amino acid substitutions to about 90 amino acid substitutions, about 4 amino acid substitutions to about 85 amino acid substitutions, about 4 amino acid substitutions to about 80 amino acid substitutions, about 4 amino acid substitutions to about 75 amino acid substitutions, about 4 amino acid substitutions to about 70 amino acid substitutions, about 4 amino acid substitutions to about 65 amino acid substitutions, about 4 amino acid substitutions to about 60 amino acid substitutions, about 4 amino acid substitutions to about 55 amino acid substitutions, about 4 amino acid substitutions to about 50 amino acid substitutions, about 4 amino acid substitutions to about 45 amino acid substitutions, about 4 amino acid substitutions to about 40 amino acid substitutions, about 4 amino acid substitutions to about 35 amino acid substitutions, about 4 amino acid substitutions to about 30 amino acid substitutions, about 4 amino acid substitutions to about 25 amino acid substitutions, about 4 amino acid substitutions to about 20 amino acid substitutions, about 4 amino acid substitutions to about 15 amino acid substitutions, about 4 amino acid substitutions to about 10 amino acid substitutions, about 4 amino acid substitutions to about 9 amino acid substitutions, about 4 amino acid substitutions to about 8 amino acid substitutions, about 4 amino acid substitutions to about 7 amino acid substitutions, about 4 amino acid substitutions to about 6 amino acid substitutions, between about 5 amino acid substitutions to about 160 amino acid substitutions, about 5 amino acid substitutions to about 155 amino acid substitutions, about 5 amino acid substitutions to about 150 amino acid substitutions, about 5 amino acid substitutions to about 145 amino acid substitutions, about 5 amino acid substitutions to about 140 amino acid substitutions, about 5 amino acid substitutions to about 135 amino acid substitutions, about 5 amino acid substitutions to about 130 amino acid substitutions, about 5 amino acid substitutions to about 125 amino acid substitutions, about 5 amino acid substitutions to about 120 amino acid substitutions, about 5 amino acid substitutions to about 115 amino acid substitutions, about 5 amino acid substitutions to about 110 amino acid substitutions, about 5 amino acid substitutions to about 105 amino acid substitutions, about 5 amino acid substitutions to about 100 amino acid substitutions, about 5 amino acid substitutions to about 95 amino acid substitutions, about 5 amino acid substitutions to about 90 amino acid substitutions, about 5 amino acid substitutions to about 85 amino acid substitutions, about 5 amino acid substitutions to about 80 amino acid substitutions, about 5 amino acid substitutions to about 75 amino acid substitutions, about 5 amino acid substitutions to about 70 amino acid substitutions, about 5 amino acid substitutions to about 65 amino acid substitutions, about 5 amino acid substitutions to about 60 amino acid substitutions, about 5 amino acid substitutions to about 55 amino acid substitutions, about 5 amino acid substitutions to about 50 amino acid substitutions, about 5 amino acid substitutions to about 45 amino acid substitutions, about 5 amino acid substitutions to about 40 amino acid substitutions, about 5 amino acid substitutions to about 35 amino acid substitutions, about 5 amino acid substitutions to about 30 amino acid substitutions, about 5 amino acid substitutions to about 25 amino acid substitutions, about 5 amino acid substitutions to about 20 amino acid substitutions, about 5 amino acid substitutions to about 15 amino acid substitutions, about 5 amino acid substitutions to about 10 amino acid substitutions, about 5 amino acid substitutions to about 9 amino acid substitutions, about 5 amino acid substitutions to about 8 amino acid substitutions, about 5 amino acid substitutions to about 7 amino acid substitutions, between about 6 amino acid substitutions to about 160 amino acid substitutions, about 6 amino acid substitutions to about 155 amino acid substitutions, about 6 amino acid substitutions to about 150 amino acid substitutions, about 6 amino acid substitutions to about 145 amino acid substitutions, about 6 amino acid substitutions to about 140 amino acid substitutions, about 6 amino acid substitutions to about 135 amino acid substitutions, about 6 amino acid substitutions to about 130 amino acid substitutions, about 6 amino acid substitutions to about 125 amino acid substitutions, about 6 amino acid substitutions to about 120 amino acid substitutions, about 6 amino acid substitutions to about 115 amino acid substitutions, about 6 amino acid substitutions to about 110 amino acid substitutions, about 6 amino acid substitutions to about 105 amino acid substitutions, about 6 amino acid substitutions to about 100 amino acid substitutions, about 6 amino acid substitutions to about 95 amino acid substitutions, about 6 amino acid substitutions to about 90 amino acid substitutions, about 6 amino acid substitutions to about 85 amino acid substitutions, about 6 amino acid substitutions to about 80 amino acid substitutions, about 6 amino acid substitutions to about 75 amino acid substitutions, about 6 amino acid substitutions to about 70 amino acid substitutions, about 6 amino acid substitutions to about 65 amino acid substitutions, about 6 amino acid substitutions to about 60 amino acid substitutions, about 6 amino acid substitutions to about 55 amino acid substitutions, about 6 amino acid substitutions to about 50 amino acid substitutions, about 6 amino acid substitutions to about 45 amino acid substitutions, about 6 amino acid substitutions to about 40 amino acid substitutions, about 6 amino acid substitutions to about 35 amino acid substitutions, about 6 amino acid substitutions to about 30 amino acid substitutions, about 6 amino acid substitutions to about 25 amino acid substitutions, about 6 amino acid substitutions to about 20 amino acid substitutions, about 6 amino acid substitutions to about 15 amino acid substitutions, about 6 amino acid substitutions to about 10 amino acid substitutions, about 6 amino acid substitutions to about 9 amino acid substitutions, about 6 amino acid substitutions to about 8 amino acid substitutions, between about 7 amino acid substitutions to about 160 amino acid substitutions, about 7 amino acid substitutions to about 155 amino acid substitutions, about 7 amino acid substitutions to about 150 amino acid substitutions, about 7 amino acid substitutions to about 145 amino acid substitutions, about 7 amino acid substitutions to about 140 amino acid substitutions, about 7 amino acid substitutions to about 135 amino acid substitutions, about 7 amino acid substitutions to about 130 amino acid substitutions, about 7 amino acid substitutions to about 125 amino acid substitutions, about 7 amino acid substitutions to about 120 amino acid substitutions, about 7 amino acid substitutions to about 115 amino acid substitutions, about 7 amino acid substitutions to about 110 amino acid substitutions, about 7 amino acid substitutions to about 105 amino acid substitutions, about 7 amino acid substitutions to about 100 amino acid substitutions, about 7 amino acid substitutions to about 95 amino acid substitutions, about 7 amino acid substitutions to about 90 amino acid substitutions, about 7 amino acid substitutions to about 85 amino acid substitutions, about 7 amino acid substitutions to about 80 amino acid substitutions, about 7 amino acid substitutions to about 75 amino acid substitutions, about 7 amino acid substitutions to about 70 amino acid substitutions, about 7 amino acid substitutions to about 65 amino acid substitutions, about 7 amino acid substitutions to about 60 amino acid substitutions, about 7 amino acid substitutions to about 55 amino acid substitutions, about 7 amino acid substitutions to about 50 amino acid substitutions, about 7 amino acid substitutions to about 45 amino acid substitutions, about 7 amino acid substitutions to about 40 amino acid substitutions, about 7 amino acid substitutions to about 35 amino acid substitutions, about 7 amino acid substitutions to about 30 amino acid substitutions, about 7 amino acid substitutions to about 25 amino acid substitutions, about 7 amino acid substitutions to about 20 amino acid substitutions, about 7 amino acid substitutions to about 15 amino acid substitutions, about 7 amino acid substitutions to about 10 amino acid substitutions, about 7 amino acid substitutions to about 9 amino acid substitutions, between about 8 amino acid substitutions to about 160 amino acid substitutions, about 8 amino acid substitutions to about 155 amino acid substitutions, about 8 amino acid substitutions to about 150 amino acid substitutions, about 8 amino acid substitutions to about 145 amino acid substitutions, about 8 amino acid substitutions to about 140 amino acid substitutions, about 8 amino acid substitutions to about 135 amino acid substitutions, about 8 amino acid substitutions to about 130 amino acid substitutions, about 8 amino acid substitutions to about 125 amino acid substitutions, about 8 amino acid substitutions to about 120 amino acid substitutions, about 8 amino acid substitutions to about 115 amino acid substitutions, about 8 amino acid substitutions to about 110 amino acid substitutions, about 8 amino acid substitutions to about 105 amino acid substitutions, about 8 amino acid substitutions to about 100 amino acid substitutions, about 8 amino acid substitutions to about 95 amino acid substitutions, about 8 amino acid substitutions to about 90 amino acid substitutions, about 8 amino acid substitutions to about 85 amino acid substitutions, about 8 amino acid substitutions to about 80 amino acid substitutions, about 8 amino acid substitutions to about 75 amino acid substitutions, about 8 amino acid substitutions to about 70 amino acid substitutions, about 8 amino acid substitutions to about 65 amino acid substitutions, about 8 amino acid substitutions to about 60 amino acid substitutions, about 8 amino acid substitutions to about 55 amino acid substitutions, about 8 amino acid substitutions to about 50 amino acid substitutions, about 8 amino acid substitutions to about 45 amino acid substitutions, about 8 amino acid substitutions to about 40 amino acid substitutions, about 8 amino acid substitutions to about 35 amino acid substitutions, about 8 amino acid substitutions to about 30 amino acid substitutions, about 8 amino acid substitutions to about 25 amino acid substitutions, about 8 amino acid substitutions to about 20 amino acid substitutions, about 8 amino acid substitutions to about 15 amino acid substitutions, about 8 amino acid substitutions to about 10 amino acid substitutions, between about 10 amino acid substitutions to about 160 amino acid substitutions, about 10 amino acid substitutions to about 155 amino acid substitutions, about 10 amino acid substitutions to about 150 amino acid substitutions, about 10 amino acid substitutions to about 145 amino acid substitutions, about 10 amino acid substitutions to about 140 amino acid substitutions, about 10 amino acid substitutions to about 135 amino acid substitutions, about 10 amino acid substitutions to about 130 amino acid substitutions, about 10 amino acid substitutions to about 125 amino acid substitutions, about 10 amino acid substitutions to about 120 amino acid substitutions, about 10 amino acid substitutions to about 115 amino acid substitutions, about 10 amino acid substitutions to about 110 amino acid substitutions, about 10 amino acid substitutions to about 105 amino acid substitutions, about 10 amino acid substitutions to about 100 amino acid substitutions, about 10 amino acid substitutions to about 95 amino acid substitutions, about 10 amino acid substitutions to about 90 amino acid substitutions, about 10 amino acid substitutions to about 85 amino acid substitutions, about 10 amino acid substitutions to about 80 amino acid substitutions, about 10 amino acid substitutions to about 75 amino acid substitutions, about 10 amino acid substitutions to about 70 amino acid substitutions, about 10 amino acid substitutions to about 65 amino acid substitutions, about 10 amino acid substitutions to about 60 amino acid substitutions, about 10 amino acid substitutions to about 55 amino acid substitutions, about 10 amino acid substitutions to about 50 amino acid substitutions, about 10 amino acid substitutions to about 45 amino acid substitutions, about 10 amino acid substitutions to about 40 amino acid substitutions, about 10 amino acid substitutions to about 35 amino acid substitutions, about 10 amino acid substitutions to about 30 amino acid substitutions, about 10 amino acid substitutions to about 25 amino acid substitutions, about 10 amino acid substitutions to about 20 amino acid substitutions, about 10 amino acid substitutions to about 15 amino acid substitutions, between about 15 amino acid substitutions to about 160 amino acid substitutions, about 15 amino acid substitutions to about 155 amino acid substitutions, about 15 amino acid substitutions to about 150 amino acid substitutions, about 15 amino acid substitutions to about 145 amino acid substitutions, about 15 amino acid substitutions to about 140 amino acid substitutions, about 15 amino acid substitutions to about 135 amino acid substitutions, about 15 amino acid substitutions to about 130 amino acid substitutions, about 15 amino acid substitutions to about 125 amino acid substitutions, about 15 amino acid substitutions to about 120 amino acid substitutions, about 15 amino acid substitutions to about 115 amino acid substitutions, about 15 amino acid substitutions to about 110 amino acid substitutions, about 15 amino acid substitutions to about 105 amino acid substitutions, about 15 amino acid substitutions to about 100 amino acid substitutions, about 15 amino acid substitutions to about 95 amino acid substitutions, about 15 amino acid substitutions to about 90 amino acid substitutions, about 15 amino acid substitutions to about 85 amino acid substitutions, about 15 amino acid substitutions to about 80 amino acid substitutions, about 15 amino acid substitutions to about 75 amino acid substitutions, about 15 amino acid substitutions to about 70 amino acid substitutions, about 15 amino acid substitutions to about 65 amino acid substitutions, about 15 amino acid substitutions to about 60 amino acid substitutions, about 15 amino acid substitutions to about 55 amino acid substitutions, about 15 amino acid substitutions to about 50 amino acid substitutions, about 15 amino acid substitutions to about 45 amino acid substitutions, about 15 amino acid substitutions to about 40 amino acid substitutions, about 15 amino acid substitutions to about 35 amino acid substitutions, about 15 amino acid substitutions to about 30 amino acid substitutions, about 15 amino acid substitutions to about 25 amino acid substitutions, about 15 amino acid substitutions to about 20 amino acid substitutions, between about 20 amino acid substitutions to about 160 amino acid substitutions, about 20 amino acid substitutions to about 155 amino acid substitutions, about 20 amino acid substitutions to about 150 amino acid substitutions, about 20 amino acid substitutions to about 145 amino acid substitutions, about 20 amino acid substitutions to about 140 amino acid substitutions, about 20 amino acid substitutions to about 135 amino acid substitutions, about 20 amino acid substitutions to about 130 amino acid substitutions, about 20 amino acid substitutions to about 125 amino acid substitutions, about 20 amino acid substitutions to about 120 amino acid substitutions, about 20 amino acid substitutions to about 115 amino acid substitutions, about 20 amino acid substitutions to about 110 amino acid substitutions, about 20 amino acid substitutions to about 105 amino acid substitutions, about 20 amino acid substitutions to about 100 amino acid substitutions, about 20 amino acid substitutions to about 95 amino acid substitutions, about 20 amino acid substitutions to about 90 amino acid substitutions, about 20 amino acid substitutions to about 85 amino acid substitutions, about 20 amino acid substitutions to about 80 amino acid substitutions, about 20 amino acid substitutions to about 75 amino acid substitutions, about 20 amino acid substitutions to about 70 amino acid substitutions, about 20 amino acid substitutions to about 65 amino acid substitutions, about 20 amino acid substitutions to about 60 amino acid substitutions, about 20 amino acid substitutions to about 55 amino acid substitutions, about 20 amino acid substitutions to about 50 amino acid substitutions, about 20 amino acid substitutions to about 45 amino acid substitutions, about 20 amino acid substitutions to about 40 amino acid substitutions, about 20 amino acid substitutions to about 35 amino acid substitutions, about 20 amino acid substitutions to about 30 amino acid substitutions, about 20 amino acid substitutions to about 25 amino acid substitutions, between about 25 amino acid substitutions to about 160 amino acid substitutions, about 25 amino acid substitutions to about 155 amino acid substitutions, about 25 amino acid substitutions to about 150 amino acid substitutions, about 25 amino acid substitutions to about 145 amino acid substitutions, about 25 amino acid substitutions to about 140 amino acid substitutions, about 25 amino acid substitutions to about 135 amino acid substitutions, about 25 amino acid substitutions to about 130 amino acid substitutions, about 25 amino acid substitutions to about 125 amino acid substitutions, about 25 amino acid substitutions to about 120 amino acid substitutions, about 25 amino acid substitutions to about 115 amino acid substitutions, about 25 amino acid substitutions to about 110 amino acid substitutions, about 25 amino acid substitutions to about 105 amino acid substitutions, about 25 amino acid substitutions to about 100 amino acid substitutions, about 25 amino acid substitutions to about 95 amino acid substitutions, about 25 amino acid substitutions to about 90 amino acid substitutions, about 25 amino acid substitutions to about 85 amino acid substitutions, about 25 amino acid substitutions to about 80 amino acid substitutions, about 25 amino acid substitutions to about 75 amino acid substitutions, about 25 amino acid substitutions to about 70 amino acid substitutions, about 25 amino acid substitutions to about 65 amino acid substitutions, about 25 amino acid substitutions to about 60 amino acid substitutions, about 25 amino acid substitutions to about 55 amino acid substitutions, about 25 amino acid substitutions to about 50 amino acid substitutions, about 25 amino acid substitutions to about 45 amino acid substitutions, about 25 amino acid substitutions to about 40 amino acid substitutions, about 25 amino acid substitutions to about 35 amino acid substitutions, about 25 amino acid substitutions to about 30 amino acid substitutions, between about 30 amino acid substitutions to about 160 amino acid substitutions, about 30 amino acid substitutions to about 155 amino acid substitutions, about 30 amino acid substitutions to about 150 amino acid substitutions, about 30 amino acid substitutions to about 145 amino acid substitutions, about 30 amino acid substitutions to about 140 amino acid substitutions, about 30 amino acid substitutions to about 135 amino acid substitutions, about 30 amino acid substitutions to about 130 amino acid substitutions, about 30 amino acid substitutions to about 125 amino acid substitutions, about 30 amino acid substitutions to about 120 amino acid substitutions, about 30 amino acid substitutions to about 115 amino acid substitutions, about 30 amino acid substitutions to about 110 amino acid substitutions, about 30 amino acid substitutions to about 105 amino acid substitutions, about 30 amino acid substitutions to about 100 amino acid substitutions, about 30 amino acid substitutions to about 95 amino acid substitutions, about 30 amino acid substitutions to about 90 amino acid substitutions, about 30 amino acid substitutions to about 85 amino acid substitutions, about 30 amino acid substitutions to about 80 amino acid substitutions, about 30 amino acid substitutions to about 75 amino acid substitutions, about 30 amino acid substitutions to about 70 amino acid substitutions, about 30 amino acid substitutions to about 65 amino acid substitutions, about 30 amino acid substitutions to about 60 amino acid substitutions, about 30 amino acid substitutions to about 55 amino acid substitutions, about 30 amino acid substitutions to about 50 amino acid substitutions, about 30 amino acid substitutions to about 45 amino acid substitutions, about 30 amino acid substitutions to about 40 amino acid substitutions, about 30 amino acid substitutions to about 35 amino acid substitutions, between about 35 amino acid substitutions to about 160 amino acid substitutions, about 35 amino acid substitutions to about 155 amino acid substitutions, about 35 amino acid substitutions to about 150 amino acid substitutions, about 35 amino acid substitutions to about 145 amino acid substitutions, about 35 amino acid substitutions to about 140 amino acid substitutions, about 35 amino acid substitutions to about 135 amino acid substitutions, about 35 amino acid substitutions to about 130 amino acid substitutions, about 35 amino acid substitutions to about 125 amino acid substitutions, about 35 amino acid substitutions to about 120 amino acid substitutions, about 35 amino acid substitutions to about 115 amino acid substitutions, about 35 amino acid substitutions to about 110 amino acid substitutions, about 35 amino acid substitutions to about 105 amino acid substitutions, about 35 amino acid substitutions to about 100 amino acid substitutions, about 35 amino acid substitutions to about 95 amino acid substitutions, about 35 amino acid substitutions to about 90 amino acid substitutions, about 35 amino acid substitutions to about 85 amino acid substitutions, about 35 amino acid substitutions to about 80 amino acid substitutions, about 35 amino acid substitutions to about 75 amino acid substitutions, about 35 amino acid substitutions to about 70 amino acid substitutions, about 35 amino acid substitutions to about 65 amino acid substitutions, about 35 amino acid substitutions to about 60 amino acid substitutions, about 35 amino acid substitutions to about 55 amino acid substitutions, about 35 amino acid substitutions to about 50 amino acid substitutions, about 35 amino acid substitutions to about 45 amino acid substitutions, about 35 amino acid substitutions to about 40 amino acid substitutions, between about 40 amino acid substitutions to about 160 amino acid substitutions, about 40 amino acid substitutions to about 155 amino acid substitutions, about 40 amino acid substitutions to about 150 amino acid substitutions, about 40 amino acid substitutions to about 145 amino acid substitutions, about 40 amino acid substitutions to about 140 amino acid substitutions, about 40 amino acid substitutions to about 135 amino acid substitutions, about 40 amino acid substitutions to about 130 amino acid substitutions, about 40 amino acid substitutions to about 125 amino acid substitutions, about 40 amino acid substitutions to about 120 amino acid substitutions, about 40 amino acid substitutions to about 115 amino acid substitutions, about 40 amino acid substitutions to about 110 amino acid substitutions, about 40 amino acid substitutions to about 105 amino acid substitutions, about 40 amino acid substitutions to about 100 amino acid substitutions, about 40 amino acid substitutions to about 95 amino acid substitutions, about 40 amino acid substitutions to about 90 amino acid substitutions, about 40 amino acid substitutions to about 85 amino acid substitutions, about 40 amino acid substitutions to about 80 amino acid substitutions, about 40 amino acid substitutions to about 75 amino acid substitutions, about 40 amino acid substitutions to about 70 amino acid substitutions, about 40 amino acid substitutions to about 65 amino acid substitutions, about 40 amino acid substitutions to about 60 amino acid substitutions, about 40 amino acid substitutions to about 55 amino acid substitutions, about 40 amino acid substitutions to about 50 amino acid substitutions, about 40 amino acid substitutions to about 45 amino acid substitutions, between about 45 amino acid substitutions to about 160 amino acid substitutions, about 45 amino acid substitutions to about 155 amino acid substitutions, about 45 amino acid substitutions to about 150 amino acid substitutions, about 45 amino acid substitutions to about 145 amino acid substitutions, about 45 amino acid substitutions to about 140 amino acid substitutions, about 45 amino acid substitutions to about 135 amino acid substitutions, about 45 amino acid substitutions to about 130 amino acid substitutions, about 45 amino acid substitutions to about 125 amino acid substitutions, about 45 amino acid substitutions to about 120 amino acid substitutions, about 45 amino acid substitutions to about 115 amino acid substitutions, about 45 amino acid substitutions to about 110 amino acid substitutions, about 45 amino acid substitutions to about 105 amino acid substitutions, about 45 amino acid substitutions to about 100 amino acid substitutions, about 45 amino acid substitutions to about 95 amino acid substitutions, about 45 amino acid substitutions to about 90 amino acid substitutions, about 45 amino acid substitutions to about 85 amino acid substitutions, about 45 amino acid substitutions to about 80 amino acid substitutions, about 45 amino acid substitutions to about 75 amino acid substitutions, about 45 amino acid substitutions to about 70 amino acid substitutions, about 45 amino acid substitutions to about 65 amino acid substitutions, about 45 amino acid substitutions to about 60 amino acid substitutions, about 45 amino acid substitutions to about 55 amino acid substitutions, about 45 amino acid substitutions to about 50 amino acid substitutions, between about 50 amino acid substitutions to about 160 amino acid substitutions, about 50 amino acid substitutions to about 155 amino acid substitutions, about 50 amino acid substitutions to about 150 amino acid substitutions, about 50 amino acid substitutions to about 145 amino acid substitutions, about 50 amino acid substitutions to about 140 amino acid substitutions, about 50 amino acid substitutions to about 135 amino acid substitutions, about 50 amino acid substitutions to about 130 amino acid substitutions, about 50 amino acid substitutions to about 125 amino acid substitutions, about 50 amino acid substitutions to about 120 amino acid substitutions, about 50 amino acid substitutions to about 115 amino acid substitutions, about 50 amino acid substitutions to about 110 amino acid substitutions, about 50 amino acid substitutions to about 105 amino acid substitutions, about 50 amino acid substitutions to about 100 amino acid substitutions, about 50 amino acid substitutions to about 95 amino acid substitutions, about 50 amino acid substitutions to about 90 amino acid substitutions, about 50 amino acid substitutions to about 85 amino acid substitutions, about 50 amino acid substitutions to about 80 amino acid substitutions, about 50 amino acid substitutions to about 75 amino acid substitutions, about 50 amino acid substitutions to about 70 amino acid substitutions, about 50 amino acid substitutions to about 65 amino acid substitutions, about 50 amino acid substitutions to about 60 amino acid substitutions, about 50 amino acid substitutions to about 55 amino acid substitutions, between about 60 amino acid substitutions to about 160 amino acid substitutions, about 60 amino acid substitutions to about 155 amino acid substitutions, about 60 amino acid substitutions to about 150 amino acid substitutions, about 60 amino acid substitutions to about 145 amino acid substitutions, about 60 amino acid substitutions to about 140 amino acid substitutions, about 60 amino acid substitutions to about 135 amino acid substitutions, about 60 amino acid substitutions to about 130 amino acid substitutions, about 60 amino acid substitutions to about 125 amino acid substitutions, about 60 amino acid substitutions to about 120 amino acid substitutions, about 60 amino acid substitutions to about 115 amino acid substitutions, about 60 amino acid substitutions to about 110 amino acid substitutions, about 60 amino acid substitutions to about 105 amino acid substitutions, about 60 amino acid substitutions to about 100 amino acid substitutions, about 60 amino acid substitutions to about 95 amino acid substitutions, about 60 amino acid substitutions to about 90 amino acid substitutions, about 60 amino acid substitutions to about 85 amino acid substitutions, about 60 amino acid substitutions to about 80 amino acid substitutions, about 60 amino acid substitutions to about 75 amino acid substitutions, about 60 amino acid substitutions to about 70 amino acid substitutions, about 60 amino acid substitutions to about 65 amino acid substitutions, between about 70 amino acid substitutions to about 160 amino acid substitutions, about 70 amino acid substitutions to about 155 amino acid substitutions, about 70 amino acid substitutions to about 150 amino acid substitutions, about 70 amino acid substitutions to about 145 amino acid substitutions, about 70 amino acid substitutions to about 140 amino acid substitutions, about 70 amino acid substitutions to about 135 amino acid substitutions, about 70 amino acid substitutions to about 130 amino acid substitutions, about 70 amino acid substitutions to about 125 amino acid substitutions, about 70 amino acid substitutions to about 120 amino acid substitutions, about 70 amino acid substitutions to about 115 amino acid substitutions, about 70 amino acid substitutions to about 110 amino acid substitutions, about 70 amino acid substitutions to about 105 amino acid substitutions, about 70 amino acid substitutions to about 100 amino acid substitutions, about 70 amino acid substitutions to about 95 amino acid substitutions, about 70 amino acid substitutions to about 90 amino acid substitutions, about 70 amino acid substitutions to about 85 amino acid substitutions, about 70 amino acid substitutions to about 80 amino acid substitutions, about 70 amino acid substitutions to about 75 amino acid substitutions, between about 80 amino acid substitutions to about 160 amino acid substitutions, about 80 amino acid substitutions to about 155 amino acid substitutions, about 80 amino acid substitutions to about 150 amino acid substitutions, about 80 amino acid substitutions to about 145 amino acid substitutions, about 80 amino acid substitutions to about 140 amino acid substitutions, about 80 amino acid substitutions to about 135 amino acid substitutions, about 80 amino acid substitutions to about 130 amino acid substitutions, about 80 amino acid substitutions to about 125 amino acid substitutions, about 80 amino acid substitutions to about 120 amino acid substitutions, about 80 amino acid substitutions to about 115 amino acid substitutions, about 80 amino acid substitutions to about 110 amino acid substitutions, about 80 amino acid substitutions to about 105 amino acid substitutions, about 80 amino acid substitutions to about 100 amino acid substitutions, about 80 amino acid substitutions to about 95 amino acid substitutions, about 80 amino acid substitutions to about 90 amino acid substitutions, about 80 amino acid substitutions to about 85 amino acid substitutions, between about 90 amino acid substitutions to about 160 amino acid substitutions, about 90 amino acid substitutions to about 155 amino acid substitutions, about 90 amino acid substitutions to about 150 amino acid substitutions, about 90 amino acid substitutions to about 145 amino acid substitutions, about 90 amino acid substitutions to about 140 amino acid substitutions, about 90 amino acid substitutions to about 135 amino acid substitutions, about 90 amino acid substitutions to about 130 amino acid substitutions, about 90 amino acid substitutions to about 125 amino acid substitutions, about 90 amino acid substitutions to about 120 amino acid substitutions, about 90 amino acid substitutions to about 115 amino acid substitutions, about 90 amino acid substitutions to about 110 amino acid substitutions, about 90 amino acid substitutions to about 105 amino acid substitutions, about 90 amino acid substitutions to about 100 amino acid substitutions, about 90 amino acid substitutions to about 95 amino acid substitutions, between about 100 amino acid substitutions to about 160 amino acid substitutions, about 100 amino acid substitutions to about 155 amino acid substitutions, about 100 amino acid substitutions to about 150 amino acid substitutions, about 100 amino acid substitutions to about 145 amino acid substitutions, about 100 amino acid substitutions to about 140 amino acid substitutions, about 100 amino acid substitutions to about 135 amino acid substitutions, about 100 amino acid substitutions to about 130 amino acid substitutions, about 100 amino acid substitutions to about 125 amino acid substitutions, about 100 amino acid substitutions to about 120 amino acid substitutions, about 100 amino acid substitutions to about 115 amino acid substitutions, about 100 amino acid substitutions to about 110 amino acid substitutions, about 100 amino acid substitutions to about 105 amino acid substitutions, between about 110 amino acid substitutions to about 160 amino acid substitutions, about 110 amino acid substitutions to about 155 amino acid substitutions, about 110 amino acid substitutions to about 150 amino acid substitutions, about 110 amino acid substitutions to about 145 amino acid substitutions, about 110 amino acid substitutions to about 140 amino acid substitutions, about 110 amino acid substitutions to about 135 amino acid substitutions, about 110 amino acid substitutions to about 130 amino acid substitutions, about 110 amino acid substitutions to about 125 amino acid substitutions, about 110 amino acid substitutions to about 120 amino acid substitutions, about 110 amino acid substitutions to about 115 amino acid substitutions, between about 120 amino acid substitutions to about 160 amino acid substitutions, about 120 amino acid substitutions to about 155 amino acid substitutions, about 120 amino acid substitutions to about 150 amino acid substitutions, about 120 amino acid substitutions to about 145 amino acid substitutions, about 120 amino acid substitutions to about 140 amino acid substitutions, about 120 amino acid substitutions to about 135 amino acid substitutions, about 120 amino acid substitutions to about 130 amino acid substitutions, about 120 amino acid substitutions to about 125 amino acid substitutions, between about 130 amino acid substitutions to about 160 amino acid substitutions, about 130 amino acid substitutions to about 155 amino acid substitutions, about 130 amino acid substitutions to about 150 amino acid substitutions, about 130 amino acid substitutions to about 145 amino acid substitutions, about 130 amino acid substitutions to about 140 amino acid substitutions, about 130 amino acid substitutions to about 135 amino acid substitutions, between about 140 amino acid substitutions to about 160 amino acid substitutions, about 140 amino acid substitutions to about 155 amino acid substitutions, about 140 amino acid substitutions to about 150 amino acid substitutions, about 140 amino acid substitutions to about 145 amino acid substitutions, between about 150 amino acid substitutions to about 160 amino acid substitutions, or about 150 amino acid substitutions to about 155 amino acid substitutions. One skilled in the art would appreciate that amino acids that are conserved between wildtype stereocilin proteins from different species can be mutated without losing activity, while those amino acids that are not conserved between wildtype stereocilin proteins from different species should not be mutated as they are more likely (than amino acids that are not conserved between different species) to be involved in activity.

An active stereocilin protein can include, e.g., a sequence of a wildtype, full-length stereocilin protein (e.g., a wildtype, human, full-length stereocilin protein) that has 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 50 amino acids, about 40 amino acids to about 45 amino acids, about 45 amino acids to about 50 amino acids, deleted. In some embodiments where two or more amino acids are deleted from the sequence of a wildtype, full-length stereocilin protein, the two or more deleted amino acids can be contiguous in the sequence of the wildtype, full-length protein. In other examples where two or more amino acids are deleted from the sequence of a wildtype, full-length stereocilin protein, the two or more deleted amino acids are not contiguous in the sequence of the wildtype, full-length protein. One skilled in the art would appreciate that amino acids that are not conserved between wildtype, full-length stereocilin proteins from different species can be deleted without losing activity, while those amino acids that are conserved between wildtype, full-length stereocilin proteins from different species should not be deleted as they are more likely (than amino acids that are not conserved between different species) to be involved in activity.

In some examples, an active stereocilin protein can, e.g., include a sequence of a wildtype, full-length stereocilin protein that has between 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 100 amino acids, about 2 amino acid to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 100 amino acids, about 3 amino acid to about 95 amino acids, about 3 amino acids to about 90 amino acids, about 3 amino acids to about 85 amino acids, about 3 amino acids to about 80 amino acids, about 3 amino acids to about 75 amino acids, about 3 amino acids to about 70 amino acids, about 3 amino acids to about 65 amino acids, about 3 amino acids to about 60 amino acids, about 3 amino acids to about 55 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 100 amino acids, about 4 amino acid to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 100 amino acids, about 5 amino acid to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 100 amino acids, about 6 amino acid to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 100 amino acids, about 7 amino acid to about 95 amino acids, about 7 amino acids to about 90 amino acids, about 7 amino acids to about 85 amino acids, about 7 amino acids to about 80 amino acids, about 7 amino acids to about 75 amino acids, about 7 amino acids to about 70 amino acids, about 7 amino acids to about 65 amino acids, about 7 amino acids to about 60 amino acids, about 7 amino acids to about 55 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 100 amino acids, about 8 amino acid to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acid to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 20 amino acids to about 100 amino acids, about 20 amino acid to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 100 amino acids, about 30 amino acid to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 40 amino acids to about 100 amino acids, about 40 amino acid to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 50 amino acids to about 100 amino acids, about 50 amino acid to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 60 amino acids to about 100 amino acids, about 60 amino acid to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 70 amino acids to about 100 amino acids, about 70 amino acid to about 95 amino acids, about 70 amino acids to about 90 amino acids, about 70 amino acids to about 85 amino acids, about 70 amino acids to about 80 amino acids, about 70 amino acids to about 75 amino acids, about 80 amino acids to about 100 amino acids, about 80 amino acid to about 95 amino acids, about 80 amino acids to about 90 amino acids, about 80 amino acids to about 85 amino acids, about 90 amino acids to about 100 amino acids, about 90 amino acids to about 95 amino acids, or about 95 amino acids to about 100 amino acids, removed from its N-terminus and/or 1 amino acid to 100 amino acids (or any of the subranges of this range described herein) removed from its C-terminus.

In some embodiments, an active stereocilin protein can, e.g., include the sequence of a wildtype, full-length stereocilin protein where 1 amino acid to 50 amino acids, 1 amino acid to 45 amino acids, 1 amino acid to 40 amino acids, 1 amino acid to 35 amino acids, 1 amino acid to 30 amino acids, 1 amino acid to 25 amino acids, 1 amino acid to 20 amino acids, 1 amino acid to 15 amino acids, 1 amino acid to 10 amino acids, 1 amino acid to 9 amino acids, 1 amino acid to 8 amino acids, 1 amino acid to 7 amino acids, 1 amino acid to 6 amino acids, 1 amino acid to 5 amino acids, 1 amino acid to 4 amino acids, 1 amino acid to 3 amino acids, about 2 amino acids to 50 amino acids, about 2 amino acids to 45 amino acids, about 2 amino acids to 40 amino acids, about 2 amino acids to 35 amino acids, about 2 amino acids to 30 amino acids, about 2 amino acids to 25 amino acids, about 2 amino acids to 20 amino acids, about 2 amino acids to 15 amino acids, about 2 amino acids to 10 amino acids, about 2 amino acids to 9 amino acids, about 2 amino acids to 8 amino acids, about 2 amino acids to 7 amino acids, about 2 amino acids to 6 amino acids, about 2 amino acids to 5 amino acids, about 2 amino acids to 4 amino acids, about 3 amino acids to 50 amino acids, about 3 amino acids to 45 amino acids, about 3 amino acids to 40 amino acids, about 3 amino acids to 35 amino acids, about 3 amino acids to 30 amino acids, about 3 amino acids to 25 amino acids, about 3 amino acids to 20 amino acids, about 3 amino acids to 15 amino acids, about 3 amino acids to 10 amino acids, about 3 amino acids to 9 amino acids, about 3 amino acids to 8 amino acids, about 3 amino acids to 7 amino acids, about 3 amino acids to 6 amino acids, about 3 amino acids to 5 amino acids, about 4 amino acids to 50 amino acids, about 4 amino acids to 45 amino acids, about 4 amino acids to 40 amino acids, about 4 amino acids to 35 amino acids, about 4 amino acids to 30 amino acids, about 4 amino acids to 25 amino acids, about 4 amino acids to 20 amino acids, about 4 amino acids to 15 amino acids, about 4 amino acids to 10 amino acids, about 4 amino acids to 9 amino acids, about 4 amino acids to 8 amino acids, about 4 amino acids to 7 amino acids, about 4 amino acids to 6 amino acids, about 5 amino acids to 50 amino acids, about 5 amino acids to 45 amino acids, about 5 amino acids to 40 amino acids, about 5 amino acids to 35 amino acids, about 5 amino acids to 30 amino acids, about 5 amino acids to 25 amino acids, about 5 amino acids to 20 amino acids, about 5 amino acids to 15 amino acids, about 5 amino acids to 10 amino acids, about 5 amino acids to 9 amino acids, about 5 amino acids to 8 amino acids, about 5 amino acids to 7 amino acids, about 6 amino acids to 50 amino acids, about 6 amino acids to 45 amino acids, about 6 amino acids to 40 amino acids, about 6 amino acids to 35 amino acids, about 6 amino acids to 30 amino acids, about 6 amino acids to 25 amino acids, about 6 amino acids to 20 amino acids, about 6 amino acids to 15 amino acids, about 6 amino acids to 10 amino acids, about 6 amino acids to 9 amino acids, about 6 amino acids to 8 amino acids, about 7 amino acids to 50 amino acids, about 7 amino acids to 45 amino acids, about 7 amino acids to 40 amino acids, about 7 amino acids to 35 amino acids, about 7 amino acids to 30 amino acids, about 7 amino acids to 25 amino acids, about 7 amino acids to 20 amino acids, about 7 amino acids to 15 amino acids, about 7 amino acids to 10 amino acids, about 7 amino acids to 9 amino acids, about 8 amino acids to 50 amino acids, about 8 amino acids to 45 amino acids, about 8 amino acids to 40 amino acids, about 8 amino acids to 35 amino acids, about 8 amino acids to 30 amino acids, about 8 amino acids to 25 amino acids, about 8 amino acids to 20 amino acids, about 8 amino acids to 15 amino acids, about 8 amino acids to 10 amino acids, about 10 amino acids to 50 amino acids, about 10 amino acids to 45 amino acids, about 10 amino acids to 40 amino acids, about 10 amino acids to 35 amino acids, about 10 amino acids to 30 amino acids, about 10 amino acids to 25 amino acids, about 10 amino acids to 20 amino acids, about 10 amino acids to 15 amino acids, about 15 amino acids to 50 amino acids, about 15 amino acids to 45 amino acids, about 15 amino acids to 40 amino acids, about 15 amino acids to 35 amino acids, about 15 amino acids to 30 amino acids, about 15 amino acids to 25 amino acids, about 15 amino acids to 20 amino acids, about 20 amino acids to 50 amino acids, about 20 amino acids to 45 amino acids, about 20 amino acids to 40 amino acids, about 20 amino acids to 35 amino acids, about 20 amino acids to 30 amino acids, about 20 amino acids to 25 amino acids, about 25 amino acids to 50 amino acids, about 25 amino acids to 45 amino acids, about 25 amino acids to 40 amino acids, about 25 amino acids to 35 amino acids, about 25 amino acids to 30 amino acids, about 30 amino acids to 50 amino acids, about 30 amino acids to 45 amino acids, about 30 amino acids to 40 amino acids, about 30 amino acids to 35 amino acids, about 35 amino acids to 50 amino acids, about 35 amino acids to 45 amino acids, about 35 amino acids to 40 amino acids, about 40 amino acids to 50 amino acids, about 40 amino acids to 45 amino acids, or about 45 amino acids to about 50 amino acids, are inserted. In some examples, the 1 amino acid to 50 amino acids (or any subrange thereof) can be inserted as a contiguous sequence into the sequence of a wildtype, full-length protein. In some examples, the 1 amino acid to 50 amino acids (or any subrange thereof) are not inserted as a contiguous sequence into the sequence of a wildtype, full-length protein. As can be appreciated in the art, the 1 amino acid to 50 amino acids can be inserted into a portion of the sequence of a wildtype, full-length protein that is not well-conserved between species.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF DRAWINGS

FIG. 1 is an exemplary schematic representation of a genetic map of a STRC vector (pITR-201; SEQ ID NO: 12; 4324 basepairs (bp)) that can be used in any of the present methods described herein. The vector includes an inverted terminal repeat (ITR) sequence (SEQ ID NO: 18), a 5′ FVIII stuffer sequence (SEQ ID NO: 19), a CMV enhancer (SEQ ID NO: 20), a CMV promoter (SEQ ID NO: 21), a 5′ untranslated region (UTR) sequence (SEQ ID NO: 24), a 5′ STRC coding sequence (SEQ ID NO: 25), a splicing donor signal sequence (SD) (SEQ ID NO: 6), a highly recombinogenic sequence from F1 phage (AK; SEQ ID NO: 26), and an ITR sequence (SEQ ID NO: 36).

FIG. 2 is an exemplary schematic representation of a genetic map of a STRC vector (pITR-202; SEQ ID NO: 13; 4434 bp) that can be used in any of the present methods described herein. The vector includes an ITR sequence (SEQ ID NO: 18), an AK sequence (SEQ ID NO: 26), a SD sequence (SEQ ID NO: 6), a 3′ STRC coding sequence (SEQ ID NO: 28), a 3′ UTR sequence (SEQ ID NO: 33), a bovine growth hormone poly-adenylation signal (bGHpA) (SEQ ID NO: 34), a 3′ FVIII stuffer sequence (SEQ ID NO: 35), and an ITR sequence (SEQ ID NO: 36).

FIG. 3 is an exemplary schematic representation of a genetic map of a STRC vector (pITR-202GFP; SEQ ID NO: 14; 4693 bp) that can be used in any of the present methods described herein. The vector includes an ITR sequence (SEQ ID NO: 18), an AK sequence (SEQ ID NO: 26), a SD sequence (SEQ ID NO: 6), a 3′ STRC coding sequence (SEQ ID NO: 28), a T2A sequence (SEQ ID NO: 29), a tGFP sequence (SEQ ID NO: 31), a 3′ UTR sequence (SEQ ID NO: 33), a bGHpA sequence (SEQ ID NO: 34), and an ITR sequence (SEQ ID NO: 36).

FIG. 4 is an exemplary schematic representation of a genetic map of a STRC vector (pITR-203; SEQ ID NO: 15; 4619 bp) that can be used in any of the present methods described herein. The vector includes an ITR sequence (SEQ ID NO: 18), a 5′ FVIII stuffer_500 sequence (SEQ ID NO: 37), a CMV enhancer (SEQ ID NO: 20), a CMV promoter sequence (SEQ ID NO: 21), a 5′ UTR sequence (SEQ ID NO: 24), a 5′ STRC coding sequence (SEQ ID NO: 25), a SD sequence (SEQ ID NO: 6), an AP sequence (AP) (SEQ ID NO: 27), and an ITR sequence (SEQ ID NO: 36).

FIG. 5 is an exemplary schematic representation of a genetic map of a STRC vector (pITR-204; SEQ ID NO: 16; 4729 bp) that can be used in any of the present methods described herein. The vector includes an ITR sequence (SEQ ID NO: 18), an AP sequence (SEQ ID NO: 27), a splicing acceptor signal (SA) sequence (SEQ ID NO: 7), a 3′ STRC coding sequence (SEQ ID NO: 28), a 3′ UTR sequence (SEQ ID NO: 33), a bGHpA sequence (SEQ ID NO: 34), and an ITR sequence (SEQ ID NO: 36).

FIG. 6 is an exemplary schematic representation of a genetic map of a STRC vector (pITR-205; SEQ ID NO: 17; 4460 bp) that can be used in any of the present methods described herein. The vector includes an ITR sequence (SEQ ID NO: 18), a CMV enhancer (SEQ ID NO: 20), a chicken R-actin (CBA) promoter (SEQ ID NO: 22), a chimeric intron sequence (SEQ ID NO: 23), a 5′ UTR sequence (SEQ ID NO: 24), a 5′ STRC coding sequence (SEQ ID NO: 25), a SD sequence (SEQ ID NO: 6), an AK sequence (SEQ ID NO: 26), and an ITR sequence (SEQ ID NO: 36).

FIG. 7 is an image of an immunoblot of STRC protein levels from transfected HEK293FT cells with single or dual STRC vectors 72-hours post-transfection using a polyclonal STRC antibody. β-actin (ACTB) was used as a loading control. Lane 1—Prestrained Page Rule; Lane 2—400 ng pITR-201 plus 400 ng pITR-202; Lane 3—400 ng pITR-201 plus 400 ng pITR-202GFP; Lane 4—400 ng pITR-203 plus 400 ng pITR-204; Lane 5—400 ng pITR-205 plus 400 ng pITR-202; Lane 6—400 ng pITR-205 plus 400 ng pITR-202GFP; Lane 7—800 ng pITR-202GFP; and Lane 8—untransfected control cell lysate.

FIG. 8 is an image of an immunoblot of STRC protein levels from transduced HEK293FT cells with dual STRC vectors (AAV2.STRC and Anc80.STRC) at different MOIs (2.6E5, 8.6E4, 2.6E5, and 8.6E4) 72-hours post-transfection using a polyclonal STRC antibody. R-actin (ACTB) was used as a loading control.

FIG. 9 is a bar graph showing the relative RNA expression of STRC (5′ STRC, 3′ STRC and full-length STRC) relative to GAPDH in HEK293FT cells transduced with AAV2.STRC (at MOI 8.6E4 and MOI 2.6E5) and Anc80.STRC (at MOI 8.6E4 and 2.6E5), respectively.

FIG. 10 is a bar graph showing the relative RNA expression of STRC (5′ STRC, 3′ STRC and full-length STRC) relative to GAPDH in P1-3 cochlear explants from WT mice infected 16-hours with AAV2.STRC (at MOI 1.3E10) and Anc80.STRC (at MOI 3.9E10), respectively.

FIG. 11A is an exemplary fluorescent image of P1-3 cochlear explants from mock-infected WT mice showing Myo7a and phalloidin staining.

FIG. 11B is an exemplary fluorescent image of P1-3 cochlear explants from mock-infected WT mice showing Myo7a and phalloidin staining. Magnification 20×.

FIG. 11C is an exemplary fluorescent image of P1-3 cochlear explants from AAV.Anc80.STRC-infected (at MOI 7.8E8 VG/cochlea) WT mice showing Myo7a and phalloidin staining.

FIG. 11D is an exemplary fluorescent image of P1-3 cochlear explants from AAVanc80.dSTRC-infected (at MOI 2.6E8 VG/cochlea) WT mice showing Myo7a and phalloidin staining. Magnification 20×.

FIG. 11E is an exemplary fluorescent image of P1-3 cochlear explants from AAV.Anc80.STRC-infected (at MOI 7.8E8 VG/cochlea) WT mice showing Myo7a and phalloidin staining. Magnification 20×.

FIG. 12A is an exemplary fluorescent image of P1-3 cochlear explants from AAV2.STRC-infected (at MOI 7.8E8 VG/cochlea) WT mice showing Myo7a and phalloidin staining.

FIG. 12B is an exemplary fluorescent image of P1-3 cochlear explants from AAV2.STRC-infected (at MOI 2.6E8 VG/cochlea) WT mice showing Myo7a and phalloidin staining. Magnification 20×.

FIG. 12C is an exemplary fluorescent image of P1-3 cochlear explants from AAV2.STRC-infected (at MOI 7.8E8 VG/cochlea) WT mice showing Myo7a and phalloidin staining. Magnification 20×.

### DETAILED DESCRIPTION

Provided herein are compositions that include at least two different nucleic acid vectors, where: each of the at least two different vectors includes a coding sequence that encodes a different portion of a stereocilin protein, each of the encoded portions being at least 30 amino acid residues in length, where the amino acid sequence of each of the encoded portions may optionally partially overlap with the amino acid sequence of a different one of the encoded portions; no single vector of the at least two different vectors encodes an active stereocilin protein (e.g., a full-length stereocilin protein); at least one of the coding sequences comprises a nucleotide sequence spanning two neighboring exons of stereocilin genomic DNA, and lacks an intronic sequence between the two neighboring exons; and, when introduced into a mammalian cell comprising chromosomal DNA, the at least two different vectors undergo homologous recombination with each other and with the chromosomal DNA of the cell, thereby forming a recombined nucleic acid inserted into the chromosomal DNA, where the recombined nucleic acid encodes an active stereocilin protein (e.g., a full-length stereocilin protein). Also provided are kits that include any of the compositions described herein. Also provided herein are methods that include introducing into a cochlea of a mammal a therapeutically effective amount of any of the compositions described herein.

Also provided herein are methods of increasing expression of an active stereocilin protein (e.g., a full-length stereocilin protein) in a mammalian cell that include introducing any of the compositions described herein into the mammalian cell. Also provided herein are methods of increasing expression of an active stereocilin protein (e.g., a full-length stereocilin protein) in an outer hair cell in a cochlea of a mammal that include introducing into the cochlea of the mammal a therapeutically effective amount of any of the compositions described herein. Also provided herein are methods of treating non-symptomatic sensorineural hearing loss in a subject identified as having a defective stereocilin gene that include: administering a therapeutically effective amount of any of the compositions described herein into the cochlea of the subject.

Additional non-limiting aspects of the compositions, kits, and methods are described herein and can be used in any combination without limitation.

### Stereocilin

The STRC gene encodes stereocilin, a protein that is normally expressed in the inner ear and is associated with the stereocilia of specialized hair cells in the inner ear (see, e.g., Verpy et al., Nature 456: 255-258, 2008; and Zhang et al., J. Med. Genet. 44: 233-240, 2007). Stereocilia, which are about 10-50 μm in length, are important for hearing and balance. Stereocilia play a mechanosensing role in hearing. By bending in response to sound, they form a structure for mechanoreception of sound stimulation.

The human STRC gene is located on chromosome 15915. It contains 29 exons encompassing ˜19 kilobases (kb) (Verpy et al., Nature Genetics 29(3): 345-349, 2001; NCBI Accession No. NG011636.1). The gene is tandemly duplicated on chromosome 15915 in a telomere-to-centromere orientation, with the tandem copies located less than 100 kb apart on the chromosome. The coding sequence of the second copy is interrupted by a stop codon in exon 20, meaning that it is a “pseudogene” that encodes a nonfunctional truncated protein. (Hereinafter, references to “the STRC gene” mean the gene that does not have that stop codon in exon 20, while references to the “pseudogene” or the “STRC pseudogene” denote the gene that does have that stop codon in exon 20.) In some examples, the full-length STRC protein is a full-length wildtype STRC protein. The full-length wildtype STRC protein expressed from the human STRC gene is 1775 residues in length, and contains a signal peptide and several hydrophobic segments. When the signal peptide is cleaved off during processing of the wildtype protein, the resulting mature wildtype stereocilin protein is 1719 residues in length. In some embodiments, a full-length STRC protein can include a heterologous signal peptide positioned N-terminal to an amino acid sequence of a mature wildtype stereocilin protein.

Various mutations in the STRC gene have been associated with hearing loss (e.g., non-symptomatic sensorineural hearing loss). For example, a homozygous 1-bp insertion was identified in exon 13 in a consanguineous Pakistani family with autosomal recessive non-syndromic sensorineural deafness-16 (DFNB16) (Verpy et al. (2001). Another example found in a family with DFNB16 includes both a 4-bp deletion in exon 5 and a larger deletion encompassing exons 17-29. The two deletions were inherited from the father and mother, respectively. The 4-bp deletion was predicted to result in the translation of 5 out-of-frame amino acids and a premature stop codon in exon 5.

Additional exemplary mutations in a stereocilin gene detected in subjects having non-symptomatic sensorineural hearing loss and methods of sequencing a nucleic acid encoding stereocilin are described in, e.g., Verpy et al., Nature 456:255-259, 2008; Verpy et al., Res. Systems Neurosci. 519:194-210, 2011; Hofrichter et al., Clinical Genetics 87:49-55, 2015; and Mandelker et al., J. Mol. Diagnostics 16:639-647, 2014. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype stereocilin protein is or includes the sequence of SEQ ID NO: 1. Non-limiting examples of nucleic acid encoding a wildtype stereocilin protein are or include SEQ ID NO: 2 and SEQ ID NO: 3. As can be appreciated in the art, at least some or all of the codons in SEQ ID NO: 2 can be codon-optimized to allow for optimal expression in a non-human mammal or in a human.

In some embodiments of any of the compositions described herein, the stereocilin protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 1 or SEQ ID NO: 11. In some embodiments, a stereocilin protein can include a sequence that is identical to SEQ ID NO: 1, except that it includes 1, 2, 3, 4, 5, 6, 7, 8, 9, 10, 11, 12, 13, 14, 15, 16, 17, 18, 19, or 20 amino acid substitutions and/or deletions.

Human Full-length Wildtype Stereocilin Protein (Signal
sequence shown in bold)
(SEQ ID NO: 1)
MALSLWPLLLLLLLLLLLSFAVTLAPTGPHSLDPGLSFLKSLLSTLDQAPQGSLSRSRFFTFLANISSS
FEPGRMGEGPVGEPPPLQPPALRLHDFLVTLRGSPDWEPMLGLLGDMLALLGQEQTPRDFLVHQAGVLG
GLVEVLLGALVPGGPPTPTRPPCTRDGPSDCVLAADWLPSLLLLLEGTRWQALVQVQPSVDPTNATGLD
GREAAPHFLQGLLGLLTPTGELGSKEALWGGLLRTVGAPLYAAFQEGLLRVTHSLQDEVFSILGQPEPD
TNGQCQGVFFLTLSLLGNLQQLLLWGVRHNLSWDVQALGFLSGSPPPPPALLHCLSTGVPLPRASQPSA
HISPRQRRAITVEALCENHLGPAPPYSISNFSIHLLCQHTKPATPQPHPSTTAICQTAVWYAVSWAPGA
QGWLQACHDQFPDEFLDAICSNLSFSALSGSNRRLVKRLCAGLLPPPTSCPEGLPPVPLTPDIFWGCFL
ENETLWAERLCGEASLQAVPPSNQAWVQHVCQGPTPDVTASPPCHIGPCGERCPDGGSFLVMVCANDTM
YEVLVPFWPWLAGQCRISRGGNDTCFLEGLLGPLLPSLPPLGPSPLCLTPGPFLLGMLSQLPRCQSSVP
ALAHPTRLHYLLRLLTFLLGPGAGGAEAQGMLGRALLLSSLPDNCSFWDAFRPEGRRSVLRTIGEYLEQ
DEEQPTPSGFEPTVNPSSGISKMELLACFSPVLWDLLQREKSVWALQILVQAYLHMPPENLQQLVLSAE
REAAQGFLTLMLQGKLQGKLQVPPSEEQALGRLTALLLQRYPRLTSQLFIDLSPLIPFLAVSDLMRFPP
SLLANDSVQQGYRGSEENSLEEDKGLRPMTPRSLAAIRDYSPGMRPEQKEALAKRLLAPELFGEVPAWP
QELLWAVLPLLPHLPLENFLQLSPHQIQALEDSWPAAGLGPGHARHVLRSLVNQSVQDGEEQVRRLGPL
ACFLSPEELQSLVPLSDPTGPVERGLLECAANGTLSPEGRVAYELLGVLRSSGGAVLSPRELRVWAPLF
SQLGLRFLQELSEPQLRAMLPVLQGTSVTPAQAVLLLGRLLPRHDLSLEELCSLHLLLPGLSPQTLQAT
PRRVLVGACSCLAPELSRLSACQTAALLQTERVKDGVKNMGTTGAGPAVCIPGQQPIPTTWPDCLLPLL
PLKLLQLDSLALLANRRRYWELPWSEQQAQFLWKKMQVPTNLTLRNLQALGTLAGGMSCEFLQQINSMV
DFLEVVHMIYQLPTRVRGSLRACIWAELQRRMAMPEPEWTTVGPELNGLDSKLLLDLPIQLMDRLSNES
IMLVVELVQRAPEQLLALTPLHQAALAERALQNLAPKETPVSGEVLETLGPLVGFLGTESTRQIPLQIL
LSHLSQLQGFCLGETFATELGWLLLQESVLGKPELWSQDEVEQAGRLVFTLSTEAISLIPREALGPETL
ERLLEKQQSWEQSRVGQLCREPQLAAKKAALVAGVVRPAAEDLPEPVPNCADVRGTFPAAWSATQTAEM
ELSDFEDCLTLFAGDPGLGPEELRAAMGKAKQLWGPPRGFRPEQILQLGRLLIGLGDRELQELILVDWG
VLSTLGQIDGWSTTQLRIVVSSFLRQSGRHVSHLDFVHLTALGYTLCGLRPEELQHISSWEFSQAALFL
GTLHLQCSEEQLEVLAHLLVLPGGFGPISNWGPEIFTEIGTIAAGIPDLALSALLRGQIQGVTPLAISV
IPPPKFAVVESPIQLSSLTSAQAVAVTPEQMAFLSPEQRRAVAWAQHEGKESPEQQGRSTAWGLQDWSR
PSWSLVLTISFLGHLL
Human Stereocilin Signal Sequence
(SEQ ID NO: 10)
MALSLWPLLLLLLLLLLLSFAV
Human Full-length Wildtype Stereocilin Protein (Signal Sequence in Bold)
(SEQ ID NO: 11)
MALSLWPLLLLLLLLLLLSFAVTLAPTGPHSLDPGLSFLKSLLSTLDQAPQGSLSRSRFFTFLANISSS
FEPGRMGEGPVGEPPPLQPPALRLHDFLVTLRGSPDWEPMLGLLGDMLALLGQEQTPRDFLVHQAGVLG
GLVEVLLGALVPGGPPTPTRPPCTRDGPSDCVLAADWLPSLLLLLEGTRWQALVQVQPSVDPTNATGLD
GREAAPHFLQGLLGLLTPTGELGSKEALWGGLLRTVGAPLYAAFQEGLLRVTHSLQDEVFSILGQPEPD
TNGQCQGGNLQQLLLWGVRHNLSWDVQALGFLSGSPPPPPALLHCLSTGVPLPRASQPSAHISPRQRRA
ITVEALCENHLGPAPPYSISNFSIHLLCQHTKPATPQPHPSTTAICQTAVWYAVSWAPGAQGWLQACHD
QFPDEFLDAICSNLSFSALSGSNRRLVKRLCAGLLPPPTSCPEGLPPVPLTPDIFWGCFLENETLWAER
LCGEASLQAVPPSNQAWVQHVCQGPTPDVTASPPCHIGPCGERCPDGGSFLVMVCANDTMYEVLVPFWP
WLAGQCRISRGGNDTCFLEGLLGPLLPSLPPLGPSPLCLTPGPFLLGMLSQLPRCQSSVPALAHPTRLH
YLLRLLTFLLGPGAGGAEAQGMLGRALLLSSLPDNCSFWDAFRPEGRRSVLRTIGEYLEQDEEQPTPSG
FEPTVNPSSGISKMELLACFSPVLWDLLQREKSVWALQILVQAYLHMPPENLQQLVLSAEREAAQGFLT
LMLQGKLQGKLQVPPSEEQALGRLTALLLQRYPRLTSQLFIDLSPLIPFLAVSDLMRFPPSLLANDSVL
AAIRDYSPGMRPEQKEALAKRLLAPELFGEVPAWPQELLWAVLPLLPHLPLENFLQLSPHQIQALEDSW
PAAGLGPGHARHVLRSLVNQSVQDGEEQVRRLGPLACFLSPEELQSLVPLSDPTGPVERGLLECAANGT
LSPEGRVAYELLGVLRSSGGAVLSPRELRVWAPLFSQLGLRFLQELSEPQLRAMLPVLQGTSVTPAQAV
LLLGRLLPRHDLSLEELCSLHLLLPGLSPQTLQAIPRRVLVGACSCLAPELSRLSACQTAALLQTFRVK
DGVKNMGTTGAGPAVCIPGQPIPTTWPDCLLPLLPLKLLQLDSLALLANRRRYWELPWSEQQAQFLWKK
MQVPTNLTLRNLQALGTLAGGMSCEFLQQINSMVDFLEVVHMIYQLPTRVRGSLRACIWAELQRRMAMP
EPEWTTVGPELNGLDSKLLLDLPIQLMDRLSNESIMLVVELVQRAPEQLLALTPLHQAALAERALQNLA
PKETPVSGEVLETLGPLVGFLGTESTRQIPLQILLSHLSQLQGFCLGETFATELGWLLLQESVLGKPEL
WSQDEVEQAGRLVFTLSTEATSLIPREALGPETLERLLEKQQSWEQSRVGQLCREPQLAAKKAALVAGV
VRPAAEDLPEPVPNCADVRGTFPAAWSATQIAEMELSDFEDCLTLFAGDPGLGPEELRAAMGKAKQLWG
PPRGFRPEQILQLGRLLIGLGDRELQELILVDWGVLSTLGQIDGWSTTQLRIVVSSFLRQSGRHVSHLD
FVHLTALGYTLCGLRPEELQHISSWEFSQAALFLGTLHLQCSEEQLEVLAHLLVLPGGFGPISNWGPEI
FTEIGTIAAGIPDLALSALLRGQIQGVTPLAISVIPPPKFAVVFSPIQLSSLTSAQAVAVTPEQMAFLS
PEQRRAVAWAQHEGKESPEQQGRSTAWGLQDWSRPSWSLVLTISFLGHLL
Human Wildtype Stereocilin cDNA
(SEQ ID NO: 2)
ATGGCTCTCAGCCTCTGGCCCCTGCTGCTGCTGCTGCTGCTGCTGCTGCTGCTGTCCTTTGCAGTGACT
CTGGCCCCTACTGGGCCTCATTCCCTGGACCCTGGTCTCTCCTTCCTGAAGTCATTGCTCTCCACTCTG
GACCAGGCTCCCCAGGGCTCCCTGAGCCGCTCACGGTTCTTTACATTCCTGGCCAACATTTCTTCTTCC
TTTGAGCCTGGGAGAATGGGGGAAGGACCAGTAGGAGAGCCCCCACCTCTCCAGCCGCCTGCTCTGCGG
CTCCATGATTTTCTAGTGACACTGAGAGGTAGCCCCGACTGGGAGCCAATGCTAGGGCTGCTAGGGGAT
ATGCTGGCACTGCTGGGACAGGAGCAGACTCCCCGAGATTTCCTGGTGCACCAGGCAGGGGTGCTGGGT
GGACTTGTGGAGGTGCTGCTGGGAGCCTTAGTTCCTGGGGGCCCCCCTACCCCAACTCGGCCCCCATGC
ACCCGTGATGGGCCGTCTGACTGTGTCCTGGCTGCTGACTGGTTGCCTTCTCTGCTGCTGTTGTTAGAG
GGCACACGCTGGCAAGCTCTGGTGCAGGTGCAGCCCAGTGTGGACCCCACCAATGCCACAGGCCTCGAT
GGGAGGGAGGCAGCTCCTCACTTTTTGCAGGGTCTGTTGGGTTTGCTTACCCCAACAGGGGAGCTAGGC
TCCAAGGAGGCTCTTTGGGGCGGTCTGCTACGCACAGTGGGGGCCCCCCTCTATGCTGCCTTTCAGGAG
GGGCTGCTCCGTGTCACTCACTCCCTGCAGGATGAGGTCTTCTCCATTTTGGGGCAGCCAGAGCCTGAT
ACCAATGGGCAGTGCCAGGGAGTTTTCTTCCTTACTCTTTCCCTCCTAGGTAACCTTCAACAGCTGCTC
TTATGGGGCGTCCGGCACAACCTTTCCTGGGATGTCCAGGCGCTGGGCTTTCTGTCTGGATCACCACCC
CCACCCCCTGCCCTCCTTCACTGCCTGAGCACGGGCGTGCCTCTGCCCAGAGCTTCTCAGCCGTCAGCC
CACATCAGCCCACGCCAACGGCGAGCCATCACTGTGGAGGCCCTCTGTGAGAACCACTTAGGCCCAGCA
CCACCCTACAGCATTTCCAACTTCTCCATCCACTTGCTCTGCCAGCACACCAAGCCTGCCACTCCACAG
CCCCATCCCAGCACCACTGCCATCTGCCAGACAGCTGTGTGGTATGCAGTGTCCTGGGCACCAGGTGCC
CAAGGCTGGCTACAGGCCTGCCACGACCAGTTTCCTGATGAGTTTTTGGATGCGATCTGCAGTAACCTC
TCCTTTTCAGCCCTGTCTGGCTCCAACCGCCGCCTGGTGAAGCGGCTCTGTGCTGGCCTGCTCCCACCC
CCTACCAGCTGCCCTGAAGGCCTGCCCCCTGTTCCCCTCACCCCAGACATCTTTTGGGGCTGCTTCTTG
GAGAATGAGACTCTGTGGGCTGAGCGACTGTGTGGGGAGGCAAGTCTACAGGCTGTGCCCCCCAGCAAC
CAGGCTTGGGTCCAGCATGTGTGCCAGGGCCCCACCCCAGATGTCACTGCCTCCCCACCATGCCACATT
GGACCCTGTGGGGAACGCTGCCCGGATGGGGGCAGCTTCCTGGTGATGGTCTGTGCCAATGACACCATG
TATGAGGTCCTGGTGCCCTTCTGGCCTTGGCTAGCAGGCCAATGCAGGATAAGTCGTGGGGGCAATGAC
ACTTGCTTCCTAGAAGGGCTGCTGGGCCCCCTTCTGCCCTCTCTGCCACCACTGGGACCATCCCCACTC
TGTCTGACCCCTGGCCCCTTCCTCCTTGGCATGCTATCCCAGTTGCCACGCTGTCAGTCCTCTGTCCCA
GCTCTTGCTCACCCCACACGCCTACACTATCTCCTCCGCCTGCTGACCTTCCTCTTGGGTCCAGGGGCT
GGGGGCGCTGAGGCCCAGGGGATGCTGGGTCGGGCCCTACTGCTCTCCAGTCTCCCAGACAACTGCTCC
TTCTGGGATGCCTTTCGCCCAGAGGGCCGGCGCAGTGTGCTACGGACGATTGGGGAATACCTGGAACAA
GATGAGGAGCAGCCAACCCCATCAGGCTTTGAACCCACTGTCAACCCCAGCTCTGGTATAAGCAAGATG
GAGCTGCTGGCCTGCTTTAGTCCTGTGCTGTGGGATCTGCTCCAGAGGGAAAAGAGTGTTTGGGCCCTG
CAGATTCTAGTGCAGGCGTACCTGCATATGCCCCCAGAAAACCTCCAGCAGCTGGTGCTTTCAGCAGAG
AGGGAGGCTGCACAGGGCTTCCTGACACTCATGCTGCAGGGGAAGCTGCAGGGGAAGCTGCAGGTACCA
CCATCCGAGGAGCAGGCCCTGGGTCGCCTGACAGCCCTGCTGCTCCAGCGGTACCCACGCCTCACCTCC
CAGCTCTTCATTGACCTGTCACCACTCATCCCTTTCTTGGCTGTCTCTGACCTGATGCGCTTCCCACCA
TCCCTGTTAGCCAACGACAGTGTACAGCAGGGCTACAGAGGGTCAGAGGAAAACAGTTTGGAGGAAGAC
AAAGGGTTAAGACCCATGACTCCTCGCAGCCTGGCTGCCATCCGGGATTACAGCCCAGGAATGAGGCCT
GAACAGAAGGAGGCTCTGGCAAAGCGACTGCTGGCCCCTGAACTGTTTGGGGAAGTGCCTGCCTGGCCC
CAGGAGCTGCTGTGGGCAGTGCTGCCCCTGCTCCCCCACCTCCCTCTGGAGAACTTTTTGCAGCTCAGC
CCTCACCAGATCCAGGCCCTGGAGGATAGCTGGCCAGCAGCAGGTCTGGGGCCAGGGCATGCCCGCCAT
GTGCTGCGCAGCCTGGTAAACCAGAGTGTCCAGGATGGTGAGGAGCAGGTACGCAGGCTTGGGCCCCTC
GCCTGTTTCCTGAGCCCTGAGGAGCTGCAGAGCCTAGTGCCCCTGAGTGATCCAACGGGGCCAGTAGAA
CGGGGGCTGCTGGAATGTGCAGCCAATGGGACCCTCAGCCCAGAAGGACGGGTGGCATATGAACTTCTG
GGTGTGTTGCGCTCATCTGGAGGAGCGGTGCTGAGCCCCCGGGAGCTGCGGGTCTGGGCCCCTCTCTTC
TCTCAGCTGGGCCTCCGCTTCCTTCAGGAGCTGTCAGAGCCCCAGCTTAGAGCCATGCTTCCTGTCCTG
CAGGGAACTAGTGTTACACCTGCTCAGGCTGTCCTGCTGCTTGGACGGCTCCTTCCTAGGCACGATCTA
TCCCTGGAGGAACTCTGCTCCTTGCACCTTCTGCTACCAGGCCTCAGCCCCCAGACACTCCAGGCCATC
CCTAGGCGAGTCCTGGTCGGGGCTTGTTCCTGCCTGGCCCCTGAACTGTCACGCCTCTCAGCCTGCCAG
ACCGCAGCACTGCTGCAGACCTTTCGGGTTAAAGATGGTGTTAAAAATATGGGTACAACAGGTGCTGGT
CCAGCTGTGTGTATCCCTGGTCAGCAGCCTATTCCCACCACCTGGCCAGACTGCCTGCTTCCCCTGCTC
CCATTAAAGCTGCTACAACTGGATTCCTTGGCTCTTCTGGCAAATCGAAGACGCTACTGGGAGCTGCCC
TGGTCTGAGCAGCAGGCACAGTTTCTCTGGAAGAAGATGCAAGTACCCACCAACCTTACCCTCAGGAAT
CTGCAGGCTCTGGGCACCCTGGCAGGAGGCATGTCCTGTGAGTTTCTGCAGCAGATCAACTCCATGGTA
GACTTCCTTGAAGTGGTGCACATGATCTATCAGCTGCCCACTAGAGTTCGAGGGAGCCTGAGGGCCTGT
ATCTGGGCAGAGCTACAGCGGAGGATGGCAATGCCAGAACCAGAATGGACAACTGTAGGGCCAGAACTG
AACGGGCTGGATAGCAAGCTACTCCTGGACTTACCGATCCAGTTGATGGACAGACTATCCAATGAATCC
ATTATGTTGGTGGTGGAGCTGGTGCAAAGAGCTCCAGAGCAGCTGCTGGCACTGACCCCCCTCCACCAG
GCAGCCCTGGCAGAGAGGGCACTACAAAACCTGGCTCCAAAGGAGACTCCAGTCTCAGGGGAAGTGCTG
GAGACCTTAGGCCCTTTGGTTGGATTCCTGGGGACAGAGAGCACACGACAGATCCCCCTACAGATCCTG
CTGTCCCATCTCAGTCAGCTGCAAGGCTTCTGCCTAGGAGAGACATTTGCCACAGAGCTGGGATGGCTG
CTATTGCAGGAGTCTGTTCTTGGGAAACCAGAGTTGTGGAGCCAGGATGAAGTAGAGCAAGCTGGACGC
CTAGTATTCACTCTGTCTACTGAGGCAATTTCCTTGATCCCCAGGGAGGCCTTGGGTCCAGAGACCCTG
GAGCGGCTTCTAGAAAAGCAGCAGAGCTGGGAGCAGAGCAGAGTTGGACAGCTGTGTAGGGAGCCACAG
CTTGCTGCCAAGAAAGCAGCCCTGGTAGCAGGGGTGGTGCGACCAGCTGCTGAGGATCTTCCAGAACCT
GTGCCAAATTGTGCAGATGTACGAGGGACATTCCCAGCAGCCTGGTCTGCAACCCAGATTGCAGAGATG
GAGCTCTCAGACTTTGAGGACTGCCTGACATTATTTGCAGGAGACCCAGGACTTGGGCCTGAGGAACTG
CGGGCAGCCATGGGCAAAGCAAAACAGTTGTGGGGTCCCCCCCGGGGATTTCGTCCTGAGCAGATCCTG
CAGCTTGGTAGGCTCTTAATAGGTCTAGGAGATCGGGAACTACAGGAGCTGATCCTAGTGGACTGGGGA
GTGCTGAGCACCCTGGGGCAGATAGATGGCTGGAGCACCACTCAGCTCCGCATTGTGGTCTCCAGTTTC
CTACGGCAGAGTGGTCGGCATGTGAGCCACCTGGACTTCGTTCATCTGACAGCGCTGGGTTATACTCTC
TGTGGACTGCGGCCAGAGGAGCTCCAGCACATCAGCAGTTGGGAGTTCAGCCAAGCAGCTCTCTTCCTC
GGCACCCTGCATCTCCAGTGCTCTGAGGAACAACTGGAGGTTCTGGCCCACCTACTTGTACTGCCTGGT
GGGTTTGGCCCAATCAGTAACTGGGGGCCTGAGATCTTCACTGAAATTGGCACCATAGCAGCTGGGATC
CCAGACCTGGCTCTTTCAGCACTGCTGCGGGGACAGATCCAGGGCGTTACTCCTCTTGCCATTTCTGTC
ATCCCTCCTCCTAAATTTGCTGTGGTGTTTAGTCCCATCCAACTATCTAGTCTCACCAGTGCTCAGGCT
GTGGCTGTCACTCCTGAGCAAATGGCCTTTCTGAGTCCTGAGCAGCGACGAGCAGTTGCATGGGCCCAA
CATGAGGGAAAGGAGAGCCCAGAACAGCAAGGTCGAAGTACAGCCTGGGGCCTCCAGGACTGGTCACGA
CCTTCCTGGTCCCTGGTATTGACTATCAGCTTCCTTGGCCACCTGCTATGA
Human Wildtype Stereocilin cDNA - Codon Optimized
(SEQ ID NO: 3)
ATGGCTCTGTCTCTGTGGCCTCTGCTGCTGCTCCTGTTGCTGCTTCTGCTGCTCAGCTTCGCCGTGACA
CTGGCTCCAACAGGACCCCACTCTCTGGATCCTGGCCTGAGCTTTCTGAAGTCCCTGCTGAGCACCCTG
GATCAGGCTCCTCAGGGCAGCCTGAGCAGATCCAGATTCTTCACCTTCCTGGCCAACATCAGCAGCAGC
TTCGAGCCTGGCAGAATGGGAGAAGGACCTGTGGGAGAACCTCCTCCACTGCAACCTCCAGCTCTGCGG
CTGCACGATTTTCTGGTCACACTGAGAGGCAGCCCCGACTGGGAACCTATGCTGGGACTGCTGGGAGAT
ATGCTGGCCCTGCTCGGACAAGAGCAGACCCCTAGAGATTTCCTGGTGCATCAGGCTGGCGTGCTCGGA
GGACTGGTTGAAGTTCTGCTTGGAGCACTGGTGCCTGGCGGACCTCCTACACCTACAAGACCTCCATGC
ACCAGAGATGGCCCCAGCGATTGTGTGCTGGCTGCTGATTGGCTGCCTAGCCTGCTCCTGCTTCTGGAA
GGCACAAGATGGCAGGCCCTGGTGCAGGTTCAGCCTTCTGTGGATCCTACCAATGCCACCGGCCTGGAT
GGAAGAGAAGCCGCTCCACACTTTCTGCAGGGACTGCTTGGACTGCTCACACCTACTGGCGAGCTGGGC
TCTAAAGAAGCCCTTTGGGGAGGCCTGCTGAGAACAGTTGGAGCCCCTCTGTACGCCGCCTTTCAAGAG
GGACTCCTGAGAGTGACACACAGCCTGCAGGACGAGGTGTTCAGCATCCTGGGACAGCCAGAGCCTGAC
ACCAATGGACAGTGTCAGGGCGTGTTCTTTCTGACCCTGAGCCTGCTGGGCAACCTGCAGCAACTGCTT
CTGTGGGGCGTCAGACACAACCTGAGCTGGGATGTGCAGGCACTGGGCTTTCTGTCTGGAAGCCCTCCA
CCTCCACCAGCACTGCTGCATTGTCTGTCTACTGGCGTGCCACTGCCTAGAGCCTCTCAGCCTAGCGCT
CACATCAGCCCCAGACAGAGAAGGGCCATCACCGTGGAAGCTCTGTGCGAGAATCACCTGGGACCTGCT
CCTCCATACAGCATCAGCAACTTCTCCATCCATCTGCTGTGTCAGCACACCAAGCCTGCCACACCTCAG
CCTCATCCTAGCACCACAGCCATCTGTCAGACCGCCGTTTGGTACGCCGTTTCTTGGGCTCCTGGTGCT
CAAGGATGGCTGCAGGCCTGTCACGATCAGTTCCCCGACGAGTTCCTGGACGCCATCTGCAGCAATCTG
AGCTTCTCTGCCCTGTCCGGCAGCAATCGGAGACTGGTCAAAAGACTGTGCGCCGGACTGCTGCCTCCT
CCAACATCTTGTCCTGAGGGACTGCCTCCTGTGCCTCTGACTCCCGATATCTTTTGGGGCTGCTTCCTG
GAAAACGAGACACTGTGGGCCGAGAGACTGTGTGGCGAAGCCTCTCTGCAAGCCGTGCCTCCATCTAAT
CAGGCCTGGGTGCAGCACGTTTGTCAGGGCCCTACACCTGACGTGACAGCCTCTCCTCCTTGTCACATC
GGACCTTGCGGCGAGAGATGTCCTGATGGCGGCAGCTTTCTCGTGATGGTCTGCGCCAACGACACTATG
TACGAGGTGCTGGTGCCCTTCTGGCCTTGGCTGGCTGGCCAGTGTAGAATCTCCAGAGGCGGCAACGAT
ACCTGTTTCCTGGAAGGACTCCTGGGACCACTGCTTCCATCTCTGCCTCCGCTTGGACCCTCTCCTCTG
TGTCTTACCCCTGGACCTTTCCTGCTGGGGATGCTGTCTCAGCTGCCTAGATGCCAGTCTAGCGTGCCA
GCTCTGGCCCATCCAACCAGACTGCACTATCTGTTGCGGCTGCTGACCTTCCTCCTTGGACCTGGCGCT
GGCGGAGCTGAAGCTCAAGGCATGCTTGGCAGAGCCCTGCTGCTGTCATCCCTGCCTGACAACTGCAGC
TTCTGGGACGCCTTCAGACCTGAAGGACGCAGAAGCGTGCTGAGGACCATCGGCGAGTACCTGGAACAG
GATGAGGAACAGCCTACACCAAGCGGCTTCGAACCCACCGTGAATCCTAGCAGCGGCATCTCCAAGATG
GAACTGCTGGCCTGCTTCAGCCCCGTGCTGTGGGATCTGCTGCAGAGGGAAAAAAGCGTGTGGGCCCTG
CAGATCCTGGTCCAGGCCTATCTGCACATGCCTCCAGAGAACCTGCAACAGCTGGTGCTGTCTGCCGAG
AGAGAAGCTGCCCAGGGATTCCTGACTCTGATGCTGCAGGGAAAGCTGCAGGGCAAACTCCAGGTGCCA
CCTAGCGAAGAACAGGCCCTTGGCAGACTGACAGCACTCCTGCTCCAGAGATACCCCAGACTGACCTCT
CAGCTGTTTATCGATCTGAGCCCTCTGATCCCATTCCTGGCCGTGTCCGACCTGATGAGATTCCCTCCT
AGTCTGCTGGCCAACGACTCCGTGCAGCAGGGATACAGAGGCAGCGAGGAAAACAGCCTGGAAGAGGAC
AAAGGCCTGCGGCCTATGACACCTAGATCTCTGGCCGCCATCCGGGATTACAGCCCTGGAATGAGGCCC
GAGCAGAAAGAGGCCCTGGCTAAGAGACTGCTGGCTCCCGAGCTGTTTGGCGAAGTTCCAGCCTGGCCT
CAAGAACTGCTGTGGGCTGTTCTGCCCCTGCTGCCACATCTGCCTCTGGAAAACTTCCTGCAGCTGAGC
CCACACCAGATTCAGGCCCTCGAGGATTCTTGGCCTGCCGCTGGACTCGGACCTGGACATGCTAGACAC
GTGCTGAGAAGCCTGGTCAACCAGAGCGTTCAGGACGGCGAGGAACAAGTGCGGAGACTTGGACCCCTG
GCCTGTTTTCTGAGCCCCGAGGAACTGCAGAGTCTGGTGCCTCTGTCTGACCCTACAGGCCCTGTTGAA
AGGGGCCTGCTGGAATGTGCCGCCAATGGAACACTGAGCCCTGAGGGCAGAGTGGCCTATGAACTTCTG
GGAGTGCTGAGATCTAGCGGCGGAGCCGTTCTGTCCCCTAGGGAACTTAGAGTGTGGGCACCCCTGTTT
AGCCAGCTGGGCCTGAGATTCCTGCAAGAGCTGTCTGAGCCACAGCTGAGGGCTATGCTGCCTGTGCTC
CAGGGCACATCTGTGACACCAGCTCAAGCCGTCCTGCTGTTGGGCAGACTGCTCCCTAGACACGATCTG
TCCCTGGAAGAACTGTGCAGCCTGCACTTGCTGCTGCCTGGACTGTCTCCTCAGACACTGCAGGCCATT
CCTCGGAGAGTGCTTGTGGGAGCCTGTAGCTGTCTGGCCCCTGAGCTGTCTAGACTGAGCGCCTGTCAA
ACAGCCGCTCTCCTGCAGACCTTCAGAGTGAAGGATGGCGTGAAGAACATGGGCACCACAGGCGCTGGA
CCTGCCGTGTGTATTCCTGGACAGCAGCCCATTCCTACCACCTGGCCAGATTGTCTGCTCCCACTGCTG
CCCCTGAAACTGCTGCAGCTGGATTCTCTGGCTCTGCTGGCTAACCGGCGGAGATATTGGGAACTGCCT
TGGAGCGAACAGCAGGCACAGTTCCTGTGGAAGAAGATGCAGGTCCCCACCAATCTGACACTGCGGAAT
CTGCAGGCTCTCGGCACACTTGCTGGCGGAATGAGCTGCGAGTTCCTCCAGCAGATCAACAGCATGGTG
GACTTTCTGGAAGTGGTGCACATGATCTACCAGCTGCCAACCAGAGTGCGGGGAAGCCTGAGAGCTTGT
ATTTGGGCTGAACTGCAGCGGCGGATGGCCATGCCTGAACCTGAATGGACAACAGTGGGCCCCGAGCTG
AACGGCCTGGACTCTAAACTTCTGCTGGATCTCCCCATCCAGCTGATGGACAGACTGAGCAACGAGAGC
ATCATGCTGGTGGTGGAACTGGTGCAGAGAGCCCCAGAACAGCTGCTGGCACTGACACCTCTGCATCAA
GCTGCTCTGGCCGAACGGGCCCTTCAGAATCTGGCTCCCAAAGAAACCCCTGTGTCCGGGGAAGTGCTG
GAAACACTGGGACCTCTTGTGGGCTTCCTGGGCACCGAGTCTACCAGACAGATTCCTCTCCAGATCCTG
CTGTCCCACCTGAGCCAGCTCCAGGGATTTTGTCTGGGCGAGACATTCGCCACCGAACTCGGATGGTTG
CTGCTCCAAGAGAGCGTGCTGGGAAAGCCCGAACTGTGGTCACAGGACGAAGTGGAACAGGCCGGCAGA
CTGGTGTTTACCCTGTCTACCGAGGCCATCAGTCTGATCCCCAGAGAAGCACTGGGCCCTGAAACACTC
GAGAGGCTGCTGGAAAAGCAGCAGTCTTGGGAACAGAGCAGAGTGGGCCAGCTGTGTAGAGAACCTCAG
CTGGCCGCTAAAAAGGCCGCACTGGTTGCTGGCGTTGTCAGACCTGCTGCCGAGGATCTGCCAGAGCCA
GTGCCTAATTGTGCCGATGTGCGGGGCACATTTCCTGCCGCTTGGAGCGCTACACAGATCGCCGAAATG
GAACTGAGCGACTTCGAGGACTGTCTGACTCTGTTTGCCGGCGATCCTGGACTGGGACCAGAAGAACTG
AGAGCCGCCATGGGCAAAGCCAAGCAACTTTGGGGACCTCCAAGAGGCTTCAGACCCGAACAGATTCTG
CAGCTCGGCCGCCTGCTTATCGGACTGGGCGATAGAGAGCTGCAAGAACTGATCCTGGTGGACTGGGGC
GTGCTGTCTACTCTGGGACAAATCGACGGCTGGTCCACCACACAGCTGCGGATTGTGGTGTCCAGCTTC
CTGAGGCAGTCTGGCAGACATGTGTCTCACCTGGACTTCGTGCACCTGACTGCCCTGGGCTACACACTG
TGTGGACTGCGGCCTGAGGAACTTCAGCACATCAGCTCTTGGGAGTTCAGCCAGGCAGCCCTGTTTCTG
GGAACCCTGCATCTGCAGTGCTCCGAAGAACAACTGGAAGTGCTCGCCCATCTGCTCGTGCTGCCAGGC
GGATTTGGCCCCATCTCTAATTGGGGACCCGAGATCTTCACCGAGATCGGCACCATTGCCGCTGGCATC
CCTGATCTGGCCCTGTCTGCACTGCTGAGAGGACAGATCCAGGGCGTGACACCACTGGCCATTAGCGTG
ATCCCTCCACCAAAGTTCGCCGTGGTGTTTAGCCCCATTCAGCTGTCCAGCCTGACATCTGCCCAAGCC
GTGGCTGTGACCCCTGAACAGATGGCATTTCTGTCTCCCGAGCAGCGGAGAGCTGTTGCTTGGGCTCAA
CACGAGGGCAAAGAGAGTCCTGAGCAACAGGGAAGAAGCACCGCTTGGGGACTGCAGGATTGGTCCAGA
CCTTCTTGGTCACTGGTGCTGACCATCAGCTTTCTGGGCCACCTCCTGTGA

A non-limiting example of a human wildtype stereocilin genomic DNA sequence is SEQ ID NO: 4. The exons in SEQ ID NO: 4 are: nucleotide positions 1-142 (exon 1), nucleotide positions 445-1230 (exon 2), nucleotide positions 1319-1343 (exon 3), nucleotide positions 2111-3368 (exon 4), nucleotide positions 4325-4387 (exon 5), nucleotide positions 4489-4605 (exon 6), nucleotide positions 4748-4914 (exon 7), nucleotide positions 5570-5756 (exon 8), nucleotide positions 5895-6010 (exon 9), nucleotide positions 6292-6424 (exon 10), nucleotide positions 6777-6959 (exon 11), nucleotide positions 7264-7302 (exon 12), nucleotide positions 7486-7653 (exon 13), nucleotide positions 7817-7882 (exon 14), nucleotide positions 8364-8489 (exon 15), nucleotide positions 9467-9525 (exon 16), nucleotide positions 10598-10721 (exon 17), nucleotide positions 10826-10938 (exon 18), nucleotide positions 13402-13537 (exon 19), nucleotide positions 13955-14151 (exon 20), nucleotide positions 14350-14440 (exon 21), nucleotide positions 14649-14805 (exon 22), nucleotide positions 15390-15559 (exon 23), nucleotide positions 17250-17405 (exon 24), nucleotide positions 17787-17929 (exon 25), nucleotide positions 18119-18267 (exon 26), nucleotide positions 18509-18605 (exon 27), nucleotide positions 18694-18841 (exon 28), and nucleotide positions 19041-19238 (exon 29). The introns are located between each of these exons in SEQ ID NO: 4, i.e., at nucleotide positions 143-444 (intron 1), nucleotide positions 1231 to 1318 (intron 2), nucleotide positions 1344-2110 (intron 3), nucleotide positions 3369-4324 (intron 4), nucleotide positions 4388-4488 (intron 5), nucleotide positions 4606-4747 (intron 6), nucleotide positions 4915-5569 (intron 7), nucleotide positions 5757-5894 (intron 8), nucleotide positions 6011-6291 (intron 9), nucleotide positions 6425-6776 (intron 10), nucleotide position 6960-7263 (intron 11), nucleotide positions 7303-7485 (intron 12), nucleotide positions 7654-7816 (intron 13), nucleotide positions 7883-8363 (intron 14), nucleotide positions 8490-9466 (intron 15), nucleotide positions 9526-10597 (intron 16), nucleotide positions 10722-10825 (intron 17), nucleotide positions 10939-13401 (intron 18), nucleotide positions 13538-13954 (intron 19), nucleotide positions 14152-14349 (intron 20), nucleotide positions 14441-14648 (intron 21), nucleotide positions 14806-15389 (intron 22), nucleotide positions 15560-17249 (intron 23), nucleotide positions 17406-17786 (intron 24), nucleotide positions 17930-18118 (intron 25), nucleotide positions 18268-18508 (intron 26), nucleotide positions 18606-18693 (intron 27), and nucleotide positions 18842-19040 (intron 28).

Human Wildtype Stereocilin Gene
(SEQ ID NO: 4)
gccctgccct cacctggcta tcccacacag gtgagaataa ccagaactca cctccggtac
cagtgttcac ttggaaacat ggctctcagc ctctggcccc tgctgctgct gctgctgctg
ctgctgctgc tgtcctttgc aggtaagaag aacagtgagc agaactgggg atgaggagga
gggtggctgg aaaaagactt taagaatatg gaggtgaacc tgttagatag aaggacaaag
gagagaggca gagacttgtg caaaagggaa aaatgagggt taagaaaagc aggccaagac
ttactgtagg ccagtgaaag gggttcagct caccatcccc tcacctcatc tttagatcca
ggtagggaac tgtgctcagg ggcagggttg agtttgggct ctgtgttcct ctccttcagt
gacctctggt ttctctcctt acagtgactc tggcccctac tgggcctcat tccctggacc
ctggtctctc cttcctgaag tcattgctct ccactctgga ccaggctccc cagggctccc
tgagccgctc acggttcttt acattcctgg ccaacatttc ttcttccttt gagcctggga
gaatggggga aggaccagta ggagagcccc cacctctcca gccgcctgct ctgcggctcc
atgattttct agtgacactg agaggtagcc ccgactggga gccaatgcta gggctgctag
gggatatgct ggcactgctg ggacaggagc agactccccg agatttcctg gtgcaccagg
caggggtgct gggtggactt gtggaggtgc tgctgggagc cttagttcct gggggccccc
ctaccccaac tcggccccca tgcacccgtg atgggccgtc tgactgtgtc ctggctgctg
actggttgcc ttctctgctg ctgttgttag agggcacacg ctggcaagct ctggtgcagg
tgcagcccag tgtggacccc accaatgcca caggcctcga tgggagggag gcagctcctc
actttttgca gggtctgttg ggtttgctta ccccaacagg ggagctaggc tccaaggagg
ctctttgggg cggtctgcta cgcacagtgg gggcccccct ctatgctgcc tttcaggagg
ggctgctccg tgtcactcac tccctgcagg atgaggtctt ctccattttg gggcagccag
agcctgatac caatgggcag tgccagggag gtgagtgtgg ccagggctgg gactgggatg
tggcagggca aggaaagtga aattggggta gttttcttcc ttactctttc cctcctaggt
aaccttcaac agctgctctt atggtaagta acaggagacc agttctgagg gattgggcct
ggaaaatctg gaggtgaaga gctgaagacc tcagcctcta gagaggaaaa ctgatgggag
gagtgtagtt tagtggtttt ggggtgtgac tgtctgggtt ggtgtcccag ctccacctct
tcctagccat atgaccttga gcaggttaca tagtctttct atacctcagt ttccccattt
ataaaatgag aatgataata ttagttacca cagagttgtt gcacccggtt aaatgagttg
atactgtgta tgcaaacgac ttaaaaccgt gctggcacat agcgcttaat aatgttagct
agtaaagatg ggatttggaa aataaggaca cagctggatt cctctacccc cttactactt
cagtacaaca atgccagaca gtagttagac atattgagtt gctgagcaga tttcctaaca
tgaggcccgc tgagggttgt gtttaagcta tctaaaagca tacgaagaaa ggagacagaa
gggggccagg tggacagaaa gaattccaac tggggcttct cctaggtgat tttggacctt
ggcagggcag ctttctcttt tttgccccgt tgcagcattt caaccagtaa cgcctaaact
ctcagggacc tcgcttgtag aaaagcctat gcttgccatg ccccttgagg gctctgagtc
agggtcagaa tcttcagctg gaggaaatgt gaactgacca gatcctgcct gctcctccct
ctgcacccag gggcgtccgg cacaaccttt cctgggatgt ccaggcgctg ggctttctgt
ctggatcacc acccccaccc cctgccctcc ttcactgcct gagcacgggc gtgcctctgc
ccagagcttc tcagccgtca gcccacatca gcccacgcca acggcgagcc atcactgtgg
aggccctctg tgagaaccac ttaggcccag caccacccta cagcatttcc aacttctcca
tccacttgct ctgccagcac accaagcctg ccactccaca gccccatccc agcaccactg
ccatctgcca gacagctgtg tggtatgcag tgtcctgggc accaggtgcc caaggctggc
tacaggcctg ccacgaccag tttcctgatg agtttttgga tgcgatctgc agtaacctct
ccttttcagc cctgtctggc tccaaccgcc gcctggtgaa gcggctctgt gctggcctgc
tcccaccccc taccagctgc cctgaaggcc tgccccctgt tcccctcacc ccagacatct
tttggggctg cttcttggag aatgagactc tgtgggctga gcgactgtgt ggggaggcaa
gtctacaggc tgtgcccccc agcaaccagg cttgggtcca gcatgtgtgc cagggcccca
ccccagatgt cactgcctcc ccaccatgcc acattggacc ctgtggggaa cgctgcccgg
atgggggcag cttcctggtg atggtctgtg ccaatgacac catgtatgag gtcctggtgc
ccttctggcc ttggctagca ggccaatgca ggataagtcg tgggggcaat gacacttgct
tcctagaagg gctgctgggc ccccttctgc cctctctgcc accactggga ccatccccac
tctgtctgac ccctggcccc ttcctccttg gcatgctatc ccagttgcca cgctgtcagt
cctctgtccc agctcttgct caccccacac gcctacacta tctcctccgc ctgctgacct
tcctcttggg tccaggggct gggggcgctg aggcccaggg gatgctgggt cgggccctac
tgctctccag tctcccagac aactgctcct tctgggatgc ctttcgccca gagggccggc
gcagtgtgct acggacgatt ggggaatacc tggaacaaga tgaggagcag ccaaccccat
caggctttga acccactgtc aaccccagct ctggtataag caagatggag ctgctggcct
gctttagtgt gagtgctctg ccagagggaa agctcctaga acagtgagaa ggccctccag
gggaattcct cgaatactca gaggcagtag tgtggggtag tagttgaagc acacagctct
agagtcagac aggcttggat tcatatcttg gttctgtgac cagccttgaa tgagttattt
aacttctctg agcaatattt ttctcgtctc atttataaac tagggatgat aatggtatat
gagataatac atgctgtggg cttagcacag tgcatgatac acaaacatgc aataaatatt
accttgttat tcttttgggc tctttgactc tctcactttc tgcaccagaa agaaaaagga
tcaagttaga ggactctaaa tttttcccct agagagtgag aattggaggc tggcagaata
caggaagata aggtaggaat gagaaagatt cagggacact accaatcaga agactttggt
tctaggttca actgtgccac aaattagtgt gatcttaggc aagcaatttc atttagtttt
tctgggcttc agtttttagt ctgtagaatg gaggggtgag aatatgttaa acaccataat
taattcactg agtgcctatt atatgcaagg cactttgcta ggttctgtag gatatataaa
gatttcttac tccatgttgg ggccaccttt ttcaaaccct gggcccagta aaatggaatt
agatagtctc atagtatttg gttcaggtct acaagtatta attgagccaa ctatggacct
ggcatgggag agggtacaag agaaattaga gatatgatcc cggacctaaa agagcttaat
atctgaagaa tcacacttga gatgatggac aagcatccca gcaagtggag ctggaatgcc
tgggggagct gcaggagaga cagagaagac agctctgttg gcatattgtc tttcttccca
ccagcctgtg ctgtgggatc tgctccagag ggaaaagagt gtttgggccc tgcagattct
agtgcaggta acaggtggag ggcacatggg tgggctgggt gacagccatg gctggaggtc
cctgccccgt gaggtgaggc catacccacc atgacctcct attcgcaggc gtacctgcat
atgcccccag aaaacctcca gcagctggtg ctttcagcag agagggaggc tgcacagggc
ttcctgacac tcatgctgca ggggaagctg caggggaagc tgcaggtgag cactgagaaa
ggggagcaag ggcacctgga gcctagtgtt cagagggctt gctttagtgg gaggaggaac
tccagagagg aaatggcagg gatactgagc atctccagag gcagaatcca ttcctgtgcc
cctacaggta ccaccatccg aggagcaggc cctgggtcgc ctgacagccc tgctgctcca
gcggtaccca cgcctcacct cccagctctt cattgacctg tcaccactca tccctttctt
ggctgtctct gacctgatgc gcttcccacc atccctgtta gccaacgaca gtgtgtaagg
ttcttgcact actcctcctg ctcctgtcac ggtcaggcca accgcatcca cctggagcag
ccccttccgg agctcctctc tgtttttttc tttcatgcca gataggcaat gtgccaacat
cgtagcaagg tttgagagag gcacatctca cgcctgagtg tgaaaaccca atcattatgc
taatgaacta caaaaggatc agagagctcc tctctattaa aaccagggag aggatgggcg
tggtggctca tgcctgtaat cccagcacgt tgggagcccg aggcaggtgg atcactaggt
ccgcctagtg agttcgagac cagcctggcc aatatggtga aaccccgtct ctattaaact
acaaaaatta gccaggcatg gtggtgggcg cttgtagtcc cagttactct ggaggctgag
gcaggaggat agcttgaacc tgggaggcag aggttgcagt gaaccaagat cgtgccactg
cactccagcc tgggtgacag agcgagactc cgtcttaaaa aaaacaaaaa acaaaacaaa
acaaaaaaac agggagagtc tccttcctat ctagacagca gggctacaga gggtcagagg
aaaacagttt ggaggaagac aaagggttaa gacccatgac tcctcgcagc ctggctgcca
tccgggatta cagcccagga atgaggcctg aacagaagga ggctctggca aagcgactgc
tggcccctga actgtttggg gaagtgcctg cctggcccca ggagctgctg tgggcagtgc
tgcccctgct cccccacctc cctctggaga actttttgca gctcagccct caccaggtat
gagaatcatc ttctttactt gactggccca tcttctgcta gtggggacaa agagtcaatg
gcatgtctct cagtggcccc tccctgcaag aaccctatag tgaccccagt gcgagctaac
cttccccatc tcagatccag gccctggagg atagctggcc agcagcaggt ctggggccag
ggcatgcccg ccatgtgctg cgcagcctgg taaaccagag tgtccaggat ggtgaggagc
aggtacgcag gtgagttgtt gtgggatcag taaccaaggc aagagtggaa gaggtagaga
gaggaaggca cagctgtcac gctgggtcgg tgttctagga agaaaggggc aagagagtag
gcagtggcct caggcagcat agagttccag gagagaggtc tatagatggt gcccctgtgt
agtggtgtag tgtcagagtg cccagtgtat gtacccatac catctgctgc caggcctgcc
ttagtgctag tcttggggac cacacaaagg tcagcttcat gccctcctca ggcttgggcc
cctcgcctgt ttcctgagcc ctgaggagct gcagagccta gtgcccctga gtgatccaac
ggggccagta gaacgggggc tgctggaatg tgcagccaat gggaccctca gcccagaagg
acgggtgagc ccctcagcac aagcctacaa gactttaggc ttcccctggg tctgtgtgga
tggctttccc attgtgtcaa cttgagcaca gtggtgccag cccccatccc acttttgcaa
cctccattcc ttactccatg gccattctta cctgttacca cctcttcctg gcccttctct
atctggtctg tagcacccca aacataccct ttgccatttt gaacctaatc tactccagtc
caatccctag ttccaaaccc tagcccaggc cctgggaaat tcagatgtgg gattagagag
gaagttcaag gttcatctgt cttttctctc cagtcctaaa ccttctttgg ttacaggtgg
catatgaact tctgggtgtg ttgcgctcat ctggaggagc ggtgctgagc ccccgggagc
tgcgggtctg ggcccctctc ttctctcagc tgggcctccg cttccttcag gagctgtcag
agccccagct tagagccatg cttcctgtcc tgcagggaac tagtgttaca cctgctcagg
tttgcctgtc tcactccctg gcatgtaccc tccatccccg cttgagcccc agtcaagaga
atcccattca gggataaaag cagcccctcc tttccctggg tgaacagtag aggtaaactc
tgtctgcagg aggacgcctt cattcccttt cctcagatca agaagggacc tgagtcactg
aggatggtta ctagggatgg ttaagaggca gcgggaagtt ttggagggtt tgccttagga
acccacttag gacctggctg ctgggtcctg agagctgttg ttttcggtcc catcccaaca
caggctgtcc tgctgcttgg acggctcctt cctaggcacg atgtgagtag cagcaacttc
tcagcctccc gccagaggtc tctatcctct tttaacctgg ctcctgcatc tgcccctcct
ctctctccgc tcccctcata cttactgcct tgctgcattg tgattgttgt cttccccaac
acccttccct tcttcttcag gcctcttgtc tctcttgctc tttagctatc cctggaggaa
ctctgctcct tgcaccttct gctaccaggc ctcagccccc agacactcca ggccatccct
aggcgagtcc tggtcggggc ttgttcctgc ctggcccctg aactgtcacg cctctcagcc
tgccagaccg cagcactgct gcagaccttt cgggtatgag agtggcaagg aggatgagat
aatcagggat accggctctt tctggttggg aggaaggcat cttccctgag gccagggaag
gcctttcata cctccccact tacacacaca cacacacaca cacacacaca cacacacaca
accaattctc atgcaggtta aagatggtgt taaaaatatg ggtacaacag gtgctggtcc
agctgtgtgt atccctggtc aggtaagtgt gagatctccc aactgagctc ctctccccat
tctggggcag tttcatatgg ctggtgctac ctcccacact accctgcagt ggccctgaga
gttctggtta gctctgtgcc cattagcagc cctccccagt gccagatgca ggacagcatg
atccactcac attgtcctag actaatgtca aagctggaag ggcctgagaa atcttccagg
ccacccaccc tgctttcaga tgaaaagacc aaggctggga gaagctaagg gactttgttt
gcctggtgcc taactagcag caacacttga ccacagcagc ctgcagtgtg aggctcttag
gcgtttattg ctacagtggc aaatgccatt ccacttctgt cctagctttg gtccctttcc
acccccatgg ttccttttct ctgagtgcta agtacagact ctctcaccta tcactacact
gctataccca tcaccgccag cagcctattc ccaccacctg gccagactgc ctgcttcccc
tgctcccatt aaagctgcta caactggatt ccttggctct tctggcaaat cgaagacgct
actgggagct gccctggtct gagcagcagg taattctccc cacttaattt cagaacttcc
tccctcaatg tagtctacct tctttaccta tcccttagcc ctatttggcc agcttatccc
tactatcctt tatttgattg tttgagatac agtctcactc tgttgcccag gctgcagtgc
agtggcatga tcagagttcg ctgtaacctc aaactcctga gctcaggcaa tctttctgcc
tcagcctcct gaatagctag gacgacaggt ggttaccacc atgcctggct aatttttaaa
tttttttttt gttttttgag atgaagtctt gctctgtcac ccaggcttga gtacagtggc
acaagcttgg ctcactgcaa cctctgtctc ccgggttcaa gcgattctcc tgcctcagcc
tcccgagtag ctgggactac aggcactccc cacaatgcct ggctaatttt ttttttgttt
tagtagagac agggtttcac catattggcc aggctggtct cgaactgctg accttgtgat
ctgcctgcct ctgcctctca aagtgctggg attacaggtg tgagccacca tgcccggcca
atttttaaat tttttgtaga gacagacaat acaaaaatgt ggacactatg tggagacact
atgttgaggt actatgctgt ccagattggt cttgaactcc tggcctcaag caatcctcct
gccttggcct cccaaagtgc tgggattaca gacctgagcc actgcaccca gccccctagt
atctcttata atgtgacttg cttttctttt tctttctcct tcccttttct ttcatttctt
tctcactctc gagagaagag tgggcatctg ggagagtggg aggctggtgg gtcccacaga
gtgaggaggc aggactgggt ccaaggcagt cctgcctctc cactctaggg ggtatccttg
gacagtgtct cttctgggaa ggggctcgtc tttctttctc ttgtaggcac agtttctctg
gaagaagatg caagtaccca ccaaccttac cctcaggaat ctgcagtgag taacttgtgt
tgagcagtgc gctgaattcg accaacattt ttttgagtgc ttactatgtg ccaggcacca
tgtgatatgg aatgggggat atagggatga atgatgcata gtccctgcct cgtggacgtt
ctcctagcac ctccctttgc cctcctttcc ttccacagtg ccatgcctat cctgactaga
gccaaaggac tcagaaaacc tggattcagg ttccagtcct gtcacctact tgtcctcttg
ggcaagtcat ttaacgtccc tgtgtcagtt ttcccttctt taaatgagaa ttacaatggc
accagcctca taggtagtta ctgtgaagat taaatgaggt aggtcatgta agatatttaa
cacagtgttt ggtccattgt aaagtcccag tagtcatttg ctactgttag tttacttcag
gatgacttca gaggcactgg ccaagcaaga ataaatagga ataagaaggt atcactttac
ttacacccac attagaagaa caatgggctt cagaatcttt tttttttttt ttttttcgag
acagtcttgc tctgttgccc aggctggagt gcagtggcgc gatttcggct cactgcaacc
tctgcctccc aggttcaagc gattctcctg tctcagcctc tggagtagct gggattacag
gaatgtgcca ccatacccag ctaatttttg tatttttagt agagatgggg tttcaccatt
ttggccaggc tggtctcaaa ctcctgacct caggtgatcc acccgcctca gcctcccaag
ggcttcagaa tctaagacat ggctctagtt tcagtttacc acatttctag cagaatgatg
ttgggaatgt cacctgactt ccataaatcc ttattttctc ctctgataaa cagcagtgat
gttatgggga gctgatgaga tatctatgta aaaacatttc tcaaaccata aattacggtg
gatgaacatc tgtacttgtg ttgagagtac tgatatcaag gagcaaacag gctgttgtat
gtgttgaatg agcctctccc cactcacaca cccacagggc tctgggcacc ctggcaggag
gcatgtcctg tgagtttctg cagcagatca actccatggt agacttcctt gaagtggtgc
acatgatcta tcagctgccc actagagttc gagggagcct ggtgagaggg ggtgcctgga
ctttagtggg agcagggagg ctgggaccct aggtatagaa cccagctcct atgttctgct
ctggcctcac actgcttccc tacagagggc ctgtatctgg gcagagctac agcggaggat
ggcaatgcca gaaccagaat ggacaactgt agggccagaa ctgaacgggc tggatagcaa
gctactcctg gacttaccgt aagtactgca gctagagata ttggcccctc agaaagctca
atctggggtg aagatctgcc cttagggaat gccctggagg aggtagtttt tctgtctggt
agttccctga cataatttat agcccaaagc agaggatttt attcaaagtt gctctatgta
ttgactggtt cccagaatat gctccagcac agggcagctg agggtggcaa cactgtattg
aagcctgcca agtaatctta caataaccta gtccacatta attgagattg agacagagca
tctgaagtga gggaggcaat gctccaaatc tgccccagag gattgtagtt tgctcagggc
actgtgttct tagtgcattc agaggagtag atcgagagaa aaatatatga aaaatgtgat
aaataccttc aaatacctga ggggctatca agtagaaatt agattgtcat atttatgagt
ggccccattg ggcaagacta agagtagtta acggagatca gatttttaca tagtataaga
aaaactaagg tagtgagttc ctggtccttg gagctgttcg agcctaagcc agatggcccc
atggcaggaa tgttgtagag cacgttcata tacaggttgt gggaagaaaa ggctatagga
acccaaggct cctccctacc catggagaaa tttattagta tgttactcat atgctgcttt
tctcatttta cccctaccac caccccgttg ccatccgcac tgtaagtcag gataggaaaa
tgctggtgtt acagtcttcc tggggaatat ggagctgaag tggagtaaaa gcagttgact
tcattcctac ttttttcttt tttttctttt tttttttttt tgagacagag ttttgctgtg
tcaccaaggc tggagtgcag tgacgtgatc tcggctcact gcaacctcca tcttccaggt
tcaagcaatt ctcctgcctc agtctcccga gtagctggga ctgtaggtgt gcaccaccat
gccaggctaa tttttgtatt tgttgtaggg acgagctttc accatgttgg ccaggctggt
cttgaactcc tggcttcaag tgatctgccc acctcggctt cccaacattc ttatattttt
ataggccttt ccacagattt cagctcttgt atgacttagc ccagttccag aactggtaat
cctaggtagg gtacaggtta tcacctctga tttcgggtaa aagggattta tttatttatt
tgtttattta tttatatttt tgagacagag tctcgctctg tcacccaggc tggagtgcaa
tggtgccatc tcggctcact gcaacctctc cctctggggt tcaagcaatt ctcctgcctc
agcctgctga gtagctggga ttacaggcgc gtgccaccac acccggctaa tttttgcatt
tttagtagag acggggtttc accatgttgc tcagggtggt ctcgaatttc tgaccctgtg
atctgcctgc ctcggcctcc caaagtgctg ggattacagg catgagccac tgcgtccggc
ctgtttttac ttttttttaa tgccattcag atctgtttaa atatgtgggt tctgtgagat
aatttagaat cccaaggtta cagatgaggt gaaagatcct agaccatgca tcaaaaaact
tgagtttctc atttgtgaaa gaaggataag agaaacacct attttgtctg ggtgcagtgg
ctcatgccta taatcccagc atttggggag gccaaggtgg gtggatcacg gaggtcaggt
gttcaagacc agactggcca acatggcaaa acaccatctc tactaaaaat acaaaagtta
gctgggcgtg gtggcacgtg cgtgtaattc cagctattcg ggaggctgag gcacgagaat
tgcttgaacc tgggaggtgc gggttgcagt gaactgagat cgcagcacca ctgtgctcca
gcctgagtga tggagtgagg ccaggtcttg ttgtaggatc aaatgagata acacctgaaa
gaactttgta aattgtatag cacgtacaaa caagaaggga cctcttcaca agcagaggaa
gggtggtcct gtggaaaaaa acgggaattg ggagtgagag acctcaacat ttgatctctg
tgaacctcag ttttttaatc tataaaatgg ggaaatgtta atggtactta atatttggag
cttttgagtc cattagatca ggtaggattg ttcgttattt ttttttttta ggaagactag
aaatatgttg ctcccttttt ctcccccact caagcttgat ggtgggaatt ggccctggag
ctgtttacta tcagttcctg tccagcttca ctaaatttgg tctggggtca catcttagct
gcggactgtg gggttttgtg gtcccttctc gacttggccc agctccacct gaatcctgtt
gttgtcaaat tgctgtaata ggatccagtt gatggacaga ctatccaatg aatccattat
gttggtggtg gagctggtgc aaagagctcc agagcagctg ctggcactga cccccctcca
ccaggcagcc ctggcagaga gggcactaca aaacctggta agagtccacc ctaccagact
cagatttgct gccctgggca attcttgctc ctcagacaat gctctctgac tgtcccccaa
ccctctactt cttgctttct tgctgccaaa cagattcctg tctacaaggc ctggcccctg
ttttgcctct gggttctgtt ccttgataat atgcttcacg ttacttgtcc atacctcttg
gagtccgaga aatctcttgg agtccacctc tcagtctttc tgcctgctcc tatctgggct
cattgcttaa ggaagtgaac aaaggtagtg agcatcatag ggtgctgagc tgggagcagg
agggagggaa ggttaggggg cttggtgtct tgatcaaggt gtctggtatt ctgagtcaga
agtgcattgt ccaagttctg atgctcttct ccaggctcca aaggagactc cagtctcagg
ggaagtgctg gagaccttag gccctttggt tggattcctg gggacagaga gcacacgaca
gatcccccta cagatcctgc tgtcccatct cagtcagctg caaggcttct gcctaggaga
gacatttgcc acagagctgg gatggctgct attgcaggag tctgttcttg ggtatggacc
ttcgagaact tcagattcta actcattcta tacccagtcc ctcagccacc atcatcagtg
gcagcctgtt ccatattctt aaggtcccct ggagccctgt gtccgaaatc ctagcatgtc
ctcttttccc cttccttttc ctcacagttc cctcagctcc ccagcccccg attttcttcc
tgtccccagg aaaccagagt tgtggagcca ggatgaagta gagcaagctg gacgcctagt
attcactctg tctactgagg caatttcctt gatccccagg gtgagatgaa ggaagaaggg
aagggagtaa atgcatagag gggactggtg agctggttat ggggacccgt ggccaaagag
ggcaaaggat atgaagccta gatctggggg gagactgcaa aacagagaca ggactttgga
cttagagcta tagcagcagg tcctgatctg tccagatctc cccactctcc ttctaccttc
tcatgcagga ggccttgggt ccagagaccc tggagcggct tctagaaaag cagcagagct
gggagcagag cagagttgga cagctgtgta gggagccaca gcttgctgcc aagaaagcag
ccctggtagc aggggtggtg cgaccagctg ctgaggatct tccaggtgaa actacccaaa
tacttatatg tccagcagga tgtacaggga gtatcaaacg gtctgggttc tacatgtgct
cttccctggg actgggtttt ctaatttata aagcaaagag tttagaggga tgatcttcaa
gcctcttgta gttctagaat tctgtagttc tgggagtttg taaactatta agttttcttt
tagcccagaa cttccatttt cctgctctct cgtgtctgct ctagactcag ctctagctcg
gctaagtgtg gagctctctg ctggggagat ccctagaagc tttgaaggag acattgtgag
gctggagaac tgggttcaaa ttcagtgcta ccattaaatc tctgaataac atcctcagtc
ttccatctat aaaagtcttg gcatctccaa tcacttcttg ttctattatc tcctaagccc
tatacatatt actctgtaat actcctttga tccctatttc tcacagtgct ctatcctcca
aaggttggaa gactcactct atctacagat atctctctgg gcatatttta ttactgcgct
gacctcctgg ccctgccttc ccccttcaga acctgtgcca aattgtgcag atgtacgagg
gacattccca gcagcctggt ctgcaaccca gattgcagag atggagctct cagactttga
ggactgcctg acattatttg caggagaccc aggacttggg cctgaggaac tgcgggcagc
catgggcaaa gcaaaacagg ttagggatgg agagccaact ggggttggcc atgaggaagc
tatttgggtg tgatgtagga cacaaagaga atggagagtt ggatgagagg tgggggaagc
aagagataga agagttagaa gatttgggtc acaagtagga ggtgaaggga gataaatatt
gaggaaagag agctagtata atgaatagag ggacgaaagc agtggttacc aaattttaat
gcatatcacg atcatcaagg gaacagattt ttttctttat ttttttttct ttcttaaaaa
aataatggca tgcttcggct gggtgcagcg gctcacgcct ataatctcag aactttggga
ggccaaggcg ggcagatcac gaggtcagga gatcaagacc atcctgtcta acacggcgaa
acacggtctc tactaaaaat acaaaaaagt tagccgggca tggtggtgca cacttgttgt
cccagctact tgggaggctg aggcaggaga atggcgtgaa cctgggaggg ggagcttgca
gtgagccgaa gtcaagccaa tgcactccat cctgggtgac agagcaagac tccatctcaa
aaaaaaaaaa aaaaaaaaaa ggcatgcttc atgaatttgc gtgttatcct tgcacaggcg
ccatgcaaat ctctgtatca ttccaatttt ttggggtatg tgctgctgaa ctgagcatgg
gaacagtgcc agtgccagat taccatgctt cactgactta ataaaaacct ttggggaggc
tgggcgcagt gactcatgcc tgtaatcaca gcactttggg aggcggaggc aggtggattg
cttgagccca ggagttagag accagactgg gcaacatggt gaaaccctgt ctctactaaa
aatagaaaaa acattagctg ggtgtggcgg cacatgcctg taatcccagc tactcaggag
gctggggtag gagaatccca tgagtgcagg aggtggaggg tgcaatgtgc caagatcgca
ccactgccct ccagcctggg tgtcagagca agaccctgtc tcataaatta aaaaataagc
ctctggggga aagagtctag acatctgcat ctcctttttt tttttttttt tttttttttg
agacagagtc tcactctgtc acccagcatc caggctggag tgcagtggtg tgatcttggc
tcactgtaac ctctacatcc tgggttcaaa cgatcctcct gcctcagcct ctcaagtagc
tgggactaca ggtgcaccac acctggctaa tttttgtatc tttggtagag atggggtttc
actatgttgc ccaggatggt ctcgaacttc tgggctcaag caatcctccc acctcagcct
cccaaagtgc tgggattaca gctgttagcc actgtgctgg gccctaggca tctgttttaa
taagcgtctc tgtgtctgat gcacataaaa gtgtggaact catggactag agttagtttg
ctcttctttt ccactgattg taatgtcttt caaaacacct tagaggaact gtaaggcaac
ggtctcattt tatagtggag gaaactaaag aaaaggcaaa tgatttacct agagttatac
agctaagggc agaggcaaga cttaaaaccc agcagtatga ctcccaatcc actgcttttc
cactcacatt gttcctgtct ttctcctagt tgtggggtcc cccccgggga tttcgtcctg
agcagatcct gcagcttggt aggctcttaa taggtctagg agatcgggaa ctacaggagc
tgatcctagt ggactgggga gtgctgagca ccctggggca gatagatggc tggagcacca
ctcaggtaac acttttcctc ctccctacgg cttcccaaac acccatccca cagacccagc
cctatagatc atctaaagcc caaggaattt ttttcctgtg accctacctg gtccttcttt
ctatcttttg ttgatacccc atactagtga ccttcaggac tctgatttat tcactctgag
gccctggaca cataatactg tctcctacct cttttcctgg aggcttcctc tttttctttc
cttttctttt ctgagtcctc agccttcccc atgactcctt aggtcttaat agtaacagaa
tataacccag taacacctat cacttccctg tccattaatt ctccataact ttcctccttc
ccctcttctc ccacccccca ccccagctcc gcattgtggt ctccagtttc ctacggcaga
gtggtcggca tgtgagccac ctggacttcg ttcatctgac agcgctgggt tatactctct
gtggactgcg gccagaggag ctccagcaca tcagcagttg ggagttcagg tcatttgtga
aggggctgag ggtggtggtg ctgaggtaaa ggtggactta ctggggaaag aaggatcatg
aaggtctggt cccatggagg aagggaactc atttgaagcc atctcttcct ttgtctcatg
accacagccc ctttcactga agccgaattc ttcttccttc cttcctactg ttctacagcc
aagcagctct cttcctcggc accctgcatc tccagtgctc tgaggaacaa ctggaggttc
tggcccacct acttgtactg cctggtgggt ttggcccaat cagtaactgg gggcctgaga
tcttcactga aattggcacc atagcaggtg gggagctggg ccactgctgg tgcaagttgg
tttggtttct ataccatggg tggactggat ggaagactgc cctgcaattc ttaaggtggg
ggcctgaggg tgtttaaata aggggctaga gacatattgg ggaaggtcta tgatagggca
ctttgggagt agttagagaa ggtctatagg tttgaagaga gggaaggtca gtctaagaca
atgtttggat gccacttgct tcaacagctg ggatcccaga cctggctctt tcagcactgc
tgcggggaca gatccagggc gttactcctc ttgccatttc tgtcatccct cctcctaaat
ttgctgtaag tattaatgga ctggggtgac cacaggagag ccagggccca atggggacta
catgcatgca ctgattccta cccctgccct caggtggtgt ttagtcccat ccaactatct
agtctcacca gtgctcaggc tgtggctgtc actcctgagc aaatggcctt tctgagtcct
gagcagcgac gagcagttgc atgggcccaa catgagggaa aggagagccc agaacagcaa
ggtgagttcc cagctgcaca gcttgatcct ccatctcctg acccagaatc aaacccctaa
tttggtgctg tctggctctt agagtgcacc cagggagatc cctggagtga aggagtctac
aggcagagcg ctaatttcca agtatcaatg ctcctggaga gctgagttgt gatattactc
ccattccctg tctattatag gtcgaagtac agcctggggc ctccaggact ggtcacgacc
ttcctggtcc ctggtattga ctatcagctt ccttggccac ctgctatgag cctgtctcta
cagtagaagg agattgtggg gagagaaatc ttaagtcata atgaataaag tgcaaacaga
agtgcatcct gattattttc agaagctgat gaggaata

The nucleotide sequence of the human STRC pseudogene is shown in SEQ ID NO: 5.

Human Stereocilin Pseudogene 1 (STRCP1)
(SEQ ID NO: 5)
gccctgccct cacctggcta tcccacacag gtgagaataa ccagaactca cctccggtac
cagtgttcac ttggaaacat ggctctcagc ctctggcccc tgctgctgct gctgctgctg
ctgctgctgc tgtcctttgc aggtaagaag aacagtgagc agaactgggg atgaggagga
gggtggctgg aaaaagactt taagaatatg gaggtgaacc tgttagatag aaggacaaag
gagagaggca gagacttgtg caaaagggaa aaatgagggt taagaaaagc aggccaagac
ttactgtagg ccagtgaaag gggttcagct caccatcccc tcacctcatc tttagatcca
ggtagggaac tgtgctcagg ggcagggttg agtttgggct ctgtgttcct ctccttcagt
gacctctggt ttctctcctt acagtgactc tggcccctac tgggcctcat tccctggacc
ctggtctctc cttcctgaag tcattgctct ccactctgga ccaggctccc cagggctccc
tgagccgctc acggttcttt acattcctgg ccaacatttc ttcttccttt gagcctggga
gaatggggga aggaccagta ggagagcccc cacctctcca gccgcctgct ctgcggctcc
atgattttct agtgacactg agaggtagcc ccgactggga gccaatgcta gggctgctag
gggatatgct ggcactgctg ggacaggagc agactccccg agatttcctg gtgcaccagg
caggggtgct gggtggactt gtggaggtgc tgctgggagc cttagttcct gggggccccc
ctaccccaac tcagccccca tgcacccgtg atgggccgtc tgactgtgtc ctggctgctg
actggttgcc ttctctgctg ctgttgttag agggcacacg ctggcaagct ctggtgcagg
tgcagcccag tgtggacccc accaatgcca caggcctcga tgggagggag gcagctcctc
actttttgca gggtctgttg ggtttgctta ccccaacagg ggagctaggc tccaaggagg
ctctttgggg cggtctgcta cgcacagtgg gggcccccct ctatgctgcc tttcaggagg
ggctgctccg tgtcactcac tccctgcagg atgaggtctt ctccattttg gggcagccag
agcctgatac caatgggcag tgccagggag gtgagtgtgg ccagggctgg gactgggatg
tggcagggca aggaaagtga aattggggta gttttcttcc ttactctttc cctcctaggt
aaccttcaac agctgctctt atggtaagta acaggagacc agttctgagg gattgggcct
ggaaaatctg gaggtgaaga gctgaagacc tcagcctcta gagaggaaaa ctgatgggag
gagtgtagtt tagtggtttt ggggtgtgac tgtctgggtt ggtgtcccag ctccacctct
tcctagccat atgaccttga gcaggttaca tagtctttct atacctcagt ttccccattt
ataaaatgag aatgataata ttagttacca cagagttgtt gcacccggtt aaatgagttg
atactgtgta tgcaaacgac ttaaaaccgt gctggcacat agcgcttaat aatgttagct
agtaaagatg ggatttggaa aataaggaca cagctggatt cctctacccc cttactactt
cagtacaaca atgccagaca gtagttagac atattgagtt gctgagcaga tttcctaaca
tgaggcccgc tgagggttgt gtttaagcta tctaaaagca tatgaagaaa ggagacagaa
gggggccagg tggacagaaa gaattccaac tggggcttct cctaggtgat tttggacctt
ggcagggcag ctttctcttt tttgccccgt tgcagcattt caaccagtaa cgcctaaact
ctcagggacc tcgcttgtag aaaagcctat gcttgccatg ccccttgagg gctctgagtc
agggtcagaa tcttcagctg gaggaaatgt gaactgacca gatcctgcct gctcctccct
ctgcacccag gggcgtccgg cacaaccttt cctgggatgt ccaggcgctg ggctttctgt
ctggatcacc acccccaccc cctgccctcc ttcactgcct gagcacgggc gtgcctctgc
ccagagcttc tcagccgtca gcccacatca gcccacgcca acggcgagcc atcactgtgg
aggccctctg tgagaaccac ttaggcccag caccacccta cagcatttcc aacttctcca
tccacttgct ctgccagcac accaagcctg ccactccaca gccccatccc agcaccactg
ccatctgcca gacagctgtg tggtatgcag tgtcctgggc accaggtgcc caaggctggc
tacaggcctg ccacgaccag tttcctgatg agtttttgga tgcgatctgc agtaacctct
ccttttcagc cctgtctggc tccaaccgcc gcctggtgaa gcggctctgt gctggcctgc
tcccaccccc taccagctgc cctgaaggcc tgccccctgt tcccctcacc ccagacatct
tttggggctg cttcttggag aatgagactc tgtgggctga gcgactgtgt ggggaggcaa
gtctacaggc tgtgcccccc agcaaccagg cttgggtcca gcatgtgtgc cagggcccca
ccccagatgt cactgcctcc ccaccatgcc acattggacc ctgtggggaa cgctgcccgg
atgggggcag cttcctggtg atggtctgtg ccaatgacac catgtatgag gtcctggtgc
ccttctggcc ttggctagca ggccaatgca ggataagtcg tgggggcaat gacacttgct
tcctagaagg gctgctgggc ccccttctgc cctctctgcc accactggga ccatccccac
tctgtctgac ccctggcccc ttcctccttg gcatgctatc ccagttgcca cgctgtcagt
cctctgtccc agctcttgct caccccacac gcctacacta tctcctccgc ctgctgacct
tcctcttggg tccaggggct gggggcgctg aggcccaggg gatgctgggt cgggccctac
tgctctccag tctcccagac aactgctcct tctgggatgc ctttcgccca gagggccggc
gcagtgtgct acggacgatt ggggaatacc tggaacaaga tgaggagcag ccaaccccat
caggctttga acccactgtc aaccccagct ctggtataag caagatggag ctgctggcct
gctttagtgt gagtgctctg ccagagggaa agctcctaga acagtgagaa ggccctccag
gggaattcct cgaatactca gaggcagtag tgtggggtag tagttgaagc acacagctct
agagtcagac aggcttggat tcatatcttg gttctgtgac cagccttgaa tgagttattt
aacttctctg agcaatattt ttctcgtctc atttataaac tagggatgat aatggtatat
gagataatac atgctgtggg cttagcacag tgcatgatac acaaacatgc aataaatatt
accttgttat tcttttgggc tctttgactc tctcactttc tgcaccagaa agaaaaagga
tcaagttaga ggactctaaa tttttcccct agagagtgag aattggaggc tggcagaata
caggaagata aggtaggaat gagaaagatt cagggacact accaatcaga agactttggt
tctaggttca actgtgccac aaattagtgt gatcttaggc aagcaatttc atttagtttt
tctgggcttc agtttttagt ctgtagaatg gaggggtgag aatatgttaa acaccataat
taattcactg agtgcctatt atatgcaagg cactttgcta ggttctgtag gatatataaa
gatttcttac tccatgttgg ggccaccttt ttcaaaccct gggcccagta aaatggaatt
agatagtctc atagtatttg gttcaggtct acaagtatta attgagccaa ctatggacct
ggcatgggag agggtacaag agaaattaga gatatgatcc cggacctaaa agagcttaat
atctgaagaa tcacacttga gatgatggac aagcatccca gcaagtggag ttggaatgcc
tgggggagct gcaggagaga cagagaagac agctctgttg gcatattgtc tttcttccca
ccagcctgtg ctgtgggatc tgctccagag ggaaaagagt gtttgggccc tgcagattct
agtgcaggta acaggtggag ggcacatggg tgggctgggt gacagccatg gctggaggtc
cctgccccgt gaggtgaggc catacccacc atgacctcct attcgcaggc gtacctgcat
atgcccccag aaaacctcca gcagctggtg ctttcagcag agagggaggc tgcacagggc
ttcctgacac tcatgctgca ggggaagctg caggggaagc tgcaggtgag cactgagaaa
ggggagcaag ggcacctgga gcctagtgtt cagagggctt gctttagtgg gaggaggaac
tccagagagg aaatggcagg gatactgagc atctccagag gcagaatcca ttcctgtgcc
cctacaggta ccaccatccg aggagcaggc cctgggtcgc ctgacagccc tgctgctcca
gcggtaccca cgcctcacct cccagctctt cattgacctg tcaccactca tccctttctt
ggctgtctct gacctgatgc gcttcccacc atccctgtta gccaacgaca gtgtgtaagg
ttcttgcact actcctcctg ctcctgtcac ggtcaggcca accgcatcca cctggagcag
ccccttccgg agctcctctc tgtttttttc tttcatgcca gataggcaat gtgccaacat
cgtagcaagg tttgagagag gcacatctca cgcctgagtg tgaaaaccca atcattatgc
taatgaacta caaaaggatc agagagctcc tctctattaa aaccagggag aggatgggcg
tggtggctca tgcctgtaat cccagcacgt tgggagcccg aggcaggtgg atcactaggt
ccgcctagtg agttcgagac cagcctggcc aatatggtga aaccctgtct ctattaaact
acaaaaatta gccaggcatg gtggtgggcg cttgtagtcc cagttactct ggaggctgag
gcaggaggat agcttgaacc tgggaggcag aggttgcagt gagccaagat cgtgccactg
cactccagcc tgggtgacag agcgagactc cgtcttaaaa aaaacaaaaa acaaaacaaa
acaaaaaaac agggagagtc tccttcctat ctagacagca gggctacaga gggtcagagg
aaaacagttt ggaggaagac aaagggttaa gacccatgac tcctcgcagc ctggctgcca
tccgggatta cagcccagga atgaggcctg aacagaagga ggctctggca aagcgactgc
tggcccctga actgtttggg gaagtgcctg cctggcccca ggagctgctg tgggcagtgc
tgcccctgct cccccacctc cctctggaga actttttgca gctcagccct caccaggtat
gagaatcatc ttctttactt gactggccca tcttctgcta gtggggacaa agagtcaatg
gcatgtctct cagtggcccc tccctgcaag aaccctatag tgaccccagt gcgagctaac
cttccccatc tcagatccag gccctggagg atagctggcc agcagcaggt ctggggccag
ggcatgcccg ccatgtgctg cgcagcctgg taaaccagag tgtccaggat ggtgaggagc
aggtacgcag gtgagttgtt gtgggatcag taaccaaggc aagagtggaa gaggtagaga
gaggaaggca cagctgtcac gctgggtcgg tgttctagga agaaaggggc aagagagtag
gcagtggcct caggcagcat agagttccag gagagaggtc tatagatggt gcccctgtgt
agtggtgtag tgtcagagtg cccagtgtat gtacccatac catctgctgc caggcctgcc
ttagtgctag tcttggggac cacacaaagg tcagcttcat gccctcctca ggcttgggcc
cctcgcctgt ttcctgagcc ctgaggagct gcagagccta gtgcccctga gtgatccaac
ggggccagta gaacgggggc tgctggaatg tgcagccaat gggaccctca gcccagaagg
acgggtgagc ccctcagcac aagcctacaa gactttaggc ttcccctggg tctgtgtgga
tggctttccc attgtgtcaa cttgagcaca gtggtgccag cccccatccc acttttgcaa
cctccattcc ttactccatg gccattctta cctgttacca cctcttcctg gcccttctct
atctggtctg tagcacccca aacataccct ttgccatttt gaacctaatc tactccagtc
caatccctag ttccaaaccc tagcccaggc cctgggaaat tcagatgtgg gattagagag
gaagttcaag gttcatctgt cttttctctc cagtcctaaa ccttctttgg ttacaggtgg
catatgaact tctgggtgtg ttgcgctcat ctggaggagc ggtgctgagc ccccgggagc
tgcgggtctg ggcccctctc ttctctcagc tgggcctccg cttccttcag gagctgtcag
agccccagct tagagccatg cttcctgtcc tgcagggaac tagtgttaca cctgctcagg
tttgcctgtc tcactccctg gcatgtaccc tccatccccg cttgagcccc agtcaagaga
atcccattca gggataaaag cagcccctcc tttccctggg tgaacagtag aggtaaactc
tgtctgcagg aggacgcctt cattcccttt cctcagatca agaagggacc tgagtcactg
aggatggtta ctggggatgg ttaagaggca gcgggaagtt ttggagggtt tgccttagga
acccacttag gacctggctg ctgggtcctg agagctgttg ttttcggtcc catcccaaca
caggctgtcc tgctgcttgg acggctcctt cctaggcacg atgtgagtag cagcaacttc
tcagcctccc gccagaggtc tctatcctct tttaacctgg ctcctgcatc tgcccctcct
ctctctccgc tcccctcata cttactgcct tgctgcattg tgattgttgt cttccccaac
acccttccct tcttcttcag gcctcttgtc tctcttgctc tttagctatc cctggaggaa
ctctgctcct tgcaccttct gctaccaggc ctcagccccc agacactcca ggccatccct
aggcgagtcc tggtcggggc ttgttcctgc ctggcccctg aactgtcacg cctctcagcc
tgccagaccg cagcactgct gcagaccttt cgggtatgag agtggcaagg aggatgagat
aatcagggat accggctctt tctggttggg aggaaggcat cttccctgag gccagggaag
gcctttcata cctccccact tacacacaca cacacacaca cacacacaca accaattctc
atgcaggtta aagatggtgt taaaaatatg ggtacaacag gtgctggtcc agctgtgtgc
atccctggtc aggtaagtgt gagatctccc aactgagctc ctctccccat tctggggcag
tttcatatgg ctggtgctac ctcccacact accctgcagt ggccctgaga gttctggtta
gctctgtgcc cattagcagc cctccccagt gccagatgca ggacagcatg atccactcac
attgtcctag actaatgtca aagctggaag ggcctgagaa atcttccagg ccacccaccc
tgctttcaga tgaaaagacc aaggctggga gaagctaagg gactttgttt gcctggtgcc
taactagcag caacacttga ccacagcagc ctgcagtgtg aggctcttag gcgtttattg
ctacagtggc aaatgccatt ccacttctgt cctagctttg gtccctttcc acccccatgg
ttccttttct ctgagtgcta agtacagact ctctcaccta tcactacact gctataccca
tcaccgccag cagcctattc ccaccacctg gccagactgc ctgcttcccc tgctcccatt
aaagctgcta caactggatt ccttggctct tctggcaaat cgaagacgct actgggagct
gccctggtct gagcagcagg taattctccc cacttaattt cagaacttcc tccctcaatg
tagtctacct tctttaccta tcccttagcc ctatttggcc agcttatccc tactatcctt
tatttgattg tttgagatac agtctcactc tgttgcccag gctgcagtgc agtggcatga
tcagagttcg ctgtaacctc aaactcatga gctcaggcaa tctttctgcc tcagcctcct
gaatagctag gatgacaggt ggttaccacc atgcctggct aatttttaaa tttttttttt
gttttttgag atgaagtctt gctctgtcac ccaggcttga gtacagtggc acaagcttgg
ctcactgcaa cctctgtctc ccgggttcaa gcgattctcc tgcctcagcc tcccgagtag
ctgggactac aggcactccc cacaatgccc ggctaatttt ttttttgttt tagtagagac
agggtttcac catattggcc aggctggtct cgaactgctg accttgtgat ctgcctgcct
ctgcctctca aagtgctggg attacaggtg tgagccacca tgcccggcca atttttaaat
tttttgtaga gacagacaat acaaaaatgt ggacactatg tggagacact atgttgaggt
actatgctgt ccagattggt cttgaactcc tggcctcaag caatcctcct gccttggcct
cccaaagtgc tgggattaca gacctgagcc actgcaccca gccccctagt atcccttata
atgtgacttg cttttctttt tctttctcct tcccttttct ttcatttctt tctcactctc
gagagaagag tgggcatctg ggagagtggg aggctggtgg gtcccacaga gtgaggaggc
aggactgggt ccaaggcagt cctgcctctc cactctaggg ggtatccttg gacagtgtct
cttctgggaa ggggctcgtc tttctttctc ttgtaggcac agtttctctg gaagaagatg
caagtaccca ccaacctgac cctcaggaat ctacagtgag taacttgtgt tgagcagtgc
gctgaattcg accaacattt ttttgagtgc ttactatgtg ccaggcacca tatgatatgg
aatgggggat atagggatga atgatgcata gtccctgcct cgtggacgtt ctcctagcac
ctccctttgc cctcctttcc ttccacagtg ccatgcctat cctgactaga gccaaaggac
tcagaaaacc tggattcagg ttccagtcct gtcacctact tgtcctcttg ggcaagtcat
ttaacgtccc tgtgtcagtt ttcccttctt taaatgagaa ttacaatggc accagcctca
taggtagtta ctgtgaagat taaatgaggt aggtcatgta agatatttaa cacagtgttt
ggtccattgt aaagttccag tagtcatttg ctactgttag tttacttcag gatgacttca
gaggcactgg ccaagcaaga ataaatagga ataagaaggt atcactttac ttatacccac
attagaagaa caatgggctt cagaatcttt tttttttttt ttttcgagac agtcttgctc
tgttgcccag gctggagtgc agtggcgcga tttcggctca ctgcaacctc tgcctcccag
gttcaagcga ttctcctgtc tcagcctctg gagtagctgg gattacagga atgtgccacc
atacccagct aatttttgta tttttagtag agatggggtt tcaccatttt ggccaggctg
gtctcaaact cctgacctca ggtgatccac ccgcctcagc ctcccaaggg cttcagaatc
taagacatgt ctctagtttc agtttaccac atttctagca gaatgatgtt gggaatgtca
cctgacttcc ataaatcctt attttctcct ctgataaaca gcagtgatgt tatggggagc
tgatgagata tctatgtaaa aacatttctc aaaccataaa ttacggtgga tgaacatctg
tacttgtgtt gagagtactg atatcaagga gcaaacaggc tgttgtatgt gttgaatgag
cctctcccca ctcacacacc cacagggctc tgggcaccct ggcaggaggc atgtcctgtg
agtttctgca gcagatcaac tccatggtag acttccttga agtggtgcac atgatctatc
agctgcccac tagagttcga gggagcctgg tgagagggga tgcctggact ttagtgggag
cagggaggct gggaccctag gtatagaacc cagctcctat gttctgctct ggcctcactc
tgcttcccta cagagggcct gtatctgggc agagctacag cggaggatgg caatgccaga
accagaatgg acaactgtag ggccagaact gaacgggctg gatagcaagc tactcctgga
cttaccgtaa gtactgcagc tagagatatt ggcccctcag aaagctcaat ctggggtgaa
gatctgccct tagggaatgc cctggaggag gtagtttttc tgtctggtag ttccctgaca
taatttatag cccaaagcag aggattttat tcaaagttgc tctatgtatt gactggttcc
cagaatatgc tccagcacag ggcagctgag ggtggcaaca ctgtattgaa gcctgccaag
taatcttaca ataacctagt ccacattaat tgagattgag acagagcatc tgaagtgagg
gaggcaatgc tccaaatctg ccccagagga ttgtagtttg ctcagggcac tgtgttctta
gtgcattcag aggagtggat cgagagaaaa atatatgaaa aatgtgataa ataccttcaa
atacctgagg ggctatcaag tagaaattag attgtcatat ttatgagtgg ccccattggg
caagactaag agtagttaac ggagatcaga tttttacata gtataagaaa aactaaggta
gtgagttcct ggtccttgga gctgttcgag cctaagccag atggccccat ggcaggaatg
ttgtagagca cgttcatata caggttgtgg gaagaaaagg ctataggaac ccaaggctcc
tccctaccca tggagaaatt tattagtatg ttactcatat gctgcttttc tcattttacc
cctaccacca ccccgttgcc atccgcactg taagtcagga taggaaaatg ctggtgttac
agtcttcctg gggaatatgg agctgaagtg gagtaaaagc agttgacttc attcttactt
ttttcttttt cttttttttt ttttttttga gacagagttt tgctgtgtca ccaaggctgg
agtgcagtga cgtgatctcg gctcactgca acctccatct tccaggttca agcaattctc
ctgcctcagt ctcccgagta gctgggactg taggtgtgca ccaccatgcc aggctaattt
ttgtatttgt tgtagggacg agctttcacc atgttggcca ggctggtctt gaactcctgg
cttcaagtga tctgcccacc tcggcttccc aacattctta tatttttata ggcctttcca
cagatttcag ctcttgtatg acttagccca gttccagaac tggtaatcct aggtagggta
caggttatca cctctgattt cgggtaaaag ggatttattt atttatttgt ttatttattt
atatttttga gacagagtct cgctctgtca cccaggctgg agtgcaatgg tgccatctcg
gctcactgca acctctccct ctggggttca agcacttctg cctcagcctc ctgagtagct
gggattacag gcgcgtgcca ccacacccgg ctaatttttg catttttagt agagacgggg
tttcaccatg ttgctcaggg tggtctcgaa tttctgaccc tgtgatctgc ctgcctcggc
ctcccaaagt gctgggatta caggcatgag ccactgcgtc cggcctgttt ttactttttt
ttaatgccat tcagatctgt ttaaatatgt gggttctgtg agataattta gaatcccaag
gttacagatg aggtgaaaga tcctagacca tgcatcaaaa aacttgagtt tctcatttgt
gaaagaagga taagagaaac acctattttg tctgggtgca gtggctcatg cctataatcc
cagcatttgg ggaggccaag gtgggtggat cacggaggtc aggtgttcaa gaccagactg
gccaacatgg caaaacacca tctctactaa aaatacaaaa gttagctggg cgtggtggca
cgtgcgtgta attccagcta ttcgggaggc tgaggcacga gaattgcttg aacctgggag
gtgcgggttg cagtgaactg agatcgcagc accactgtgc tccagcctga gtgatggagt
gaggccaggt cttgttgtag gatcaaatga gataacacct gaaagaactt tgtaaattgt
atagcacgta caaacaagaa gggacctctt cacaagcaga ggaagggtgg tcctgtggaa
aaaaacggga attgggagtg agagacctca acatttgatc tctgtgaacc tcagtttttt
aatctataaa atggggaaat gttaatggta cttaatattt ggagcttttg agtccattag
atcaggtagg attgttcgtt attttttttt tttaggaaga ctagaaatat gttgctccct
ttttctcccc cactcaagct tgatggtggg aattggccct ggagctgttt actgtcagtt
cctgtccagc ttcactaaat ttggtctggg gtcacatctt agctggggac tgtggggttc
tgtggtccct tctcgacttg gcccagctcc acttgaatga tattgttgtc aaattgctat
aatagaatcc agttgatgga cagactatcc aatgaatcca ttatgttggt ggtggagctg
gtgcaaagag ctccagagca gctgctggca ctgacccccc tccaccaggc agccctggca
gagagggcac tacaaaacct ggtaagagtc caccctacca gactcagatt tgctgccctg
ggcaattctt gctcctcaga caatgctctc tgactgtcct ccaaccctct acttcttgct
ttcttgctgc caaacaggtt cctgtctaca aggcctggcc cgttttgcct ctgggttctg
ttccttgata atatgcttca cgttacttgt ccatacctct tggagtccga gaaatctctt
ggagtccacc tctcagtctt tctgcctgct cctatctggt ctcattgctt aagaaagtga
acaaaggtag tgagcatcat agggtgctga gttgggagca ggagggaggg aaggtcaggg
ggcttggtgt cttgatcaag gtgtctggta ttctgagtca gaagtgcatt gtccaagttc
tgatgctctt ctccaggctc caaaggagac tccagtctca ggggaagtgc tggagacctt
aggccctttg gttggattcc tggggacaga gagcacacga cagatccccc tacagatcct
gctgtcccat ctcagtcagc tgtaaggctt ctgcctagga gagacatttg ccacagagct
gggatggctg ctattgcagg agtctgttct tgggtatgga tcttcgagaa cttcagattc
taactcattc tatacccagt ccctcagcca ccatcatcag tggcagcctg ttccatattc
ctaagggccc ttggagccct gtgtccgaaa tcctagcatg tcctcttttc cccttccttt
tcctcacagt tccctcagct ccccagcccc cgattttctt cctgtcccca ggaaaccaga
gttgtggagc caggatgaag tagagcaagc tggacgccta gtattcactc tgtctactga
ggcaatttcc ttgatcccca gggtgagatg aaggaagaag ggaagggagt aaatgcatag
aggggactgg tgagctggtt atggggaccc gtggccaacg agggcaaagg atatgaagcc
tagatctggg gggatactgc gaaacagaga caggactttg gacttatagc tatagcagca
ggtcctgatc tgtccagatc tccccactct ccttctacct tctcatgcag gaggccttgg
gtccagagac cctggagcgg cttctagaaa agcagcagag ctgggagcag agcagagttg
gacagctgtg tagggggcca cagcttgctg ccaagaaagc agccctggta gcaggggtgg
tgcgaccagc tgctgaggat cttccaggtg aaactaccca aatacttata tgtccagcag
gatgtacagg gagtatcaaa cggtctgggt tctacatgtg cttttctctg ggactgggtt
ttctaattta taaagcaaag agtttagagg gatgatcttc aagtctcgtc tagttctaaa
attctatagc tctgggagtg tgtaaactat taagttttcc tttagcccag aacttccatt
ttcctgctgt cttgtctctt taaacagctg actcagctct agctcggcta agtgtggagc
tctctgctgg ggagatccct cgaaggagac attgtgaggc tagagaactg gtttcaaatt
cagctctgcc atgaaatccc tgaccaacat cctcagtctt ccatctataa aatgagggac
ttggcatctc caatcacttc ttgttcaatt cacttctaag ccctatacat cttaacctgt
aatactccct cgatccctac ttcccacagt gctccatcct ccaaaggttg gaagagtcat
tccatctaca gatacctcct ggccctgcct tcccccttca gaacctgtgc caaattgtgc
agatgtacga gggacattcc cagcagcctg ctctgcaacc cagattgctg agatggagct
ctcagacttt aaggactgcc tgacactatt tgcaggagac ccaggacttg ggcctgagga
accacgggca gccatgggca aagcaaaatg ggtcagggac ggagagccaa ctgaggttgg
ccatgaggaa gctgtgtggg tgtgatgtag gacacgaaga gaatagagag ttggatgaga
ggtgggggaa gcagaagata aaagagttag aagatttggg tcacaagtag aaggtgagga
gagataaata ttgaggaaag agagctacta taatgaatag agggactaaa gcagtgatta
ccaaatttta atgcatatca caatcatcaa gggaacagat ttttttcttt tttctttttt
tttctttctt aaaaaataat ggcatacttc atgaatttgt gtgttatcct tgcgcaggcg
ccatgctaat ctctgtatca ttccaatttt tttttgtata tgtgctgctg aaccgagcac
gggaacagtg ccaatgctgg attaccatgc tagactgact taataaaaac ctttgcggag
gctggacgga gtggctcgtg cctgtaatca cagcactttg ggaggctgag gcaggtggat
tgcttgagcc caggagtttg agaccagact gggcaacatg gtgaagccct gtctctacta
aaaatagaaa aaaaattagc tgggtgtggc ggcacatgcc tgtaatccca gctactcagg
aggctggggt aggagaatcc cctgagcgca ggaggtggag ggtgcaatga gccaagatca
caccactgca ctccagcctg ggtgtcagag caagaccctg tctcataaat taaaaaataa
gcctctggga gaaagtctag acatctgcat ctgctttttt tttttttttt ttttttgaga
cacagtctca ctctgtcacc cagcatccag gctggagtgc ggtggtgtga tcttggctca
ctgtaacctc tacatcctgg gctcaaacga tcctcctgcc tcagcctctc gagaagctgg
gactacaggt gcacaccact acacctggct aatttttgta tttttggtag agatgaggtt
tcgctgtgtt gcccaggatg gtcttgaact cctgggctca agcattcctc ccacctcagc
ctcccaaagt gctgggatta cagctgtgag ccactgtgct gggccccagg catctgcatt
ttaataagcg tctctgtgtc tgatgcacat aaaagtgtgg aactcatgga ctagagttag
tttgctcttc ttttccactg attgtaatgt ctttcaaaac accttagagg aactgtaagg
caacggtctc attttatagt ggaggaaacc aaagaaaagg caagtgactt gcctagagtt
atacagctaa ggtcagaggc aagacttaaa acccagcatt ctgactccca atctactggt
tttccactca cattgttcct ctctttctcc tagttgtggg gtcccccccg gggatttggt
cctgagcaga tcctgcagct cggtagactc ttaataggtc tgggagatca ggaactacag
gagctgatcc tagtggactg gggagtgctg agcaccctgg ggcagataga tggctggagc
tccactcagg taacactttt actcctccct accgcttccc aaacacccat cccacagacc
cagccctata gatcatctaa agcccaagga attttttcct gtgaccctac ctggtcattc
tttctgtctt ttgttaatat cccatactag tgaccttcag gactcttgat ttattcactc
tgaggccctg gacacataat actgtctcct acctcttttc ctggaggctt cctctttttc
tttccttttc ttttctgagt cctcagcctt ccccatgact ccttaggtct taatagtaac
agaatataac ccagtaacac ctatcacttc cctgtccatt aattctccat aactttcctc
cttcccctct tctcccaccc cccaccccag ctccgcattg tggtctccag tttcctacgg
cagagtggtc ggcatgtgag ccacctggac ttcgttcatc tgacagcgct gggttatact
ctctgtggac tgcggccaga ggagctccag cacatcagca gttgggagtt taggtcattt
gtgaaggggc tgagggtggt cgtgttcagg taaaggtgga cttgctgggg aaaggaggat
catgaagtta tagtcccatg gaggaaggga actcatttga agccatctct tcctttgtct
catgaccaca gtccctttca ctgaagccga attcttcttc cttccttccc actgttctac
agccaagcag ctctcttcct gggcaccctg catctgcagt gctctgagga acaactggag
tttctggccc acctctttgt actgcctggt gggtttggcc caatcagtaa ctgggggcct
gagatcttca ctgaaattgg caccatagca ggtggggagc tgggccactg ctggtgcaag
ttggtttggt ttctatacca tgggtggact ggatggaaga ctgccctgca attcttcagg
tgtgggcctg agagggtgtt taaataaggg gctagagaca tattggggaa ggtctatgat
agggcacttt gggagtagtt agagaaggtc tataggtttg aagagaggga aggtcagtct
aagacaatgt ttggatgcca cttgcttcaa cagctgggat cccagacctg gctctttcag
cactgctgcg gggacagatc cagggcgtta ctcctcttgc catttctgtc atccctcctc
ctaaatttgc tgtaagtatt aatggactgg ggtgaccaca ggagagccag ggcccaatgg
ggactacatg catgcactga ttcctacccc tgccctcagg tggtgtttag tcccatccaa
ctatctagtc tcgccagtgc tcaggctgtg gctgtcactc ctgagcaaat ggcctttctg
agtcctgagc agcgacgagc agttgcatgg gcccaacatg agggaaagga gagcccagaa
cagcaaggtg agttcccagc tgcacagctt gatcctccat ctcctgaccc agaatcaaac
ccctaatttg gtgctgtctg gctcttagag tgcacccagg gagatccctg gagtgaagga
gtctacaggc agagcgctaa tttccaagta tcaatgctcc tggagagctg agttgtgata
ttactcccat tccctgtcta ttataggtcg aagtacagcc tggggcctcc aggactggtc
acgaccttcc tggtccctgg tattgactat cagcttcctt ggccacctgc tatgagcctg
tctctacagt agaaggagat tgtggggaga gaaatcttaa gtcataatga ataaagtgca
aacagaagtg catcctgatt attttcagaa gctgatgagg aata

In some embodiments, a stereocilin can be encoded by a sequence that is at least 70%, at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 98%, at least 99%, or 100% identical to SEQ ID NO: 8 or SEQ ID NO: 9.

One skilled in the art would appreciate that mutation of amino acids that are not conserved between the same protein from different species is less likely to have an effect on the function of a protein and therefore, these amino acids should be selected for mutation. Amino acids that are conserved between the same protein from different species should not be mutated, as these mutations are more likely to result in a change in the function of the protein. Non-limiting examples of stereocilin from other mammalian species are shown below.

Mouse Stereocilin Protein
(SEQ ID NO: 8)
malslqpqll lllsllpqev tsaptgpqsl daglsllksf vatldqapqr slsqsrfsaf
lanisssfql grmgegpvge ppplqppalr lhdflvtlrg spdwepmlgl lgdvlallgq
eqtprdflvh qagvlgglve allgalvpgg ppaptrppct rdgpsdcvla adwlpslmll
legtrwqalv qlqpsvdptn atgldgrepa phflqgllgl ltpagelgse ealwggllrt
vgaplyaafq egllrvthsl qdevfsimgq pepdasgqcq ggnlqqlllw gmrnnlswda
ralgflsgsp ppppallhcl srgvplpras qpaahisprq rraisvealc enhsgpeppy
sisnfsiyll cqhikpatpr pppttprppp ttpqpppttt qpipdttqpp pvtprppptt
pqpppstavi cqtavwyavs wapgargwlq achdqfpdqf ldmicgnlsf salsgpnrrl
vkqlcagllp pptscppgli pvpltpeifw gcflenetlw aerlcvedsl qavpprnqaw
vqhvorgptl datdfppcrv gpcgercpdg gsfllmvcan dtlyealvpf wawlagqcri
srggndtcfl egmlgpllps llplgpsplc lapgpfllgm lsqlprcqss vpalahptrl
hyllrlltfl lgpgtggaet qgmlgqalll sslpdncsfw dafrpegrrs vlrtvgeylq
reeptppgld sslslgsgms kmellscfsp vlwdllqrek svwalrtlvk aylrmppedl
qqlvlsaeme aaqgfltlml rswaklkvqp seeqamgrlt alllqryprl tsqlfidmsp
lipflavpdl mrfppsllan dsvlaairdh ssgmkpeqke alakrllape lfgevpdwpq
ellwaalpll phlplesflq lsphqiqale dswpvaglgp gharhvlrsl vnqsmedgee
qvlrlgslac flspeelqsl vplsdpmgpv eggllecaan gtlspegrva yellgvlrss
ggtvlsprel rvwaplfpql glrflgelse tqlramlpal qgasvtpaga vllfgrllpk
hdlsleelcs lhpllpglsp qtlqaipkrv lvgacsclgp elsrlsacqi aallqtfrvk
dgvknmgaag agsavcipgq pttwpdcllp llplkllqld aaallanrrl yrqlpwseqq
aqflwkkmqv ptnlslrnlq algnlaggmt ceflqqissm vdfldvvhml yqlptgvres
lraciwtelq rrmtmpepel ttlgpelsel dtkllldlpi qlmdrlsnds imlvvemvqg
apeqllaltp lhqtalaera lknlapketp iskevletlg plvgflgies trriplpill
shlsqlqgfc lgetfatelg wlllqepvlg kpelwsqdei eqagrlvftl saeaissipr
ealgpetler llgkhqsweq srvghlcges qlahkkaalv agivhpaaeg lqepvpncad
irgtfpaaws atqisemels dfedclslfa gdpglgpeel raamgkakql wgpprgfrpe
qilqlgrlli glgerelgel tlvdwgvlss lgqidgwssm qlravvssfl rqsgrhvshl
dfiyltalgy tlcglrpeel qhisswefsq aalflgslhl pcseeqlevl ayllvlpggf
gpvsnwgpei fteigtiaag ipdlalsall rgqiqgltpl aisvipapkf avvfnpiqls
sltrgqavav tpeqlaylsp egrravawaq hegkeipeql grnsawglyd wfqaswalal
pvsifghll
Dog Stereocilin Protein
(SEQ ID NO: 9)
malslwpvll lltcaatlvs sgiqsldpdl sllksllstm dqapqgslsr sqfsaflani
sssfesgrmg egpvgepppl qspalrlhdf lvtlrgspdw epmlgllgdv lallgqeqtp
rdflghqagv lgglaevllg alvpigpptp trppcirdgp sdcvlvadwl pslllllegt
rwqalvqvqp svdptnatgl dgrepaphfl qgllglltpv gelgseealw ggllrtvgap
lyaafqegll rvtdslrnev fsilgqpepd angqcqggnl rqlllwgirh nlswdvqalg
flsgsppppp allhclstgv plprasqpsa hinprqrrai svealcenhs gpappysisn
fsihllcqha qpatpqppps taavcqtamw yavswapgaq gwlqachdqf pdqfleaics
nlsfsalsgp nrrlvkqlca gllppptncp eglppapltp evfwgcflen etlwaerlcg
eaglqavpps ngawvqhvcq gptpdvtafp pchvgpcger cpdggsflmm vcandtmyea
lvpfwpwlag qcrisrggnd tcflegllgp llpslpplgp splclapapf llgmlsqlpr
cqssvpalah strlhyllrl ltfllgpgag gteaqgmlgq almlsslpdn csfwdafrpe
grrsvlrtvg eylereeqlt ppgfeptasp ssgitkmell acfspvlwdl lqreksvwal
qilvqaylhm ppenlqqlvl saereaaqgf ltlmhrswaq lqvppseeqa lgrltalllq
ryprltsqlf idlsplipfl avsdlmrfpp sllandsvla airdyspgmr peqkealakr
llapelfgev pawsqellwa vlpllphlpl enflqlsphq iqaledswpa aglgpgharh
vlrslvnqsv qdgeeqvrrl gplacflspe elqslvplsd pmgpvergll ecaangtlsp
qgrvayellg vlrssggavl splelrvwap lfpqlglrfl qelsepqlra mlpalqgtsv
tpaqavlllg rllprhdlsl eelcslhpll pglssqtlqa iprrvligac sclapelsrl
sacqtaallq tfrvkdgvkn igttgasaav cipgqqpipt twpdcllpll plkllqldsa
allanrrryr dlpwseqqaq flwkkmqvpt nltlrnlqal gtlaggmsce flqqinlmad
flevvhmiyq lptgvrgslr aciwaelqrk mtmpepelat lgselsgldt kllldspiyl
mdrlsnesim Imvelvrrap eqllaltplh rvalaeralq nlapkettvs revletlgpl
vgflgiestr riplpillah Inqlqgfclg epfatelgwl lsqepilgkp elwsegeveq
agrlvltlst eaisliprea lgpetlerll ekqqsweqsr vgqlcgtpql apkkaalvag
vvrptaedls epvpncadvr gtfpaawsat qiaemelsdf edclalfagd pglgpeelra
amgkakqlwg pprgfrpeqi lqlsrlligl gerelqelil vdwgvlstlg qidgwssiql
rvvvssflrq sgrhvshldf lhltalgytl cglrpeelqh isswefsqaa lflgnlhlqc
seeqlevlaq llvlpggfgp vsnwgpeift eigtiaagip dlalsallqe qiqgltplai
svipapkfav vfsptqlssl tsvqamavtp eqmaflspeq rravvwaqhe gkespeqqgr
stawglqdws qpswamalti cflgnli

### Vectors

The compositions provided herein include at least two (e.g., two, three, four, five, or six) nucleic acid vectors, where: each of the at least two different vectors includes a coding sequence that encodes a different portion of a stereocilin protein, each of the encoded portions being at least 30 amino acids (e.g., between about 30 amino acids to about 1600 amino acids, about 30 amino acids to about 1550 amino acids, about 30 amino acids to about 1500 amino acids, about 30 amino acids to about 1450 amino acids, about 30 amino acids to about 1400 amino acids, about 30 amino acids to about 1350 amino acids, about 30 amino acids to about 1300 amino acids, about 30 amino acids to about 1250 amino acids, about 30 amino acids to about 1200 amino acids, about 30 amino acids to about 1150 amino acids, about 30 amino acids to about 1100 amino acids, about 30 amino acids to about 1050 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 250 amino acids, about 30 amino acids to about 200 amino acids, about 30 amino acids to about 150 amino acids, about 30 amino acids to about 100 amino acids, about 30 amino acids to about 50 amino acids, about 50 amino acids to about 1600 amino acids, about 50 amino acids to about 1550 amino acids, about 50 amino acids to about 1500 amino acids, about 50 amino acids to about 1450 amino acids, about 50 amino acids to about 1400 amino acids, about 50 amino acids to about 1350 amino acids, about 50 amino acids to about 1300 amino acids, about 50 amino acids to about 1250 amino acids, about 50 amino acids to about 1200 amino acids, about 50 amino acids to about 1150 amino acids, about 50 amino acids to about 1100 amino acids, about 50 amino acids to about 1050 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 1600 amino acids, about 100 amino acids to about 1550 amino acids, about 100 amino acids to about 1500 amino acids, about 100 amino acids to about 1450 amino acids, about 100 amino acids to about 1400 amino acids, about 100 amino acids to about 1350 amino acids, about 100 amino acids to about 1300 amino acids, about 100 amino acids to about 1250 amino acids, about 100 amino acids to about 1200 amino acids, about 100 amino acids to about 1150 amino acids, about 100 amino acids to about 1100 amino acids, about 100 amino acids to about 1050 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 1600 amino acids, about 150 amino acids to about 1550 amino acids, about 150 amino acids to about 1500 amino acids, about 150 amino acids to about 1450 amino acids, about 150 amino acids to about 1400 amino acids, about 150 amino acids to about 1350 amino acids, about 150 amino acids to about 1300 amino acids, about 150 amino acids to about 1250 amino acids, about 150 amino acids to about 1200 amino acids, about 150 amino acids to about 1150 amino acids, about 150 amino acids to about 1100 amino acids, about 150 amino acids to about 1050 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 1600 amino acids, about 200 amino acids to about 1550 amino acids, about 200 amino acids to about 1500 amino acids, about 200 amino acids to about 1450 amino acids, about 200 amino acids to about 1400 amino acids, about 200 amino acids to about 1350 amino acids, about 200 amino acids to about 1300 amino acids, about 200 amino acids to about 1250 amino acids, about 200 amino acids to about 1200 amino acids, about 200 amino acids to about 1150 amino acids, about 200 amino acids to about 1100 amino acids, about 200 amino acids to about 1050 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 1600 amino acids, about 250 amino acids to about 1550 amino acids, about 250 amino acids to about 1500 amino acids, about 250 amino acids to about 1450 amino acids, about 250 amino acids to about 1400 amino acids, about 250 amino acids to about 1350 amino acids, about 250 amino acids to about 1300 amino acids, about 250 amino acids to about 1250 amino acids, about 250 amino acids to about 1200 amino acids, about 250 amino acids to about 1150 amino acids, about 250 amino acids to about 1100 amino acids, about 250 amino acids to about 1050 amino acids, about 250 amino acids to about 1000 amino acids, about 250 amino acids to about 950 amino acids, about 250 amino acids to about 900 amino acids, about 250 amino acids to about 850 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 1600 amino acids, about 300 amino acids to about 1550 amino acids, about 300 amino acids to about 1500 amino acids, about 300 amino acids to about 1450 amino acids, about 300 amino acids to about 1400 amino acids, about 300 amino acids to about 1350 amino acids, about 300 amino acids to about 1300 amino acids, about 300 amino acids to about 1250 amino acids, about 300 amino acids to about 1200 amino acids, about 300 amino acids to about 1150 amino acids, about 300 amino acids to about 1100 amino acids, about 300 amino acids to about 1050 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1600 amino acids, about 350 amino acids to about 1550 amino acids, about 350 amino acids to about 1500 amino acids, about 350 amino acids to about 1450 amino acids, about 350 amino acids to about 1400 amino acids, about 350 amino acids to about 1350 amino acids, about 350 amino acids to about 1300 amino acids, about 350 amino acids to about 1250 amino acids, about 350 amino acids to about 1200 amino acids, about 350 amino acids to about 1150 amino acids, about 350 amino acids to about 1100 amino acids, about 350 amino acids to about 1050 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1600 amino acids, about 400 amino acids to about 1550 amino acids, about 400 amino acids to about 1500 amino acids, about 400 amino acids to about 1450 amino acids, about 400 amino acids to about 1400 amino acids, about 400 amino acids to about 1350 amino acids, about 400 amino acids to about 1300 amino acids, about 400 amino acids to about 1250 amino acids, about 400 amino acids to about 1200 amino acids, about 400 amino acids to about 1150 amino acids, about 400 amino acids to about 1100 amino acids, about 400 amino acids to about 1050 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1600 amino acids, about 450 amino acids to about 1550 amino acids, about 450 amino acids to about 1500 amino acids, about 450 amino acids to about 1450 amino acids, about 450 amino acids to about 1400 amino acids, about 450 amino acids to about 1350 amino acids, about 450 amino acids to about 1300 amino acids, about 450 amino acids to about 1250 amino acids, about 450 amino acids to about 1200 amino acids, about 450 amino acids to about 1150 amino acids, about 450 amino acids to about 1100 amino acids, about 450 amino acids to about 1050 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1600 amino acids, about 500 amino acids to about 1550 amino acids, about 500 amino acids to about 1500 amino acids, about 500 amino acids to about 1450 amino acids, about 500 amino acids to about 1400 amino acids, about 500 amino acids to about 1350 amino acids, about 500 amino acids to about 1300 amino acids, about 500 amino acids to about 1250 amino acids, about 500 amino acids to about 1200 amino acids, about 500 amino acids to about 1150 amino acids, about 500 amino acids to about 1100 amino acids, about 500 amino acids to about 1050 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1600 amino acids, about 550 amino acids to about 1550 amino acids, about 550 amino acids to about 1500 amino acids, about 550 amino acids to about 1450 amino acids, about 550 amino acids to about 1400 amino acids, about 550 amino acids to about 1350 amino acids, about 550 amino acids to about 1300 amino acids, about 550 amino acids to about 1250 amino acids, about 550 amino acids to about 1200 amino acids, about 550 amino acids to about 1150 amino acids, about 550 amino acids to about 1100 amino acids, about 550 amino acids to about 1050 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1600 amino acids, about 600 amino acids to about 1550 amino acids, about 600 amino acids to about 1500 amino acids, about 600 amino acids to about 1450 amino acids, about 600 amino acids to about 1400 amino acids, about 600 amino acids to about 1350 amino acids, about 600 amino acids to about 1300 amino acids, about 600 amino acids to about 1250 amino acids, about 600 amino acids to about 1200 amino acids, about 600 amino acids to about 1150 amino acids, about 600 amino acids to about 1100 amino acids, about 600 amino acids to about 1050 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1600 amino acids, about 650 amino acids to about 1550 amino acids, about 650 amino acids to about 1500 amino acids, about 650 amino acids to about 1450 amino acids, about 650 amino acids to about 1400 amino acids, about 650 amino acids to about 1350 amino acids, about 650 amino acids to about 1300 amino acids, about 650 amino acids to about 1250 amino acids, about 650 amino acids to about 1200 amino acids, about 650 amino acids to about 1150 amino acids, about 650 amino acids to about 1100 amino acids, about 650 amino acids to about 1050 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1600 amino acids, about 700 amino acids to about 1550 amino acids, about 700 amino acids to about 1500 amino acids, about 700 amino acids to about 1450 amino acids, about 700 amino acids to about 1400 amino acids, about 700 amino acids to about 1350 amino acids, about 700 amino acids to about 1300 amino acids, about 700 amino acids to about 1250 amino acids, about 700 amino acids to about 1200 amino acids, about 700 amino acids to about 1150 amino acids, about 700 amino acids to about 1100 amino acids, about 700 amino acids to about 1050 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1600 amino acids, about 750 amino acids to about 1550 amino acids, about 750 amino acids to about 1500 amino acids, about 750 amino acids to about 1450 amino acids, about 750 amino acids to about 1400 amino acids, about 750 amino acids to about 1350 amino acids, about 750 amino acids to about 1250 amino acids, about 750 amino acids to about 1200 amino acids, about 750 amino acids to about 1150 amino acids, about 750 amino acids to about 1100 amino acids, about 750 amino acids to about 1050 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1600 amino acids, about 800 amino acids to about 1550 amino acids, about 800 amino acids to about 1500 amino acids, about 800 amino acids to about 1450 amino acids, about 800 amino acids to about 1400 amino acids, about 800 amino acids to about 1350 amino acids, about 800 amino acids to about 1300 amino acids, about 800 amino acids to about 1250 amino acids, about 800 amino acids to about 1200 amino acids, about 800 amino acids to about 1150 amino acids, about 800 amino acids to about 1100 amino acids, about 800 amino acids to about 1050 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1600 amino acids, about 850 amino acids to about 1550 amino acids, about 850 amino acids to about 1500 amino acids, about 850 amino acids to about 1450 amino acids, about 850 amino acids to about 1400 amino acids, about 850 amino acids to about 1350 amino acids, about 850 amino acids to about 1300 amino acids, about 850 amino acids to about 1250 amino acids, about 850 amino acids to about 1200 amino acids, about 850 amino acids to about 1150 amino acids, about 850 amino acids to about 1100 amino acids, about 850 amino acids to about 1050 amino acids, about 850 amino acids to about 1000 amino acids, about 850 amino acids to about 950 amino acids, about 850 amino acids to about 900 amino acids, about 900 amino acids to about 1600 amino acids, about 900 amino acids to about 1550 amino acids, about 900 amino acids to about 1500 amino acids, about 900 amino acids to about 1450 amino acids, about 900 amino acids to about 1400 amino acids, about 900 amino acids to about 1350 amino acids, about 900 amino acids to about 1300 amino acids, about 900 amino acids to about 1250 amino acids, about 900 amino acids to about 1200 amino acids, about 900 amino acids to about 1150 amino acids, about 900 amino acids to about 1100 amino acids, about 900 amino acids to about 1050 amino acids, about 900 amino acids to about 1000 amino acids, about 900 amino acids to about 950 amino acids, about 950 amino acids to about 1600 amino acids, about 950 amino acids to about 1550 amino acids, about 950 amino acids to about 1500 amino acids, about 950 amino acids to about 1450 amino acids, about 950 amino acids to about 1400 amino acids, about 950 amino acids to about 1350 amino acids, about 950 amino acids to about 1300 amino acids, about 950 amino acids to about 1250 amino acids, about 950 amino acids to about 1200 amino acids, about 950 amino acids to about 1150 amino acids, about 950 amino acids to about 1100 amino acids, about 950 amino acids to about 1050 amino acids, about 950 amino acids to about 1000 amino acids, about 1000 amino acids to about 1600 amino acids, about 1000 amino acids to about 1550 amino acids, about 1000 amino acids to about 1500 amino acids, about 1000 amino acids to about 1450 amino acids, about 1000 amino acids to about 1400 amino acids, about 1000 amino acids to about 1350 amino acids, about 1000 amino acids to about 1300 amino acids, about 1000 amino acids to about 1250 amino acids, about 1000 amino acids to about 1200 amino acids, about 1000 amino acids to about 1150 amino acids, about 1000 amino acids to about 1100 amino acids, about 1000 amino acids to about 1050 amino acids, about 1050 amino acids to about 1600 amino acids, about 1050 amino acids to about 1550 amino acids, about 1050 amino acids to about 1500 amino acids, about 1050 amino acids to about 1450 amino acids, about 1050 amino acids to about 1400 amino acids, about 1050 amino acids to about 1350 amino acids, about 1050 amino acids to about 1300 amino acids, about 1050 amino acids to about 1250 amino acids, about 1050 amino acids to about 1200 amino acids, about 1050 amino acids to about 1150 amino acids, about 1050 amino acids to about 1100 amino acids, about 1100 amino acids to about 1600 amino acids, about 1100 amino acids to about 1550 amino acids, about 1100 amino acids to about 1500 amino acids, about 1100 amino acids to about 1450 amino acids, about 1100 amino acids to about 1400 amino acids, about 1100 amino acids to about 1350 amino acids, about 1100 amino acids to about 1300 amino acids, about 1100 amino acids to about 1250 amino acids, about 1100 amino acids to about 1200 amino acids, about 1100 amino acids to about 1150 amino acids, about 1150 amino acids to about 1600 amino acids, about 1150 amino acids to about 1550 amino acids, about 1150 amino acids to about 1500 amino acids, about 1150 amino acids to about 1450 amino acids, about 1150 amino acids to about 1400 amino acids, about 1150 amino acids to about 1350 amino acids, about 1150 amino acids to about 1300 amino acids, about 1150 amino acids to about 1250 amino acids, about 1150 amino acids to about 1200 amino acids, about 1200 amino acids to about 1600 amino acids, about 1200 amino acids to about 1550 amino acids, about 1200 amino acids to about 1500 amino acids, about 1200 amino acids to about 1450 amino acids, about 1200 amino acids to about 1400 amino acids, about 1200 amino acids to about 1350 amino acids, about 1200 amino acids to about 1300 amino acids, about 1200 amino acids to about 1250 amino acids, about 1250 amino acids to about 1600 amino acids, about 1250 amino acids to about 1550 amino acids, about 1250 amino acids to about 1500 amino acids, about 1250 amino acids to about 1450 amino acids, about 1250 amino acids to about 1400 amino acids, about 1250 amino acids to about 1350 amino acids, about 1250 amino acids to about 1300 amino acids, about 1300 amino acids to about 1600 amino acids, about 1300 amino acids to about 1550 amino acids, about 1300 amino acids to about 1500 amino acids, about 1300 amino acids to about 1450 amino acids, about 1300 amino acids to about 1400 amino acids, about 1300 amino acids to about 1350 amino acids, about 1350 amino acids to about 1600 amino acids, about 1350 amino acids to about 1550 amino acids, about 1350 amino acids to about 1500 amino acids, about 1350 amino acids to about 1450 amino acids, about 1350 amino acids to about 1400 amino acids, about 1400 amino acids to about 1600 amino acids, about 1400 amino acids to about 1550 amino acids, about 1400 amino acids to about 1500 amino acids, about 1400 amino acids to about 1450 amino acids, about 1450 amino acids to about 1600 amino acids, about 1450 amino acids to about 1550 amino acids, about 1450 amino acids to about 1500 amino acids, about 1500 amino acids to about 1600 amino acids, about 1500 amino acids to about 1550 amino acids, or about 1550 amino acids to about 1600 amino acids), wherein the amino acid sequence of each of the encoded portions may optionally partially overlap with the amino acid sequence of a different one of the encoded portions; no single vector of the at least two different vectors encodes an active stereocilin protein (e.g., a full-length stereocilin protein (e.g., a full-length wildtype stereocilin protein)); and, when introduced into a mammalian cell that contains chromosomal DNA, the at least two different vectors undergo homologous recombination with each other and with the chromosomal DNA of the cell, thereby forming a recombined nucleic acid inserted into the chromosomal DNA, where the recombined nucleic acid encodes an active stereocilin protein (e.g., a full-length stereocilin protein). In some embodiments, one of the nucleic acid vectors can include a coding sequence that encodes a portion of a stereocilin protein, where the encoded portion is, e.g., about 900 amino acids to about 1600 amino acids, about 900 amino acids to about 1550 amino acids, about 900 amino acids to about 1500 amino acids, about 900 amino acids to about 1450 amino acids, about 900 amino acids to about 1400 amino acids, about 900 amino acids to about 1350 amino acids, about 900 amino acids to about 1300 amino acids, about 900 amino acids to about 1250 amino acids, about 900 amino acids to about 1200 amino acids, about 900 amino acids to about 1150 amino acids, about 900 amino acids to about 1100 amino acids, about 900 amino acids to about 1050 amino acids, about 900 amino acids to about 1000 amino acids, about 900 amino acids to about 950 amino acids, about 950 amino acids to about 1600 amino acids, about 950 amino acids to about 1550 amino acids, about 950 amino acids to about 1500 amino acids, about 950 amino acids to about 1450 amino acids, about 950 amino acids to about 1400 amino acids, about 950 amino acids to about 1350 amino acids, about 950 amino acids to about 1300 amino acids, about 950 amino acids to about 1250 amino acids, about 950 amino acids to about 1200 amino acids, about 950 amino acids to about 1150 amino acids, about 950 amino acids to about 1100 amino acids, about 950 amino acids to about 1050 amino acids, about 950 amino acids to about 1000 amino acids, about 1000 amino acids to about 1600 amino acids, about 1000 amino acids to about 1550 amino acids, about 1000 amino acids to about 1500 amino acids, about 1000 amino acids to about 1450 amino acids, about 1000 amino acids to about 1400 amino acids, about 1000 amino acids to about 1350 amino acids, about 1000 amino acids to about 1300 amino acids, about 1000 amino acids to about 1250 amino acids, about 1000 amino acids to about 1200 amino acids, about 1000 amino acids to about 1150 amino acids, about 1000 amino acids to about 1100 amino acids, about 1000 amino acids to about 1050 amino acids, about 1050 amino acids to about 1600 amino acids, about 1050 amino acids to about 1550 amino acids, about 1050 amino acids to about 1500 amino acids, about 1050 amino acids to about 1450 amino acids, about 1050 amino acids to about 1400 amino acids, about 1050 amino acids to about 1350 amino acids, about 1050 amino acids to about 1300 amino acids, about 1050 amino acids to about 1250 amino acids, about 1050 amino acids to about 1200 amino acids, about 1050 amino acids to about 1150 amino acids, about 1050 amino acids to about 1100 amino acids, about 1100 amino acids to about 1600 amino acids, about 1100 amino acids to about 1550 amino acids, about 1100 amino acids to about 1500 amino acids, about 1100 amino acids to about 1450 amino acids, about 1100 amino acids to about 1400 amino acids, about 1100 amino acids to about 1350 amino acids, about 1100 amino acids to about 1300 amino acids, about 1100 amino acids to about 1250 amino acids, about 1100 amino acids to about 1200 amino acids, about 1100 amino acids to about 1150 amino acids, about 1150 amino acids to about 1600 amino acids, about 1150 amino acids to about 1550 amino acids, about 1150 amino acids to about 1500 amino acids, about 1150 amino acids to about 1450 amino acids, about 1150 amino acids to about 1400 amino acids, about 1150 amino acids to about 1350 amino acids, about 1150 amino acids to about 1300 amino acids, about 1150 amino acids to about 1250 amino acids, about 1150 amino acids to about 1200 amino acids, about 1200 amino acids to about 1600 amino acids, about 1200 amino acids to about 1550 amino acids, about 1200 amino acids to about 1500 amino acids, about 1200 amino acids to about 1450 amino acids, about 1200 amino acids to about 1400 amino acids, about 1200 amino acids to about 1350 amino acids, about 1200 amino acids to about 1300 amino acids, about 1200 amino acids to about 1250 amino acids, about 1250 amino acids to about 1600 amino acids, about 1250 amino acids to about 1550 amino acids, about 1250 amino acids to about 1500 amino acids, about 1250 amino acids to about 1450 amino acids, about 1250 amino acids to about 1400 amino acids, about 1250 amino acids to about 1350 amino acids, about 1250 amino acids to about 1300 amino acids, about 1300 amino acids to about 1600 amino acids, about 1300 amino acids to about 1550 amino acids, about 1300 amino acids to about 1500 amino acids, about 1300 amino acids to about 1450 amino acids, about 1300 amino acids to about 1400 amino acids, about 1300 amino acids to about 1350 amino acids, about 1350 amino acids to about 1600 amino acids, about 1350 amino acids to about 1550 amino acids, about 1350 amino acids to about 1500 amino acids, about 1350 amino acids to about 1450 amino acids, about 1350 amino acids to about 1400 amino acids, about 1400 amino acids to about 1600 amino acids, about 1400 amino acids to about 1550 amino acids, about 1400 amino acids to about 1500 amino acids, about 1400 amino acids to about 1450 amino acids, about 1450 amino acids to about 1600 amino acids, about 1450 amino acids to about 1550 amino acids, about 1450 amino acids to about 1500 amino acids, about 1500 amino acids to about 1600 amino acids, about 1500 amino acids to about 1550 amino acids, or about 1550 amino acids to about 1600 amino acids in length.

In some embodiments of these compositions, at least one of the coding sequences includes a nucleotide sequence spanning two neighboring exons of stereocilin genomic DNA, and lacks the intronic sequence that naturally occurs between the two neighboring exons.

In some embodiments, the amino acid sequence of none of the encoded portions overlaps even in part with the amino acid sequence of a different one of the encoded portions. In some embodiments, the amino acid sequence of one or more of the encoded portions partially overlaps with the amino acid sequence of a different one of the encoded portions. In some embodiments, the amino acid sequence of each of the encoded portions partially overlaps with the amino acid sequence of a different one of the encoded portions.

In some embodiments, the overlapping amino acid sequence is between about 30 amino acid residues to about 1000 amino acids (e.g., or any of the subranges of this range described herein) in length.

In some examples, the vectors include two different vectors, each of which comprises a different segment of an intron, wherein the intron includes the nucleotide sequence of an intron that is present in a stereocilin genomic DNA (e.g., any of the exemplary introns in SEQ ID NO: 4 described herein), and wherein the two different segments overlap in sequence by at least 100 nucleotides (e.g., about 100 nucleotides to about 5,000 nucleotides, about 100 nucleotides to about 4,500 nucleotides, about 100 nucleotides to about 4,000 nucleotides, about 100 nucleotides to about 3,500 nucleotides, about 100 nucleotides to about 3,000 nucleotides, about 100 nucleotides to about 2,500 nucleotides, about 100 nucleotides to about 2,000 nucleotides, about 100 nucleotides to about 1,500 nucleotides, about 100 nucleotides to about 1,000 nucleotides, about 100 nucleotides to about 800 nucleotides, about 100 nucleotides to about 600 nucleotides, about 100 nucleotides to about 400 nucleotides, about 100 nucleotides to about 200 nucleotides, about 200 nucleotides to about 5,000 nucleotides, about 200 nucleotides to about 4,500 nucleotides, about 200 nucleotides to about 4,000 nucleotides, about 200 nucleotides to about 3,500 nucleotides, about 200 nucleotides to about 3,000 nucleotides, about 200 nucleotides to about 2,500 nucleotides, about 200 nucleotides to about 2,000 nucleotides, about 200 nucleotides to about 1,500 nucleotides, about 200 nucleotides to about 1,000 nucleotides, about 200 nucleotides to about 800 nucleotides, about 200 nucleotides to about 600 nucleotides, about 200 nucleotides to about 400 nucleotides, about 400 nucleotides to about 5,000 nucleotides, about 400 nucleotides to about 4,500 nucleotides, about 400 nucleotides to about 4,000 nucleotides, about 400 nucleotides to about 3,500 nucleotides, about 400 nucleotides to about 3,000 nucleotides, about 400 nucleotides to about 2,500 nucleotides, about 400 nucleotides to about 2,000 nucleotides, about 400 nucleotides to about 1,500 nucleotides, about 400 nucleotides to about 1,000 nucleotides, about 400 nucleotides to about 800 nucleotides, about 400 nucleotides to about 600 nucleotides, about 600 nucleotides to about 5,000 nucleotides, about 600 nucleotides to about 4,500 nucleotides, about 600 nucleotides to about 4,000 nucleotides, about 600 nucleotides to about 3,500 nucleotides, about 600 nucleotides to about 3,000 nucleotides, about 600 nucleotides to about 2,500 nucleotides, about 600 nucleotides to about 2,000 nucleotides, about 600 nucleotides to about 1,500 nucleotides, about 600 nucleotides to about 1,000 nucleotides, about 600 nucleotides to about 800 nucleotides, about 800 nucleotides to about 5,000 nucleotides, about 800 nucleotides to about 4,500 nucleotides, about 800 nucleotides to about 4,000 nucleotides, about 800 nucleotides to about 3,500 nucleotides, about 800 nucleotides to about 3,000 nucleotides, about 800 nucleotides to about 2,500 nucleotides, about 800 nucleotides to about 2,000 nucleotides, about 800 nucleotides to about 1,500 nucleotides, about 800 nucleotides to about 1,000 nucleotides, about 1,000 nucleotides to about 5,000 nucleotides, about 1,000 nucleotides to about 4,500 nucleotides, about 1,000 nucleotides to about 4,000 nucleotides, about 1,000 nucleotides to about 3,500 nucleotides, about 1,000 nucleotides to about 3,000 nucleotides, about 1,000 nucleotides to about 2,500 nucleotides, about 1,000 nucleotides to about 2,000 nucleotides, about 1,000 nucleotides to about 1,500 nucleotides, about 1,500 nucleotides to about 5,000 nucleotides, about 1,500 nucleotides to about 4,500 nucleotides, about 1,500 nucleotides to about 4,000 nucleotides, about 1,500 nucleotides to about 3,500 nucleotides, about 1,500 nucleotides to about 3,000 nucleotides, about 1,500 nucleotides to about 2,500 nucleotides, about 1,500 nucleotides to about 2,000 nucleotides, about 2,000 nucleotides to about 5,000 nucleotides, about 2,000 nucleotides to about 4,500 nucleotides, about 2,000 nucleotides to about 4,000 nucleotides, about 2,000 nucleotides to about 3,500 nucleotides, about 2,000 nucleotides to about 3,000 nucleotides, about 2,000 nucleotides to about 2,500 nucleotides, about 2,500 nucleotides to about 5,000 nucleotides, about 2,500 nucleotides to about 4,500 nucleotides, about 2,500 nucleotides to about 4,000 nucleotides, about 2,500 nucleotides to about 3,500 nucleotides, about 2,500 nucleotides to about 3,000 nucleotides, about 3,000 nucleotides to about 5,000 nucleotides, about 3,000 nucleotides to about 4,500 nucleotides, about 3,000 nucleotides to about 4,000 nucleotides, about 3,000 nucleotides to about 3,500 nucleotides, about 3,500 nucleotides to about 5,000 nucleotides, about 3,500 nucleotides to about 4,500 nucleotides, about 3,500 nucleotides to about 4,000 nucleotides, about 4,000 nucleotides to about 5,000 nucleotides, about 4,000 nucleotides to about 4,500 nucleotides, about 4,500 nucleotides to about 5,000 nucleotides), in length.

The overlapping nucleotide sequence in any two of the different vectors can include part or all of one or more exons of a stereocilin gene (e.g., any one or more of the exemplary exons in SEQ ID NO: 4 described herein).

In some embodiments, the number of different vectors in the composition is two, three, four, or five. In compositions where the number of different vectors in the composition is two, the first of the two different vectors can include a coding sequence that encodes an N-terminal portion of the stereocilin protein. In some examples, the N-terminal portion of the stereocilin gene is between about 30 amino acids to about 1780 amino acids (or any of the subranges of this range described above) in length. In some examples, the first vector further includes one or both of a promoter (e.g., any of the promoters described herein or known in the art) and a Kozak sequence (e.g., any of the exemplary Kozak sequences described herein or known in the art). In some examples, the first vector includes a promoter that is an inducible promoter, a constitutive promoter, or a tissue-specific promoter. In some examples, the second of the two different vectors includes a coding sequence that encodes a C-terminal portion of the stereocilin protein. In some examples, the C-terminal portion of the stereocilin protein is between 30 amino acids to about 1780 amino acids (or any of the subranges of this range described above) in length. In some examples, the second vector further includes a poly(A) sequence.

In some examples where the number of different vectors in the composition is two, the N-terminal portion encoded by one of the two vectors can include a portion comprising amino acid position 1 to about amino acid position 1,500, about amino acid position 1,490, about amino acid position 1,480, about amino acid position 1,470, about amino acid position 1,460, about amino acid position 1,450, about amino acid position 1,440, about amino acid position 1,430, about amino acid position 1,420, about amino acid position 1,410, about amino acid position 1,400, about amino acid position 1,390, about amino acid position 1,380, about amino acid position 1,370, about amino acid position 1,360, about amino acid position 1,350, about amino acid position 1,340, about amino acid position 1,330, about amino acid position 1,320, about amino acid position 1,310, about amino acid position 1,300, about amino acid position 1,290, about amino acid position 1,280, about amino acid position 1,270, about amino acid position 1,260, about amino acid position 1,250, about amino acid position 1,240, about amino acid position 1,230, about amino acid position 1,220, about amino acid position 1,210, about amino acid position 1,200, about amino acid position 1,190, about amino acid position 1,180, about amino acid position 1,170, about amino acid position 1,160, about amino acid position 1,150, about amino acid position 1,140, about amino acid position 1,130, about amino acid position 1,120, about amino acid position 1,110, about amino acid position 1,100, about amino acid position 1,090, about amino acid position 1,080, about amino acid position 1,070, about amino acid position 1,060, about amino acid position 1,050, about amino acid position 1,040, about amino acid position 1,030, about amino acid position 1,020, about amino acid position 1,010, about amino acid position 1,000, about amino acid position 990, about amino acid position 980, about amino acid position 970, about amino acid position 960, about amino acid position 950, about amino acid position 940, about amino acid position 930, about amino acid position 920, about amino acid position 910, about amino acid position 900, about amino acid position 890, about amino acid position 880, about amino acid position 870, about amino acid position 860, about amino acid position 850, about amino acid position 840, about amino acid position 830, about amino acid position 820, about amino acid position 810, about amino acid position 800, about amino acid position 790, about amino acid position 780, about amino acid position 770, about amino acid position 760, about amino acid position 750, about amino acid position 740, about amino acid position 730, about amino acid position 720, about amino acid position 710, about amino acid position 700, about amino acid position 690, about amino acid position 680, about amino acid position 670, about amino acid position 660, about amino acid position 650, about amino acid position 640, about amino acid position 630, about amino acid position 620, about amino acid position 610, about amino acid position 600, about amino acid position 590, about amino acid position 580, about amino acid position 570, about amino acid position 560, about amino acid position 550, about amino acid position 540, about amino acid position 530, about amino acid position 520, about amino acid position 510, about amino acid position 500, about amino acid position 490, about amino acid position 480, about amino acid position 470, about amino acid position 460, about amino acid position 450, about amino acid position 440, about amino acid position 430, about amino acid position 420, about amino acid position 410, about amino acid position 400, about amino acid position 390, about amino acid position 380, about amino acid position 370, about amino acid position 360, about amino acid position 350, about amino acid position 340, about amino acid position 330, about amino acid position 320, about amino acid position 310, about amino acid position 300, about amino acid position 290, about amino acid position 280, about amino acid position 270, about amino acid position 260, about amino acid position 250, about amino acid position 240, about amino acid position 230, about amino acid position 220, about amino acid position 210, about amino acid position 200, about amino acid position 190, about amino acid position 180, about amino acid position 170, about amino acid position 160, about amino acid position 150, about amino acid position 140, about amino acid position 130, about amino acid position 120, about amino acid position 110, about amino acid position 100, about amino acid position 90, about amino acid position 80, about amino acid position 70, about amino acid position 60, about amino acid position 50, or about amino acid position 40 of a wildtype stereocilin protein (e.g., SEQ ID NO: 1).

In some examples where the number of different vectors in the composition is two, the N-terminal portion of the precursor stereocilin protein can include a portion comprising amino acid position 1 to amino acid position 310, amino acid position 1 to about amino acid position 320, amino acid position 1 to about amino acid position 330, amino acid position 1 to about amino acid position 340, amino acid position 1 to about amino acid position 350, amino acid position 1 to about amino acid position 360, amino acid position 1 to about amino acid position 370, amino acid position 1 to about amino acid position 380, amino acid position 1 to about amino acid position 390, amino acid position 1 to about amino acid position 400, amino acid position 1 to about amino acid position 410, amino acid position 1 to about amino acid position 420, amino acid position 1 to about amino acid position 430, amino acid position 1 to about amino acid position 440, amino acid position 1 to about amino acid position 450, amino acid position 1 to about amino acid position 460, amino acid position 1 to about amino acid position 470, amino acid position 1 to about amino acid position 480, amino acid position 1 to about amino acid position 490, amino acid position 1 to about amino acid position 500, amino acid position 1 to about amino acid position 510, amino acid position 1 to about amino acid position 520, amino acid position 1 to about amino acid position 530, amino acid position 1 to about amino acid position 540, amino acid position 1 to about amino acid position 550, amino acid position 1 to about amino acid position 560, amino acid position 1 to about amino acid position 570, amino acid position 1 to about amino acid position 580, amino acid position 1 to about amino acid position 590, amino acid position 1 to about amino acid position 600, amino acid position 1 to about amino acid position 610, amino acid position 1 to about amino acid position 620, amino acid position 1 to about amino acid position 630, amino acid position 1 to about amino acid position 640, amino acid position 1 to about amino acid position 650, amino acid position 1 to about amino acid position 660, amino acid position 1 to about amino acid position 670, amino acid position 1 to about amino acid position 680, amino acid position 1 to about amino acid position 690, amino acid position 1 to about amino acid position 700, amino acid position 1 to about amino acid position 710, amino acid position 1 to about amino acid position 720, amino acid position 1 to about amino acid position 730, amino acid position 1 to about amino acid position 740, amino acid position 1 to about amino acid position 750, amino acid position 1 to about amino acid position 760, amino acid position 1 to about amino acid position 770, amino acid position 1 to about amino acid position 780, amino acid position 1 to about amino acid position 790, amino acid position 1 to about amino acid position 800, amino acid position 1 to about amino acid position 810, amino acid position 1 to about amino acid position 820, amino acid position 1 to about amino acid position 830, amino acid position 1 to about amino acid position 840, amino acid position 1 to about amino acid position 850, amino acid position 1 to about amino acid position 860, amino acid position 1 to about amino acid position 870, amino acid position 1 to about amino acid position 880, amino acid position 1 to about amino acid position 890, amino acid position 1 to about amino acid position 900, amino acid position 1 to about amino acid position 910, amino acid position 1 to about amino acid position 920, amino acid position 1 to about amino acid position 930, amino acid position 1 to about amino acid position 940, amino acid position 1 to about amino acid position 950, amino acid position 1 to about amino acid position 960, amino acid position 1 to about amino acid position 970, amino acid position 1 to about amino acid position 980, amino acid position 1 to about amino acid position 990, amino acid position 1 to about amino acid position 1,000, amino acid position 1 to about amino acid position 1,010, amino acid position 1 to about amino acid position 1,020, amino acid position 1 to about amino acid position 1,030, amino acid position 1 to about amino acid position 1,040, amino acid position 1 to about amino acid position 1,050, amino acid position 1 to about amino acid position 1,060, amino acid position 1 to about amino acid position 1,070, amino acid position 1 to about amino acid position 1,080, amino acid position 1 to about amino acid position 1,090, amino acid position 1 to about amino acid position 1,100, amino acid position 1 to about amino acid position 1,110, amino acid position 1 to about amino acid position 1,120, amino acid position 1 to about amino acid position 1,130, amino acid position 1 to about amino acid position 1,140, amino acid position 1 to about amino acid position 1,150, amino acid position 1 to about amino acid position 1,160, amino acid position 1 to about amino acid position 1,170, amino acid position 1 to about amino acid position 1,180, amino acid position 1 to about amino acid position 1,190, amino acid position 1 to about amino acid position 1,200, amino acid position 1 to about amino acid position 1,210, amino acid position 1 to about amino acid position 1,220, amino acid position 1 to about amino acid position 1,230, amino acid position 1 to about amino acid position 1,240, amino acid position 1 to about amino acid position 1,250, amino acid position 1 to about amino acid position 1,260, amino acid position 1 to about amino acid position 1,270, amino acid position 1 to about amino acid position 1,280, amino acid position 1 to about amino acid position 1,290, amino acid position 1 to about amino acid position 1,300, amino acid position 1 to about amino acid position 1,310, amino acid position 1 to about amino acid position 1,320, amino acid position 1 to about amino acid position 1,330, amino acid position 1 to about amino acid position 1,340, amino acid position 1 to about amino acid position 1,350, amino acid position 1 to about amino acid position 1,360, amino acid position 1 to about amino acid position 1,370, amino acid position 1 to about amino acid position 1,380, amino acid position 1 to about amino acid position 1,390, amino acid position 1 to about amino acid position 1,400, amino acid position 1 to about amino acid position 1,410, amino acid position 1 to about amino acid position 1,420, amino acid position 1 to about amino acid position 1,430, amino acid position 1 to about amino acid position 1,440, amino acid position 1 to about amino acid position 1,450, amino acid position 1 to about amino acid position 1,460, amino acid position 1 to about amino acid position 1,470, amino acid position 1 to about amino acid position 1,480, amino acid position 1 to about amino acid position 1,490, amino acid position 1 to about amino acid position 1,500, amino acid position 1 to about amino acid position 1,510, amino acid position 1 to about amino acid position 1,520, amino acid position 1 to about amino acid position 1,530, amino acid position 1 to about amino acid position 1,540, amino acid position 1 to about amino acid position 1,550, amino acid position 1 to about amino acid position 1,560, amino acid position 1 to about amino acid position 1,570, amino acid position 1 to about amino acid position 1,580, amino acid position 1 to about amino acid position 1,590, or amino acid position 1 to about amino acid position 1,600 of a wildtype stereocilin protein (e.g., SEQ ID NO: 1).

As used herein, the term “vector” means a composition including a polynucleotide capable of carrying at least one exogenous nucleic acid fragment, e.g., a plasmid vector, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector (e.g., any adenoviral vectors (e.g., pSV or pCMV vectors), any retroviral vectors as described herein) and any Gateway® vectors. A vector can, e.g., include sufficient cis-acting elements for expression; other elements for expression can be supplied by the host mammalian cell or in an in vitro expression system. The term “vector” includes any genetic element (e.g., a plasmid, a transposon, a cosmid, an artificial chromosome, or a viral vector, etc.) that is capable of replicating when associated with the proper control elements. Thus, the term includes cloning and expression vectors, as well as viral vectors (e.g., an adeno-associated virus (AAV) vector, an adenovirus vector, a lentivirus vector, or a retrovirus vector).

Vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Skilled practitioners will be capable of selecting suitable vectors and mammalian cells for making any of the nucleic acids described herein. In some embodiments, the vector is a plasmid (i.e. a circular DNA molecule that can autonomously replicate inside a cell). In some embodiments, the vector can be a cosmid (e.g., pWE and sCos series (Wahl et al. (1987), Evans et al. (1989)).

In some embodiments, the vector(s) is an artificial chromosome. An artificial chromosome is a genetically engineered chromosome that can be used as a vector to carry large DNA inserts. In some embodiments, the artificial chromosome is human artificial chromosome (HAC) (see, e.g., Kouprina et al., Expert Opin. Drug Deliv 11(4): 517-535, 2014; Basu et al., Pediatr. Clin. North Am. 53: 843-853, 2006; Ren et al., Stem. Cell Rev. 2(1):43-50, 2006; Kazuki et al., Mol. Ther. 19(9):1591-1601, 2011; Kazuki et al., Gen. Ther. 18: 384-393, 2011; and Katoh et al., Biochem. Biophys. Res. Commun. 321:280-290, 2004).

In some embodiments, the vector(s) is a yeast artificial chromosome (YAC) (see, e.g., Murray et al., Nature 305: 189-193, 1983; Ikeno et al. (1998) Nat. Biotech. 16:431-439, 1998). In some embodiments, the vector(s) is a bacterial artificial chromosome (BAC) (e.g., pBeloBAC11, pECBAC1, and pBAC108L). In some embodiments, the vector(s) is a P1-derived artificial chromosome (PAC). Examples of artificial chromosome are known in the art.

In some embodiments, the vector(s) is a viral vector (e.g., adeno-associated virus, adenovirus, lentivirus, and retrovirus). Non-limiting examples of viral vectors are described herein. In some embodiments, the vector(s) is an adeno-associated viral vector (AAV) (see, e.g., Asokan et al., Mol. Ther. 20: 699-7080, 2012). “Recombinant AAV vectors” or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., an outer hair cell).

The AAV sequences of the vector typically comprise the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein are flanked by 5′ and 3′ AAV ITR sequences in the AAV vectors. The AAV ITR sequences may be obtained from any known AAV, including presently identified AAV types.

A non-limiting example of a 5′ AAV ITR sequence is SEQ ID NO: 18. A non-limiting example of a 3′ AAV ITR sequence is SEQ ID NO: 36.

AAV vectors as described herein may include any of the regulatory elements described herein (e.g., one or more of a promoter, a polyA sequence, and an IRES).

In some embodiments, the AAV vector is selected from the group consisting of: an AAV1 vector, an AAV2 vector, an AAV3 vector, an AAV4 vector, an AAV5 vector, an AAV6 vector, an AAV7 vector, an AAV8 vector, an AAV9 vector, an AAV2.7m8 vector, an AAV8BP2 vector, and an AAV293 vector. Additional exemplary AAV vectors that can be used herein are known in the art. See, e.g., Kanaan et al., Mol. Ther. Nucleic Acids 8:184-197, 2017; Li et al., Mol. Ther. 16(7): 1252-1260; Adachi et al., Nat. Commun. 5: 3075, 2014; Isgrig et al., Nat. Commun. 10(1): 427, 2019; and Gao et al., J. Virol. 78(12): 6381-6388.

In some embodiments, an AAV vector provided herein includes or consists of a sequence that is at least 80% identical (e.g., at least 82%, at least 84%, at least 85%, at least 86%, at least 88%, at least 90%, at least 92%, at least 94%, at least 95%, at least 96%, at least 98%, at least 99%, or 100% identical) to SEQ ID NOs: 12-17.

pITR-201
(SEQ ID NO: 12)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtagacttgcccaaaaactctggcaaagttgaattgcttccaaaagttcaactttatcagaagga
cctattccctacggaaactagctgaaggtctcctggcactctggatctcgtggaagggagccttcttca
gggaacagagggagcgattaagtggtgaaaagcaaacagacctggaaaagttccctttctgagagtagc
aacagaaagctctgcaaagactccctccaagctattggatcctcttgcttgggataaccacttgagtac
tcagataccaaaagaagagtggaaatcccaagagaagtcaccagaaaaaacagcttttaagaaaaagga
tacacttttgtccctgaacgcttgtgaaagcaatctgacaatagcagcaattgaaaagggacaaaataa
gcccgaaatagaagtcacctgggcaaagcaaggtaggactgaaaggctgtgctctcaaaacccaccagt
cttgaaacgcactcaacgggtgaaagtagtaggaaagggtgaatttacaaaggacgtaggactcaaaga
gtgagtttttccaagcagcagaaacctatttcttactaacttggataatttactgaaaaataatacaca
caatcaagaaaaaaaaattcaggaagaaatagaaaagaaggaaaacttaatccaagagtgaatagtttt
gcctcagataactacagtgactggcactaagaatttctgaaagaaccttttcttactgagcactaggct
gaaaatagaaggttacttgaacggggacttgactccagtacttcaagattttaggtactttgaaaattc
aacaaatagaacaaagaaacacacagctactttctcaaaaaaaggggaggaagaaaacttggaaggctt
gggaaatcaaaccaagcaaattgtagagaaattgactgacaccacaaggatatctcctaatacaagcca
gcagaattttgtcacgcaacgtagtaagagagctttgaaactagatcccatatatggagttccgcgtta
cataacttacggtaaatggcccgcctggctgaccgcccaacgacccccgcccattgacgtcaataatga
cgtatgttcccatagtaacgccaatagggactttccattgacgtcaatgggtggagtatttacggtaaa
ctgcccacttggcagtacatcaagtgtatcatatgccaagtacgccccctattgacgtcaatgacggta
aatggcccgcctggcattatgcccagtacatgaccttatgggactttcctacttggcagtacatctacg
tattagtcatcgctattaccatggtgatgcggttttggtgatgcggttttggcagtacatcaatgggcg
tggatagcggtttgactcacggggatttccaagtctccaccccattgacgtcaatgggagtttgttttg
gcaccaaaatcaacgggactttccaaaatgtcgtaacaactccgccccattgacgcaaatgggcggtag
gcgtgtacggtgggaggtctatataagcagagctcgtttagtgaaccgtcaccggtggtgccctgccct
cacctggctatcccacacaggtgagaataaccagaactcacctccggtaccagtgttcacttggccacc
atggctctcagcctctggcccctgctgctgctgctgctgctgctgctgctgctgtcctttgcagtgact
ctggcccctactgggcctcattccctggaccctggtctctccttcctgaagtcattgctctccactctg
gaccaggctccccagggctccctgagccgctcacggttctttacattcctggccaacatttcttcttcc
tttgagcctgggagaatgggggaaggaccagtaggagagcccccacctctccagccgcctgctctgcgg
ctccatgattttctagtgacactgagaggtagccccgactgggagccaatgctagggctgctaggggat
atgctggcactgctgggacaggagcagactccccgagatttcctggtgcaccaggcaggggtgctgggt
ggacttgtggaggtgctgctgggagccttagttcctgggggcccccctaccccaactcggcccccatgc
acccgtgatgggccgtctgactgtgtcctggctgctgactggttgccttctctgctgctgttgttagag
ggcacacgctggcaagctctggtgcaggtgcagcccagtgtggaccccaccaatgccacaggcctcgat
gggagggaggcagctcctcactttttgcagggtctgttgggtttgcttaccccaacaggggagctaggc
tccaaggaggctctttggggcggtctgctacgcacagtgggggcccccctctatgctgcctttcaggag
gggctgctccgtgtcactcactccttgcaggatgaggtcttctccattttggggcagccagagcctgat
accaatgggcagtgccagggaggtaaccttcaacagctgctcttatggggcgtccggcacaacctttcc
tgggatgtccaggcgctgggctttctgtctggatcaccacccccaccccctgccctccttcactgcctg
agcacgggcgtgcctctgcccagagcttctcagccgtcagcccacatcagcccacgccaacggcgagcc
atcactgtggaggccctctgtgagaaccacttaggcccagcaccaccctacagcatttccaacttctcc
atccacttgctctgccagcacaccaagcctgccactccacagccccatcccagcaccactgccatctgc
cagacagctgtgtggtatgcagtgtcctgggcaccaggtgcccaaggctggctacaggcctgccacgac
cagtttcctgatgagtttttggatgcgatctgcagtaacctctccttttcagccctgtctggctccaac
cgccgcctggtgaagcggctctgtgctggcctgctcccaccccctaccagctgccctgaaggcctgccc
cctgttcccctcaccccagacatcttttggggctgcttcttggagaatgagactctgtgggctgagcga
ctgtgtggggaggcaagtctacaggctgtgccccccagcaaccaggcttgggtccagcatgtgtgccag
ggccccaccccagatgtcactgcctccccaccatgccacattggaccctgtggggaacgctgcccggat
gggggcagcttcctggtgatggtctgtgccaatgacaccatgtatgaggtcctggtgcccttctggcct
tggctagcaggccaatgcaggataagtcgtgggggcaatgacacttgcttcctagaagggctgctgggc
ccccttctgccctctctgccaccactgggaccatccccactctgtctgacccctggccccttcctcctt
ggcatgctatcccagttgccacgctgtcagtcctctgtcccagctcttgctcaccccacacgcctacac
tatctcctccgcctgctgaccttcctcttgggtccaggggctgggggcgctgaggcccaggggatgctg
ggtcgggccctactgctctccagtctcccagacaactgctccttctgggatgcctttcgcccagagggc
cggcgcagtgtgctacggacgattggggaatacctggaacaagatgaggagcagccaaccccatcaggc
tttgaacccactgtcaaccccagctctggtataagcaagatggagctgctggcctgctttagtcctgtg
ctgtgggatctgctccagagggaaaagagtgtttgggccctgcagattctagtgcaggtaagtatcaag
gttacaagacaggtttaaggagaccaatagaaactgggcttgtcgagacagagaagactcttgcgtttc
tgggattttgccgatttcggcctattggttaaaaaatgagctgatttaacaaaaatttaacgcgaattt
taacaaaataagcttgaattcagctgacgtgcctcggaccgctaggaacccctagtgatggagttggcc
actccctctctgcgcgctcgctcgctcactgaggccgggcgaccaaaggtcgcccgacgcccgggcttt
gcccgggcggcctcagtgagcgagcgagcgcgcagctgcctgcagg
pITR-202
(SEQ ID NO: 13)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtgggattttgccgatttcggcctattggttaaaaaatgagctgatttaacaaaaatttaacgcg
aattttaacaaaatgataggcacctattggtcttactgacatccactttgcctttctctccacaggcgt
acctgcatatgcccccagaaaacctccagcagctggtgctttcagcagagagggaggctgcacagggct
tcctgacactcatgctgcaggggaagctgcaggggaagctgcaggtaccaccatccgaggagcaggccc
tgggtcgcctgacagccctgctgctccagcggtacccacgcctcacctcccagctcttcattgacctgt
caccactcatccctttcttggctgtctctgacctgatgcgcttcccaccatccctgttagccaacgaca
gtgtcctggctgccatccgggattacagcccaggaatgaggcctgaacagaaggaggctctggcaaagc
gactgctggcccctgaactgtttggggaagtgcctgcctggccccaggagctgctgtgggcagtgctgc
ccctgctcccccacctccctctggagaactttttgcagctcagccctcaccagatccaggccctggagg
atagctggccagcagcaggtctggggccagggcatgcccgccatgtgctgcgcagcctggtaaaccaga
gtgtccaggatggtgaggagcaggtacgcaggcttgggcccctcgcctgtttcctgagccctgaggagc
tgcagagcctagtgcccctgagtgatccaacggggccagtagaacgggggctgctggaatgtgcagcca
atgggaccctcagcccagaaggacgggtggcatatgaacttctgggtgtgttgcgctcatctggaggag
cggtgctgagcccccgggagctgcgggtctgggcccctctcttctctcagctgggcctccgcttccttc
aggagctgtcagagccccagcttagagccatgcttcctgtcctccagggaactagtgttacacctgctc
aggctgtcctgctgcttggacggctccttcctaggcacgatctatccctggaggaactctgctccttgc
accttctgctaccaggcctcagcccccagacactccaggccatccctaggcgagtcctggtcggggctt
gttcctgcctggcccctgaactgtcacgcctctcagcctgccagaccgcagcactgctgcagacctttc
gggttaaagatggtgttaaaaatatgggtacaacaggtgctggtccagctgtgtgtatccctggtcagc
ctattcccaccacctggccagactgcctgcttcccctgctcccattaaagctgctacaactggattcct
tggctcttctggcaaatcgaagacgctactgggagctgccctggtctgagcagcaggcacagtttctct
ggaagaagatgcaagtacccaccaaccttaccctcaggaatctgcaggctctgggcaccctggcaggag
gcatgtcctgtgagtttctgcagcagatcaactccatggtagacttccttgaagtggtgcacatgatct
atcagctgcccactagagttcgagggagcctgagggcctgtatctgggcagagctacagcggaggatgg
caatgccagaaccagaatggacaactgtagggccagaactgaacgggctggatagcaagctactcctgg
acttaccgatccagttgatggacagactatccaatgaatccattatgttggtggtggagctggtgcaaa
gagctccagagcagctgctggcactgacccccctccaccaggcagccctggcagagagggcactacaaa
acctggctccaaaggagactccagtctcaggggaagtgctggagaccttaggccctttggttggattcc
tggggacagagagcacacgacagatccccctacagatcctgctgtcccatctcagtcagctgcaaggct
tctgcctaggagagacatttgccacagagctgggatggctgctattgcaggagtctgttcttgggaaac
cagagttgtggagccaggatgaagtagagcaagctggacgcctagtattcactctgtctactgaggcaa
tttccttgatccccagggaggccttgggtccagagaccctggagcggcttctagaaaagcagcagagct
gggagcagagcagagttggacagctgtgtagggagccacagcttgctgccaagaaagcagccctggtag
caggggtggtgcgaccagctgctgaggatcttccagaacctgtgccaaattgtgcagatgtacgaggga
cattcccagcagcctggtctgcaacccagattgcagagatggagctctcagactttgaggactgcctga
cattatttgcaggagacccaggacttgggcctgaggaactgcgggcagccatgggcaaagcaaaacagt
tgtggggtcccccccggggatttcgtcctgagcagatcctgcagcttggtaggctcttaataggtctag
gagatcgggaactacaggagctgatcctagtggactggggagtgctgagcaccctggggcagatagatg
gctggagcaccactcagctccgcattgtggtctccagtttcctacggcagagtggtcggcatgtgagcc
acctggacttcgttcatctgacagcgctgggttatactctctgtggactgcggccagaggagctccagc
acatcagcagttgggagttcagccaagcagctctcttcctcggcaccctgcatctccagtgctctgagg
aacaactggaggttctggcccacctacttgtactgcctggtgggtttggcccaatcagtaactgggggc
ctgagatcttcactgaaattggcaccatagcagctgggatcccagacctggctctttcagcactgctgc
ggggacagatccagggcgttactcctcttgccatttctgtcatccctcctcctaaatttgctgtggtgt
ttagtcccatccaactatctagtctcaccagtgctcaggctgtggctgtcactcctgagcaaatggcct
ttctgagtcctgagcagcgacgagcagttgcatgggcccaacatgagggaaaggagagcccagaacagc
aaggtcgaagtacagcctggggcctccaggactggtcacgaccttcctggtccctggtattgactatca
gcttccttggccacctgctatgagcctgtctctacagtagaaggagattgtggggagagaaatcttaag
tcataatgaataaagtgcaaacagaagtgcatcctgattattttcagaagctgatgaggaataactagt
gctgatcagcctcgactgtgccttctagttgccagccatctgttgtttgcccctcccccgtgccttcct
tgaccctggaaggtgccactcccactgtcctttcctaataaaatgaggaaattgcatcgcattgtctga
gtaggtgtcattctattctggggggtggggtggggcaggacagcaagggggaggattgggaagacaata
gcaggcatgctggggatgcggtgggctctatggcaattcagactcccactagaagaaacagaacttgaa
aaaaggataattgtggtgaacacctcaacccagtggtccaaaaactgaaaaactttgaccccgagcacc
ctcacacagatagactactgaaagaaggagaaaggggcacttactcagtctcccttatcagattgcctt
acgaggagtactagactccctcaagcaaatagatctcacttaccacttgcaaaggtatactactttcac
tctattagacctatatatctgaccagggtcctattccaagacaactcttctactcttccagcagactct
tatagaaagaaagattctggggtccaagaaagcagtactttcttacaaggagccaaaaaaaataacctt
tctttagcacttctaaccttggagtgaactggtgatcaaagagaggttggctccctggggacaagtgcc
acaaattcagtcaactacaagaaagttgagaacactgttctcccgaaaccaagcttgaattcagctgac
gtgcctcggaccgctaggaacccctagtgatggagttggccactccctctctgcgcgctcgctcgctca
ctgaggccgggcgaccaaaggtcgcccgacgcccgggctttgcccgggcggcctcagtgagcgagcgag
cgcgcagctgcctgcagg
pITR-202GFP
(SEQ ID NO: 14)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtgggattttgccgatttcggcctattggttaaaaaatgagctgatttaacaaaaatttaacgcg
aattttaacaaaatgataggcacctattggtcttactgacatccactttgcctttctctccacaggcgt
acctgcatatgcccccagaaaacctccagcagctggtgctttcagcagagagggaggctgcacagggct
tcctgacactcatgctgcaggggaagctgcaggggaagctgcaggtaccaccatccgaggagcaggccc
tgggtcgcctgacagccctgctgctccagcggtacccacgcctcacctcccagctcttcattgacctgt
caccactcatccctttcttggctgtctctgacctgatgcgcttcccaccatccctgttagccaacgaca
gtgtcctggctgccatccgggattacagcccaggaatgaggcctgaacagaaggaggctctggcaaagc
gactgctggcccctgaactgtttggggaagtgcctgcctggccccaggagctgctgtgggcagtgctgc
ccctgctcccccacctccctctggagaactttttgcagctcagccctcaccagatccaggccctggagg
atagctggccagcagcaggtctggggccagggcatgcccgccatgtgctgcgcagcctggtaaaccaga
gtgtccaggatggtgaggagcaggtacgcaggcttgggcccctcgcctgtttcctgagccctgaggagc
tgcagagcctagtgcccctgagtgatccaacggggccagtagaacgggggctgctggaatgtgcagcca
atgggaccctcagcccagaaggacgggtggcatatgaacttctgggtgtgttgcgctcatctggaggag
cggtgctgagcccccgggagctgcgggtctgggcccctctcttctctcagctgggcctccgcttccttc
aggagctgtcagagccccagcttagagccatgcttcctgtcctccagggaactagtgttacacctgctc
aggctgtcctgctgcttggacggctccttcctaggcacgatctatccctggaggaactctgctccttgc
accttctgctaccaggcctcagcccccagacactccaggccatccctaggcgagtcctggtcggggctt
gttcctgcctggcccctgaactgtcacgcctctcagcctgccagaccgcagcactgctgcagacctttc
gggttaaagatggtgttaaaaatatgggtacaacaggtgctggtccagctgtgtgtatccctggtcagc
ctattcccaccacctggccagactgcctgcttcccctgctcccattaaagctgctacaactggattcct
tggctcttctggcaaatcgaagacgctactgggagctgccctggtctgagcagcaggcacagtttctct
ggaagaagatgcaagtacccaccaaccttaccctcaggaatctgcaggctctgggcaccctggcaggag
gcatgtcctgtgagtttctgcagcagatcaactccatggtagacttccttgaagtggtgcacatgatct
atcagctgcccactagagttcgagggagcctgagggcctgtatctgggcagagctacagcggaggatgg
caatgccagaaccagaatggacaactgtagggccagaactgaacgggctggatagcaagctactcctgg
acttaccgatccagttgatggacagactatccaatgaatccattatgttggtggtggagctggtgcaaa
gagctccagagcagctgctggcactgacccccctccaccaggcagccctggcagagagggcactacaaa
acctggctccaaaggagactccagtctcaggggaagtgctggagaccttaggccctttggttggattcc
tggggacagagagcacacgacagatccccctacagatcctgctgtcccatctcagtcagctgcaaggct
tctgcctaggagagacatttgccacagagctgggatggctgctattgcaggagtctgttcttgggaaac
cagagttgtggagccaggatgaagtagagcaagctggacgcctagtattcactctgtctactgaggcaa
tttccttgatccccagggaggccttgggtccagagaccctggagcggcttctagaaaagcagcagagct
gggagcagagcagagttggacagctgtgtagggagccacagcttgctgccaagaaagcagccctggtag
caggggtggtgcgaccagctgctgaggatcttccagaacctgtgccaaattgtgcagatgtacgaggga
cattcccagcagcctggtctgcaacccagattgcagagatggagctctcagactttgaggactgcctga
cattatttgcaggagacccaggacttgggcctgaggaactgcgggcagccatgggcaaagcaaaacagt
tgtggggtcccccccggggatttcgtcctgagcagatcctgcagcttggtaggctcttaataggtctag
gagatcgggaactacaggagctgatcctagtggactggggagtgctgagcaccctggggcagatagatg
gctggagcaccactcagctccgcattgtggtctccagtttcctacggcagagtggtcggcatgtgagcc
acctggacttcgttcatctgacagcgctgggttatactctctgtggactgcggccagaggagctccagc
acatcagcagttgggagttcagccaagcagctctcttcctcggcaccctgcatctccagtgctctgagg
aacaactggaggttctggcccacctacttgtactgcctggtgggtttggcccaatcagtaactgggggc
ctgagatcttcactgaaattggcaccatagcagctgggatcccagacctggctctttcagcactgctgc
ggggacagatccagggcgttactcctcttgccatttctgtcatccctcctcctaaatttgctgtggtgt
ttagtcccatccaactatctagtctcaccagtgctcaggctgtggctgtcactcctgagcaaatggcct
ttctgagtcctgagcagcgacgagcagttgcatgggcccaacatgagggaaaggagagcccagaacagc
aaggtcgaagtacagcctggggcctccaggactggtcacgaccttcctggtccctggtattgactatca
gcttccttggccacctgctaggctccggagagggcagaggaagtctgctaacatgcggtgacgtcgagg
agaatcctggcccaatggagagcgacgagagcggcctgcccgccatggagatcgagtgccgcatcaccg
gcaccctgaacggcgtggagttcgagctggtgggcggcggagagggcacccccgagcagggccgcatga
ccaacaagatgaagagcaccaaaggcgccctgaccttcagcccctacctgctgagccacgtgatgggct
acggcttctaccacttcggcacctaccccagcggctacgagaaccccttcctgcacgccatcaacaacg
gcggctacaccaacacccgcatcgagaagtacgaggacggcggcgtgctgcacgtgagcttcagctacc
gctacgaggccggccgcgtgatcggcgacttcaaggtgatgggcaccggcttccccgaggacagcgtga
tcttcaccgacaagatcatccgcagcaacgccaccgtggagcacctgcaccccatgggcgataacgatc
tggatggcagcttcacccgcaccttcagcctgcgcgacggcggctactacagctccgtggtggacagcc
acatgcacttcaagagcgccatccaccccagcatcctgcagaacgggggccccatgttcgccttccgcc
gcgtggaggaggatcacagcaacaccgagctgggcatcgtggagtaccagcacgccttcaagaccccgg
atgcagatgccggtgaagaataagcctgtctctacagtagaaggagattgtggggagagaaatcttaag
tcataatgaataaagtgcaaacagaagtgcatcctgattattttcagaagctgatgaggaataactagt
gctgatcagcctcgactgtgccttctagttgccagccatctgttgtttgcccctcccccgtgccttcct
tgaccctggaaggtgccactcccactgtcctttcctaataaaatgaggaaattgcatcgcattgtctga
gtaggtgtcattctattctggggggtggggtggggcaggacagcaagggggaggattgggaagacaata
gcaggcatgctggggatgcggtgggctctatggaagcttgaattcagctgacgtgcctcggaccgctag
gaacccctagtgatggagttggccactccctctctgcgcgctcgctcgctcactgaggccgggcgacca
aaggtcgcccgacgcccgggctttgcccgggcggcctcagtgagcgagcgagcgcgcagctgcctgcag
g
pITR-203
(SEQ ID NO: 15)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtagacttgcccaaaaactctggcaaagttgaattgcttccaaaagttcaactttatcagaagga
cctattccctacggaaactagctgaaggtctcctggcactctggatctcgtggaagggagccttcttca
gggaacagagggagcgattaagtggtgaaaagcaaacagacctggaaaagttccctttctgagagtagc
aacagaaagctctgcaaagactccctccaagctattggatcctcttgcttgggataaccacttgagtac
tcagataccaaaagaagagtggaaatcccaagagaagtcaccagaaaaaacagcttttaagaaaaagga
tacacttttgtccctgaacgcttgtgaaagcaatctgacaatagcagcaattgaaaagggacaaaataa
gcccgaaatagaagtcacctgggcaaagcaaggtaggactgaaaggctgtgctctcaaaacccaccagt
cttgaaacgcactcaacgggtgactagatcccatatatggagttccgcgttacataacttacggtaaat
ggcccgcctggctgaccgcccaacgacccccgcccattgacgtcaataatgacgtatgttcccatagta
acgccaatagggactttccattgacgtcaatgggtggagtatttacggtaaactgcccacttggcagta
catcaagtgtatcatatgccaagtacgccccctattgacgtcaatgacggtaaatggcccgcctggcat
tatgcccagtacatgaccttatgggactttcctacttggcagtacatctacgtattagtcatcgctatt
accatggtgatgcggttttggtgatgcggttttggcagtacatcaatgggcgtggatagcggtttgact
cacggggatttccaagtctccaccccattgacgtcaatgggagtttgttttggcaccaaaatcaacggg
actttccaaaatgtcgtaacaactccgccccattgacgcaaatgggcggtaggcgtgtacggtgggagg
tctatataagcagagctcgtttagtgaaccgtcaccggtggtgccctgccctcacctggctatcccaca
caggtgagaataaccagaactcacctccggtaccagtgttcacttggccaccatggctctcagcctctg
gcccctgctgctgctgctgctgctgctgctgctgctgtcctttgcagtgactctggcccctactgggcc
tcattccctggaccctggtctctccttcctgaagtcattgctctccactctggaccaggctccccaggg
ctccctgagccgctcacggttctttacattcctggccaacatttcttcttcctttgagcctgggagaat
gggggaaggaccagtaggagagcccccacctctccagccgcctgctctgcggctccatgattttctagt
gacactgagaggtagccccgactgggagccaatgctagggctgctaggggatatgctggcactgctggg
acaggagcagactccccgagatttcctggtgcaccaggcaggggtgctgggtggacttgtggaggtgct
gctgggagccttagttcctgggggcccccctaccccaactcggcccccatgcacccgtgatgggccgtc
tgactgtgtcctggctgctgactggttgccttctctgctgctgttgttagagggcacacgctggcaagc
tctggtgcaggtgcagcccagtgtggaccccaccaatgccacaggcctcgatgggagggaggcagctcc
tcactttttgcagggtctgttgggtttgcttaccccaacaggggagctaggctccaaggaggctctttg
gggcggtctgctacgcacagtgggggcccccctctatgctgcctttcaggaggggctgctccgtgtcac
tcactccttgcaggatgaggtcttctccattttggggcagccagagcctgataccaatgggcagtgcca
gggaggtaaccttcaacagctgctcttatggggcgtccggcacaacctttcctgggatgtccaggcgct
gggctttctgtctggatcaccacccccaccccctgccctccttcactgcctgagcacgggcgtgcctct
gcccagagcttctcagccgtcagcccacatcagcccacgccaacggcgagccatcactgtggaggccct
ctgtgagaaccacttaggcccagcaccaccctacagcatttccaacttctccatccacttgctctgcca
gcacaccaagcctgccactccacagccccatcccagcaccactgccatctgccagacagctgtgtggta
tgcagtgtcctgggcaccaggtgcccaaggctggctacaggcctgccacgaccagtttcctgatgagtt
tttggatgcgatctgcagtaacctctccttttcagccctgtctggctccaaccgccgcctggtgaagcg
gctctgtgctggcctgctcccaccccctaccagctgccctgaaggcctgccccctgttcccctcacccc
agacatcttttggggctgcttcttggagaatgagactctgtgggctgagcgactgtgtggggaggcaag
tctacaggctgtgccccccagcaaccaggcttgggtccagcatgtgtgccagggccccaccccagatgt
cactgcctccccaccatgccacattggaccctgtggggaacgctgcccggatgggggcagcttcctggt
gatggtctgtgccaatgacaccatgtatgaggtcctggtgcccttctggccttggctagcaggccaatg
caggataagtcgtgggggcaatgacacttgcttcctagaagggctgctgggcccccttctgccctctct
gccaccactgggaccatccccactctgtctgacccctggccccttcctccttggcatgctatcccagtt
gccacgctgtcagtcctctgtcccagctcttgctcaccccacacgcctacactatctcctccgcctgct
gaccttcctcttgggtccaggggctgggggcgctgaggcccaggggatgctgggtcgggccctactgct
ctccagtctcccagacaactgctccttctgggatgcctttcgcccagagggccggcgcagtgtgctacg
gacgattggggaatacctggaacaagatgaggagcagccaaccccatcaggctttgaacccactgtcaa
ccccagctctggtataagcaagatggagctgctggcctgctttagtcctgtgctgtgggatctgctcca
gagggaaaagagtgtttgggccctgcagattctagtgcaggtaagtatcaaggttacaagacaggttta
aggagaccaatagaaactgggcttgtcgagacagagaagactcttgcgtttctctaggtggaggccgaa
agtacatgtttcgcatgggaaccccagaccctgagtacccagatgactacagccaaggtgggaccaggc
tggacgggaagaatctggtgcaggaatggctggcgaagcgccagggtgcccggtatgtgtggaaccgca
ctgagctcatgcaggcttccctggacccgtctgtgacccatctcatgggtctctttgagcctggagaca
tgaaatacgagatccaccgagactccacactggacccctccctgatggagatgacagaggctgccctgc
gcctgctgagcaggaacccccgcggcttcttcctcttcgtggagggtggtcgcatcgaccatggtcatc
atgaaagcagggcttaccgggcactgactgagacgatcatgttcgacgacgccattgagaggggggcc
agctcaccagcgaggaggacacgctgagcctcgtcactgccgaccactcccacgtcttctccttcggag
gctaccccctgcgagggagctccatcttcgggctggcccctggcaaggcccgggacaggaaggcctaca
cggtcctcctatacggaaacggtccaggctatgtgctcaaggacggcgcccggccggatgttaccgaga
gcgagagcgggagccccgagtatcggcagcagtcagcagtgcccctggacgaagagacccacgcaggcg
aggacgtggcggtgttcgcgcgcggcccgcaggcgcacctggttcacggcgtgcaggagcagaccttca
tagcgcacgtcatggccttcgccgcctgcctggagccctacaccgcctgcgacctggcgccccccgccg
gcaccaccgacgccgcgcacccggggcgaagcttgaattcagctgacgtgcctcggaccgctaggaacc
cctagtgatggagttggccactccctctctgcgcgctcgctcgctcactgaggccgggcgaccaaaggt
cgcccgacgcccgggctttgcccgggcggcctcagtgagcgagcgagcgcgcagctgcctgcagg
pITR-204
(SEQ ID NO: 16)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtctaggtggaggccgaaagtacatgtttcgcatgggaaccccagaccctgagtacccagatgac
tacagccaaggtgggaccaggctggacgggaagaatctggtgcaggaatggctggcgaagcgccagggt
gcccggtatgtgtggaaccgcactgagctcatgcaggcttccctggacccgtctgtgacccatctcatg
ggtctctttgagcctggagacatgaaatacgagatccaccgagactccacactggacccctccctgatg
gagatgacagaggctgccctgcgcctgctgagcaggaacccccgcggcttcttcctcttcgtggagggt
ggtcgcatcgaccatggtcatcatgaaagcagggcttaccgggcactgactgagacgatcatgttcgac
gacgccattgagagggcgggccagctcaccagcgaggaggacacgctgagcctcgtcactgccgaccac
tcccacgtcttctccttcggaggctaccccctgcgagggagctccatcttcgggctggcccctggcaag
gcccgggacaggaaggcctacacggtcctcctatacggaaacggtccaggctatgtgctcaaggacggc
gcccggccggatgttaccgagagcgagagcgggagccccgagtatcggcagcagtcagcagtgcccctg
gacgaagagacccacgcaggcgaggacgtggcggtgttcgcgcgcggcccgcaggcgcacctggttcac
ggcgtgcaggagcagaccttcatagcgcacgtcatggccttcgccgcctgcctggagccctacaccgcc
tgcgacctggcgccccccgccggcaccaccgacgccgcgcacccggggcggataggcacctattggtct
tactgacatccactttgcctttctctccacaggcgtacctgcatatgcccccagaaaacctccagcagc
tggtgctttcagcagagagggaggctgcacagggcttcctgacactcatgctgcaggggaagctgcagg
ggaagctgcaggtaccaccatccgaggagcaggccctgggtcgcctgacagccctgctgctccagcggt
acccacgcctcacctcccagctcttcattgacctgtcaccactcatccctttcttggctgtctctgacc
tgatgcgcttcccaccatccctgttagccaacgacagtgtcctggctgccatccgggattacagcccag
gaatgaggcctgaacagaaggaggctctggcaaagcgactgctggcccctgaactgtttggggaagtgc
ctgcctggccccaggagctgctgtgggcagtgctgcccctgctcccccacctccctctggagaactttt
tgcagctcagccctcaccagatccaggccctggaggatagctggccagcagcaggtctggggccagggc
atgcccgccatgtgctgcgcagcctggtaaaccagagtgtccaggatggtgaggagcaggtacgcaggc
ttgggcccctcgcctgtttcctgagccctgaggagctgcagagcctagtgcccctgagtgatccaacgg
ggccagtagaacgggggctgctggaatgtgcagccaatgggaccctcagcccagaaggacgggtggcat
atgaacttctgggtgtgttgcgctcatctggaggagcggtgctgagcccccgggagctgcgggtctggg
cccctctcttctctcagctgggcctccgcttccttcaggagctgtcagagccccagcttagagccatgc
ttcctgtcctccagggaactagtgttacacctgctcaggctgtcctgctgcttggacggctccttccta
ggcacgatctatccctggaggaactctgctccttgcaccttctgctaccaggcctcagcccccagacac
tccaggccatccctaggcgagtcctggtcggggcttgttcctgcctggcccctgaactgtcacgcctct
cagcctgccagaccgcagcactgctgcagacctttcgggttaaagatggtgttaaaaatatgggtacaa
caggtgctggtccagctgtgtgtatccctggtcagcctattcccaccacctggccagactgcctgcttc
ccctgctcccattaaagctgctacaactggattccttggctcttctggcaaatcgaagacgctactggg
agctgccctggtctgagcagcaggcacagtttctctggaagaagatgcaagtacccaccaaccttaccc
tcaggaatctgcaggctctgggcaccctggcaggaggcatgtcctgtgagtttctgcagcagatcaact
ccatggtagacttccttgaagtggtgcacatgatctatcagctgcccactagagttcgagggagcctga
gggcctgtatctgggcagagctacagcggaggatggcaatgccagaaccagaatggacaactgtagggc
cagaactgaacgggctggatagcaagctactcctggacttaccgatccagttgatggacagactatcca
atgaatccattatgttggtggtggagctggtgcaaagagctccagagcagctgctggcactgacccccc
tccaccaggcagccctggcagagagggcactacaaaacctggctccaaaggagactccagtctcagggg
aagtgctggagaccttaggccctttggttggattcctggggacagagagcacacgacagatccccctac
agatcctgctgtcccatctcagtcagctgcaaggcttctgcctaggagagacatttgccacagagctgg
gatggctgctattgcaggagtctgttcttgggaaaccagagttgtggagccaggatgaagtagagcaag
ctggacgcctagtattcactctgtctactgaggcaatttccttgatccccagggaggccttgggtccag
agaccctggagcggcttctagaaaagcagcagagctgggagcagagcagagttggacagctgtgtaggg
agccacagcttgctgccaagaaagcagccctggtagcaggggtggtgcgaccagctgctgaggatcttc
cagaacctgtgccaaattgtgcagatgtacgagggacattcccagcagcctggtctgcaacccagattg
cagagatggagctctcagactttgaggactgcctgacattatttgcaggagacccaggacttgggcctg
aggaactgcgggcagccatgggcaaagcaaaacagttgtggggtcccccccggggatttcgtcctgagc
agatcctgcagcttggtaggctcttaataggtctaggagatcgggaactacaggagctgatcctagtgg
actggggagtgctgagcaccctggggcagatagatggctggagcaccactcagctccgcattgtggtct
ccagtttcctacggcagagtggtcggcatgtgagccacctggacttcgttcatctgacagcgctgggtt
atactctctgtggactgcggccagaggagctccagcacatcagcagttgggagttcagccaagcagctc
tcttcctcggcaccctgcatctccagtgctctgaggaacaactggaggttctggcccacctacttgtac
tgcctggtgggtttggcccaatcagtaactgggggcctgagatcttcactgaaattggcaccatagcag
ctgggatcccagacctggctctttcagcactgctgcggggacagatccagggcgttactcctcttgcca
tttctgtcatccctcctcctaaatttgctgtggtgtttagtcccatccaactatctagtctcaccagtg
ctcaggctgtggctgtcactcctgagcaaatggcctttctgagtcctgagcagcgacgagcagttgcat
gggcccaacatgagggaaaggagagcccagaacagcaaggtcgaagtacagcctggggcctccaggact
ggtcacgaccttcctggtccctggtattgactatcagcttccttggccacctgctatgagcctgtctct
acagtagaaggagattgtggggagagaaatcttaagtcataatgaataaagtgcaaacagaagtgcatc
ctgattattttcagaagctgatgaggaataactagtgctgatcagcctcgactgtgccttctagttgcc
agccatctgttgtttgcccctcccccgtgccttccttgaccctggaaggtgccactcccactgtccttt
cctaataaaatgaggaaattgcatcgcattgtctgagtaggtgtcattctattctggggggtggggtgg
ggcaggacagcaagggggaggattgggaagacaatagcaggcatgctggggatgcggtgggctctatgg
aagcttgaattcagctgacgtgcctcggaccgctaggaacccctagtgatggagttggccactccctct
ctgcgcgctcgctcgctcactgaggccgggcgaccaaaggtcgcccgacgcccgggctttgcccgggcg
gcctcagtgagcgagcgagcgcgcagctgcctgcagg
pITR-205
(SEQ ID NO: 17)
cctgcaggcagctgcgcgctcgctcgctcactgaggccgcccgggcgtcgggcgacctttggtcgcccg
gcctcagtgagcgagcgagcgcgcagagagggagtggccaactccatcactaggggttcctgcggccgc
acgcgtgacattgattattgactagttattaatagtaatcaattacggggtcattagttcatagcccat
atatggagttccgcgttacataacttacggtaaatggcccgcctggctgaccgcccaacgacccccgcc
cattgacgtcaataatgacgtatgttcccatagtaacgccaatagggactttccattgacgtcaatggg
tggactatttacggtaaactgcccacttggcagtacatcaagtgtatcatatgccaagtacgcccccta
ttgacgtcaatgacggtaaatggcccgcctggcattatgcccagtacatgaccttatgggactttccta
cttggcagtacatctacgtattagtcatcgctattaccatgggtcgaggtgagccccacgttctgcttc
actctccccatctcccccccctccccacccccaattttgtatttatttattttttaattattttgtgca
gcgatgggggcggggggggggggggcgcgcgccaggcggggcggggcggggcgaggggcggggcggggc
gaggcggagaggtgcggcggcagccaatcagagcggcgcgctccgaaagtttccttttatggcgaggcg
gcggcggcggcggccctataaaaagcgaagcgcgcggcgggcgggagtcgctgcgttgccttcgccccg
tgccccgctccgcgccgcctcgcgccgcccgccccggctctgactgaccgcgttactcccacaggtgag
cgggcgggacggcccttctcctccgggctgtaattagcgcttggtttaatgacggctcgtttcttttct
gtggctgcgtgaaagccttaaagggctccgggagggccctttgtgcgggggggagcggctcggggggtg
cgtgcgtgtgtgtgtgcgtggggagcgccgcgtgcggcccgcgctgcccggcggctgtgagcgctgcgg
gcgcggcgcggggctttgtgcgctccgcgtgtgcgcgaggggagcgcggccgggggcggtgccccgcgg
tgcgggggggctgcgaggggaacaaaggctgcgtgcggggtgtgtgcgtgggggggtgagcagggggtg
tgggcgcggcggtcgggctgtaacccccccctgcacccccctccccgagttgctgagcacggcccggct
tcgggtgcggggctccgtgcggggcgtggcgcggggctcgccgtgccgggcggggggtggcggcaggtg
ggggtgccgggcggggcggggccgcctcgggccggggagggctcgggggaggggcgcggcggcccccgg
agcgccggcggctgtcgaggcgcggcgagccgcagccattgccttttatggtaatcgtgcgagagggcg
cagggacttcctttgtcccaaatctgtgcggagccgaaatctgggaggcgccgccgcaccccctctagc
gggcgcggggcgaagcggtgcggcgccggcaggaaggaaatgggcggggagggccttcgtgcgtcgccg
cgccgccgtccccttctccctctccagcctcggggctgtccgcggggggacggctgccttcggggggga
cggggcagggcggggttcggcttctggcgtgtgaccggcggctctagagcctctgctaaccatgttcat
gccttcttctttttcctacagctcctgggcaacgtgctggttattgtgaccggtggtgccctgccctca
cctggctatcccacacaggtgagaataaccagaactcacctccggtaccagtgttcacttggccaccat
ggctctcagcctctggcccctgctgctgctgctgctgctgctgctgctgctgtcctttgcagtgactct
ggcccctactgggcctcattccctggaccctggtctctccttcctgaagtcattgctctccactctgga
ccaggctccccagggctccctgagccgctcacggttctttacattcctggccaacatttcttcttcctt
tgagcctgggagaatgggggaaggaccagtaggagagcccccacctctccagccgcctgctctgcggct
ccatgattttctagtgacactgagaggtagccccgactgggagccaatgctagggctgctaggggatat
gctggcactgctgggacaggagcagactccccgagatttcctggtgcaccaggcaggggtgctgggtgg
acttgtggaggtgctgctgggagccttagttcctgggggcccccctaccccaactcggcccccatgcac
ccgtgatgggccgtctgactgtgtcctggctgctgactggttgccttctctgctgctgttgttagaggg
cacacgctggcaagctctggtgcaggtgcagcccagtgtggaccccaccaatgccacaggcctcgatgg
gagggaggcagctcctcactttttgcagggtctgttgggtttgcttaccccaacaggggagctaggctc
caaggaggctctttggggcggtctgctacgcacagtgggggcccccctctatgctgcctttcaggaggg
gctgctccgtgtcactcactccttgcaggatgaggtcttctccattttggggcagccagagcctgatac
caatgggcagtgccagggaggtaaccttcaacagctgctcttatggggcgtccggcacaacctttcctg
ggatgtccaggcgctgggctttctgtctggatcaccacccccaccccctgccctccttcactgcctgag
cacgggcgtgcctctgcccagagcttctcagccgtcagcccacatcagcccacgccaacggcgagccat
cactgtggaggccctctgtgagaaccacttaggcccagcaccaccctacagcatttccaacttctccat
ccacttgctctgccagcacaccaagcctgccactccacagccccatcccagcaccactgccatctgcca
gacagctgtgtggtatgcagtgtcctgggcaccaggtgcccaaggctggctacaggcctgccacgacca
gtttcctgatgagtttttggatgcgatctgcagtaacctctccttttcagccctgtctggctccaaccg
ccgcctggtgaagcggctctgtgctggcctgctcccaccccctaccagctgccctgaaggcctgccccc
tgttcccctcaccccagacatcttttggggctgcttcttggagaatgagactctgtgggctgagcgact
gtgtggggaggcaagtctacaggctgtgccccccagcaaccaggcttgggtccagcatgtgtgccaggg
ccccaccccagatgtcactgcctccccaccatgccacattggaccctgtggggaacgctgcccggatgg
gggcagcttcctggtgatggtctgtgccaatgacaccatgtatgaggtcctggtgcccttctggccttg
gctagcaggccaatgcaggataagtcgtgggggcaatgacacttgcttcctagaagggctgctgggccc
ccttctgccctctctgccaccactgggaccatccccactctgtctgacccctggccccttcctccttgg
catgctatcccagttgccacgctgtcagtcctctgtcccagctcttgctcaccccacacgcctacacta
tctcctccgcctgctgaccttcctcttgggtccaggggctgggggcgctgaggcccaggggatgctggg
tcgggccctactgctctccagtctcccagacaactgctccttctgggatgcctttcgcccagagggccg
gcgcagtgtgctacggacgattggggaatacctggaacaagatgaggagcagccaaccccatcaggctt
tgaacccactgtcaaccccagctctggtataagcaagatggagctgctggcctgctttagtcctgtgct
gtgggatctgctccagagggaaaagagtgtttgggccctgcagattctagtgcaggtaagtatcaaggt
tacaagacaggtttaaggagaccaatagaaactgggcttgtcgagacagagaagactcttgcgtttctg
ggattttgccgatttcggcctattggttaaaaaatgagctgatttaacaaaaatttaacgcgaatttta
acaaaataagcttgaattcagctgacgtgcctcggaccgctaggaacccctagtgatggagttggccac
tccctctctgcgcgctcgctcgctcactgaggccgggcgaccaaaggtcgcccgacgcccgggctttgc
ccgggcggcctcagtgagcgagcgagcgcgcagctgcctgcagg

In some embodiments, the vector(s) is an adenovirus (see, e.g., Dmitriev et al. (1998) J. Virol. 72: 9706-9713; and Poulin et al., J. Virol 8: 10074-10086, 2010). In some embodiments, the vector(s) is a retrovirus (see, e.g., Maier et al. (2010) Future Microbiol 5: 1507-23).

In some embodiments, the vector(s) is a lentivirus (see, e.g., Matrai et al. (2010) Mol Ther. 18: 477-490; Banasik et al. (2010) Gene Ther. 17:150-7; and Wanisch et al. (2009) Mol. Ther. 17: 1316-32). A lentiviral vector refers to a vector derived from at least a portion of a lentivirus genome, including especially a self-inactivating lentiviral vector as provided in Milone et al., Mol. Ther. 17(8): 1453-1464 (2009). Non-limiting lentivirus vectors that may be used in the clinic include the LENTIVECTOR® gene delivery technology from Oxford BioMedica, the LENTIMAX™ vector system from Lentigen, and the like. Other types of lentiviral vectors are also available and would be known to one skilled in the art.

The vectors provided herein can be of different sizes. The choice of vector that is used in any of the compositions, kits, and methods described herein may depend on the size of the vector.

In some embodiments, the vector(s) is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, the vector(s) is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, the vector(s) is a transposon (e.g., PiggyBac transposon) and can include greater than 200 kb. In some examples, the vector(s) is a transposon having a total length in the range of about 1 kb to about 10 kb, about 1 kb to about 20 kb, about 1 kb to about 30 kb, about 1 kb to about 40 kb, about 1 kb to about 50 kb, about 1 kb to about 60 kb, about 1 kb to about 70 kb, about 1 kb to about 80 kb, about 1 kb to about 90 kb, about 10 kb to about 20 kb, about 10 kb to about 30 kb, about 10 kb to about 40 kb, about 10 kb to about 50 kb, about 10 kb to about 60 kb, about 10 kb to about 70 kb, about 10 kb to about 90 kb, about 10 kb to about 100 kb, about 20 kb to about 30 kb, about 20 kb to about 40 kb, about 20 kb to about 50 kb, about 20 kb to about 60 kb, about 20 kb to about 70 kb, about 20 kb to about 80 kb, about 20 kb to about 90 kb, about 20 kb to about 100 kb, about 30 kb to about 40 kb, about 30 kb to about 50 kb, about 30 kb to about 60 kb, about 30 kb to about 70 kb, about 30 kb to about 80 kb, about 30 kb to about 90 kb, about 30 kb to about 100 kb, about 40 kb to about 50 kb, about 40 kb to about 60 kb, about 40 kb to about 70 kb, about 40 kb to about 80 kb, about 40 kb to about 90 kb, about 40 kb to about 100 kb, about 50 kb to about 60 kb, about 50 kb to about 70 kb, about 50 kb to about 80 kb, about 50 kb to about 90 kb, about 50 kb to about 100 kb, about 60 kb to about 70 kb, about 60 kb to about 80 kb, about 60 kb to about 90 kb, about 60 kb to about 100 kb, about 70 kb to about 80 kb, about 70 kb to about 90 kb, about 70 kb to about 100 kb, about 80 kb to about 90 kb, about 80 kb to about 100 kb, about 90 kb to about 100 kb, about 1 kb to about 100 kb, about 100 kb to about 200 kb, about 100 kb to about 300 kb, about 100 kb to about 400 kb, or about 100 kb to about 500 kb.

In some embodiments, the vector is a cosmid and can have a total length of up to 55 kb. In some examples, the vector is a cosmid and has a total number of nucleotides of about 1 kb to about 10 kb, about 1 kb to about 20 kb, about 1 kb to about 30 kb, about 1 kb to about 40 kb, about 1 kb to about 50 kb, about 1 kb to about 55 kb, about 10 kb to about 20 kb, about 10 kb to about 30 kb, about 10 kb to about 40 kb, about 10 kb to about 50 kb, about 10 kb to about 55 kb, about 15 kb to about 55 kb, about 15 kb to about 50 kb, about 15 kb to about 40 kb, about 15 kb to about 30 kb, about 15 kb to about 20 kb, about 20 kb to about 55 kb, about 20 kb to about 50 kb, about 20 kb to about 40 kb, about 20 kb to about 30 kb, about 25 kb to about 55 kb, about 25 kb to about 50 kb, about 25 kb to about 40 kb, about 25 kb to about 30 kb, about 30 kb to about 55 kb, about 30 kb to about 50 kb, about 30 kb to about 40 kb, about 35 kb to about 55 kb, about 40 kb to about 55 kb, about 40 kb to about 50 kb, or about 45 kb to about 55 kb.

In some embodiments, the vector(s) is an artificial chromosome and can have a total number of nucleotides of about 100 kb to about 2000 kb. In some embodiments, the artificial chromosome(s) is a human artificial chromosome (HAC) and can have a total number of nucleotides in the range of about 1 kb to about 10 kb, 1 kb to about 20 kb, about 1 kb to about 30 kb, about 1 kb to about 40 kb, about 1 kb to about 50 kb, about 1 kb to about 60 kb, about 10 kb to about 20 kb, about 10 kb to about 30 kb, about 10 kb to about 40 kb, about 10 kb to about 50 kb, about 10 kb to about 60 kb, about 20 kb to about 30 kb, about 20 kb to about 40 kb, about 20 kb to about 50 kb, about 20 kb to about 60 kb, about 30 kb to about 40 kb, about 30 kb to about 50 kb, about 30 kb to about 60 kb, about 40 kb to about 50 kb, about 40 kb to about 60 kb, or about 50 kb to about 60 kb.

In some embodiments, the artificial chromosome(s) is a yeast artificial chromosome (YAC) and can have a total number of nucleotides up to 1000 kb. In some embodiments, the artificial chromosome(s) is a YAC having a total number of nucleotides in the range of about 100 kb to about 1,000 kb, about 100 kb to about 900 kb, about 100 kb to about 800 kb, about 100 kb to about 700 kb, about 100 kb to about 600 kb, about 100 kb to about 500 kb, about 100 kb to about 400 kb, about 100 kb to about 300 kb, about 100 kb to about 200 kb, about 200 kb to about 1,000 kb, about 200 kb to about 900 kb, about 200 kb to about 800 kb, about 200 kb to about 700 kb, about 200 kb to about 600 kb, about 200 kb to about 500 kb, about 200 kb to about 400 kb, about 200 kb to about 300 kb, about 300 kb to about 1,000 kb, about 300 kb to about 900 kb, about 300 kb to about 800 kb, about 300 kb to about 700 kb, about 300 kb to about 600 kb, about 300 kb to about 500 kb, about 300 kb to about 400 kb, about 400 kb to about 1,000 kb, about 400 kb to about 900 kb, about 400 kb to about 800 kb, about 400 kb to about 700 kb, about 400 kb to about 600 kb, about 400 kb to about 500 kb, about 500 kb to about 1,000 kb, about 500 kb to about 900 kb, about 500 kb to about 800 kb, about 500 kb to about 700 kb, about 500 kb to about 600 kb, about 600 kb to about 1,000 kb, about 600 kb to about 900 kb, about 600 kb to about 800 kb, about 600 kb to about 700 kb, about 700 kb to about 1,000 kb, about 700 kb to about 900 kb, about 700 kb to about 800 kb, about 800 kb to about 1,000 kb, about 800 kb to about 900 kb, or about 900 kb to about 1,000 kb.

In some embodiments, the artificial chromosome(s) is a bacterial artificial chromosome (BAC) and can have a total number of nucleotides of up to 750 kb. In some embodiments, the artificial chromosome(s) is a BAC and can have a total number of nucleotides in the range of about 100 kb to about 750 kb, about 100 kb to about 700 kb, about 100 kb to about 600 kb, about 100 kb to about 500 kb, about 100 kb to about 400 kb, about 100 kb to about 300 kb, about 100 kb to about 200 kb, about 150 kb to about 750 kb, about 150 kb to about 700 kb, about 150 kb to about 600 kb, about 150 kb to about 500 kb, about 150 kb to about 400 kb, about 150 kb to about 300 kb, about 150 kb to about 200 kb, about 200 kb to about 750 kb, about 200 kb to about 700 kb, about 200 kb to about 600 kb, about 200 kb to about 500 kb, about 200 kb to about 400 kb, about 200 kb to about 300 kb, about 250 kb to about 750 kb, about 250 kb to about 700 kb, about 250 kb to about 600 kb, about 250 kb to about 500 kb, about 250 kb to about 400 kb, about 250 kb to about 300 kb, about 300 kb to about 750 kb, about 300 kb to about 700 kb, about 300 kb to about 600 kb, about 300 kb to about 500 kb, about 300 kb to about 400 kb, about 350 kb to about 750 kb, about 350 kb to about 700 kb, about 350 kb to about 600 kb, about 350 kb to about 500 kb, about 350 kb to about 400 kb, about 400 kb to about 750 kb, about 400 kb to about 700 kb, about 450 kb to about 600 kb, about 450 kb to about 500 kb, about 500 kb to about 750 kb, about 500 kb to about 700 kb, about 500 kb to about 600 kb, about 550 kb to about 750 kb, about 550 kb to about 700 kb, about 550 kb to about 600 kb, about 600 kb to about 750 kb, about 600 kb to about 700 kb, or about 650 kb to about 750 kb.

In some embodiments, the artificial chromosome(s) is a P1-derived artificial chromosome (PAC) and can have a total number of nucleotides of up to 300 kb. In some embodiments, the P1-derived artificial chromosome(s) can have a total number of nucleotides in the range of about 100 kb to about 300 kb, about 100 kb to about 200 kb, or about 200 kb to about 300 kb.

In some embodiments, the vector(s) is a viral vector and can have a total number of nucleotides of up to 10 kb. In some embodiments, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, the vector(s) is a lentivirus and can have a total number of nucleotides of up to 8 kb. In some examples, the lentivirus(es) can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some embodiments, the vector(s) is an adenovirus and can have a total number of nucleotides of up to 8 kb. In some embodiments, the adenovirus(es) can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kh, about 6 kb to about 8 kb, or about 7 kb to about 8 kb.

In some embodiments, the vector(s) is an adeno-associated virus (AAV vector) and can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments, the vector(s) is a Gateway® vector and can include a total number of nucleotides of up to 5 kb. In some embodiments, each Gateway® vector(s) includes a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments of any of the compositions, kits, and methods provided herein, the at least two different vectors can be substantially the same type of vector and may differ in size. In some embodiments, the at least two different vectors can be different types of vector, and may have substantially the same size or have different sizes.

In some embodiments, any of the at least two vectors can have a total number of nucleotides in the range of about 500 nucleotides to about 10,000 nucleotides, about 500 nucleotides to about 9,500 nucleotides, about 500 nucleotides to about 9,000 nucleotides, about 500 nucleotides to about 8,500 nucleotides, about 500 nucleotides to about 8,000 nucleotides, about 500 nucleotides to about 7,800 nucleotides, about 500 nucleotides to about 7,600 nucleotides, about 500 nucleotides to about 7,400 nucleotides, about 500 nucleotides to about 7,200 nucleotides, about 500 nucleotides to about 7,000 nucleotides, about 500 nucleotides to about 6,800 nucleotides, about 500 nucleotides to about 6,600 nucleotides, about 500 nucleotides to about 6,400 nucleotides, about 500 nucleotides to about 6,200 nucleotides, about 500 nucleotides to about 6,000 nucleotides, about 500 nucleotides to about 5,800 nucleotides, about 500 nucleotides to about 5,600 nucleotides, about 500 nucleotides to about 5,400 nucleotides, about 500 nucleotides to about 5,200 nucleotides, about 500 nucleotides to about 5,000 nucleotides, about 500 nucleotides to about 4,800 nucleotides, about 4,600 nucleotides, about 500 nucleotides to about 4,400 nucleotides, about 500 nucleotides to about 4,200 nucleotides, about 500 nucleotides to about 4,000 nucleotides, about 500 nucleotides to about 3,800 nucleotides, about 500 nucleotides to about 3,600 nucleotides, about 500 nucleotides to about 3,400 nucleotides, about 500 nucleotides to about 3,200 nucleotides, about 500 nucleotides to about 3,000 nucleotides, about 500 nucleotides to about 2,800 nucleotides, about 500 nucleotides to about 2,600 nucleotides, about 500 nucleotides to about 2,400 nucleotides, about 500 nucleotides to about 2,200 nucleotides, about 500 nucleotides to about 2,000 nucleotides, about 500 nucleotides to about 1,800 nucleotides, about 500 nucleotides to about 1,600 nucleotides, about 500 nucleotides to about 1,400 nucleotides, about 500 nucleotides to about 1,200 nucleotides, about 500 nucleotides to about 1,000 nucleotides, about 500 nucleotides to about 800 nucleotides, about 800 nucleotides to about 10,000 nucleotides, about 800 nucleotides to about 9,500 nucleotides, about 800 nucleotides to about 9,000 nucleotides, about 800 nucleotides to about 8,500 nucleotides, about 800 nucleotides to about 8,000 nucleotides, about 800 nucleotides to about 7,800 nucleotides, about 800 nucleotides to about 7,600 nucleotides, about 800 nucleotides to about 7,400 nucleotides, about 800 nucleotides to about 7,200 nucleotides, about 800 nucleotides to about 7,000 nucleotides, about 800 nucleotides to about 6,800 nucleotides, about 800 nucleotides to about 6,600 nucleotides, about 800 nucleotides to about 6,400 nucleotides, about 800 nucleotides to about 6,200 nucleotides, about 800 nucleotides to about 6,000 nucleotides, about 800 nucleotides to about 5,800 nucleotides, about 800 nucleotides to about 5,600 nucleotides, about 800 nucleotides to about 5,400 nucleotides, about 800 nucleotides to about 5,200 nucleotides, about 800 nucleotides to about 5,000 nucleotides, about 800 nucleotides to about 4,800 nucleotides, about 800 nucleotides to about 4,600 nucleotides, about 800 nucleotides to about 4,400 nucleotides, about 800 nucleotides to about 4,200 nucleotides, about 800 nucleotides to about 4,000 nucleotides, about 800 nucleotides to about 3,800 nucleotides, about 800 nucleotides to about 3,600 nucleotides, about 800 nucleotides to about 3,400 nucleotides, about 800 nucleotides to about 3,200 nucleotides, about 800 nucleotides to about 3,000 nucleotides, about 800 nucleotides to about 2,800 nucleotides, about 800 nucleotides to about 2,600 nucleotides, about 800 nucleotides to about 2,400 nucleotides, about 800 nucleotides to about 2,200 nucleotides, about 800 nucleotides to about 2,000 nucleotides, about 800 nucleotides to about 1,800 nucleotides, about 800 nucleotides to about 1,600 nucleotides, about 800 nucleotides to about 1,400 nucleotides, about 800 nucleotides to about 1,200 nucleotides, about 800 nucleotides to about 1,000 nucleotides, about 1,000 nucleotides to about 10,000 nucleotides, about 1,000 nucleotides to about 9,000 nucleotides, about 1,000 nucleotides to about 8,500 nucleotides, about 1,000 nucleotides to about 8,000 nucleotides, about 1,000 nucleotides to about 7,800 nucleotides, about 1,000 nucleotides to about 7,600 nucleotides, about 1,000 nucleotides to about 7,400 nucleotides, about 1,000 nucleotides to about 7,200 nucleotides, about 1,000 nucleotides to about 7,000 nucleotides, about 1,000 nucleotides to about 6,800 nucleotides, about 1,000 nucleotides to about 6,600 nucleotides, about 1,000 nucleotides to about 6,400 nucleotides, about 1,000 nucleotides to about 6,200 nucleotides, about 1,000 nucleotides to about 6,000 nucleotides, about 1,000 nucleotides to about 5,800 nucleotides, about 1,000 nucleotides to about 5,600 nucleotides, about 1,000 nucleotides to about 5,400 nucleotides, about 1,000 nucleotides to about 5,200 nucleotides, about 1,000 nucleotides to about 5,000 nucleotides, about 1,000 nucleotides to about 4,800 nucleotides, about 1,000 nucleotides to about 4,600 nucleotides, about 1,000 nucleotides to about 4,400 nucleotides, about 1,000 nucleotides to about 4,200 nucleotides, about 1,000 nucleotides to about 4,000 nucleotides, about 1,000 nucleotides to about 3,800 nucleotides, about 1,000 nucleotides to about 3,600 nucleotides, about 1,000 nucleotides to about 3,400 nucleotides, about 1,000 nucleotides to about 3,200 nucleotides, about 1,000 nucleotides to about 3,000 nucleotides, about 1,000 nucleotides to about 2,600 nucleotides, about 1,000 nucleotides to about 2,400 nucleotides, about 1,000 nucleotides to about 2,200 nucleotides, about 1,000 nucleotides to about 2,000 nucleotides, about 1,000 nucleotides to about 1,800 nucleotides, about 1,000 nucleotides to about 1,600 nucleotides, about 1,000 nucleotides to about 1,400 nucleotides, about 1,000 nucleotides to about 1,200 nucleotides, about 1,200 nucleotides to about 10,000 nucleotides, about 1,200 nucleotides to about 9,500 nucleotides, about 1,200 nucleotides to about 9,000 nucleotides, about 1,200 nucleotides to about 8,500 nucleotides, about 1,200 nucleotides to about 8,000 nucleotides, about 1,200 nucleotides to about 7,800 nucleotides, about 1,200 nucleotides to about 7,600 nucleotides, about 1,200 nucleotides to about 7,400 nucleotides, about 1,200 nucleotides to about 7,200 nucleotides, about 1,200 nucleotides to about 7,000 nucleotides, about 1,200 nucleotides to about 6,800 nucleotides, about 1,200 nucleotides to about 6,600 nucleotides, about 1,200 nucleotides to about 6,400 nucleotides, about 1,200 nucleotides to about 6,200 nucleotides, about 1,200 nucleotides to about 6,000 nucleotides, about 1,200 nucleotides to about 5,800 nucleotides, about 1,200 nucleotides to about 5,600 nucleotides, about 1,200 nucleotides to about 5,400 nucleotides, about 1,200 nucleotides to about 5,000 nucleotides, about 1,200 nucleotides to about 4,800 nucleotides, about 1,200 nucleotides to about 4,600 nucleotides, about 1,200 nucleotides to about 4,400 nucleotides, about 1,200 nucleotides to about 4,200 nucleotides, about 1,200 nucleotides to about 4,000 nucleotides, about 1,200 nucleotides to about 3,800 nucleotides, about 1,200 nucleotides to about 3,600 nucleotides, about 1,200 nucleotides to about 3,400 nucleotides, about 1,200 nucleotides to about 3,200 nucleotides, about 1,200 nucleotides to about 3,000 nucleotides, about 1,200 nucleotides to about 2,800 nucleotides, about 1,200 nucleotides to about 2,600 nucleotides, about 1,200 nucleotides to about 2,400 nucleotides, about 1,200 nucleotides to about 2,200 nucleotides, about 1,200 nucleotides to about 2,000 nucleotides, about 1,200 nucleotides to about 1,800 nucleotides, about 1,200 nucleotides to about 1,600 nucleotides, about 1,200 nucleotides to about 1,400 nucleotides, about 1,400 nucleotides to about 10,000 nucleotides, about 1,400 nucleotides to about 9,500 nucleotides, about 1,400 nucleotides to about 9,000 nucleotides, about 1,400 nucleotides to about 8,500 nucleotides, about 1,400 nucleotides to about 8,000 nucleotides, about 1,400 nucleotides to about 7,800 nucleotides, about 1,400 nucleotides to about 7,600 nucleotides, about 1,400 nucleotides to about 7,400 nucleotides, about 1,400 nucleotides to about 7,200 nucleotides, about 1,400 nucleotides to about 7,000 nucleotides, about 1,400 nucleotides to about 6,800 nucleotides, about 1,400 nucleotides to about 6,600 nucleotides, about 1,400 nucleotides to about 6,400 nucleotides, about 1,400 nucleotides to about 6,200 nucleotides, about 1,400 nucleotides to about 6,000 nucleotides, about 1,400 nucleotides to about 5,800 nucleotides, about 1,400 nucleotides to about 5,600 nucleotides, about 1,400 nucleotides to about 5,400 nucleotides, about 1,400 nucleotides to about 5,200 nucleotides, about 1,400 nucleotides to about 5,000 nucleotides, about 1,400 nucleotides to about 4,800 nucleotides, about 1,400 nucleotides to about 4,600 nucleotides, about 1,400 nucleotides to about 4,400 nucleotides, about 1,400 nucleotides to about 4,200 nucleotides, about 1,400 nucleotides to about 4,000 nucleotides, about 1,400 nucleotides to about 3,800 nucleotides, about 1,400 nucleotides to about 3,600 nucleotides, about 1,400 nucleotides to about 3,400 nucleotides, about 1,400 nucleotides to about 3,200 nucleotides, about 1,400 nucleotides to about 3,000 nucleotides, about 1,400 nucleotides to about 2,600 nucleotides, about 1,400 nucleotides to about 2,400 nucleotides, about 1,400 nucleotides to about 2,200 nucleotides, about 1,400 nucleotides to about 2,000 nucleotides, about 1,400 nucleotides to about 1,800 nucleotides, about 1,400 nucleotides to about 1,600 nucleotides, about 1,600 nucleotides to about 10,000 nucleotides, about 1,600 nucleotides to about 9,500 nucleotides, about 1,600 nucleotides to about 9,000 nucleotides, about 1,600 nucleotides to about 8,500 nucleotides, about 1,600 nucleotides to about 8,000 nucleotides, about 1,600 nucleotides to about 7,800 nucleotides, about 1,600 nucleotides to about 7,600 nucleotides, about 1,600 nucleotides to about 7,400 nucleotides, about 1,600 nucleotides to about 7,200 nucleotides, about 1,600 nucleotides to about 7,000 nucleotides, about 1,600 nucleotides to about 6,800 nucleotides, about 1,600 nucleotides to about 6,400 nucleotides, about 1,600 nucleotides to about 6,200 nucleotides, about 1,600 nucleotides to about 6,000 nucleotides, about 1,600 nucleotides to about 5,800 nucleotides, about 1,600 nucleotides to about 5,600 nucleotides, about 1,600 nucleotides to about 5,400 nucleotides, about 1,600 nucleotides to about 5,200 nucleotides, about 1,600 nucleotides to about 5,000 nucleotides, about 1,600 nucleotides to about 4,800 nucleotides, about 1,600 nucleotides to about 4,600 nucleotides, about 1,600 nucleotides to about 4,400 nucleotides, about 1,600 nucleotides to about 4,200 nucleotides, about 1,600 nucleotides to about 4,000 nucleotides, about 1,600 nucleotides to about 3,800 nucleotides, about 1,600 nucleotides to about 3,600 nucleotides, about 1,600 nucleotides to about 3,400 nucleotides, about 1,600 nucleotides to about 3,200 nucleotides, about 1,600 nucleotides to about 3,000 nucleotides, about 1,600 nucleotides to about 2,800 nucleotides, about 1,600 nucleotides to about 2,600 nucleotides, about 1,600 nucleotides to about 2,400 nucleotides, about 1,600 nucleotides to about 2,200 nucleotides, about 1,600 nucleotides to about 2,000 nucleotides, about 1,600 nucleotides to about 1,800 nucleotides, about 1,800 nucleotides to about 10,000 nucleotides, about 1,800 nucleotides to about 9,500 nucleotides, about 1,800 nucleotides to about 9,000 nucleotides, about 1,800 nucleotides to about 8,500 nucleotides, about 1,800 nucleotides to about 8,000 nucleotides, about 1,800 nucleotides to about 7,800 nucleotides, about 1,800 nucleotides to about 7,600 nucleotides, about 1,800 nucleotides to about 7,400 nucleotides, about 1,800 nucleotides to about 7,200 nucleotides, about 1,800 nucleotides to about 7,000 nucleotides, about 1,800 nucleotides to about 6,800 nucleotides, about 1,800 nucleotides to about 6,600 nucleotides, about 1,800 nucleotides to about 6,400 nucleotides, about 1,800 nucleotides to about 6,200 nucleotides, about 1,800 nucleotides to about 6,000 nucleotides, about 1,800 nucleotides to about 5,800 nucleotides, about 1,800 nucleotides to about 5,600 nucleotides, about 1,800 nucleotides to about 5,400 nucleotides, about 1,800 nucleotides to about 5,200 nucleotides, about 1,800 nucleotides to about 5,000 nucleotides, about 1,800 nucleotides to about 4,800 nucleotides, about 1,800 nucleotides to about 4,600 nucleotides, about 1,800 nucleotides to about 4,400 nucleotides, about 1,800 nucleotides to about 4,200 nucleotides, about 1,800 nucleotides to about 4,000 nucleotides, about 1,800 nucleotides to about 3,800 nucleotides, about 1,800 nucleotides to about 3,600 nucleotides, about 1,800 nucleotides to about 3,400 nucleotides, about 1,800 nucleotides to about 3,200 nucleotides, about 1,800 nucleotides to about 3,000 nucleotides, about 1,800 nucleotides to about 2,800 nucleotides, about 1,800 nucleotides to about 2,600 nucleotides, about 1,800 nucleotides to about 2,400 nucleotides, about 1,800 nucleotides to about 2,200 nucleotides, about 1,800 nucleotides to about 2,000 nucleotides, about 2,000 nucleotides to about 10,000 nucleotides, about 2,000 nucleotides to about 9,500 nucleotides, about 2,000 nucleotides to about 9,000 nucleotides, about 2,000 nucleotides to about 8,500 nucleotides, about 2,000 nucleotides to about 8,000 nucleotides, about 2,000 nucleotides to about 7,800 nucleotides, about 2,000 nucleotides to about 7,600 nucleotides, about 2,000 nucleotides to about 7,400 nucleotides, about 2,000 nucleotides to about 7,200 nucleotides, about 2,000 nucleotides to about 7,000 nucleotides, about 2,000 nucleotides to about 6,800 nucleotides, about 2,000 nucleotides to about 6,600 nucleotides, about 2,000 nucleotides to about 6,400 nucleotides, about 2,000 nucleotides to about 6,200 nucleotides, about 2,000 nucleotides to about 6,000 nucleotides, about 2,000 nucleotides to about 5,800 nucleotides, about 2,000 nucleotides to about 5,600 nucleotides, about 2,000 nucleotides to about 5,400 nucleotides, about 2,000 nucleotides to about 5,200 nucleotides, about 2,000 nucleotides to about 5,000 nucleotides, about 2,000 nucleotides to about 4,800 nucleotides, about 2,000 nucleotides to about 4,600 nucleotides, about 2,000 nucleotides to about 4,400 nucleotides, about 2,000 nucleotides to about 4,200 nucleotides, about 2,000 nucleotides to about 4,000 nucleotides, about 2,000 nucleotides to about 3,800 nucleotides, about 2,000 nucleotides to about 3,600 nucleotides, about 2,000 nucleotides to about 3,400 nucleotides, about 2,000 nucleotides to about 3,200 nucleotides, about 2,000 nucleotides to about 3,000 nucleotides, about 2,000 nucleotides to about 2,800 nucleotides, about 2,000 nucleotides to about 2,600 nucleotides, about 2,000 nucleotides to about 2,400 nucleotides, about 2,000 nucleotides to about 2,200 nucleotides, about 2,200 nucleotides to about 10,000 nucleotides, about 9,500 nucleotides, about 9,000 nucleotides, about 8,500 nucleotides, about 8,000 nucleotides, about 7,800 nucleotides, about 7,600 nucleotides, about 7,400 nucleotides, about 7,200 nucleotides, about 7,000 nucleotides, about 6,800 nucleotides, about 6,600 nucleotides, about 6,400 nucleotides, about 6,200 nucleotides, about 6,000 nucleotides, about 5,800 nucleotides, about 5,600 nucleotides, about 5,400 nucleotides, about 5,200 nucleotides, about 5,000 nucleotides, about 4,800 nucleotides, about 4,600 nucleotides, about 4,400 nucleotides, about 4,200 nucleotides, about 4,000 nucleotides, about 3,800 nucleotides, about 3,600 nucleotides, about 3,400 nucleotides, about 3,200 nucleotides, about 3,000 nucleotides, about 2,800 nucleotides, about 2,600 nucleotides, about 2,400 nucleotides, about 2,400 nucleotides to about 10,000 nucleotides, about 2,400 nucleotides to about 9,500 nucleotides, about 2,400 nucleotides to about 9,000 nucleotides, about 2,400 nucleotides to about 8,500 nucleotides, about 2,400 nucleotides to about 8,000 nucleotides, about 2,400 nucleotides to about 7,800 nucleotides, about 2,400 nucleotides to about 7,600 nucleotides, about 2,400 nucleotides to about 7,400 nucleotides, about 2,400 nucleotides to about 7,200 nucleotides, about 2,400 nucleotides to about 7,000 nucleotides, about 2,400 nucleotides to about 6,800 nucleotides, about 2,400 nucleotides to about 6,600 nucleotides, about 2,400 nucleotides to about 6,400 nucleotides, about 2,400 nucleotides to about 6,200 nucleotides, about 2,400 nucleotides to about 6,000 nucleotides, about 2,400 nucleotides to about 5,800 nucleotides, about 2,400 nucleotides to about 5,600 nucleotides, about 2,400 nucleotides to about 5,400 nucleotides, about 2,400 nucleotides to about 5,200 nucleotides, about 2,400 nucleotides to about 5,000 nucleotides, about 2,400 nucleotides to about 4,800 nucleotides, about 2,400 nucleotides to about 4,600 nucleotides, about 2,400 nucleotides to about 4,400 nucleotides, about 2,400 nucleotides to about 4,200 nucleotides, about 2,400 nucleotides to about 4,000 nucleotides, about 2,400 nucleotides to about 3,800 nucleotides, about 2,400 nucleotides to about 3,600 nucleotides, about 2,400 nucleotides to about 3,400 nucleotides, about 2,400 nucleotides to about 3,200 nucleotides, about 2,400 nucleotides to about 3,000 nucleotides, about 2,400 nucleotides to about 2,800 nucleotides, about 2,400 nucleotides to about 2,600 nucleotides, about 2,600 nucleotides to about 10,000 nucleotides, about 2,600 nucleotides to about 9,500 nucleotides, about 2,600 nucleotides to about 9,000 nucleotides, about 2,600 nucleotides to about 8,500 nucleotides, about 2,600 nucleotides to about 8,000 nucleotides, about 2,600 nucleotides to about 7,800 nucleotides, about 2,600 nucleotides to about 7,600 nucleotides, about 2,600 nucleotides to about 7,400 nucleotides, about 2,600 nucleotides to about 7,200 nucleotides, about 2,600 nucleotides to about 7,000 nucleotides, about 2,600 nucleotides to about 6,800 nucleotides, about 2,600 nucleotides to about 6,600 nucleotides, about 2,600 nucleotides to about 6,400 nucleotides, about 2,600 nucleotides to about 6,200 nucleotides, about 2,600 nucleotides to about 6,000 nucleotides, about 2,600 nucleotides to about 5,800 nucleotides, about 2,600 nucleotides to about 5,600 nucleotides, about 2,600 nucleotides to about 5,400 nucleotides, about 2,600 nucleotides to about 5,200 nucleotides, about 2,600 nucleotides to about 5,000 nucleotides, about 2,600 nucleotides to about 4,800 nucleotides, about 2,600 nucleotides to about 4,600 nucleotides, about 2,600 nucleotides to about 4,400 nucleotides, about 2,600 nucleotides to about 4,200 nucleotides, about 2,600 nucleotides to about 4,000 nucleotides, about 2,600 nucleotides to about 3,800 nucleotides, about 2,600 nucleotides to about 3,600 nucleotides, about 2,600 nucleotides to about 3,400 nucleotides, about 2,600 nucleotides to about 3,200 nucleotides, about 2,600 nucleotides to about 3,000 nucleotides, about 2,600 nucleotides to about 2,800 nucleotides, about 2,800 nucleotides to about 10,000 nucleotides, about 2,800 nucleotides to about 9,500 nucleotides, about 2,800 nucleotides to about 9,000 nucleotides, about 2,800 nucleotides to about 8,500 nucleotides, about 2,800 nucleotides to about 8,000 nucleotides, about 2,800 nucleotides to about 7,800 nucleotides, about 2,800 nucleotides to about 7,600 nucleotides, about 2,800 nucleotides to about 7,400 nucleotides, about 2,800 nucleotides to about 7,200 nucleotides, about 2,800 nucleotides to about 7,000 nucleotides, about 2,800 nucleotides to about 6,800 nucleotides, about 2,800 nucleotides to about 6,600 nucleotides, about 2,800 nucleotides to about 6,400 nucleotides, about 2,800 nucleotides to about 6,200 nucleotides, about 2,800 nucleotides to about 6,000 nucleotides, about 2,800 nucleotides to about 5,800 nucleotides, about 2,800 nucleotides to about 5,600 nucleotides, about 2,800 nucleotides to about 5,400 nucleotides, about 2,800 nucleotides to about 5,200 nucleotides, about 2,800 nucleotides to about 5,000 nucleotides, about 2,800 nucleotides to about 4,800 nucleotides, about 2,800 nucleotides to about 4,600 nucleotides, about 2,800 nucleotides to about 4,400 nucleotides, about 2,800 nucleotides to about 4,200 nucleotides, about 2,800 nucleotides to about 4,000 nucleotides, about 2,800 nucleotides to about 3,800 nucleotides, about 2,800 nucleotides to about 3,600 nucleotides, about 2,800 nucleotides to about 3,400 nucleotides, about 2,800 nucleotides to about 3,200 nucleotides, about 2,800 nucleotides to about 3,000 nucleotides, about 3,000 nucleotides to about 10,000 nucleotides, about 3,000 nucleotides to about 9,500 nucleotides, about 3,000 nucleotides to about 9,000 nucleotides, about 3,000 nucleotides to about 8,500 nucleotides, about 3,000 nucleotides to about 8,000 nucleotides, about 3,000 nucleotides to about 7,800 nucleotides, about 3,000 nucleotides to about 7,600 nucleotides, about 3,000 nucleotides to about 7,400 nucleotides, about 3,000 nucleotides to about 7,200 nucleotides, about 3,000 nucleotides to about 7,000 nucleotides, about 3,000 nucleotides to about 6,800 nucleotides, about 3,000 nucleotides to about 6,600 nucleotides, about 3,000 nucleotides to about 6,400 nucleotides, about 3,000 nucleotides to about 6,200 nucleotides, about 3,000 nucleotides to about 6,000 nucleotides, about 3,000 nucleotides to about 5,800 nucleotides, about 3,000 nucleotides to about 5,600 nucleotides, about 3,000 nucleotides to about 5,400 nucleotides, about 3,000 nucleotides to about 5,200 nucleotides, about 3,000 nucleotides to about 5,000 nucleotides, about 3,000 nucleotides to about 4,800 nucleotides, about 3,000 nucleotides to about 4,600 nucleotides, about 3,000 nucleotides to about 4,400 nucleotides, about 3,000 nucleotides to about 4,200 nucleotides, about 3,000 nucleotides to about 4,000 nucleotides, about 3,000 nucleotides to about 3,800 nucleotides, about 3,000 nucleotides to about 3,600 nucleotides, about 3,000 nucleotides to about 3,400 nucleotides, about 3,000 nucleotides to about 3,200 nucleotides, about 3,200 nucleotides to about 10,000 nucleotides, about 3,200 nucleotides to about 9,500 nucleotides, about 3,200 nucleotides to about 9,000 nucleotides, about 3,200 nucleotides to about 8,500 nucleotides, about 3,200 nucleotides to about 8,000 nucleotides, about 3,200 nucleotides to about 7,800 nucleotides, about 3,200 nucleotides to about 7,600 nucleotides, about 3,200 nucleotides to about 7,400 nucleotides, about 3,200 nucleotides to about 7,200 nucleotides, about 3,200 nucleotides to about 7,000 nucleotides, about 3,200 nucleotides to about 6,800 nucleotides, about 3,200 nucleotides to about 6,600 nucleotides, about 3,200 nucleotides to about 6,400 nucleotides, about 3,200 nucleotides to about 6,200 nucleotides, about 3,200 nucleotides to about 6,000 nucleotides, about 3,200 nucleotides to about 5,800 nucleotides, about 3,200 nucleotides to about 5,600 nucleotides, about 3,200 nucleotides to about 5,400 nucleotides, about 3,200 nucleotides to about 5,200 nucleotides, about 3,200 nucleotides to about 5,000 nucleotides, about 3,200 nucleotides to about 4,800 nucleotides, about 3,200 nucleotides to about 4,600 nucleotides, about 3,200 nucleotides to about 4,400 nucleotides, about 3,200 nucleotides to about 4,200 nucleotides, about 3,200 nucleotides to about 4,000 nucleotides, about 3,200 nucleotides to about 3,800 nucleotides, about 3,200 nucleotides to about 3,600 nucleotides, about 3,200 nucleotides to about 3,400 nucleotides, about 3,400 nucleotides to about 10,000 nucleotides, about 3,400 nucleotides to about 9,500 nucleotides, about 3,400 nucleotides to about 9,000 nucleotides, about 3,400 nucleotides to about 8,500 nucleotides, about 3,400 nucleotides to about 8,000 nucleotides, about 3,400 nucleotides to about 7,800 nucleotides, about 3,400 nucleotides to about 7,600 nucleotides, about 3,400 nucleotides to about 7,400 nucleotides, about 3,400 nucleotides to about 7,200 nucleotides, about 3,400 nucleotides to about 7,000 nucleotides, about 3,400 nucleotides to about 6,800 nucleotides, about 3,400 nucleotides to about 6,600 nucleotides, about 3,400 nucleotides to about 6,400 nucleotides, about 3,400 nucleotides to about 6,200 nucleotides, about 3,400 nucleotides to about 6,000 nucleotides, about 3,400 nucleotides to about 5,800 nucleotides, about 3,400 nucleotides to about 5,600 nucleotides, about 3,400 nucleotides to about 5,400 nucleotides, about 3,400 nucleotides to about 5,200 nucleotides, about 3,400 nucleotides to about 5,000 nucleotides, about 3,400 nucleotides to about 4,800 nucleotides, about 3,400 nucleotides to about 4,600 nucleotides, about 3,400 nucleotides to about 4,400 nucleotides, about 3,400 nucleotides to about 4,200 nucleotides, about 3,400 nucleotides to about 4,000 nucleotides, about 3,400 nucleotides to about 3,800 nucleotides, about 3,400 nucleotides to about 3,600 nucleotides, about 3,600 nucleotides to about 10,000 nucleotides, about 3,600 nucleotides to about 9,500 nucleotides, about 3,600 nucleotides to about 9,000 nucleotides, about 3,600 nucleotides to about 8,500 nucleotides, about 3,600 nucleotides to about 8,000 nucleotides, about 3,600 nucleotides to about 7,800 nucleotides, about 3,600 nucleotides to about 7,600 nucleotides, about 3,600 nucleotides to about 7,400 nucleotides, about 3,600 nucleotides to about 7,200 nucleotides, about 3,600 nucleotides to about 7,000 nucleotides, about 3,600 nucleotides to about 6,800 nucleotides, about 3,600 nucleotides to about 6,600 nucleotides, about 3,600 nucleotides to about 6,400 nucleotides, about 3,600 nucleotides to about 6,200 nucleotides, about 3,600 nucleotides to about 6,000 nucleotides, about 3,600 nucleotides to about 5,800 nucleotides, about 3,600 nucleotides to about 5,600 nucleotides, about 3,600 nucleotides to about 5,400 nucleotides, about 3,600 nucleotides to about 5,200 nucleotides, about 3,600 nucleotides to about 5,000 nucleotides, about 3,600 nucleotides to about 4,800 nucleotides, about 3,600 nucleotides to about 4,600 nucleotides, about 3,600 nucleotides to about 4,400 nucleotides, about 3,600 nucleotides to about 4,200 nucleotides, about 3,600 nucleotides to about 4,000 nucleotides, about 3,600 nucleotides to about 3,800 nucleotides, about 3,800 nucleotides to about 10,000 nucleotides, about 3,800 nucleotides to about 9,500 nucleotides, about 3,800 nucleotides to about 9,000 nucleotides, about 3,800 nucleotides to about 8,500 nucleotides, about 3,800 nucleotides to about 8,000 nucleotides, about 3,800 nucleotides to about 7,800 nucleotides, about 3,800 nucleotides to about 7,600 nucleotides, about 3,800 nucleotides to about 7,400 nucleotides, about 3,800 nucleotides to about 7,200 nucleotides, about 3,800 nucleotides to about 7,000 nucleotides, about 3,800 nucleotides to about 6,800 nucleotides, about 3,800 nucleotides to about 6,600 nucleotides, about 3,800 nucleotides to about 6,400 nucleotides, about 3,800 nucleotides to about 6,200 nucleotides, about 3,800 nucleotides to about 6,000 nucleotides, about 3,800 nucleotides to about 5,800 nucleotides, about 3,800 nucleotides to about 5,600 nucleotides, about 3,800 nucleotides to about 5,400 nucleotides, about 3,800 nucleotides to about 5,200 nucleotides, about 3,800 nucleotides to about 5,000 nucleotides, about 3,800 nucleotides to about 4,800 nucleotides, about 3,800 nucleotides to about 4,600 nucleotides, about 3,800 nucleotides to about 4,200 nucleotides, about 3,800 nucleotides to about 4,000 nucleotides, about 4,000 nucleotides to about 10,000 nucleotides, about 4,000 nucleotides to about 9,500 nucleotides, about 4,000 nucleotides to about 9,000 nucleotides, about 4,000 nucleotides to about 8,500 nucleotides, about 4,000 nucleotides to about 8,000 nucleotides, about 4,000 nucleotides to about 7,800 nucleotides, about 4,000 nucleotides to about 7,600 nucleotides, about 4,000 nucleotides to about 7,400 nucleotides, about 4,000 nucleotides to about 7,200 nucleotides, about 4,000 nucleotides to about 7,000 nucleotides, about 4,000 nucleotides to about 6,800 nucleotides, about 4,000 nucleotides to about 6,600 nucleotides, about 4,000 nucleotides to about 6,400 nucleotides, about 4,000 nucleotides to about 6,200 nucleotides, about 4,000 nucleotides to about 6,000 nucleotides, about 4,000 nucleotides to about 5,800 nucleotides, about 4,000 nucleotides to about 5,600 nucleotides, about 4,000 nucleotides to about 5,400 nucleotides, about 4,000 nucleotides to about 5,200 nucleotides, about 4,000 nucleotides to about 5,000 nucleotides, about 4,000 nucleotides to about 4,800 nucleotides, about 4,000 nucleotides to about 4,600 nucleotides, about 4,000 nucleotides to about 4,400 nucleotides, about 4,000 nucleotides to about 4,200 nucleotides, about 4,200 nucleotides to about 10,000 nucleotides, about 4,200 nucleotides to about 9,500 nucleotides, about 4,200 nucleotides to about 9,000 nucleotides, about 4,200 nucleotides to about 8,500 nucleotides, about 4,200 nucleotides to about 8,000 nucleotides, about 4,200 nucleotides to about 7,800 nucleotides, about 4,200 nucleotides to about 7,600 nucleotides, about 4,200 nucleotides to about 7,400 nucleotides, about 4,200 nucleotides to about 7,200 nucleotides, about 4,200 nucleotides to about 7,000 nucleotides, about 4,200 nucleotides to about 6,800 nucleotides, about 4,200 nucleotides to about 6,600 nucleotides, about 4,200 nucleotides to about 6,400 nucleotides, about 4,200 nucleotides to about 6,200 nucleotides, about 4,200 nucleotides to about 6,000 nucleotides, about 4,200 nucleotides to about 5,800 nucleotides, about 4,200 nucleotides to about 5,600 nucleotides, about 4,200 nucleotides to about 5,400 nucleotides, about 4,200 nucleotides to about 5,200 nucleotides, about 4,200 nucleotides to about 5,000 nucleotides, about 4,200 nucleotides to about 4,800 nucleotides, about 4,200 nucleotides to about 4,600 nucleotides, about 4,200 nucleotides to about 4,400 nucleotides, about 4,400 nucleotides to about 10,000 nucleotides, about 4,400 nucleotides to about 9,500 nucleotides, about 4,400 nucleotides to about 9,000 nucleotides, about 4,400 nucleotides to about 8,500 nucleotides, about 4,400 nucleotides to about 8,000 nucleotides, about 4,400 nucleotides to about 7,800 nucleotides, about 4,400 nucleotides to about 7,600 nucleotides, about 4,400 nucleotides to about 7,400 nucleotides, about 4,400 nucleotides to about 7,200 nucleotides, about 4,400 nucleotides to about 7,000 nucleotides, about 4,400 nucleotides to about 6,800 nucleotides, about 4,400 nucleotides to about 6,600 nucleotides, about 4,400 nucleotides to about 6,400 nucleotides, about 4,400 nucleotides to about 6,200 nucleotides, about 4,400 nucleotides to about 6,000 nucleotides, about 4,400 nucleotides to about 5,800 nucleotides, about 4,400 nucleotides to about 5,600 nucleotides, about 4,400 nucleotides to about 5,400 nucleotides, about 4,400 nucleotides to about 5,200 nucleotides, about 4,400 nucleotides to about 5,000 nucleotides, about 4,400 nucleotides to about 4,800 nucleotides, about 4,400 nucleotides to about 4,600 nucleotides, about 4,600 nucleotides to about 10,000 nucleotides, about 4,600 nucleotides to about 9,500 nucleotides, about 4,600 nucleotides to about 9,000 nucleotides, about 4,600 nucleotides to about 8,500 nucleotides, about 4,600 nucleotides to about 8,000 nucleotides, about 4,600 nucleotides to about 7,800 nucleotides, about 4,600 nucleotides to about 7,600 nucleotides, about 4,600 nucleotides to about 7,400 nucleotides, about 4,600 nucleotides to about 7,200 nucleotides, about 4,600 nucleotides to about 7,000 nucleotides, about 4,600 nucleotides to about 6,800 nucleotides, about 4,600 nucleotides to about 6,600 nucleotides, about 4,600 nucleotides to about 6,400 nucleotides, about 4,600 nucleotides to about 6,200 nucleotides, about 4,600 nucleotides to about 6,000 nucleotides, about 4,600 nucleotides to about 5,800 nucleotides, about 4,600 nucleotides to about 5,600 nucleotides, about 4,600 nucleotides to about 5,400 nucleotides, about 4,600 nucleotides to about 5,200 nucleotides, about 4,600 nucleotides to about 5,000 nucleotides, about 4,600 nucleotides to about 4,800 nucleotides, about 4,800 nucleotides to about 10,000 nucleotides, about 4,800 nucleotides to about 9,500 nucleotides, about 4,800 nucleotides to about 9,000 nucleotides, about 4,800 nucleotides to about 8,500 nucleotides, about 4,800 nucleotides to about 8,000 nucleotides, about 4,800 nucleotides to about 7,800 nucleotides, about 4,800 nucleotides to about 7,600 nucleotides, about 4,800 nucleotides to about 7,400 nucleotides, about 4,800 nucleotides to about 7,200 nucleotides, about 4,800 nucleotides to about 7,000 nucleotides, about 4,800 nucleotides to about 6,800 nucleotides, about 4,800 nucleotides to about 6,600 nucleotides, about 4,800 nucleotides to about 6,400 nucleotides, about 4,800 nucleotides to about 6,200 nucleotides, about 4,800 nucleotides to about 6,000 nucleotides, about 4,800 nucleotides to about 5,800 nucleotides, about 4,800 nucleotides to about 5,600 nucleotides, about 4,800 nucleotides to about 5,400 nucleotides, about 4,800 nucleotides to about 5,200 nucleotides, about 4,800 nucleotides to about 5,000 nucleotides, about 5,000 nucleotides to about 10,000 nucleotides, about 5,000 nucleotides to about 9,500 nucleotides, about 5,000 nucleotides to about 9,000 nucleotides, about 5,000 nucleotides to about 8,500 nucleotides, about 5,000 nucleotides to about 8,000 nucleotides, about 5,000 nucleotides to about 7,800 nucleotides, about 5,000 nucleotides to about 7,600 nucleotides, about 5,000 nucleotides to about 7,400 nucleotides, about 5,000 nucleotides to about 7,200 nucleotides, about 5,000 nucleotides to about 7,000 nucleotides, about 5,000 nucleotides to about 6,800 nucleotides, about 5,000 nucleotides to about 6,600 nucleotides, about 5,000 nucleotides to about 6,400 nucleotides, about 5,000 nucleotides to about 6,200 nucleotides, about 5,000 nucleotides to about 6,000 nucleotides, about 5,000 nucleotides to about 5,800 nucleotides, about 5,000 nucleotides to about 5,600 nucleotides, about 5,000 nucleotides to about 5,400 nucleotides, about 5,000 nucleotides to about 5,200 nucleotides, about 5,200 nucleotides to about 10,000 nucleotides, about 5,200 nucleotides to about 9,500 nucleotides, about 5,200 nucleotides to about 9,000 nucleotides, about 5,200 nucleotides to about 8,500 nucleotides, about 5,200 nucleotides to about 8,000 nucleotides, about 5,200 nucleotides to about 7,800 nucleotides, about 5,200 nucleotides to about 7,600 nucleotides, about 5,200 nucleotides to about 7,400 nucleotides, about 5,200 nucleotides to about 7,200 nucleotides, about 5,200 nucleotides to about 7,000 nucleotides, about 5,200 nucleotides to about 6,800 nucleotides, about 5,200 nucleotides to about 6,600 nucleotides, about 5,200 nucleotides to about 6,400 nucleotides, about 5,200 nucleotides to about 6,200 nucleotides, about 5,200 nucleotides to about 6,000 nucleotides, about 5,200 nucleotides to about 5,800 nucleotides, about 5,200 nucleotides to about 5,600 nucleotides, about 5,200 nucleotides to about 5,400 nucleotides, about 5,400 nucleotides to about 10,000 nucleotides, about 5,400 nucleotides to about 9,500 nucleotides, about 5,400 nucleotides to about 9,000 nucleotides, about 5,400 nucleotides to about 8,500 nucleotides, about 5,400 nucleotides to about 8,000 nucleotides, about 5,400 nucleotides to about 7,800 nucleotides, about 5,400 nucleotides to about 7,600 nucleotides, about 5,400 nucleotides to about 7,400 nucleotides, about 5,400 nucleotides to about 7,200 nucleotides, about 5,400 nucleotides to about 7,000 nucleotides, about 5,400 nucleotides to about 6,800 nucleotides, about 5,400 nucleotides to about 6,600 nucleotides, about 5,400 nucleotides to about 6,400 nucleotides, about 5,400 nucleotides to about 6,200 nucleotides, about 5,400 nucleotides to about 6,000 nucleotides, about 5,400 nucleotides to about 5,800 nucleotides, about 5,400 nucleotides to about 5,600 nucleotides, about 5,600 nucleotides to about 10,000 nucleotides, about 5,600 nucleotides to about 9,500 nucleotides, about 5,600 nucleotides to about 9,000 nucleotides, about 5,600 nucleotides to about 8,500 nucleotides, about 5,600 nucleotides to about 8,000 nucleotides, about 5,600 nucleotides to about 7,800 nucleotides, about 5,600 nucleotides to about 7,600 nucleotides, about 5,600 nucleotides to about 7,400 nucleotides, about 5,600 nucleotides to about 7,200 nucleotides, about 5,600 nucleotides to about 7,000 nucleotides, about 5,600 nucleotides to about 6,800 nucleotides, about 5,600 nucleotides to about 6,600 nucleotides, about 5,600 nucleotides to about 6,400 nucleotides, about 5,600 nucleotides to about 6,200 nucleotides, about 5,600 nucleotides to about 6,000 nucleotides, about 5,600 nucleotides to about 5,800 nucleotides, about 5,800 nucleotides to about 10,000 nucleotides, about 5,800 nucleotides to about 9,500 nucleotides, about 5,800 nucleotides to about 9,000 nucleotides, about 5,800 nucleotides to about 8,500 nucleotides, about 5,800 nucleotides to about 8,000 nucleotides, about 5,800 nucleotides to about 7,800 nucleotides, about 5,800 nucleotides to about 7,600 nucleotides, about 5,800 nucleotides to about 7,400 nucleotides, about 5,800 nucleotides to about 7,200 nucleotides, about 5,800 nucleotides to about 7,000 nucleotides, about 5,800 nucleotides to about 6,800 nucleotides, about 5,800 nucleotides to about 6,600 nucleotides, about 5,800 nucleotides to about 6,400 nucleotides, about 5,800 nucleotides to about 6,200 nucleotides, about 5,800 nucleotides to about 6,000 nucleotides, about 6,000 nucleotides to about 10,000 nucleotides, about 6,000 nucleotides to about 9,500 nucleotides, about 6,000 nucleotides to about 9,000 nucleotides, about 6,000 nucleotides to about 8,500 nucleotides, about 6,000 nucleotides to about 8,000 nucleotides, about 6,000 nucleotides to about 7,800 nucleotides, about 6,000 nucleotides to about 7,600 nucleotides, about 6,000 nucleotides to about 7,400 nucleotides, about 6,000 nucleotides to about 7,200 nucleotides, about 6,000 nucleotides to about 7,000 nucleotides, about 6,000 nucleotides to about 6,800 nucleotides, about 6,000 nucleotides to about 6,600 nucleotides, about 6,000 nucleotides to about 6,400 nucleotides, about 6,000 nucleotides to about 6,200 nucleotides, about 6,200 nucleotides to about 10,000 nucleotides, about 6,200 nucleotides to about 9,000 nucleotides, about 6,200 nucleotides to about 8,500 nucleotides, about 6,200 nucleotides to about 8,000 nucleotides, about 6,200 nucleotides to about 7,800 nucleotides, about 6,200 nucleotides to about 7,600 nucleotides, about 6,200 nucleotides to about 7,400 nucleotides, about 6,200 nucleotides to about 7,200 nucleotides, about 6,200 nucleotides to about 7,000 nucleotides, about 6,200 nucleotides to about 6,800 nucleotides, about 6,200 nucleotides to about 6,600 nucleotides, about 6,200 nucleotides to about 6,400 nucleotides, about 6,400 nucleotides to about 10,000 nucleotides, about 6,400 nucleotides to about 9,500 nucleotides, about 6,400 nucleotides to about 9,000 nucleotides, about 6,400 nucleotides to about 8,500 nucleotides, about 6,400 nucleotides to about 8,000 nucleotides, about 6,400 nucleotides to about 7,800 nucleotides, about 6,400 nucleotides to about 7,600 nucleotides, about 6,400 nucleotides to about 7,400 nucleotides, about 6,400 nucleotides to about 7,200 nucleotides, about 6,400 nucleotides to about 7,000 nucleotides, about 6,400 nucleotides to about 6,800 nucleotides, about 6,400 nucleotides to about 6,600 nucleotides, about 6,600 nucleotides to about 10,000 nucleotides, about 6,600 nucleotides to about 9,500 nucleotides, about 6,600 nucleotides to about 9,000 nucleotides, about 6,600 nucleotides to about 8,500 nucleotides, about 6,600 nucleotides to about 8,000 nucleotides, about 6,600 nucleotides to about 7,800 nucleotides, about 6,600 nucleotides to about 7,600 nucleotides, about 6,600 nucleotides to about 7,400 nucleotides, about 6,600 nucleotides to about 7,200 nucleotides, about 6,600 nucleotides to about 7,000 nucleotides, about 6,600 nucleotides to about 6,800 nucleotides, about 6,800 nucleotides to about 10,000 nucleotides, about 6,800 nucleotides to about 9,500 nucleotides, about 6,800 nucleotides to about 9,000 nucleotides, about 6,800 nucleotides to about 8,500 nucleotides, about 6,800 nucleotides to about 8,000 nucleotides, about 6,800 nucleotides to about 7,800 nucleotides, about 6,800 nucleotides to about 7,600 nucleotides, about 6,800 nucleotides to about 7,400 nucleotides, about 6,800 nucleotides to about 7,200 nucleotides, about 6,800 nucleotides to about 7,000 nucleotides, about 7,000 nucleotides to about 10,000 nucleotides, about 7,000 nucleotides to about 9,500 nucleotides, about 7,000 nucleotides to about 9,000 nucleotides, about 7,000 nucleotides to about 8,500 nucleotides, about 7,000 nucleotides to about 8,000 nucleotides, about 7,000 nucleotides to about 7,800 nucleotides, about 7,000 nucleotides to about 7,600 nucleotides, about 7,000 nucleotides to about 7,400 nucleotides, about 7,000 nucleotides to about 7,200 nucleotides, about 7,200 nucleotides to about 10,000 nucleotides, about 7,200 nucleotides to about 9,500 nucleotides, about 7,200 nucleotides to about 9,000 nucleotides, about 7,200 nucleotides to about 8,500 nucleotides, about 7,200 nucleotides to about 8,000 nucleotides, about 7,200 nucleotides to about 7,800 nucleotides, about 7,200 nucleotides to about 7,600 nucleotides, about 7,200 nucleotides to about 7,400 nucleotides, about 7,400 nucleotides to about 10,000 nucleotides, about 7,400 nucleotides to about 9,500 nucleotides, about 7,400 nucleotides to about 9,000 nucleotides, about 7,400 nucleotides to about 8,500 nucleotides, about 7,400 nucleotides to about 8,000 nucleotides, about 7,400 nucleotides to about 7,800 nucleotides, about 7,400 nucleotides to about 7,600 nucleotides, about 7,600 nucleotides to about 10,000 nucleotides, about 7,600 nucleotides to about 9,500 nucleotides, about 7,600 nucleotides to about 9,000 nucleotides, about 7,600 nucleotides to about 8,500 nucleotides, about 7,600 nucleotides to about 8,000 nucleotides, about 7,600 nucleotides to about 7,800 nucleotides, about 7,800 nucleotides to about 10,000 nucleotides, about 7,800 nucleotides to about 9,500 nucleotides, about 7,800 nucleotides to about 9,000 nucleotides, about 7,800 nucleotides to about 8,500 nucleotides, about 7,800 nucleotides to about 8,000 nucleotides, about 8,000 nucleotides to about 10,000 nucleotides, about 8,000 nucleotides to about 9,500 nucleotides, about 8,000 nucleotides to about 9,000 nucleotides, about 8,000 nucleotides to about 8,500 nucleotides, about 8,500 nucleotides to about 10,000 nucleotides, about 8,500 nucleotides to about 9,500 nucleotides, about 8,500 nucleotides to about 9,000 nucleotides, about 9,000 nucleotides to about 10,000 nucleotides, about 9,000 nucleotides to about 9,500 nucleotides, or about 9,500 nucleotides to about 10,000 nucleotides (inclusive).

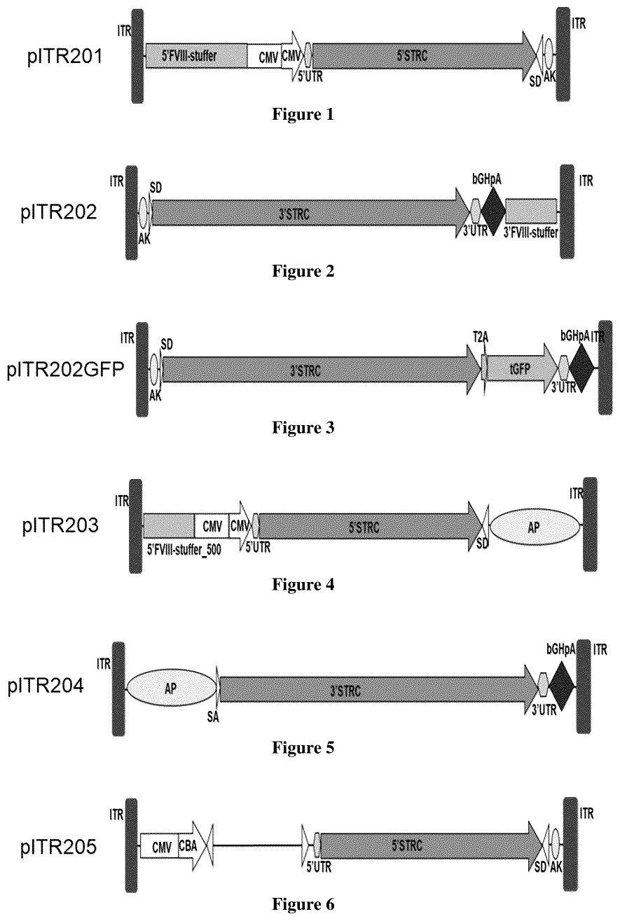

Provided herein are exemplary vectors that can be used in any of the compositions and methods described herein. See, e.g., FIGS. 1-6.

A variety of different methods known in the art can be used to introduce any of vectors disclosed herein into a mammalian cell (e.g., a cochlear outer hair cell). Non-limiting examples of methods for introducing nucleic acid into a mammalian cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the vectors described herein can be introduced into a mammalian cell by, for example, lipofection, and can be stably integrated into an endogenous gene locus (e.g., a stereocilin gene locus or a stereocilin pseudogene 1 gene locus). In some embodiments, the vectors provided herein stably integrate into an endogenous defective stereocilin gene locus, and thereby replace the defective stereocilin gene with a nucleic acid encoding a functioning (e.g., wildtype) stereocilin protein.

Various molecular biology techniques that can be used to introduce a mutation(s) and/or a deletion(s) into an endogenous gene are also known in the art. Non-limiting examples of such techniques include site-directed mutagenesis, CRISPR (e.g., CRISPR/Cas9-induced knock-in mutations and CRISPR/Cas9-induced knock-out mutations), and TALENs. These methods can be used to correct the sequence of a defective endogenous gene present in a chromosome of a target cell.

Any of the vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, and a Kozak consensus sequence. Non-limiting examples of these control sequences are described herein. In some embodiments, a promoter can be a native promoter, a constitutive promoter, an inducible promoter, and/or a tissue-specific promoter.

### Promoters

The term “promoter” means a DNA sequence recognized by enzymes/proteins in a mammalian cell required to initiate the transcription of a specific gene (e.g., a stereocilin gene). A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, a vector encoding an N-terminal portion of a stereocilin protein (e.g., a human stereocilin protein) can include a promoter and/or an enhancer. The vector encoding the N-terminal portion of the stereocilin protein can include any of the promoters and/or enhancers described herein or known in the art.

In some embodiments, the promoter is an inducible promoter, a constitutive promoter, a mammalian cell promoter, a viral promoter, a chimeric promoter, an engineered promoter, a tissue-specific promoter, or any other type of promoter known in the art. In some embodiments, the promoter is a RNA polymerase II promoter, such as a mammalian RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in cochlear cells such as hair cells. In some examples, the promoter is a cochlea-specific promoter or a cochlea-oriented promoter.

A variety of promoters are known in the art that can be used herein. Non-limiting examples of promoters that can be used herein include: human EF1a, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CMV promoter, e.g., a CMV promoter comprising or consisting of SEQ ID NO: 21. In some embodiments, the promoter is a CAG promoter or a CAG/CBA promoter. In some embodiments, the promoter is a CBA promoter, e.g., a CBA promoter comprising or consisting of SEQ ID NO: 22.

The term “constitutive” promoter refers to a nucleotide sequence that, when operably linked with a nucleic acid encoding a protein (e.g., a stereocilin protein), causes RNA to be transcribed from the nucleic acid in a mammalian cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen®, Clontech®, and Ariad®. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory proteins that bind to the tissue-specific promoter).

In some embodiments, the regulatory sequences impart tissue-specific gene expression capabilities. In some cases, the tissue-specific regulatory sequences bind tissue-specific transcription factors that induce transcription in a tissue-specific manner.

Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, an alpha-myosin heavy chain (a-MHC) promoter, and a cardiac Troponin T (cTnT) promoter. Additional exemplary promoters include Beta-actin promoter, hepatitis B virus core promoter (Sandig et al., Gene Ther. 3:1002-1009, 1996), alpha-fetoprotein (AFP) promoter (Arbuthnot et al., Hum. Gene Ther. 7:1503-1514, 1996), bone osteocalcin promoter (Stein et al., Mol. Biol. Rep. 24:185-196, 1997); bone sialoprotein promoter (Chen et al., J. Bone Miner. Res. 11:654-664, 1996), CD2 promoter (Hansal et al., J. Immunol. 161:1063-1068, 1998); immunoglobulin heavy chain promoter; T cell receptor alpha-chain promoter, neuronal such as neuron-specific enolase (NSE) promoter (Andersen et al., Cell. Mol. Neurobiol. 13:503-515, 1993), neurofilament light-chain gene promoter (Piccioli et al., Proc. Natl. Acad. Sci. U.S.A. 88:5611-5615, 1991), and the neuron-specific vgf gene promoter (Piccioli et al., Neuron 15:373-384, 1995).

In some embodiments, the tissue-specific promoter is a cochlea-specific promoter. In some embodiments, the tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter. In some embodiments, the promoter is an outer hair cell-specific promoter such as a PRESTIN promoter or an ONCOMOD promoter. See, e.g., Zheng et al., Nature 405:149-155, 2000; Tian et al. Dev. Dyn. 231:199-203, 2004; and Ryan et al., Adv. Otorhinolaryngol. 66: 99-115, 2009.

### Enhancers and 5′ Cap

In some instances, a vector can include a promoter sequence and/or an enhancer sequence. The term “enhancer” refers to a nucleotide sequence that can increase the level of transcription of a nucleic acid encoding a protein of interest (e.g., a stereocilin protein). Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer. In some embodiments, the CMV enhancer sequence comprises or consists of SEQ ID NO: 20.

### Poly(A) Sequences

In some embodiments, any of the vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) sequence is positioned 3′ to the nucleic acid sequence encoding the C-terminus of the stereocilin protein.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation signal or “poly(A) sequence.” The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) sequence” is a sequence that triggers the endonuclease cleavage of an mRNA and the additional of a series of adenosines to the 3′ end of the cleaved mRNA.

There are several poly(A) sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7):1340-1347, 2007), the group consisting of SV40 poly(A) site, such as the SV40 late and early poly(A) site (Schek et al., Mol. Cell Biol. 12(12):5386-5393, 1992). In some embodiments, the bGH polyA sequence comprises or consists of SEQ ID NO: 34.

The poly(A) sequence can a sequence of AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) sequence can be a synthetic polyadenylation site (see, e.g., the pCl-neo expression vector of Promega which is based on Levitt et al, Genes Dev. 3(7):1019-1025, 1989). In some embodiments, the poly(A) sequence is the polyadenylation signal of soluble neuropilin-1 (sNRP) (AAATAAAATACGAAATG, SEQ ID NO: 38) (see, e.g., WO 05/073384). Additional examples of poly(A) sequences are known in the art.

### Internal Ribosome Entry Site (IRES)

In some embodiments, a vector encoding the C-terminus of the stereocilin protein can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the vector that encodes the C-terminus of a stereocilin protein is the foot and mouth disease virus (FMDV). The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

### Reporter Sequences

Any of the vectors provided herein can optionally include a sequence encoding a reporter protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with regulatory elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence is turbo green fluorescent protein (tGFP) (SEQ ID NO: 31).

tGFP cDNA
(SEQ ID NO: 31)
ATGGAGAGCGACGAGAGCGGCCTGCCCGCCATGGAGATCGAGTGCCGCA
TCACCGGCACCCTGAACGGCGTGGAGTTCGAGCTGGTGGGCGGCGGAGA
GGGCACCCCCGAGCAGGGCCGCATGACCAACAAGATGAAGAGCACCAAA
GGCGCCCTGACCTTCAGCCCCTACCTGCTGAGCCACGTGATGGGCTACG
GCTTCTACCACTTCGGCACCTACCCCAGCGGCTACGAGAACCCCTTCCT
GCACGCCATCAACAACGGCGGCTACACCAACACCCGCATCGAGAAGTAC
GAGGACGGCGGCGTGCTGCACGTGAGCTTCAGCTACCGCTACGAGGCCG
GCCGCGTGATCGGCGACTTCAAGGTGATGGGCACCGGCTTCCCCGAGGA
CAGCGTGATCTTCACCGACAAGATCATCCGCAGCAACGCCACCGTGGAG
CACCTGCACCCCATGGGCGATAACGATCTGGATGGCAGCTTCACCCGCA
CCTTCAGCCTGCGCGACGGCGGCTACTACAGCTCCGTGGTGGACAGCCA
CATGCACTTCAAGAGCGCCATCCACCCCAGCATCCTGCAGAACGGGGGC
CCCATGTTCGCCTTCCGCCGCGTGGAGGAGGATCACAGCAACACCGAGC
TGGGCATCGTGGAGTACCAGCACGCCTTCAAGACCCCGGATGCAGATGC
CGGTGAAGAA
tGFP Protein
(SEQ ID NO: 32)
MESDESGLPAMEIECRITGTLNGVEFELVGGGEGTPEQGRMTNKMKSTK
GALTFSPYLLSHVMGYGFYHFGTYPSGYENPFLHAINNGGYTNTRIEKY
EDGGVLHVSFSYRYEAGRVIGDFKVMGTGFPEDSVIFTDKIIRSNATVE
HLHPMGDNDLDGSFTRTFSLRDGGYYSSVVDSHMHFKSAIHPSILQNGG
PMFAFRRVEEDHSNTELGIVEYQHAFKTPDADAGEE

In some embodiments, the reporter sequence is the LacZ gene, and the presence of a vector carrying the LacZ gene in a mammalian cell (e.g., a cochlear outer hair cell) is detected by assays for beta-galactosidase activity. In other embodiments, the reporter is a fluorescent protein (e.g., green fluorescent protein) or luciferase, the presence of a vector carrying the fluorescent protein or luciferase in a mammalian cell (e.g., a cochlear outer hair cell) may be measured by fluorescent techniques (e.g., fluorescent microscopy or FACS) or light production in a luminometer (e.g., a spectrophotometer or an IVIS imaging instrument). In some embodiments, the reporter sequence can be used to verify the tissue-specific targeting capabilities and tissue-specific promoter regulatory activity of any of the vectors described herein.

### Flanking Regions Untranslated Regions (UTRs)

In some embodiments, any of the vectors described herein (e.g., any of the at least two different vectors) can include an untranslated region. In some embodiments, a vector can includes a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the stability of a stereocilin protein.

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), which is followed by another “G”. The 5′ UTR have also been known, e.g., to form secondary structures that are involved in elongation factor binding.

For example, in some embodiments, a 5′ UTR is included in any of the vectors described herein. Non-limiting examples of 5′ UTRs including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the cochlea can be included in any of the vectors, compositions, kits, and methods described herein.

3′ UTRs are known to have stretches of adenosines and uridines embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

An exemplary human wildtype 5′ UTR is or includes the sequence of SEQ ID NO: 24. An exemplary human wildtype 3′ UTR is or includes the sequence of SEQ ID NO: 33.

In some embodiments of any of the compositions described herein, a 5′ UTR, a 3′ UTR, or both are included in a vector (e.g., any of the vectors described herein). For example, any of the 5′ UTRs described herein can be operatively linked to the start codon in any of the coding sequences described herein. For example, any of the 3′ UTRs can be operatively linked to the 3′-terminal codon (last codon) in any of the coding sequences described herein.

In some embodiments of any of the compositions described herein, the 5′ UTR comprises at least 10 contiguous (e.g., at least 15 contiguous, at least 20 contiguous, at least 25 contiguous, at least 30 contiguous, at least 35 contiguous, at least 40 contiguous, at least 45 contiguous, at least 50 contiguous, at least 55 contiguous, at least 60 contiguous, at least 65 contiguous, or at least 70 contiguous) nucleotides from anywhere within SEQ ID NO: 24.

For example, a 5′ UTR can include or consist of one or more of: nucleotide positions 1 to 70, nucleotide positions 1 to 60, nucleotide positions 1 to 50, nucleotide positions 1 to 40, nucleotide positions 1 to 30, nucleotide positions 1 to 20, nucleotide positions 1 to 10, nucleotide positions 10 to 70, nucleotide positions 10 to 60, nucleotide positions 10 to 50, nucleotide positions 10 to 40, nucleotide positions 10 to 30, nucleotide positions 10 to 20, nucleotide positions 20 to 70, nucleotide positions 20 to 60, nucleotide positions 20 to 50, nucleotide positions 20 to 40, nucleotide positions 20 to 30, nucleotide positions 30 to 70, nucleotide positions 30 to 60, nucleotide positions 30 to 50, nucleotide positions 30 to 40, nucleotide positions 40 to 70, nucleotide positions 40 to 60, nucleotide positions 40 to 50, nucleotide positions 50 to 70, nucleotide positions 50 to 60, nucleotide positions 60 to 70, nucleotide positions 100 to 105, of SEQ ID NO: 24.

In some embodiments of any of the compositions described herein, the 5′ UTR comprises a sequence that is at least 70% (e.g., at least 75%, at least 80%, at least 90%, at least 91%, at least 92%, at least 93%, at least 94%, at least 95%, at least 96%, at least 97%, at least 98%, or at least 99%) identical to SEQ ID NO: 24.

In some embodiments of any of the compositions described herein, the 3′ UTR comprises at least 10 contiguous (e.g., at least 15 contiguous, at least 20 contiguous, at least 25 contiguous, at least 30 contiguous, at least 35 contiguous, at least 40 contiguous, at least 45 contiguous, at least 50 contiguous, at least 55 contiguous, at least 60 contiguous, at least 65 contiguous, at least 70 contiguous, at least 75 contiguous, at least 80 contiguous, at least 85 contiguous, at least 90 contiguous, at least 95 contiguous, at least 100 contiguous, or at least 105 contiguous) nucleotides from anywhere within SEQ ID NO: 33.

For example, a 3′ UTR can include or consist of one or more of: nucleotide positions 1 to 107, nucleotide positions 1 to 105, nucleotide positions 1 to 100, nucleotide positions 1 to 90, nucleotide positions 1 to 80, nucleotide positions 1 to 70, nucleotide positions 1 to 60, nucleotide positions 1 to 50, nucleotide positions 1 to 40, nucleotide positions 1 to 30, nucleotide positions 1 to 20, nucleotide positions 1 to 10, nucleotide positions 10 to 107, nucleotide positions 10 to 105, nucleotide positions 10 to 100, nucleotide positions 10 to 90, nucleotide positions 10 to 80, nucleotide positions 10 to 70, nucleotide positions 10 to 60, nucleotide positions 10 to 50, nucleotide positions 10 to 40, nucleotide positions 10 to 30, nucleotide positions 10 to 20, nucleotide positions 20 to 107, nucleotide positions 20 to 105, nucleotide positions 20 to 100, nucleotide positions 20 to 90, nucleotide positions 20 to 80, nucleotide positions 20 to 70, nucleotide positions 20 to 60, nucleotide positions 20 to 50, nucleotide positions 20 to 40, nucleotide positions 20 to 30, nucleotide positions 30 to 107, nucleotide positions 30 to 105, nucleotide positions 30 to 100, nucleotide positions 30 to 90, nucleotide positions 30 to 80, nucleotide positions 30 to 70, nucleotide positions 30 to 60, nucleotide positions 30 to 50, nucleotide positions 30 to 40, nucleotide positions 40 to 107, nucleotide positions 40 to 105, nucleotide positions 40 to 100, nucleotide positions 40 to 90, nucleotide positions 40 to 80, nucleotide positions 40 to 70, nucleotide positions 40 to 60, nucleotide positions 40 to 50, nucleotide positions 50 to 107, nucleotide positions 50 to 105, nucleotide positions 50 to 100, nucleotide positions 50 to 90, nucleotide positions 50 to 80, nucleotide positions 50 to 70, nucleotide positions 50 to 60, nucleotide positions 60 to, nucleotide positions 60 to 107, nucleotide positions 60 to 105, nucleotide positions 60 to 100, nucleotide positions 60 to 90, nucleotide positions 60 to 80, nucleotide positions 60 to 70, nucleotide positions 70 to 107, nucleotide positions 70 to 105, nucleotide positions 70 to 100, nucleotide positions 70 to 90, nucleotide positions 70 to 80, nucleotide positions 80 to 107, nucleotide positions 80 to 105, nucleotide positions 80 to 100, nucleotide positions 80 to 90, nucleotide positions 90 to 107, nucleotide positions 90 to 105, nucleotide positions 90 to 100, nucleotide positions 100 to 107, nucleotide positions 100 to 105, of SEQ ID NO: 33.

In some embodiments of any of the compositions described herein, the 3′ UTR comprises a sequence that is at least 70% (e.g., at least 75%, at least 80%, at least 90%, at least 91%, at least 92%, at least 93%, at least 94%, at least 95%, at least 96%, at least 97%, at least 98%, or at least 99%) identical to SEQ ID NO: 33.

Human 5′ UTR of STRC
(SEQ ID NO: 24)
GCCCTGCCCTCACCTGGCTATCCCACACAGGTGAGAATAACCAGAACT
CACCTCCGGTACCAGTGTTCACTTG
Human 3′ UTR of STRC
(SEQ ID NO: 33)
GCCTGTCTCTACAGTAGAAGGAGATTGTGGGGAGAGAAATCTTAAGTC
ATAATGAATAAAGTGCAAACAGAAGTGCATCCTGATTATTTTCAGAAG
CTGATGAGGAATA

In some embodiments, the introduction, removal, or modification of 3′ UTR AREs can be used to modulate the stability of an mRNA encoding a stereocilin protein. In other embodiments, AREs can be removed or mutated to increase the intracellular stability and thus increase translation and production of a stereocilin protein.

In other embodiments, non-UTR sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels. An intron can be an intron from a stereocilin gene or can be an intron from a heterologous gene, e.g., a hybrid adenovirus/mouse immunoglobulin intron (Yew et al., Human Gene Ter. 8(5):575-584, 1997), an SV40 intron (Ostedgaard et al., Proc. Natl. Acad. Sci. U.S.A. 102(8):2952-2957, 2005) an MVM intron (Wu et al., Mol. Ther. 16(2):280-289, 2008), a factor IX truncated intron 1 (Wu et al., Mol. Ther. 16(2):280-289, 2008; Kurachi et al., J. Biol. Chem. 270(10):5276-5281, 1995), a chimeric β-globulin splice donor/immunoglobulin heavy chain spice acceptor intron (Wu et al., Mol. Ther. 16(2):280-289, 2008; Choi et al., Mol. Brain 7:17, 2014) a SV40 late splice donor/splice acceptor intron (19S/16S) (Yew et al., Human Gene Ther. 8(5):575-584, 1997), a hybrid adenovirus spice donor/Igg splice acceptor (Choi et al., Mol. Brain 7:17, 1991; Huang and Gorman, Mol. Cell Biol. 10(4):1805-1810, 1990).

Non-limiting examples of a splice donor and splice acceptor sequences are SEQ ID NOs: 6 and 7, respectively.

In some embodiments of any of the vectors described herein, the vector includes a chimeric intron sequence (SEQ ID NO: 23).

In some embodiments of any of the vectors described herein, the vector includes a T2A sequence (SEQ ID NO: 29).

T2A cDNA sequence
(SEQ ID NO: 29)
GAGGGCAGAGGAAGTCTGCTAACATGCGGTGACGTCGAGGAGAATCCT
GGCCCA
T2A Protein
(SEQ ID NO: 30)
GSGEGRGSLLTCGDVEENPGP

### Additional Sequences

Any of the vectors provided herein can optionally include additional nucleotide sequences (“a stuffer sequence”) in order to optimize the total number of basepairs in the vector. For example, in order to optimize packaging, each vector can be designed to contain a total of about 4,000 base pairs to about 4,700 base pairs, e.g., about 4,000 base pairs to about 4,650 base pairs, about 4,000 base pairs to about 4,600 base pairs, about 4,000 base pairs to about 4,550 base pairs, about 4,000 base pairs to about 4,500 base pairs, about 4,000 base pairs to about 4,450 base pairs, about 4,000 base pairs to about 4,400 base pairs, about 4,000 base pairs to about 4,350 base pairs, about 4,000 base pairs to about 4,300 base pairs, about 4,000 base pairs to about 4,250 base pairs, about 4,000 base pairs to about 4,200 base pairs, about 4,000 base pairs to about 4,150 base pairs, about 4,000 base pairs to about 4,100 base pairs, about 4,000 base pairs to about 4,050 base pairs, about 4,050 base pairs to about 4,700 base pairs, about 4,050 base pairs to about 4,650 base pairs, about 4,050 base pairs to about 4,600 base pairs, about 4,050 base pairs to about 4,550 base pairs, about 4,050 base pairs to about 4,500 base pairs, about 4,050 base pairs to about 4,450 base pairs, about 4,050 base pairs to about 4,400 base pairs, about 4,050 base pairs to about 4,350 base pairs, about 4,050 base pairs to about 4,300 base pairs, about 4,050 base pairs to about 4,250 base pairs, about 4,050 base pairs to about 4,200 base pairs, about 4,050 base pairs to about 4,150 base pairs, about 4,050 base pairs to about 4,100 base pairs, about 4,100 base pairs to about 4,700 base pairs, about 4,100 base pairs to about 4,650 base pairs, about 4,100 base pairs to about 4,600 base pairs, about 4,100 base pairs to about 4,550 base pairs, about 4,100 base pairs to about 4,500 base pairs, about 4,100 base pairs to about 4,450 base pairs, about 4,100 base pairs to about 4,400 base pairs, about 4,100 base pairs to about 4,350 base pairs, about 4,100 base pairs to about 4,300 base pairs, about 4,100 base pairs to about 4,250 base pairs, about 4,100 base pairs to about 4,200 base pairs, about 4,100 base pairs to about 4,150 base pairs, about 4,150 base pairs to about 4,700 base pairs, about 4,150 base pairs to about 4,650 base pairs, about 4,150 base pairs to about 4,600 base pairs, about 4,150 base pairs to about 4,550 base pairs, about 4,150 base pairs to about 4,500 base pairs, about 4,150 base pairs to about 4,450 base pairs, about 4,150 base pairs to about 4,400 base pairs, about 4,150 base pairs to about 4,350 base pairs, about 4,150 base pairs to about 4,300 base pairs, about 4,150 base pairs to about 4,250 base pairs, about 4,150 base pairs to about 4,200 base pairs, about 4,200 base pairs to about 4,700 base pairs, about 4,200 base pairs to about 4,650 base pairs, about 4,200 base pairs to about 4,600 base pairs, about 4,200 base pairs to about 4,550 base pairs, about 4,200 base pairs to about 4,500 base pairs, about 4,200 base pairs to about 4,450 base pairs, about 4,200 base pairs to about 4,400 base pairs, about 4,200 base pairs to about 4,350 base pairs, about 4,200 base pairs to about 4,300 base pairs, about 4,200 base pairs to about 4,250 base pairs, about 4,250 base pairs to about 4,700 base pairs, about 4,250 base pairs to about 4,650 base pairs, about 4,250 base pairs to about 4,600 base pairs, about 4,250 base pairs to about 4,550 base pairs, about 4,250 base pairs to about 4,500 base pairs, about 4,250 base pairs to about 4,450 base pairs, about 4,250 base pairs to about 4,400 base pairs, about 4,250 base pairs to about 4,350 base pairs, about 4,250 base pairs to about 4,300 base pairs, about 4,300 base pairs to about 4,700 base pairs, about 4,300 base pairs to about 4,650 base pairs, about 4,300 base pairs to about 4,600 base pairs, about 4,300 base pairs to about 4,550 base pairs, about 4,300 base pairs to about 4,500 base pairs, about 4,300 base pairs to about 4,450 base pairs, about 4,300 base pairs to about 4,400 base pairs, about 4,300 base pairs to about 4,350 base pairs, about 4,350 base pairs to about 4,700 base pairs, about 4,350 base pairs to about 4,650 base pairs, about 4,350 base pairs to about 4,600 base pairs, about 4,350 base pairs to about 4,550 base pairs, about 4,350 base pairs to about 4,500 base pairs, about 4,350 base pairs to about 4,450 base pairs, about 4,350 base pairs to about 4,400 base pairs, about 4,400 base pairs to about 4,700 base pairs, about 4,400 base pairs to about 4,650 base pairs, about 4,400 base pairs to about 4,600 base pairs, about 4,400 base pairs to about 4,550 base pairs, about 4,400 base pairs to about 4,500 base pairs, about 4,400 base pairs to about 4,450 base pairs, about 4,450 base pairs to about 4,700 base pairs, about 4,450 base pairs to about 4,650 base pairs, about 4,450 base pairs to about 4,600 base pairs, about 4,450 base pairs to about 4,550 base pairs, about 4,450 base pairs to about 4,500 base pairs, about 4,500 base pairs to about 4,700 base pairs, about 4,500 base pairs to about 4,650 base pairs, about 4,500 base pairs to about 4,600 base pairs, about 4,500 base pairs to about 4,550 base pairs, about 4,550 base pairs to about 4,700 base pairs, about 4,550 base pairs to about 4,650 base pairs, about 4,550 base pairs to about 4,600 base pairs, about 4,600 base pairs to about 4,700 base pairs, about 4,600 base pairs to about 4,650 base pairs, or about 4,650 base pairs to about 4,700 base pairs (inclusive).

A stuffer sequence can be any nucleotide sequence, e.g., up to 1000 bp, that can be included in any of the vectors described herein that is not transcribed and that does not serve a regulatory function in order to achieve a desirable vector size (e.g., a vector size of about 4 kb to about 5 kb, or any of the vector sizes provided herein). For example, a stuffer sequence can by any nucleotide sequences of about 100 bp to about 1000 bp (e.g., about 100 bp to about 900 bp, about 100 bp to about 850 bp, about 100 bp to about 800 bp, about 100 bp to about 750 bp, about 100 bp to about 700 bp, about 100 bp to about 650 bp, about 100 bp to about 600 bp, about 100 bp to about 550 bp, about 100 bp to about 500 bp, about 100 bp to about 450 bp, about 100 bp to about 400 bp, about 100 bp to about 350 bp, about 100 bp to about 300 bp, about 100 bp to about 250 bp, about 100 bp to about 200 bp, about 100 bp to about 150 bp, about 150 bp to about 1000 bp, about 150 bp to about 900 bp, about 150 bp to about 850 bp, about 150 bp to about 800 bp, about 150 bp to about 750 bp, about 150 bp to about 700 bp, about 150 bp to about 650 bp, about 150 bp to about 600 bp, about 150 bp to about 550 bp, about 150 bp to about 500 bp, about 150 bp to about 450 bp, about 150 bp to about 400 bp, about 150 bp to about 350 bp, about 150 bp to about 300 bp, about 150 bp to about 250 bp, about 150 bp to about 200 bp, about 200 bp to about 1000 bp, about 200 bp to about 900 bp, about 200 bp to about 850 bp, about 200 bp to about 800 bp, about 200 bp to about 750 bp, about 200 bp to about 700 bp, about 200 bp to about 650 bp, about 200 bp to about 600 bp, about 200 bp to about 550 bp, about 200 bp to about 500 bp, about 200 bp to about 450 bp, about 200 bp to about 400 bp, about 200 bp to about 350 bp, about 200 bp to about 300 bp, about 200 bp to about 250 bp, about 250 bp to about 1000 bp, about 250 bp to about 900 bp, about 250 bp to about 850 bp, about 250 bp to about 800 bp, about 250 bp to about 750 bp, about 250 bp to about 700 bp, about 250 bp to about 650 bp, about 250 bp to about 600 bp, about 250 bp to about 550 bp, about 250 bp to about 500 bp, about 250 bp to about 450 bp, about 250 bp to about 400 bp, about 250 bp to about 350 bp, about 250 bp to about 300 bp, about 300 bp to about 1000 bp, about 300 bp to about 900 bp, about 300 bp to about 850 bp, about 300 bp to about 800 bp, about 300 bp to about 750 bp, about 300 bp to about 700 bp, about 300 bp to about 650 bp, about 300 bp to about 600 bp, about 300 bp to about 550 bp, about 300 bp to about 500 bp, about 300 bp to about 450 bp, about 300 bp to about 400 bp, about 300 bp to about 350 bp, about 350 bp to about 1000 bp, about 350 bp to about 900 bp, about 350 bp to about 850 bp, about 350 bp to about 800 bp, about 350 bp to about 750 bp, about 350 bp to about 700 bp, about 350 bp to about 650 bp, about 350 bp to about 600 bp, about 350 bp to about 550 bp, about 350 bp to about 500 bp, about 350 bp to about 450 bp, about 350 bp to about 400 bp, about 400 bp to about 1000 bp, about 400 bp to about 900 bp, about 400 bp to about 850 bp, about 400 bp to about 800 bp, about 400 bp to about 750 bp, about 400 bp to about 700 bp, about 400 bp to about 650 bp, about 400 bp to about 600 bp, about 400 bp to about 550 bp, about 400 bp to about 500 bp, about 400 bp to about 450 bp, about 450 bp to about 1000 bp, about 450 bp to about 900 bp, about 450 bp to about 850 bp, about 450 bp to about 800 bp, about 450 bp to about 750 bp, about 450 bp to about 700 bp, about 450 bp to about 650 bp, about 450 bp to about 600 bp, about 450 bp to about 550 bp, about 450 bp to about 500 bp, about 500 bp to about 1000 bp, about 500 bp to about 900 bp, about 500 bp to about 850 bp, about 500 bp to about 800 bp, about 500 bp to about 750 bp, about 500 bp to about 700 bp, about 500 bp to about 650 bp, about 500 bp to about 600 bp, about 500 bp to about 550 bp, about 550 bp to about 1000 bp, about 550 bp to about 900 bp, about 550 bp to about 850 bp, about 550 bp to about 800 bp, about 550 bp to about 750 bp, about 550 bp to about 700 bp, about 550 bp to about 650 bp, about 550 bp to about 600 bp, about 600 bp to about 1000 bp, about 600 bp to about 900 bp, about 600 bp to about 850 bp, about 600 bp to about 800 bp, about 600 bp to about 750 bp, about 600 bp to about 700 bp, about 600 bp to about 650 bp, about 650 bp to about 1000 bp, about 650 bp to about 900 bp, about 650 bp to about 850 bp, about 650 bp to about 800 bp, about 650 bp to about 750 bp, about 650 bp to about 700 bp, about 700 bp to about 1000 bp, about 700 bp to about 900 bp, about 700 bp to about 850 bp, about 700 bp to about 800 bp, about 700 bp to about 750 bp, about 750 bp to about 1000 bp, about 750 bp to about 900 bp, about 750 bp to about 850 bp, about 750 bp to about 800 bp, about 800 bp to about 1000 bp, about 800 bp to about 900 bp, about 800 bp to about 850 bp, about 850 bp to about 1000 bp, about 850 bp to about 900 bp, about 900 bp to about 1000 bp, about 900 to about 950 bp, or about 950 bp to about 1000 bp). SEQ ID NOs. 19, 35 and 37 are exemplary human factor FVIII stuffer sequences that can be used in any of the vectors described herein. Additional stuffer sequences are known in the art. Exemplary vectors that include stuffer sequences are shown in FIGS. 1, 2 and 4.

### Mammalian Cells

Also provided herein is a cell (e.g., a mammalian cell) that includes any of the nucleic acids, vectors (e.g., at least two different vectors described herein), or compositions described herein. Skilled practitioners will appreciate that the nucleic acids and vectors described herein can be introduced into any mammalian cell. Non-limiting examples of vectors and methods for introducing vectors into mammalian cells are described herein.

In some embodiments, the cell is a human cell, a mouse cell, a porcine cell, a rabbit cell, a dog cell, a cat cell, a rat cell, or a non-human primate cell. In some embodiments, the cell is a specialized cell of the cochlea. In some embodiments, the cell is a cochlear inner hair cell or a cochlear outer hair cell. In some embodiments, the cell is a cochlear inner hair cell. In some embodiments, the cell is a cochlear outer hair cell.

In some embodiments, the mammalian cell is in vitro. In some embodiments, the mammalian cell is present in a mammal. In some embodiments, the mammalian cell is autologous cell obtained from a subject and cultured ex vivo.

### Methods

Also provided herein is a method of introducing into a cochlea of a mammal (e.g., a human) a therapeutically effective amount of any of the compositions described herein. Also provided are methods of increasing expression of an active stereocilin protein (e.g., a full-length stereocilin protein) in an outer hair cell in a cochlea of a mammal (e.g., a human) that include introducing into the cochlea of the mammal a therapeutically effective amount of any of the compositions described herein. Also provided are methods of treating non-symptomatic sensorineural hearing loss in a subject (e.g., a human) identified as having a defective stereocilin gene, where the methods include administering a therapeutically effective amount of any of the compositions described herein into the cochlea of a subject.

In some embodiments of any of these methods, the mammal has been previously identified as having a defective stereocilin gene (e.g., a stereocilin gene having a mutation that results in a decrease in the expression and/or activity of a stereocilin protein encoded by the gene). Some embodiments of any of these methods further include, prior to the introducing or administering step, determining that the subject has a defective stereocilin gene. Some embodiments of any of these methods can further include detecting a mutation in a stereocilin gene in a subject. Some embodiments of any of the methods can further include identifying or diagnosing a subject as having non-symptomatic sensorineural hearing loss.

In some embodiments of any of these methods, two or more doses of any of the compositions described herein are introduced or administered into the cochlea of the mammal or subject. Some embodiments of any of these methods can include introducing or administering a first dose of the composition into the cochlea of the mammal or subject, assessing hearing function of the mammal or subject following the introducing or the administering of the first dose, and administering an additional dose of the composition into the cochlea of the mammal or subject found not to have a hearing function within a normal range (e.g., as determined using any test for hearing known in the art).

In some embodiments of any of the methods described herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any of the methods described herein, the subject or mammal is a rodent, a non-human primate, or a human. In some embodiments of any of the methods described herein, the subject or mammal is an adult, a teenager, a juvenile, a child, a toddler, an infant, or a newborn. In some embodiments of any of the methods described herein, the subject or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the subject or mammal has or is at risk of developing non-syndromic sensorineural hearing loss. In some embodiments of any of the methods described herein, the subject or mammal has been previously identified as having a mutation in a stereocilin gene. (All references here to “stereocilin gene” refer to the gene that is not the stereocilin pseudogene.) In some embodiments of any of the methods described herein, the subject or mammal has any of the mutations in a stereocilin gene that are described herein or are known in the art to be associated with non-symptomatic sensorineural hearing loss.

In some embodiments of any of the methods described herein, the subject or mammal has been identified as being a carrier of a mutation in a stereocilin gene (e.g., via genetic testing). In some embodiments of any of the methods described herein, the subject or human has been identified as having a mutation in a stereocilin gene and has been diagnosed with non-symptomatic sensorineural hearing loss. In some embodiments of any of the methods described herein, the subject or human has been identified as having non-symptomatic sensorineural hearing loss.

In some embodiments, successful treatment of non-symptomatic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

Also provided herein are methods of increasing expression of an active stereocilin protein (e.g., a full-length stereocilin protein) in a mammalian cell that include introducing any of the compositions described herein into the mammalian cell. In some embodiments of these methods, the mammalian cell is a cochlear outer hair cell. In some embodiments of these methods, the mammalian cell is a human cell (e.g., a human cochlear outer hair cell). In some embodiments of these methods, the mammalian cell is in vitro. In some embodiments of these methods, the mammalian cell is in a mammal. In some embodiments of these methods, the mammalian cell is originally obtained from a mammal and is cultured ex vivo. In some embodiments, the mammalian cell has previously been determined to have a defective stereocilin gene.

Methods for introducing any of the compositions described herein into a mammalian cell are known in the art (e.g., via lipofection or through the use of a viral vector, e.g., any of the viral vectors described herein).

An increase in expression of an active stereocilin protein (e.g., a full-length stereocilin protein) as described herein is, e.g., as compared to a control or to the level of expression of an active stereocilin protein (e.g., a full-length stereocilin protein) prior to the introduction of the vector(s).

Methods of detecting expression and/or activity of stereocilin are known in the art. In some embodiments, the level of expression of a stereocilin protein can be detected directly (e.g., detecting stereocilin protein or detecting stereocilin mRNA). Non-limiting examples of techniques that can be used to detect expression and/or activity of stereocilin directly include: real-time PCR, Western blotting, immunoprecipitation, immunohistochemistry, or immunofluorescence. In some embodiments, expression of a stereocilin protein can be detected indirectly (e.g., through functional hearing tests).

### Pharmaceutical Compositions and Kits

In some embodiments, any of the compositions described herein can further include one or more agents that promote the entry of a nucleic acid or any of the vectors described herein into a mammalian cell (e.g., a liposome or cationic lipid). In some embodiments, any of the vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio® (Madison, Wis.) and Roche® Madison (Madison, Wis.), PhaseRX® polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation®, Pasadena, Calif.), and pH responsive co-block polymers, such as, but not limited to, those produced by PhaseRX® (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

Any of the compositions described herein can be, e.g., a pharmaceutical composition. A pharmaceutical composition can include any of the compositions described herein and one or more pharmaceutically or physiologically acceptable carriers, diluents, or excipients. Such compositions may comprise one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein.

In some embodiments, a single dose of any of the compositions described herein can include a total sum amount of the at least two different vectors of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 μg, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 μg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions provided herein can be, e.g., formulated to be compatible with their intended route of administration. A non-limiting example of an intended route of administration is local administration (e.g., intra-cochlear administration).

In some embodiments, the therapeutic compositions are formulated to include a lipid nanoparticle. In some embodiments, the therapeutic compositions are formulated to include a polymeric nanoparticle. In some embodiments, the therapeutic compositions are formulated to comprise a mini-circle DNA. In some embodiments, the therapeutic compositions are formulated to comprise a CELiD DNA. In some embodiments, the therapeutic compositions are formulated to comprise a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the compositions described herein. In some embodiments, a kit can include a solid composition (e.g., a lyophilized composition including the at least two different vectors described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Routes of Administration

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active AAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Any of the compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the vector delivered trangenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or any of the vectors disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trails examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 Tm. Sonication of MLVs results in the formation of small unilamellar vesicles (SUVs) with diameters in the range of 200 to 500 .ANG., containing an aqueous solution in the core.

Alternatively, nanocapsule formulations of any of the vectors described herein may be used. Nanocapsules can generally entrap substances in a stable and reproducible way. To avoid side effects due to intracellular polymeric overloading, such ultrafine particles (sized around 0.1 Tm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering any of the compositions described herein to a host. Sonophoresis (ie., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intranodally, intramedullary, intramuscularly, by intravenous (i.v.) injection, or intraperitoneally. In one aspect, the nucleic acid compositions of the present invention are administered to a patient by intradermal or subcutaneous injection. In one aspect, the nucleic compositions of the present invention are administered by i.v. injection.

### Devices and Surgical Methods

Provided herein are therapeutic delivery systems for treating non-symptomatic sensorineural hearing loss. In one aspect, the therapeutic delivery systems include i) a medical device capable of creating one or a plurality of incisions in a round window membrane of an inner ear of a human subject in need thereof, and ii) an effective dose of a composition (e.g., any of the compositions described herein). In some embodiments, the medical device includes a plurality of micro-needles.

Also provided herein are surgical methods for treatment of hearing loss (e.g., non-symptomatic sensorineural hearing loss). In some embodiments, the methods include the steps of: introducing into a cochlea of a human subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any of the compositions described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations that become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### EXAMPLES

### Example 1: Construction of Viral Vectors

Recombinant AAV is generated by transfection with an adenovirus-free method as used by Xiao et al. J. Virol. 73(5):3994-4003, 1999. The cis plasmids with AAV ITRs, the trans plasmid with AAV Rep and Cap genes, and a helper plasmid with an essential region from an adenovirus genome are co-transfected in 293 cells in a ratio of 1:1:2. The AAV vectors used here express human stereocilin or mouse stereocilin under multiple dual vector strategies using the constructs described below. AAV serotypes 1, 2, 3, 4, 5, 6, 7, 8, 9, rh8, rh10, rh39, rh43, and Anc80 are each prepared to encapsulate three sets of stereocilin constructs to test (i) a concatemerization-transplicing strategy, (ii) a hybrid intronic-homologous recombination-transplicing strategy, and (iii) an exonic homologous recombination strategy, as summarized by Pryadkina et al., Meth. Clin. Devel. 2:15009, 2015.

### Example 2: Generating and Purifying Viral Particles

Recombinant AAV-1 is produced using a triple transfection protocol and purified by two sequential cesium chloride (CsCl) density gradients, as described by Pryadkina et al., Mol. Ther. 2:15009, 2015. At the end of second centrifugation, 11 fractions of 500 μl are recovered from the CsCl density gradient tube and purified through dialysis in 1×PBS. The fractions are analyzed by dot blot to determine those containing rAAV genomes. The viral genome number (vg) of each preparation is determined by a quantitative real-time PCR-based titration method using primers and probe corresponding to the ITR region of the AAV vector genome (Bartoli et al. Gene. Ther. 13:20-28, 2006).

### Example 3: Formulation of Viral Particles

AAV produced at a titer of 1e14 vg/mL is prepared at dilutions of 3.2e13, 1.0e13, 3.2e12, 1.0e12 vg/mL in artificial perilymph. Artificial perilymph is prepared by combining the following reagents: NaCl, 120 mM; KCl, 3.5 mM; CaCl₂), 1.5 mM; glucose, 5.5 mM; HEPES, 20 mM. The artificial perilymph is titrated with NaOH to adjust its pH to 7.5 (total Na⁺ concentration of 130 mM) (Chen et al., J. Controlled Rel. 110:1-19, 2005).

### Example 4: Device Description

The AAV-STRC formulation is delivered to the cochlea using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter is comprised of at least one microneedle with diameter between 10 and 1,000 microns, which produces perforations in the RWM that are sufficient to allow AAV-STRC to enter the cochlear perilymph of the scala tympani at a rate of approximately 1 uL/min, but small enough to heal without surgical repair. The remaining portion of the microcatheter, proximal to the microneedle(s), is loaded with the AAV-STRC/artificial perilymph formulation at a titer of approximately 1e13 vg/mL. The proximal end of the microcatheter is connected to a micromanipulator that allows for precise, low volume infusions of approximately 1 uL/min.

### Example 5: Animal Model 1A: Surgical Method in Aged Mice

AAV-STRC prepared in artificial perilymph is administered to the scala tympani in mice as described by Shu et al. (Human Gene Therapy, doi:10.1089/hum.2016.053, June 2016,). Six-week-old male mice are anesthetized using an intraperitoneal injection of xylazine (20 mg/kg) and ketamine (100 mg/kg). Body temperature is maintained at 37° C. using an electric heating pad. An incision is made from the right post-auricular region and the tympanic bulla is exposed. The bulla is perforated with a surgical needle and the small hole is expanded to provide access to the cochlea. The bone of the cochlear lateral wall of the scala tympani is thinned with a dental drill so that the membranous lateral wall is left intact. A Nanoliter Microinjection System in conjunction with glass micropipette is used to deliver a total of approximately 300 nL of AAV-STRC in artificial perilymph to the scala tympani at a rate of 2 nL/second. The glass micropipette is left in place for 5 minutes post-injection. Following cochleostomy and injection, the opening in the tympanic bulla is sealed with dental cement, and the muscle and skin are sutured. The mice are allowed to awaken from anesthesia and their pain is controlled with 0.15 mg/kg buprenorphine hydrochloride for 3 days.

### Example 6: Animal Model 2: Reciprocating Micropump in Guinea Pig

### Surgical Procedure

AAV-STRC prepared in artificial perilymph is administered to guinea pigs to assess distribution and toxicity following intracochlear delivery with a reciprocating micropump as described by Tandon et al., Lab Chip, DOI: 10.1039/c51c01396h, 2015. Male guinea pigs weighing approximately 350 g each (n=16) are anesthetized with a combination of pentobarbital sodium (Nembutal; 25 mg kg−1, injected intraperitoneally), fentanyl (0.2 mg kg−1, intramuscularly), and haloperidol (10 mg kg−1, intramuscularly). Lidocaine with epinephrine is given subcutaneously at the incision site as a topical anesthetic. Using a dorsal approach, a 5 mm diameter hole is made in the bulla and a cochleostomy is created approximately 0.5 mm distal to the round window membrane. The cannula of the micropump (described below) is inserted into the cochleostomy, threaded into the cochlea 3 mm apically, and glued to the bulla with a common cyanoacrylate glue. For compound action potential (CAP) measurements, a perfluoroalkoxy-alkane-insulated silver wire electrode (203 μm uncoated diameter) is inserted near the round window niche and glued to the bulla.

Procedures for measurement of distortion product otoacoustic emissions (DPOAEs) and CAPs are performed as previously described in Tandon et al. Biomed Microdevices 17:3-21, 2015. DPOAEs are measured before and after the cochleostomy procedure at the characteristic frequencies: 32, 24, 16, 12, 8, 5.6, 4, and 2.78 kHz in order to monitor any damage that occurs as a result of the surgery.

AAV-STRC at a maximum titer of 1e14 vg/mL is administered to the guinea pig using a micropump as described by Tandon et al. Lab Chip, DOI: 10.1039/c5lc01396h, 2015. The micropump system has 4 selectable ports. These ports are connected to: (i) a large fluidic capacitor used for artificial perilymph storage; (ii) an outlet that connects to the cochlea; (iii) the outlet from an integrated AAV-STRC reservoir; (iv) the inlet to the integrated AAV-STRC reservoir. Each port is fluidically connected to a central pump chamber, and each is individually addressed with a valve. The sequence of events for reciprocating AAV-STRC delivery is as follows: (i) an internal AAV-STRC-refresh loop is run, transferring AAV-STRC from the AAV-STRC reservoir into the main infuse-withdraw line; (ii) AAV-STRC is infused into the cochlea and some artificial perilymph is drained from the artificial perilymph storage capacitor; (iii) the first two steps can be repeated several times for additional doses; (iv) after the AAV-STRC has been allowed to diffuse for some time, a volume of perilymph is withdrawn from the cochlea that is equal to the volume infused in steps (i)-(iii), refilling the artificial perilymph storage capacitor. This process results in net delivery of drug with zero net fluid volume added to the cochlea.

The fluidic capacitors in the micropump are cylindrical chambers whose ceilings are a thin (25.4 μm), flexible, polyimide membrane. The pump chamber has a diameter of 3.5 mm, the fluidic storage capacitor has a diameter of 14 mm, and all of the remaining capacitors have diameters of 4 mm. The same membrane is deflected to block flow at each of the valves. The valve chambers have diameters of 3.1 mm. The serpentine channel that comprises the drug reservoir has a square cross section of width 762 μm and a length of 410 mm for a total volume of 238 μL. All of the other microchannels in the pump have a width of 400 μm and a height of 254 μm. Acute drug delivery in guinea pigs

The micropump is loaded with AAV-STRC and artificial perilymph, and the cannula inserted into a cochleostomy made in the region of the cochlea between the locations with characteristic frequency sensitivity of 24 and 32 kHz, and threaded apically 3 mm, terminating in the 12-16 kHz region. Baseline DPOAE and CAP hearing tests are performed prior to the start of AAV-STRC/artificial perilymph infusion. The pump is then activated and approximately 1 μL of artificial perilymph is infused every 5 min until a total of approximately 10 μL of artificial perilymph is delivered to the cochlea. After a 20 min wait time, approximately 10 μL of perilymph is withdrawn from the cochlea. AAV-STRC delivery is then initiated at a rate of approximately 1 μL every 5 min until a total of approximately 10 μL of fluid delivered.

Animals are sacrificed at 1 week, 1 month, 3 months, and 6 months post-treatment (n=4 per group) and their cochleae extracted. Extent of AAV transduction and STRC expression along the organ of Corti is assessed via immunostaining with anti-STRC antibodies. Antibodies against markers for hair cells (Myo7a) and supporting cells (Sox2) are used to quantify IHCs, OHCs, supporting cells and stereocilia morphology. Annexin V staining is used to assess evidence of apoptosis in cells along the cochlear sensory epithelium.

### Example 7: Animal Model 3: Sheep

AAV-STRC prepared in artificial perilymph is administered to juvenile sheep to assess distribution and toxicity following delivery to the cochlea via trans-RWM infusion. Baseline auditory brainstem response (ABR) and distortion product optoacoustic emissions (DPOAEs) are measured in female sheep at 3 months of age (n=40), bilaterally, to assess pre-treatment inner hair cell (IHC) and outer hair cell (OHC) function. Following baseline ABR and DPOAE measurements, 20 uL of AAV1-STRC at titers of 1.0e14, 3.2e13, 1.0e13 and 3.2e12 vg/mL is injected into the left scala tympani of the sheep (n=10 per group). Each animal's right ear is left as an untreated control. ABR and DPOAE measurements are taken again bilaterally 1, 5 and 10 days following the surgical procedure. At 6 months post-procedure, additional bilateral ABR and DPOAE measurements are taken from all animals, and the animals are subsequently sacrificed and their cochleae removed.

In half of the sacrificed animals (n=5 from each of the dose cohorts), immunostaining is performed to identify hair cell structures and to assess STRC protein expression along the cochlear sensory epithelium. Antibodies against markers for hair cells (Myo7a), supporting cells (Sox2) and stereocilin are used as described previously (Duncker et al. 2013, J Neurosci 33(22):9508-9519). At the basal, middle and apical turns of the organ of corti, total numbers of hair cells and those hair cells expressing STRC are counted within 200 um regions.

In the remaining half of the sacrificed animals (remaining 5 animals from each dose cohort), cochlear tissue samples are collected from the same basal, middle and apical regions as described above, and assayed for stereocilin mRNA transcript.

### Example 8: Animal Model 3A: CRISPR Generated Transgenic Large Animal Model (Sheep)

Generation of Plasmid Co-Expressing Cas9 and sgRNA

The pX330-U6-Chimeric_BB-CBh-hSpCas9 plasmid (Addgene plasmid #42230) is digested with BsbI, dephosphorylated using Antartic Phosphatase, and the linearized vector is gel purified. To generate the bicistronic vector (pX330-cas9-STRC) expressing Cas9 and sgRNA against STRC, a pair of oligos for targeting stereocilin exon 1 is annealed, phosphorylated and ligated to a linearized vector (Cong et al., Science 339(6121):819-23, 2013).

### Genome Editing Assay in Cells

The A15 astroglial sheep cell line (Vilette et al., 2000 In Vitro Cell Dev Biol Anim 36(1):45-9) is maintained in DMEM in 10% Fetal Bovine Serum, 2 mM glutamine, 1% sodium pyruvate and 1% penicillin/streptomycin. Cells are transfected in 24-well plates with 2 μg of pX330-cas9-STRC co-expressing Cas9 and sgRNA against stereocilin using Lipofectamine® LTX reagent. Three days later, genomic DNA from transfected cells is extracted and quantified using a NanoDrop™ 2000 spectrophotometer, measuring A260/A280 and A260/A230 ratios to account for sample purity.

Gene mutation activity of sgRNA sequence at the target locus of STRC exon 1 is quantified using the T7EI mismatch detection assay. DNA sequence of interest is PCR-amplified with a high-fidelity polymerase (Herculase II fusion polymerase) using specific primers. The resultant PCR product is then denatured and slowly re-annealed (95° C., 2 min; 95° C. to 85° C., −2° C./see; 85° C. to 25° C., −1° C./sec) to produce a homoduplex/heteroduplex mix. This is then digested by 5U of T7EI restriction enzyme at 37° C. for 30 minutes. Digestion products are separated by 2% agarose gel electrophoresis. The ratio of cleaved to uncleaved products is used to calculate NHEJ frequency as previously described using Image J software (Menoret et al. 2011 Advanced protocols for Animal Transgenesis. An ISTT Manual. Heidelberg: Springer. p 117-36). NHEJ frequency is calculated as % gene modification=100×(1−(1−fraction cleaved)\hat{ }(½).

Production of sgRNA and Cas9 mRNA

As described previously (Bellec et al., Current Gene Ther., 2015), T7 promoter is added to sgRNA template by PCR amplification of the pX330-cas9-STRC plasmid. The PCR product is purified using NucleoSpin® Gel and PCR Clean-up. It is used as the template for in vitro transcription using MEGAshortscript™ T7 kit according to the manufacturer's manual. Following completion of transcription, DNase I treatment is performed.

The Cas9 mRNA is transcribed using a PmeI-digested Cas9 expression JDS246 plasmid (Addgene plasmid #43861) and the mMESSAGE mMACHINE® T7 ULTRA Transcription Kit according to the manufacturer's manual. Following completion of transcription, the poly(A) tailing reaction and DNase I treatment are performed. Both the Cas9 mRNA and the sgRNAs are purified using a MEGAclear™ kit and eluted in elution buffer.

### In Vitro Production of Embryos

The sheep embryos are produced by in vitro fertilization according to routine procedure as described previously (Crispo et al. 2014 Transgenic Res, 24(1):31-41). Briefly, sheep ovaries from a slaughterhouse are transported to the laboratory and cumulus oocyte complexes (COCs) are aspirated in recovery medium. The selected COCs are placed in maturation medium for 24 h in 5% CO2 in humidified air atmosphere at 39° C. Then, expanded COCs are inseminated in 100 μl drops with 1×106 dose of frozen-thawed semen selected by ascendant migration on a swim up method. Fertilization is carried out in 5% CO2 with humidified atmosphere at 39° C. for 22 h.

Microinjection into Zygotes

Soon after fertilization, 600 presumptive zygotes are randomly assigned to three experimental groups to be microinjected (CRISPR group, n=200; and Buffer group, n=200) or not (Control group, n=200). Microinjection of the CRISPR group is performed by microinjection into the cytoplasm with 5 ng/μl of sgRNA and 20 ng/μl of Cas9 mRNA diluted in injection buffer (10 mM Tris pH 7.5, 0.1 mM EDTA), while the Buffer group is injected with the same procedure but with buffer alone. Lastly, injected and non-injected embryos are transferred to culture medium under mineral oil, in 5% CO2, 5% 02 and 90% N2 in humidified atmosphere at 39° C. Cleavage rate on Day 2 (cleaved zygotes per total oocytes) and development rate on Day 6 (morulae and blastocysts per total oocytes) are recorded for all experimental groups. After Day 6, DNA from 20 CRISPR group embryos are analyzed by Sanger sequencing to detect the mutation at the STRC gene level.

To determine the in vivo efficiency of the system, blastocysts produced by CRISPR/Cas9 zygote microinjection are transferred to recipient females. Only early blastocysts, blastocysts and expanded blastocysts classified as excellent or good (i.e. Grade 1 as defined in Stringfellow et al. 2010, Manual of the International Embryo Transfer Society) are transferred on Day 6 after fertilization. Embryo transfer is performed by minimally invasive surgery assisted by laparoscopy to place the embryos into the cranial side of the ipsilateral uterine horn to the corpus luteum. Recipient ewes are previously synchronized to be on Day 6 of the estrous cycle using a standard protocol to control ovulation described previously, as described by Menchaca et al. 2004, Reprod Fertil Dev. 16(4):403-413.

### Monitoring of Fetuses and Lambs

Pregnancy diagnosis and fetal development are performed on Day 30 and 105, respectively, by using B-mode ultrasonography equipped with a 5 and 3.5 MHz probe. Day 0 of the experiment is defined as the moment of embryo fertilization. Several parameters are measured to study the development of fetuses at Day 105 of gestation: thoracic diameter, biparietal diameter, occipitonasal length and heart rate. At delivery, length of gestation, gender, rectal temperature, heart and respiratory rates, body weight, thoracic perimeter, biparietal diameter, crown-rump and occipitonasal length, height at withers, height at hips, width at hips and width at chest were recorded. Body weight and morphometric variables are determined at birth, and 15, 30 and 60 days later.

### Identification and Genotyping of Transgenic Animals

Samples from skin and limb muscle of the lambs are taken seven days after birth and T7EI assay, western blot test and histology examinations are performed in order to identify and characterize KO founders and off-target sites. Total DNA is isolated from skin biopsies for all animals and from muscle for some animals. Samples are analyzed using capillary electrophoresis. Genotyping of STRC exon 1 is performed by direct sequencing of PCR amplicons and in muscle biopsies by additional sequencing of isolated bacterial clones with individual amplicon sequences.

### Analysis of Stereocilin Expression

Western blotting is performed to determine the presence of myostatin in the muscle fiber. Equal amounts of total proteins are run on 12% (v/v) gel electrophoresis and electrophoretically transferred to a PVDF membrane. Monoclonal mouse anti-stereocilin antibody is used in the western blotting. The washed membranes are incubated with 1:50000 dilution of secondary antibody linked to horseradish peroxidase (HPR). HPR activity is detected using western blot chemiluminescence.

### AAV-STRC Rescue Therapy in Transgenic Sheep Model

AAV-STRC prepared in artificial perilymph is administered to STRC knockout transgenic sheep to assess the ability to restore normal hearing function following delivery to the cochlea via trans-RWM infusion. Baseline auditory brainstem response (ABR) and distortion product optoacoustic emissions (DPOAEs) are measured in female sheep at 3 months of age (n=30), bilaterally, to assess pre-treatment inner hair cell (IHC) and outer hair cell (OHC) function. Following baseline ABR and DPOAE measurements, 20 uL of AAV1-STRC at titers of 1.0e14, 3.2e13, and 1.0e13 vg/mL is injected into the left scala tympani of the sheep (n=10 per group). Each animal's right ear is left as an untreated control. ABR and DPOAE measurements are taken again bilaterally 1, 5 and 10 days following the surgical procedure. At 6 months post-procedure, additional bilateral ABR and DPOAE measurements are taken from all animals, and the animals are subsequently sacrificed and their cochleae removed.

In half of the sacrificed animals (n=5 from each of the dose cohorts), immunostaining is performed to identify hair cell structures and to assess STRC protein expression along the cochlear sensory epithelium. Antibodies against markers for hair cells (Myo7a), supporting cells (Sox2) and stereocilin are used as described previously (Duncker et al., J Neurosci 33(22):9508-9519, 2013). At the basal, middle and apical turns of the organ of corti, total numbers of hair cells and those hair cells expressing STRC are counted within 200 um regions.

In the remaining half of the sacrificed animals (remaining 5 animals from each dose cohort), cochlear tissue samples are collected from the same basal, middle and apical regions as described above, and assayed for stereocilin mRNA transcript as described previously (Duncker et al. 2013, J Neurosci 33(22):9508-9519, 2013; Heidrych et al., Hum. Mol. Genet. 17:3814-3821, 2008; Heidrych et al., Hum. Mol. Genet. 18:2779-2790, 2009).

### Example 9: Human Clinical Example (Pediatric Treatment)

The patient is put under general anesthesia. The surgeon approaches the tympanic membrane from external auditory canal, makes a small incision at the inferior edge of the external auditory canal where it meets the tympani membrane, and lifts the tympanic membrane as a flap to expose the middle ear space. A surgical laser is used to make a small opening (approximately 2 mm) in the stapes footplate. The surgeon then penetrates the round window membrane with a microcatheter loaded with a solution of AAV-STRC prepared in artificial perilymph at a titer of 1e13 vg/mL. The microcatheter is connected to a micromanipulator that infuses approximately 20 uL of the AAV-STRC solution at a rate of approximately 1 uL/min. At the conclusion of the AAV-STRC infusion, the surgeon withdraws the microcatheter and patches the holes in the stapes foot plate and RWM with a gel foam patch. The procedure concludes with replacement of the tympanic membrane flap.

### Example 10: Non-Invasive Prenatal Testing of Maternal Blood to Detect STRC Mutation

Maternal blood samples (20-40 mL) are collected into Cell-free DNA tubes. At least 7 mL of plasma is isolated from each sample via a double centrifugation protocol of 2,000 g for 20 minutes, followed by 3,220 g for 30 minutes, with supernatant transfer following the first spin. cfDNA is isolated from 7-20 mL plasma using a QIAGEN QIAmp® Circulating Nuclei Acid kit and eluted in 45 μL TE buffer. Pure maternal genomic DNA is isolated from the buffy coat obtained following the first centrifugation.

By combining thermodynamic modeling of the assays to select probes with minimized likelihood of probe-probe interaction with amplification approaches described previously (Stiller et al., Genome Res. 19(10):1843-1848, 2009), multiplexing of 11,000 assays can be achieved. Maternal cfDNA and maternal genomic DNA samples are pre-amplified for 15 cycles using 11,000 target-specific assays and an aliquot is transferred to a second PCR reaction of 15 cycles using nested primers. Samples are prepared for sequencing by adding barcoded tags in a third 12-cycle round of PCR. The targets include a genomic sequence in chromosome 15 known to be the site of a stereocilin loss-of-function mutation (Mandelker et al., J. Mol. Diagnostics 16(6):639-647, 2014). The amplicons are then sequenced using an Illumina HiSeq™ sequencer. Genome sequence alignment is performed using commercially available software.

### Example 11: Adenovirus (AAV) Trans-Splicing Strategy

At least two different nucleic acid vectors (e.g., AAV vectors) can be to reconstitute an active stereocilin gene (e.g., a full-length stereocilin gene) within a cell following intermolecular concatemerization and trans-splicing. See, e.g., Yan et al., Proc. Natl. Acad. Sci. U.S.A. 97:12; 6716-6721, 2000, incorporated in its entirety herein.

In some examples, two different nucleic acid vectors will be used. A first nucleic acid vector can include a promoter (e.g., any of the promoters described herein), a first coding sequence that encodes an N-terminal portion of a stereocilin protein positioned 3′ of the promoter (e.g., any of the sizes of a portion of a stereocilin protein described herein and/or any of the N-terminal portions of a stereocilin protein described herein), and a splicing donor signal sequence positioned at the 3′ end of the first coding sequence. A second nucleic acid vector can include a splicing acceptor signal sequence, a second coding sequence that encodes a C-terminal portion of a stereocilin protein (i.e., the entire portion of the stereocilin protein that is not included in the N-terminal portion) positioned at the 3′ end of the splicing acceptor signal sequence (e.g., any of the sizes of a portion of a stereocilin protein described herein and/or any of the C-terminal portions of a stereocilin protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequence of each of the encoded portions does not overlap with the sequence of the other encoded portion, and no single vector of the two different vectors encodes an active stereocilin protein (e.g., a full-length stereocilin protein). When introduced into a mammalian cell (e.g., any of the mammalian cells described herein) splicing occurs between the splicing donor signal sequence and the splicing acceptor signal sequence, thereby forming a recombined nucleic acid that encodes an active stereocilin protein (e.g., a full-length stereocilin protein).

In another example, three different nucleic acid vectors can be used. A first nucleic acid vector can include a portion of a promoter sequence (e.g., any of the promoter sequences described herein), a first coding sequence of a stereocilin gene that encodes a first portion of a stereocilin protein (e.g., any of the stereocilin coding sequences described herein) positioned 3′ of the promoter, and a first splicing donor signal sequence positioned at the 3′ end of the first coding sequence. A second nucleic acid vector can include a first splicing acceptor signal sequence, a second coding sequence of a stereocilin gene that encodes a second portion of a stereocilin protein positioned at the 3′ end of the first splicing acceptor signal sequence, and a second splicing donor signal sequence positioned at the 3′ end of the second coding sequence (e.g., any of the splicing donor signals described herein). A feature of the second nucleic acid vector will be that self-splicing cannot occur (i.e., splicing will not occur between the second splicing donor signal sequence and the first splicing acceptor signal sequence of the second nucleic acid vector). In some embodiments, the splicing donor signal sequence of the first nucleic acid vector and the second splicing donor signal of the second nucleic acid vector are the same (e.g., any of the splicing donor signals described herein or known in the art). In some embodiments, the first splicing donor signal sequence of the first nucleic acid vector and the second splicing donor signal sequence of the second nucleic acid vector are different (e.g., any of the splicing donor signal sequences described herein or known in the art). A third nucleic acid vector will include a second splicing acceptor signal sequence, a third coding sequence of a stereocilin gene that encodes a third portion of a stereocilin protein positioned at the 3′ end of the second splicing acceptor signal sequence, and a polyadenylation sequence positioned at the 3′ end of the third coding sequence (e.g., any of the polyadenylation sequences described herein). In such methods where three nucleic acid vectors are used, the first splicing donor sequence and the first splicing acceptor sequence can assemble together (recombine) and the second slicing donor sequence and the second slicing acceptor sequence can assemble together (recombine), and the portion of stereocilin protein encoded by the first, second, and third coding sequences do not overlap, and when introduced into a mammalian cell (e.g., any of the mammalian cells described herein), splicing occurs between the first splicing donor sequence and the first splicing acceptor sequence, and between the second splicing donor sequence and the second splicing acceptor sequence, to form a recombined nucleic acid that encodes an active stereocilin protein (e.g., a full-length stereocilin protein). Based on the strategies provided above, one skilled in the art would understand how to develop a strategy using four, five, or six different nucleic acid vectors.

In any of the examples of these methods, the amino acid sequence of each of the encoded portions do not overlap, and no single vector encodes an active stereocilin protein (e.g., a full-length stereocilin protein).

Each of the at least two different vectors includes a coding sequence that encodes a different portion of a stereocilin protein, each of the encoded portions can be at least 30 amino acids (e.g., between about 30 amino acids to about 1600 amino acids, or any of the other subranges of this range described herein).

In some embodiments, each of the coding sequences can include at least one exon and at least one intron of SEQ ID NO: 4 (e.g., at least two exons and at least one intron, at least two exons and at least two introns, at least three exons and at least one intron, at least three exons and at least two introns, or at least three exons and at least three introns). In some embodiments, each of the at least two different vectors includes a coding sequence that encodes a different portion of a stereocilin protein, each of the encoded portions can encode up to 80% of the amino acid sequence of SEQ ID NO: 1 (e.g., up to 10%, up to 20%, up to 30%, up to 40%, up to 50%, up to 60%, or up to 70% of SEQ ID NO: 1) such that each of the encoded portions is non-overlapping. In some embodiments, each of the at least two different vectors includes a coding sequence that encodes a different portion of a stereocilin protein, each of the encoded portions encoding up to 80% of the amino acid sequence of SEQ ID NO: 1 (e.g., up to 10%, up to 20%, up to 30%, up to 40%, up to 50%, up to 60%, or up to 70% of SEQ ID NO: 1), such that each of the encoded portions is non-overlapping.

Each of the at least two nucleic acid vectors may further include an inverted terminal repeat (ITR) to allow head-to-tail recombination. The ITR will be subsequently removed via splicing. For example, the ITR could be a palindromic double-D ITR as described in Yan et al., Proc. Natl. Acad. Sci. U.S.A. 97(12):6716-6721, 2000, incorporated in its entirety herein. For example, the ITR could be a AAV serotype-2 ITR as described in Gosh et al., Mol. Ther. 16:124-130, 2008, and Gosh et al., Human Gene Ther. 22: 77-83, 2011. In some embodiments of any of the vectors described herein, the vector can include a 5′ ITR and/or a 3′ ITR. In some embodiments, the 5′ ITR includes or consists of SEQ ID NO: 18. In some embodiments, the 3′ ITR includes or consists of SEQ ID NO: 36.

Non-limiting examples of splicing acceptor and/or donor signal sequences are known in the art. See, e.g., Reich et al., Human Gene Ther. 14(1):37-44, 2003, and Lai et al. (2005) Nat. Biotechnol. 23(11):1435-1439, 2005, 2005. The splicing donor and acceptor signal sequences can be any endogenous intron splicing signal of a gene (e.g., a stereocilin gene). For example, the splicing donor signal sequence can be: 5′-GTAAGTATCAAGGTTACAAGACAGGTTTAAGGAGACCAATAGA AACTGGGCTTGTCGAGACAGAGAAGACTCTTGCGTTTCT-3′ (SEQ ID NO: 6) and the splicing acceptor signal can be 5′-GATAGGCACCTATTGGTCTTACTG ACATCCACTTTGCCTTTCTCTCCACAG-3′ (SEQ ID NO: 7) (see, e.g., Trapani et al., EMBO Mol. Med. 6(2):194-211, 2014). Methods of evaluating splicing and splicing efficiency are known in the art (see, e.g., Lai et al., Nat. Biotechnol. 23(11): 1435-1439, 2005).

### Example 12: Hybrid Vector Trans-Splicing Strategy Using an Alkaline Phosphatase (AP) Highly Recombinogenic Exogenous Gene Region

At least two (e.g., two, three, four, five, or six) different nucleic acid vectors (e.g., AAV vectors) can also be used in any of the methods described herein to reconstitute an active stereocilin gene (e.g., a full-length stereocilin gene) within a cell following intermolecular concatemerization, marker gene-mediated recombination, and trans-splicing. This strategy is a hybrid strategy as it will include homologous recombination and/or trans-splicing. See, e.g., Gosh et al., Mol. Ther. 16: 124-130, 2008; Gosh et al., Human Gene Ther. 22: 77-83, 2011; and Duan et al., Mol. Ther. 4: 383-391, 2001, each incorporated in its entirety herein. As used herein, a detectable marker gene can be a highly recombinogenic DNA sequence that will allow for coding sequence-independent recombination. An non-limiting example of a detectable marker gene is an alkaline phosphatase (AP) gene. For example, the detectable marker gene can be the middle one-third of the human placental AP complementary DNA, which is 872 bp in length (see, e.g., Gosh et al., 2008). At least two different nucleic acid vectors will contain a detectable marker gene (e.g., any of the detectable marker genes described herein). Since the hybrid vector will be constructed based on a trans-splicing vector as described in Example 10, an active stereocilin gene (e.g., a full-length stereocilin gene) may be reconstituted using either ITR-mediated recombination and trans-splicing or detectable marker gene-mediated (e.g., AP-gene mediated) recombination and trans-splicing. After trans-splicing, an active stereocilin gene (e.g., a full-length stereocilin gene) will be reconstituted in the genomic DNA of a mammalian cell (e.g., any mammalian cell described herein).

In one example, two different nucleic acid vectors will be used. A first nucleic acid vector can include a promoter (e.g., any of the promoters described herein), a first coding sequence that encodes an N-terminal portion of a stereocilin protein positioned 3′ of the promoter (e.g., any of the sizes of a portion of a stereocilin protein described herein and/or any of the N-terminal portions of a stereocilin protein described herein), a splicing donor signal sequence positioned at the 3′ end of the first coding sequence, and a first detectable marker gene positioned 3′ of the splicing donor signal sequence. A second nucleic acid vector can include a second detectable marker gene, a splicing acceptor signal sequence positioned 3′ of the second detectable marker gene, a second coding sequence that encodes a C-terminal portion of a stereocilin protein positioned at the 3′ end of the splicing acceptor signal sequence (e.g., any of the sizes of a portion of a stereocilin protein described herein and/or any of the C-terminal portions of a stereocilin protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequence of each of the encoded portions do not overlap, and no single vector of the two different vectors encodes an active stereocilin protein (e.g., a full-length stereocilin protein). When introduced into a mammalian cell (e.g., any of the mammalian cells described herein) splicing occurs between the splicing donor signal sequence and the splicing acceptor signal sequence, thereby forming a recombined nucleic acid that encodes an active stereocilin protein (e.g., a full-length stereocilin protein).

In another example, three different nucleic acid vectors can be used. A first nucleic acid vector can include a portion of promoter sequence (e.g., any of the promoter sequences described herein), a first coding sequence of a stereocilin gene that encodes a first portion of a stereocilin protein (e.g., any of the stereocilin coding sequences described herein) positioned 3′ of the promoter, a first splicing donor signal sequence positioned at the 3′ end of the first coding sequence, and a first detectable marker gene. A second nucleic acid vector can include a second detectable marker gene, a first splicing acceptor signal sequence positioned 3′ of the second detectable marker gene, a second coding sequence of a stereocilin gene that encodes a second portion of a stereocilin protein positioned at the 3′ end of the first splicing acceptor signal sequence, a second splicing donor signal sequence positioned at the 3′ end of the second coding sequence (e.g., any of the splicing donor signals described herein), and a third detectable marker gene. A feature of the second nucleic acid vector will be that self-splicing cannot occur (i.e., splicing will not occur between the second splicing donor signal sequence and the first splicing acceptor signal sequence of the second nucleic acid vector). In some embodiments, the splicing donor signal sequence of the first nucleic acid vector and the second splicing donor signal of the second nucleic acid vector are the same (e.g., any of the splicing donor signals described herein or known in the art). In some embodiments, the first splicing donor signal sequence of the first nucleic acid vector and the second splicing donor signal sequence of the second nucleic acid vector are different (e.g., any of the splicing donor signal sequences described herein or known in the art). A third nucleic acid vector can include a fourth detectable marker gene, a second splicing acceptor signal sequence positioned 3′ of the fourth detectable marker gene, a third coding sequence of a stereocilin gene that encodes a third portion of a stereocilin protein positioned at the 3′ end of the second splicing acceptor signal sequence, and a polyadenylation sequence positioned at the 3′ end of the third coding sequence (e.g., any of the polyadenylation sequences described herein). In such methods where three nucleic acid vectors are used, the first splicing donor sequence and the first splicing acceptor sequence can assemble together (recombine) and the second slicing donor sequence and the second slicing acceptor sequence can assemble together (recombine), and the portion of stereocilin protein encoded by the first, second, and third coding sequences do not overlap, and when introduced into a mammalian cell (e.g., any of the mammalian cells described herein), splicing occurs between the first splicing donor sequence and the first splicing acceptor sequence, and between the second splicing donor sequence and the second splicing acceptor sequence, to form a recombined nucleic acid that encodes an active stereocilin protein (e.g., a full-length stereocilin protein). As can be appreciated in the art when three nucleic acid vectors are used, two of the at least two different nucleic acid vectors can include a detectable marker gene (e.g., an AP marker gene) and one of the at least two different nucleic acid vectors may include a splicing acceptor signal sequence that is complementary to a splicing donor signal sequence in a nucleic acid vector that includes a detectable marker gene. For example, in some embodiments, the first and second nucleic acid vectors can include a detectable marker gene (e.g., an AP marker gene), and the third nucleic acid vector will include a splicing acceptor signal sequence that is complementary to the splicing donor signal sequence in the second nucleic acid vector, and the third nucleic acid vector will not include a detectable marker gene (e.g., an AP marker gene). In other examples, the second and third nucleic acid vector can include a detectable marker gene (e.g., an AP marker gene), and the first nucleic acid vector will include a splicing donor signal sequence that is complementary to the splicing acceptor signal sequence in the second nucleic acid vector and the first nucleic acid vector will not include a detectable marker gene (e.g., an AP marker gene).

Based on the strategies provided above, one skilled in the art would understand how to develop a strategy using four, five, or six vectors.

The coding sequences provided in the at least two nucleic acid vectors (e.g., two, three, four, five or six) will not be overlapping. Each of the at least two different vectors can include a coding sequence that encodes a different portion of a stereocilin protein, each of the encoded portions being, e.g., at least 30 amino acids (e.g., about 30 amino acids to about 1600 amino acids, or any of the other subranges of this range described herein).

In some embodiments, each of the at least two different vectors includes a coding sequence that encodes a different portion of a stereocilin protein, each of the encoded portions encoding at least one exon and at least one intron of SEQ ID NO: 4 (e.g., at least two exons and at least one intron, at least two exons and at least two introns, at least three exons at least one intron, at least three exons and at least two introns, or at least three exons and at least three introns). In some embodiments, each of the at least two different vectors include a coding sequence that encodes a different portion of a stereocilin protein, each of the encoded portions encoding up to 80% of SEQ ID NO: 1 (e.g., up to 10%, up to 20%, up to 30%, up to 40%, up to 50%, up to 60%, up to 70% of SEQ ID NO:1) such that each of the encoded portions is non-overlapping. In some embodiments, each of the at least two different vectors include a coding sequence that encodes a different portion of a stereocilin protein, each of the encoded portions encoding up to 80% of SEQ ID NO: 1 (e.g., up to 10%, up to 20%, up to 30%, up to 40%, up to 50%, up to 60%, or up to 70% of SEQ ID NO:1), such that each of the encoded portions is non-overlapping.

As described in Example 10, each of the at least two nucleic acid vectors may further include an inverted terminal repeat (ITR) to allow head-to-tail recombination. The ITR will be subsequently removed via splicing. Examples of ITRs and splicing acceptor and/or donor signal sequences are known in the art and have been described in Example 10.

### Example 13: Hybrid Vector Trans-Splicing Strategy Using a F1 Phage Highly Recombinogenic Exogenous Gene Region (AK)

At least two (e.g., two, three, four, five, or six) different nucleic acid vectors (e.g., AAV vectors) can also be used in any of the methods described herein to reconstitute an active stereocilin gene (e.g., a full-length stereocilin gene) within a cell following intermolecular concatemerization, marker gene-mediated recombination, and trans-splicing. This strategy is a hybrid strategy as it will include homologous recombination and/or trans-splicing. See, e.g., Trapani et al., EMBO Mol. Med. 6(2):194-211, 2014, incorporated in its entirety herein. As used herein, an F1 phage recombinogenic region (AK) will be used to allow coding sequence-independent recombination. The F1 phage recombinogenic region may be a 77 bp recombinogenic region from the F1 phage genome as described in Trapani et al. (2014). At least two different nucleic acid vectors will contain an F1 phage recombinogenic region. Since the hybrid vector will be constructed based on a trans-splicing vector as described in Example 10, a nucleic acid encoding an active stereocilin protein (e.g., a full-length stereocilin protein) may be generated using either ITR-mediated recombination and trans-splicing or F1 phage recombinogenic region-induced recombination and trans-splicing. After trans-splicing, a nucleic acid encoding an active stereocilin protein (e.g., a full-length stereocilin protein) will be generated in a mammalian cell (e.g., any of the mammalian cells described herein).

In one example, two different nucleic acid vectors will be used. A first nucleic acid vector can include a promoter (e.g., any of the promoters described herein), a first coding sequence that encodes an N-terminal portion of a stereocilin protein positioned 3′ of the promoter (e.g., any of the sizes of a portion of a stereocilin protein described herein and/or any of the N-terminal portions of a stereocilin protein described herein), a splicing donor signal sequence positioned at the 3′ end of the first coding sequence, and an F1 phage recombinogenic region positioned 3′ of the splicing donor signal sequence. A second nucleic acid vector can include an F1 phage recombinogenic region, a splicing acceptor signal sequence positioned 3′ of the F1 phage recombinogenic region, a second coding sequence that encodes a C-terminal portion of a stereocilin protein positioned at the 3′ end of the splicing acceptor signal sequence (e.g., any of the sizes of a portion of a stereocilin protein described herein and/or any of the C-terminal portions of a stereocilin protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequence of each of the encoded portions do not overlap, and no single vector of the two different vectors encodes an active stereocilin protein (e.g., a full-length stereocilin protein). When introduced into a mammalian cell (e.g., any of the mammalian cells described herein) splicing occurs between the splicing donor signal sequence and the splicing acceptor signal sequence, thereby forming a recombined nucleic acid that encodes an active stereocilin protein (e.g., a full-length stereocilin protein).

In another example, three different nucleic acid vectors will be used. A first nucleic acid vector can include a portion of promoter sequence (e.g., any of the promoter sequences described herein), a first coding sequence of a stereocilin gene that encodes a first portion of a stereocilin protein (e.g., any of the stereocilin coding sequences described herein) positioned 5′ of the promoter, a first splicing donor signal sequence positioned at the 3′ end of the first coding sequence, and an F1 phage recombinogenic region. A second nucleic acid vector can include an F1 phage recombinogenic region, a first splicing acceptor signal sequence positioned 3′ of the F1 phage recombinogenic region, a second coding sequence of a stereocilin gene that encodes a second portion of a stereocilin protein positioned at the 3′ end of the first splicing acceptor signal sequence, a second splicing donor signal sequence positioned at the 3′ end of the second coding sequence (e.g., any of the splicing donor signals described herein), and an F1 phage recombinogenic region. A feature of the second nucleic acid vector will be that self-splicing cannot occur (i.e., splicing will not occur between the second splicing donor signal sequence and the first splicing acceptor signal sequence of the second nucleic acid vector). In some embodiments, the splicing donor signal sequence of the first nucleic acid vector and the second splicing donor signal of the second nucleic acid vector are the same (e.g., any of the splicing donor signals described herein or known in the art). In some embodiments, the first splicing donor signal sequence of the first nucleic acid vector and the second splicing donor signal sequence of the second nucleic acid vector are different (e.g., any of the splicing donor signal sequences described herein or known in the art). A third nucleic acid vector can include an F1 phage recombinogenic region, a second splicing acceptor signal sequence positioned 3′ of the F1 phage recombinogenic region, a third coding sequence of a stereocilin gene that encodes a third portion of a stereocilin protein positioned at the 3′ end of the second splicing acceptor signal sequence, and a polyadenylation sequence positioned at the 3′ end of the third coding sequence (e.g., any of the polyadenylation sequences described herein). In such methods where three nucleic acid vectors are used, the first splicing donor sequence and the first splicing acceptor sequence can assemble together (recombine) and the second slicing donor sequence and the second slicing acceptor sequence can assemble together (recombine), and the portion of stereocilin protein encoded by the first, second, and third coding sequences do not overlap, and when introduced into a mammalian cell (e.g., any of the mammalian cells described herein), splicing occurs between the first splicing donor sequence and the first splicing acceptor sequence, and between the second splicing donor sequence and the second splicing acceptor sequence, to form a recombined nucleic acid that encodes an active stereocilin protein (e.g., a full-length stereocilin protein). As can be appreciated in the art when three nucleic acid vectors are used, two of the different nucleic acid vectors can include an F1 phage recombinogenic region and one of the different nucleic acid vectors may include a splicing acceptor signal sequence that is complementary to a splicing donor signal sequence in a nucleic acid vector that includes an F1 phage recombinogenic region. For example, in some embodiments, the first and second nucleic acid vectors can include an F1 phage recombinogenic region, and the third nucleic acid vector will include a splicing acceptor signal that is complementary to the splicing donor signal sequence in the second nucleic acid vector, and the third nucleic acid vector will not include an F1 phage recombinogenic region (e.g., an AP marker gene). In other examples, the second and third nucleic acid vector can include an F1 phage recombinogenic region and the first nucleic acid vector will include a splicing donor signal sequence that is complementary to the splicing acceptor signal sequence in the second nucleic acid vector and the first nucleic acid vector will not include an F1 phage recombinogenic region. Based on the strategies provided above, one skilled in the art would understand how to develop a strategy using four, five, or six vectors.

The coding sequences provided in each of the at least two nucleic acid vectors (e.g., two, three, four, five or six) will not be overlapping. Each of the at least two different vectors include a coding sequence that encodes a different portion of a stereocilin protein, each of the encoded portions being at least 30 amino acids (e.g., about 30 amino acids to about 1600 amino acids, or any of the subranges of this range described herein).

In some embodiments, each of the at least two different vectors include a coding sequence that encodes a different portion of a stereocilin protein, each of the encoded portions encoding at least one exon and at least one intron of SEQ ID NO: 4 (e.g., at least two exons and at least one intron, at least two exons and at least two introns, at least three exons at least one intron, at least three exons and at least two introns, or at least three exons and at least three introns). In some embodiments, each of the at least two different vectors includes a coding sequence that encodes a different portion of a stereocilin protein, each of the encoded portions encoding up to 80% of SEQ ID NO: 1 (e.g., up to 10%, up to 20%, up to 30%, up to 40%, up to 50%, up to 60%, or up to 70% of SEQ ID NO: 1) such that each of the encoded portions is non-overlapping. In some embodiments, each of the at least two different vectors include a coding sequence that encodes a different portion of a stereocilin protein, each of the encoded portions encoding up to 80% of SEQ ID NO: 1 (e.g., up to 10%, up to 20%, up to 30%, up to 40%, up to 50%, up to 60%, or up to 70% of SEQ ID NO: 1), such that each of the encoded portions is non-overlapping.

As described in Example 10, each of the at least two nucleic acid vectors may further include an inverted terminal repeat (ITR) to allow head-to-tail recombination. The ITR will be subsequently removed via splicing. Examples of ITRs and splicing acceptor and/or donor signals are known in the art and have been described in Example 10.

### Example 14: CRISPR-Cas9-Mediated Knockin STRC Pseudogene Lacking Undesired Residues

The genomic sequences of the functional STRC gene and the STRC pseudogene are 98.9% identical and the coding regions are 99.6% homologous (Francey et al., Am. J. Med. Genet. A. 158A(2):298-308, 2012). Francey et al. determined that 56 bp between exons 16 and 28 were divergent. Verpy et al. determined that a nonsense mutation in exon 20 of the STRC pseudogene rendered the protein expressed from the pseudogene inactive (Verpy et al., Nat. Genet. 29:345-349, 2001). Expression of a functional STRC gene may be obtained using a composition including a CRISPR-Cas9 vector to correct (e.g., modify, replace) the nonsense mutation in exon 20 of the STRC pseudogene (SEQ ID NO: 5) to generate a sequence encoding an active stereocilin protein (e.g., a full-length stereocilin protein) in a mammalian cell, and thereby treat non-syndromic sensorineural hearing loss in a subject in need thereof. Various CRISRP-Cas9 techniques that can be used to introduce a mutation into an endogenous gene are also known in the art. See, e.g., Merkle et al., Cell Rep. 11(6):875-883, 2015; He et al., Nucleic Acids Res. 44(9):e85, 2016; Wang et al., Mol. Ther. Nucleic Acids 5: e396, 2016, and Verma et al., Methods Mol. Biol. 1513:119-140, 2017. For example, a composition including a Cas9 nuclease (e.g., Streptococcus pyogenes) and a guide RNA that includes at the 5′ end of the guide RNA a complementary region consisting of about 20 nucleotides (e.g., 20, 21 or 22) that is complementary to about 20 (e.g., 20, 21 or 22) consecutive nucleotides within positions 13955-14151 of SEQ ID NO:5 can be used in CRISPR/Cas9 RNA-guided genome editing to remove a nonsense mutation at a predetermined site in an endogenous stereocilin pseudogene sequence of SEQ ID NO: 5; and when introduced into a mammalian cell, a nucleic acid encoding an active stereocilin protein (e.g., a full-length stereocilin protein) is reconstituted at the locus of the stereocilin pseudogene.

### Example 15: AAV-mediated Antisense Oligonucleotide Delivery

An additional approach to generate a functional STRC transcript from the STRC pseudogene will include using AAV-mediated antisense oligonucleotide delivery via exon skipping. See, e.g., Chamorro et al., Mol. Ther. Nucleic Acids 5: e307, 2016; Kawecka et al., Curr. Gene Ther. 15(4):395-415, 2015; Bremer et al., Mol. Ther. 5:e3379, 2016; Tei et al., Biochem. Biophys. Res. Commun. 461(3):481-486, 2015; and Jirka et al., Nucleic Acid Ther. 24(1):25-36, 2014. As shown in Bremer et al., antisense oligonucleotides can be designed to bind to a particular exon of interest and lead to in-frame exon skipping at the RNA level, thereby restoring gene function.

In the case of a stereocilin gene, a study can be conducted to determine the eligibility of exon 20 of the STRC pseudogene as a target for exon skipping, using techniques such as those used in Bremer et al. (2016). Upon determination of the eligibility of exon 20 of the STRC pseudogene, antisense oligonucleotides with sequences corresponding to 17-23 base pairs in exon 20 can be generated and analyzed for their melting temperature, GC-content, off-target binding and binding energy. In some embodiments, the oligonucleotides will be synthesized as 2′-O-methyl phosphorothioate antisense oligonucleotides and analyzed for exon skipping by RT-PCR. To determine de novo expression of the corrected STRC pseudogene in a cell, cells (e.g., a mammalian cell, a human cell, or any cell described herein) will be transfected within a nucleic acid vector (e.g., an AAV vector) including an antisense oligonucleotide of a stereocilin gene (e.g., an antisense oligonucleotide corresponding to part of exon 20 of a STRC pseudogene) using known transfection methods in the art, and expression of the corrected STRC pseudogene will be determined using methods such as RT-PCR, immunofluorescence, or Western blot, as well as functional biochemical assays for a stereocilin protein. For example, in some embodiments, a composition including a nucleic acid vector that includes an antisense oligonucleotide that is complementary to at least a portion (e.g., at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 45 contiguous nucleotides, at least 50 contiguous nucleotides, at least 55 contiguous nucleotides, at least 60 contiguous nucleotides, at least 65 contiguous nucleotides, at least 70 contiguous nucleotides, at least 75 contiguous nucleotides, at least 80 contiguous nucleotides, at least 85 contiguous nucleotides, at least 90 contiguous nucleotides, at least 95 contiguous nucleotides, at least 100 contiguous nucleotides, at least 105 contiguous nucleotides, at least 110 contiguous nucleotides, at least 115 contiguous nucleotides, at least 120 contiguous nucleotides, at least 125 contiguous nucleotides, at least 130 contiguous nucleotides, at least 135 contiguous nucleotides, at least 140 contiguous nucleotides, at least 145 contiguous nucleotides, at least 150 contiguous nucleotides, at least 155 contiguous nucleotides, at least 160 contiguous nucleotides, at least 165 contiguous nucleotides, at least 170 contiguous nucleotides, at least 175 contiguous nucleotides, at least 180 contiguous nucleotides, at least 185 contiguous nucleotides, or at least 190 contiguous nucleotides) of the nucleotide sequence of SEQ ID NO: 5 within positions 13955-14151 will be introduced into a mammalian cell and a functional stereocilin protein will be generated. One skilled in the art will appreciate that an exon 20 antisense oligonucleotide (e.g., an antisense oligonucleotide of SEQ ID NO: 5 complementary to sequence within the region defined by positions 13955-14141) will be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range of numbers having one of those numbers at the low end and a higher number at the high end. In some embodiments, the antisense oligonucleotides of exon 20 will be 20 nucleotides in length. In some embodiments, the antisense oligonucleotides of exon 20 (e.g., an antisense oligonucleotide of SEQ ID NO: 5 complementary to sequence comprising of positions 13955-14141) will be 15 to 30 nucleotides in length. In some embodiments, the antisense oligonucleotides of exon 20 (e.g., an antisense oligonucleotide of SEQ ID NO: 5) will be at least 80% identical (e.g., at least 85%, at least 90%, at least 95%, at least 97%, at least 98%, at least 99%, 80%, 81%, 82%, 83%, 84%, 85%, 86%, 87%, 88%, 89%, 90%, 91%, 92%, 93%, 94%, 95%, 96%, 97%, 98%, or 99%) to the complement of a portion of the region defined by positions 13955-14151 of SEQ ID NO: 5.

### Example 16. STRC Expression in HEK293FT Cells

HEK293FT cells were seeded overnight at 7E4 cells/well in a 24-well format and were transfected with exemplary single or dual STRC vectors using jetprime reagent. 72 hours post-transfection, HEK293FT cells were harvested with 200 μL RIPA buffer/well. Thirty microliters of samples were loaded into each well onto a 4-12% Bis-Tris protein gel. STRC protein expression was determined using a STRC polyclonal antibody. Beta-actin (ACTB) was used as a loading control.

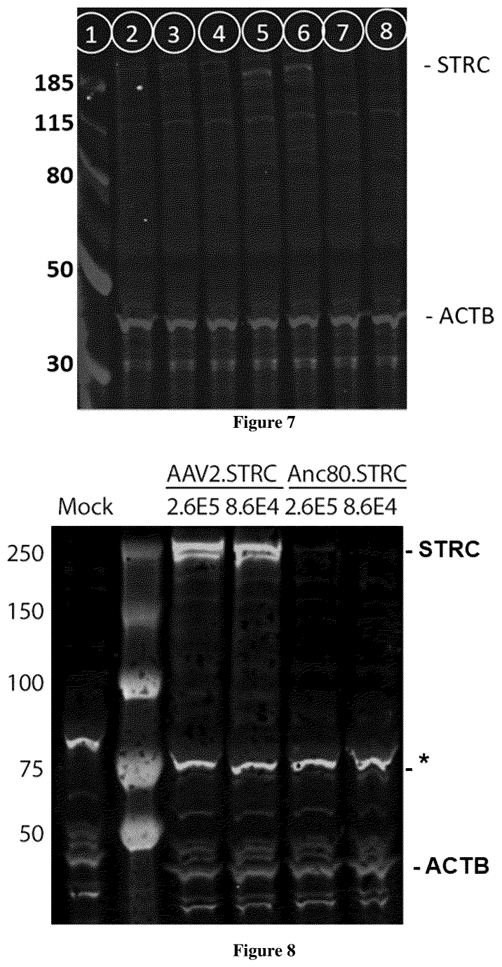

As shown in FIG. 7, the dual STRC vectors using CBA/CAG promoter (pITR-205 plus pITR-202 and pITR-205 plus pITR-202GFP) resulted in more intense full-length STRC protein in HEK293FT cells. This result confirmed that STRC protein can be expressed by exemplary STRC vectors described herein.

Next, HEK293FT cells were transduced with dual STRC vectors at different MOIs and were seeded overnight at 1.2E5 cells/well in a 24-well format with etoposide reagent. HEK293FT cells were harvested 72 hours post-infection with 200 μL RIPA buffer/well. As shown in FIG. 8, HEK293FT cells transduced with AAV2 vectors showed higher expression of full-length STRC protein as compared to HEK293FT cells transduced with AAV.Anc80 vector.

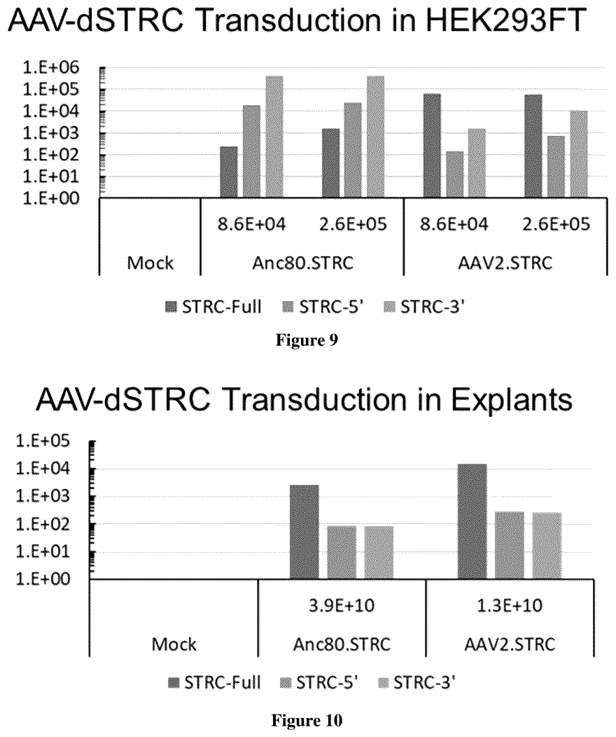

FIG. 9 showed high levels of STRC RNA expression relative to GAPDH in HEK293FT cells infected with either Anc80.STRC (at MOI 8.6E4 and MOI 2.6E5) or AAV2.STRC (at MOI 8.6E4 and MOI 2.6E5).

### Example 17. STRC Expression in P1-3 Cochlear Mouse Explants

P2 cochlear from WT mice were infected after plating and were harvested for RNA and immunofluorescence 72 hours after infection. As shown in FIG. 10, full-length STRC was efficiently expressed in cochlear explants infected with Anc80.STRC at MOI 3.9E10 and AAV2.STRC at MOI 1.3E10, respectively.

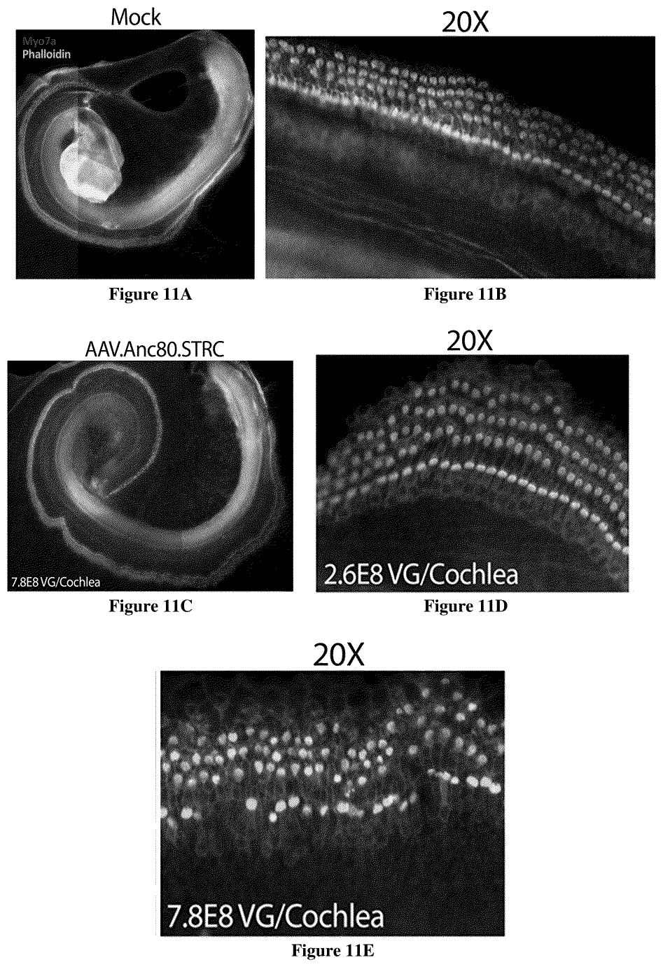

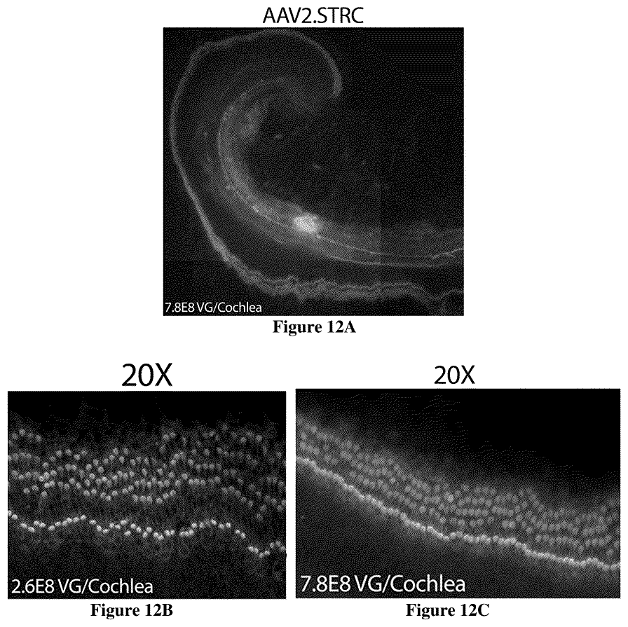

As shown in FIGS. 11A-E and 12A-C, outer hair cells and inner cells of P1-3 cochlear explants expressed Myo7a and phalloidin when transduced with AAV.Anc80.dSTRC (7.8E VG/cochlea, 2.6E8 VG/cochlea) and AAV2.dSTRC (7.8E8 VG/cochlea and 2.6E8 VG/cochlea). FIGS. 11A and 11B showed the natural cochlear structure, in which Myo7a is expressed in both outer and inner hair cells, while phalloidin is expressed exclusively in the inner hair cells. A similar staining pattern was observed in P1-3 cochlear explants transduced with AAV.Anc80.STRC (FIGS. 11C-E), and in P1-3 cochlear explants transduced with AAV2.STRC (FIGS. 12A-C).

Transfection with either vector did not disrupt the structural integrity of outer hair cells or inner hair cells of the cochlea. Comparison between non-infected with AAV2.dSTRC and AAV.Anc80.dSTRC infected explants showed robust numbers of living hair cells suggesting the relative safety of the vectors. Thus, FIGS. 11A-E and 12A-C showed lack of toxicity of STRC vectors with viable and organized outer hair cells, inner hair cells, and stereociliary bundles.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1.-66. (canceled)

67. A method of increasing expression of a full-length stereocilin protein in a human cell, the method comprising introducing a composition into the human cell, wherein the composition comprises a first nucleic acid vector and a second nucleic acid vector, wherein:
the first nucleic acid vector comprises (i) a promoter and (ii) a 5′ coding sequence of a stereocilin protein according to SEQ ID NO: 25, and
the second nucleic acid vector comprises a 3′ coding sequence of a stereocilin protein according to SEQ ID NO: 28.

68. The method of claim 67, wherein the human cell is a human cochlear outer hair cell.

69. The method of claim 67, wherein the human cell has previously been determined to have a defective stereocilin gene.

70. The method of claim 67, wherein each of the first nucleic acid vector and the second nucleic acid vector is a plasmid, a transposon, a cosmid, or a viral vector.

71. The method of claim 67, wherein each of the first nucleic acid vector and the second nucleic acid vector is a viral vector selected from an adeno-associated virus (AAV) vector, an adenovirus vector, a lentivirus vector, or a retrovirus vector.

72. The method of claim 71, wherein the viral vector is an AAV vector.

73. The method of claim 67, wherein the first nucleic acid vector further comprises a Kozak sequence.

74. The method of claim 67, wherein the first nucleic acid vector comprises a promoter that is an inducible promoter, a constitutive promoter, or a tissue-specific promoter.

75. The method of claim 67, wherein the second nucleic acid vector further comprises a poly(dA) sequence.

76. A method of treating non-symptomatic sensorineural hearing loss in a human subject in need thereof, wherein the human subject has been identified as having a defective stereocilin gene, the method comprising:
administering a therapeutically effective amount of a composition into the cochlea of the human subject, wherein the composition comprises a first nucleic acid vector and a second nucleic acid vector, wherein:
the first nucleic acid vector comprises (i) a promoter and (ii) a 5′ coding sequence of a stereocilin protein according to SEQ ID NO: 25, and
the second nucleic acid vector comprises a 3′ coding sequence of a stereocilin protein according to SEQ ID NO: 28.

77. The method of claim 76, further comprising, prior to the administering step, determining that the human subject has a defective stereocilin gene.

78. The method of claim 76, wherein each of the first nucleic acid vector and the second nucleic acid vector is a plasmid, a transposon, a cosmid, or a viral vector.

79. The method of claim 76, wherein each of the first nucleic acid vector and the second nucleic acid vector is a viral vector selected from an adeno-associated virus (AAV) vector, an adenovirus vector, a lentivirus vector, or a retrovirus vector.

80. The method of claim 79, wherein the viral vector is an AAV vector.

81. The method of claim 76, wherein the first nucleic acid vector further comprises a Kozak sequence.

82. The method of claim 76, wherein the first nucleic acid vector comprises a promoter that is an inducible promoter, a constitutive promoter, or a tissue-specific promoter.

83. The method of claim 76, wherein the second nucleic acid vector further comprises a poly(dA) sequence.

84. A kit comprising a composition that comprises a first nucleic acid vector and a second nucleic acid vector, wherein:
the first nucleic acid vector comprises (i) a promoter and (ii) a 5′ coding sequence of a stereocilin protein according to SEQ ID NO: 25, and
the second nucleic acid vector comprises a 3′ coding sequence of a stereocilin protein according to SEQ ID NO: 28.

85. A kit of claim 84, further comprising a pre-loaded syringe or vial comprising the composition.
